(19) United States
(12) Patent Application Publication
(10) Pub. No.: US 2022/0273715 A1
(43) Pub. Date: Sep. 1, 2022

(54) COMPOSITIONS AND METHODS FOR SEQUENTIAL STACKING OF NUCLEIC ACID SEQUENCES INTO A GENOMIC LOCUS

(71) Applicant: Precision BioSciences, Inc., Durham, NC (US)

(72) Inventors: Joann Hux, Scotland Neck, NC (US); James Jefferson Smith, Morrisville, NC (US)

(73) Assignee: Precision BioSciences, Inc., Durham, NC (US)

(21) Appl. No.: 17/630,064

(22) PCT Filed: Jul. 24, 2020
(86) PCT No.: PCT/US2020/043628

§ 371 Date: Jan. 25, 2022

Related U.S. Application Data
(60) Provisional application No. 62/878,529, filed on Jul. 25, 2019.

(51) Int. Cl. A61K 35/17; C12N 5/0783; C12N 9/22; C12N 15/62; C12N 15/86; C12N 15/90; A61K 38/17; A61P 35/00

(52) U.S. Cl. CPC A61K 35/17; C12N 5/0636; C12N 9/22; C12N 15/625; C12N 15/86; C12N 15/907; A61K 38/1774; A61P 35/00; C12N 2750/14143; C12N 2800/80; C12N 2800/40

(57) ABSTRACT

The present invention encompasses compositions and methods for the sequential stacking of donor nucleic acids into a single genomic locus within a cell to allow for the introduction of relatively long nucleic sequences. This allows for insertion into the genome of a donor nucleic acid sequence that exceeds the packaging capacity of a single adeno-associated viral vector.

### FIELD OF THE INVENTION

The invention relates to the field of virology, molecular biology, and recombinant nucleic acid technology. In particular, the invention relates to compositions comprising vectors for sequential incorporation of nucleic acid sequences into a genome.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jul. 24, 2020, is named P1090.70041WO00-SEQ-EPG, and is 9,838 bytes in size.

### BACKGROUND OF THE INVENTION

Viral vectors are used to deliver genetic material into host cells for various applications, including gene editing and gene therapy. Adeno-associated virus (AAV) is a small, replication-defective virus belonging to the family of Parvoviridae that has shown promise for use in delivering genes to both dividing and quiescent cells. AAV is not pathogenic to humans, only replicates in the presence of a helper virus, and transgene expression by rAAV is potentially long lasting.

Transduction using AAV vectors can be used to supply cargo DNA for insertion by homologous directed recombination (HDR) into double-stranded breaks generated by site-specific engineered nucleases. For efficient insertion to occur, the desired DNA cargo is typically flanked by sequences homologous to the chromosomal location of the double stranded DNA break, including half of the nuclease recognition sequence. The size of the AAV DNA cargo, however, is limited by the necessity for DNA encoding flanking homology for HDR to occur and the ˜4.7 kb cloning capacity of a single AAV.

Thus, there remains a need for a mechanism by which to increase the amount of DNA that can be successfully incorporated into a targeted genomic location during gene editing.

### SUMMARY OF THE INVENTION

The present invention provides compositions comprising one or more polynucleotides, along with one or more engineered nucleases or nucleic acids encoding the same, that are useful for producing genetically-modified cells. For example, the present invention provides for the introduction into a cell's genome of a long transgene that exceeds the packaging capacity of a single recombinant virus, such as an adeno-associated virus (AAV), by utilizing a first recombinant virus comprising a first portion of the transgene and a second recombinant virus comprising a second portion of the transgene. The introduction of the two viruses can occur ex vivo, where cells can be isolated and contacted with multiple vectors in culture. The resultant genetically-modified cells can then be administered to a subject. In other embodiments, the introduction of the polynucleotides comprising heterologous nucleic acid molecules can occur in vivo where the one or two polynucleotides (e.g., AAV) are administered to a subject, along with one or more nucleic acids encoding one or more engineered nucleases, and in those cells comprising all three components, the heterologous nucleic acid molecules are inserted into the genome.

Thus, in one aspect, the invention provides a composition comprising: (a) a first polynucleotide comprising a first nucleic acid sequence comprising: (i) a first donor nucleic acid sequence comprising a first nuclease recognition sequence for a first engineered nuclease; and (ii) a first homology region positioned 3′ downstream of the first nuclease recognition sequence; (b) a second polynucleotide comprising a second nucleic acid sequence comprising: (i) a 5′ homology arm having homology to at least a portion of the first donor nucleic acid sequence and to a 5′ portion of the first nuclease recognition sequence; (ii) a 3′ homology arm having homology to a 3′ portion of the first nuclease recognition sequence and to the first homology region; and (iii) a second donor nucleic acid sequence positioned between the 5′ homology arm and the 3′ homology arm; and (c) one or more engineered nucleases, or one or more nucleic acids encoding the one or more engineered nucleases, comprising the first engineered nuclease.

In some embodiments, the first nuclease recognition sequence is positioned at the 3′ end of the first donor nucleic acid sequence.

In some embodiments, the one or more engineered nucleases is an engineered meganuclease, a TALEN, a compact TALEN, a zinc finger nuclease, a CRISPR system nuclease, or a megaTAL. In certain embodiments, the one or more engineered nucleases is an engineered meganuclease.

In certain embodiments, the first engineered nuclease is not capable of binding and cleaving an endogenous nuclease recognition sequence normally present in the genome of a eukaryotic cell of interest. In some embodiments, the one or more engineered nucleases comprises a second engineered nuclease capable of binding and cleaving the endogenous nuclease recognition sequence.

In certain embodiments, the first engineered nuclease is capable of binding and cleaving the first nuclease recognition sequence and an endogenous nuclease recognition sequence normally present in the genome of a eukaryotic cell of interest. In some embodiments, the first nuclease recognition sequence is identical to the endogenous nuclease recognition sequence.

In certain embodiments, the endogenous nuclease recognition sequence is within a T cell receptor (TCR) alpha gene. In certain embodiments, the endogenous nuclease recognition sequence is within a TCR beta gene. In some embodiments, the endogenous nuclease recognition sequence is within a TCR alpha constant (TRAC) gene. In some embodiments, the endogenous nuclease recognition sequence is within a TCR beta constant (TRBC) gene. In particular embodiments, the first nuclease recognition sequence comprises SEQ ID NO: 1.

In certain embodiments, the one or more nucleic acids encoding the one or more engineered nucleases are mRNA. In some embodiments, the one or more nucleic acids encoding the one or more engineered nucleases are comprised within one or more nuclease adeno-associated viruses (AAVs).

In certain embodiments, the second donor nucleic acid sequence does not comprise a second nuclease recognition sequence. In some embodiments, the second donor nucleic acid does not comprise a 5′ portion of a nuclease recognition sequence that is capable of pairing with the 3′ portion of the first nuclease recognition sequence to generate a second nuclease recognition sequence. In some embodiments, the second donor nucleic acid does not comprise a 3′ portion of a nuclease recognition sequence that is capable of pairing with the 5′ portion of the first nuclease recognition sequence to generate a second nuclease recognition sequence.

In certain embodiments, the second donor nucleic acid sequence comprises a second nuclease recognition sequence. In some embodiments, the second donor nucleic acid sequence comprises a 5′ portion of a nuclease recognition sequence capable of pairing with the 3′ portion of the first nuclease recognition sequence to generate a second nuclease recognition sequence. In some embodiments, the second donor nucleic acid sequence comprises a 3′ portion of a nuclease recognition sequence capable of pairing with the 5′ portion of the first nuclease recognition sequence to generate a second nuclease recognition sequence.

In certain embodiments, the first engineered nuclease is not capable of binding and cleaving the second nuclease recognition sequence. In some embodiments, the second nuclease recognition sequence is not identical to the first nuclease recognition sequence.

In certain embodiments, the one or more engineered nucleases comprises a third engineered nuclease capable of binding and cleaving the second nuclease recognition sequence.

In certain embodiments, the first engineered nuclease is capable of binding and cleaving the first nuclease recognition sequence, the second nuclease recognition sequence, and the endogenous nuclease recognition sequence. In some embodiments, the first nuclease recognition sequence, the second nuclease recognition sequence, and the endogenous nuclease recognition sequence are identical.

In certain embodiments, the first donor nucleic acid sequence comprises a first transgene.

In certain embodiments, the first donor nucleic acid sequence comprises a first promoter that is operably linked to the first transgene. In some embodiments, the first donor nucleic acid sequence comprises a sequence capable of operably linking the first transgene to an endogenous promoter.

In certain embodiments, first donor nucleic acid sequence comprises, from 5′ to 3′, a first portion of the first transgene, a first untranslated sequence, the first recognition sequence, and the first homology region, wherein the second donor nucleic acid sequence comprises, from 5′ to 3′, the 5′ homology arm, a second untranslated sequence, and a second portion of the first transgene, wherein the 5′ homology arm comprises, from 5′ to 3′, a sequence having homology to at least a portion of the first transgene, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first portion of the first transgene, the 5′ portion of the first nuclease recognition sequence flanked by the first and the second untranslated sequence, and the second portion of the first transgene.

In certain embodiments, the first untranslated sequence is a first intron sequence comprising a splice donor sequence at its 5′ end, and the second untranslated sequence is a second intron sequence comprising a splice acceptor sequence at its 3′ end, wherein the splice donor sequence and the splice acceptor sequence are capable of being recognized by a splicing complex, and the first intron sequence, the 5′ portion of the first nuclease recognition sequence, and the second intron sequence are capable of being spliced from the first polynucleotide upon insertion of the second donor nucleic acid sequence into the first nuclease recognition sequence and expression of the first transgene.

In certain embodiments, the second donor nucleic acid sequence comprises a second transgene.

In certain embodiments, the second donor nucleic acid sequence further comprises a second promoter which is operably linked to the second transgene.

In certain embodiments, the first donor nucleic acid sequence comprises, from 5′ to 3′, the first transgene, an IRES or 2A element, a first untranslated sequence, the first recognition sequence, and the first homology region, wherein the second donor nucleic acid sequence comprises, from 5′ to 3′, the 5′ homology arm, a second untranslated sequence, and a second transgene, wherein the 5′ homology arm comprises, from 5′ to 3′, a sequence having homology to at least a portion of the first transgene, a sequence having homology to the IRES or 2A element, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first transgene, the 2A or IRES element, the 5′ portion of the first nuclease recognition sequence flanked by the first and the second untranslated sequence, and the second transgene, such that the first transgene and the second transgene are operably linked to a single promoter.

In certain embodiments, the first donor nucleic acid sequence comprises, from 5′ to 3′, the first transgene, a first untranslated sequence, the first recognition sequence, and the first homology region, wherein the second donor nucleic acid sequence comprises, from 5′ to 3′, the 5′ homology arm, a second untranslated sequence, an IRES or 2A element, and a second transgene, wherein the 5′ homology arm comprises, from 5′ to 3′, a sequence having homology to at least a portion of the first transgene, a sequence having homology to the IRES or 2A element, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first transgene, the 2A or IRES element, the 5′ portion of the first nuclease recognition sequence flanked by the first and the second untranslated sequence, and the second transgene, such that the first transgene and the second transgene are operably linked to a single promoter.

In certain embodiments, the first untranslated sequence is a first intron sequence comprising a splice donor sequence at its 5′ end, and the second untranslated sequence is a second intron sequence comprising a splice acceptor sequence at its 3′ end, wherein the splice donor sequence and the splice acceptor sequence are capable of being recognized by a splicing complex, and the first intron sequence, the 5′ portion of the first nuclease recognition sequence, and the second intron sequence are capable of being spliced from the first polynucleotide upon insertion of the second donor nucleic acid sequence into the first nuclease recognition sequence and expression of the first transgene and the second transgene.

In certain embodiments, the first donor nucleic acid sequence comprises, from 5′ to 3′, the first transgene, the first recognition sequence, and the first homology region, wherein the second donor nucleic acid sequence comprises, from 5′ to 3′, the 5′ homology arm, a second promoter, and a second transgene operably linked to the second promoter, and wherein insertion of the second donor nucleic acid sequence into the first nuclease recognition sequence would generate a sequence comprising, from 5′ to 3′, the first transgene, the 5′ portion of the first nuclease recognition sequence, the second promoter, and the second transgene.

In certain embodiments, the first donor nucleic acid sequence comprises, from 5′ to 3′, the first transgene, a second promoter, and a first untranslated sequence, wherein the second donor nucleic acid sequence comprises, from 5′ to 3′, the 5′ homology arm and a second transgene, wherein the 5′ homology arm has homology to at least a portion of the first untranslated sequence and to the 5′ portion of the first nuclease recognition sequence, and wherein insertion of the second donor nucleic acid sequence into the first nuclease recognition sequence would generate a sequence comprising, from 5′ to 3′, the first transgene, the second promoter, the first untranslated sequence, the 5′ portion of the first nuclease recognition sequence, and the second transgene.

In certain embodiments, the first untranslated sequence is an intron sequence comprising a splice donor sequence at its 5′ end and a splice acceptor sequence at its 3′ end, wherein the splice donor sequence and the splice acceptor sequence are capable of being recognized by a splicing complex, and the intron sequence is capable of being spliced from the first polynucleotide upon insertion of the second donor nucleic acid sequence into the first nuclease recognition sequence and expression of the second transgene.

In certain embodiments, the first transgene encodes a chimeric antigen receptor. In some embodiments, the first transgene encodes an exogenous TCR. In some embodiments, the first transgene encodes an inhibitory nucleic acid. In some embodiments, the first transgene encodes a reporter protein. In some embodiments, the first transgene encodes a protein useful for purification of a eukaryotic cell of interest. In some embodiments, the first transgene encodes a therapeutic protein. In some embodiments, the first transgene encodes a suicide protein.

In certain embodiments, the second transgene encodes a chimeric antigen receptor. In some embodiments, the second transgene encodes an exogenous TCR. In some embodiments, the second transgene encodes an inhibitory nucleic acid. In some embodiments, the second transgene encodes a reporter protein. In some embodiments, the second transgene encodes a protein useful for purification of a eukaryotic cell of interest. In some embodiments, the second transgene encodes a therapeutic protein. In some embodiments, the second transgene encodes a suicide protein.

In certain embodiments, the inhibitory nucleic acid comprises an shRNA or a microRNA-adapted shRNA.

In certain embodiments, the first transgene encodes a protein that exceeds 5 kilobases in size.

In certain embodiments, the second polynucleotide is comprised within a recombinant virus or a lipid nanoparticle. In some embodiments, the recombinant virus is a recombinant adeno-associated virus (AAV). In some embodiments, the AAV vector has a serotype of AAV1, AAV2, AAV3, AAV4, AAV5, AAV6, AAV7, AAV8, AAV9, AAV10, or AAV11. In some embodiments, the recombinant AAV has a serotype of AAV6.

In certain embodiments, the second polynucleotide comprises only one D sequence. In some embodiments, the D sequence is positioned within a 5′ inverted terminal repeat (ITR). In some embodiments, the D sequence overlaps the 5′ ITR. In some embodiments, the D sequence is positioned 3′ downstream of the 5′ ITR and 5′ upstream of the 5′ homology arm. In some embodiments, the D sequence is positioned 5′ upstream of a 3′ ITR and 3′ downstream of the 3′ homology arm. In some embodiments, the D sequence overlaps the 3′ ITR. In some embodiments, the D sequence is positioned within the 3′ ITR.

In certain embodiments, the first polynucleotide is comprised within a first recombinant virus. In some embodiments, the first polynucleotide is comprised within a first lipid nanoparticle. In some embodiments, the second polynucleotide is comprised within a second recombinant virus. In some embodiments, the second polynucleotide is comprised within a second lipid nanoparticle.

In some embodiments, the first heterologous nucleic acid sequence further comprises: (a) a 5′ homology arm that is homologous to a sequence 5′ upstream of the endogenous nuclease recognition sequence and to a 5′ portion of the endogenous nuclease recognition sequence; and (b) a 3′ homology arm that is homologous to a sequence 3′ downstream of the endogenous nuclease recognition sequence and to a 3′ portion of the endogenous nuclease recognition sequence; or wherein the first homology region is homologous to a sequence 3′ downstream of the endogenous nuclease recognition sequence; wherein the 5′ homology arm and the 3′ homology arm flank the first heterologous nucleic acid sequence.

In certain embodiments, the first polynucleotide is comprised within a first recombinant virus. In some embodiments, the first polynucleotide is comprised within a first lipid nanoparticle. In some embodiments, the second polynucleotide is comprised within a second recombinant virus. In some embodiments, the second polynucleotide is comprised within a second lipid nanoparticle.

In certain embodiments, the first heterologous nucleic acid sequence further comprises a 5′ homology arm that is homologous to a sequence 5′ upstream of the endogenous nuclease recognition sequence and to a 5′ portion of the endogenous nuclease recognition sequence, and wherein the first homology region is homologous to a sequence 3′ downstream of the endogenous nuclease recognition sequence.

In certain embodiments, the first recombinant virus is a first recombinant AAV. In some embodiments, the second recombinant virus is a second recombinant AAV. In certain embodiments, the first AAV vector and/or the second AAV vector has a serotype of AAV1, AAV2, AAV3, AAV4, AAV5, AAV6, AAV7, AAV8, AAV9, AAV10, or AAV11. In some embodiments, the first AAV vector and/or the second AAV vector has a serotype of AAV6.

In certain embodiments, the first polynucleotide comprises only one D sequence. In certain embodiments, the second polynucleotide comprises only one D sequence.

In some embodiments, the D sequence comprised by the first polynucleotide is positioned within a 5′ inverted terminal repeat (ITR). In some embodiments, the D sequence comprised by the first polynucleotide overlaps the 5′ ITR. In some embodiments, the D sequence comprised by the first polynucleotide is positioned 3′ downstream of the 5′ ITR and 5′ upstream of the 5′ homology arm. In some embodiments, the D sequence comprised by the first polynucleotide is positioned 5′ upstream of a 3′ ITR and 3′ downstream of the 3′ homology arm. In some embodiments, the D sequence comprised by the first polynucleotide overlaps the 3′ ITR. In some embodiments, the D sequence comprised by the first polynucleotide is positioned within the 3′ ITR.

In some embodiments, the D sequence comprised by the second polynucleotide is positioned within a 5′ inverted terminal repeat (ITR). In some embodiments, the D sequence comprised by the second polynucleotide overlaps the 5′ ITR. In some embodiments, the D sequence comprised by the second polynucleotide is positioned 3′ downstream of the 5′ ITR and 5′ upstream of the 5′ homology arm. In some embodiments, the D sequence comprised by the second polynucleotide is positioned 5′ upstream of a 3′ ITR and 3′ downstream of the 3′ homology arm. In some embodiments, the D sequence comprised by the second polynucleotide overlaps the 3′ ITR. In some embodiments, the D sequence comprised by the second polynucleotide is positioned within the 3′ ITR.

In certain embodiments, the D sequence comprised by the first polynucleotide: (a) is positioned within a 5′ inverted terminal repeat (ITR); (b) overlaps the 5′ ITR; or (c) is positioned 3′ downstream of the 5′ ITR and 5′ upstream of the 5′ homology arm; and wherein the D sequence comprised by the second polynucleotide: (d) is positioned within a 5′ inverted terminal repeat (ITR); (e) overlaps the 5′ ITR; or (f) is positioned 3′ downstream of the 5′ ITR and 5′ upstream of the 5′ homology arm. Embodiments include any combination of any one of (a)-(c) with any one of (d)-(f).

In certain embodiments, the D sequence comprised by the first polynucleotide: (a) is positioned 5′ upstream of a 3′ ITR and 3′ downstream of the 3′ homology arm; (b) overlaps the 3′ ITR; or (c) is positioned within the 3′ ITR; and wherein the D sequence comprised by the second polynucleotide: (d) is positioned 5′ upstream of a 3′ ITR and 3′ downstream of the 3′ homology arm; (e) overlaps the 3′ ITR; or (f) is positioned within the 3′ ITR. Embodiments include any combination of any one of (a)-(c) with any one of (d)-(f).

In certain embodiments, the composition is a eukaryotic cell. In some embodiments, the eukaryotic cell is mammalian cell. In some embodiments, the mammalian cell is human cell. In some embodiments, the human cell is a human immune cell. In some embodiments, the human immune cell is a human T cell or a human natural killer cell. In some embodiments, the human cell is an induced pluripotent stem cell (iPSC). In some embodiments, the eukaryotic cell is a plant cell.

In another aspect, the invention provides a eukaryotic cell comprising: (a) a first polynucleotide comprising a first heterologous nucleic acid sequence comprising: (i) a first donor nucleic acid sequence comprising a first nuclease recognition sequence for a first engineered nuclease; and (ii) a first homology region positioned 3′ downstream of the first nuclease recognition sequence; (b) a second polynucleotide comprising a second heterologous nucleic acid sequence comprising: (i) a 5′ homology arm having homology to at least a portion of the first donor nucleic acid sequence and to a 5′ portion of the first nuclease recognition sequence; (ii) a 3′ homology arm having homology to a 3′ portion of the first nuclease recognition sequence and to the first homology region; and (iii) a second donor nucleic acid sequence positioned between the 5′ homology arm and the 3′ homology arm; and (c) one or more engineered nucleases, or one or more nucleic acids encoding the one or more engineered nucleases, comprising the first engineered nuclease.

In some embodiments, the first nuclease recognition sequence is positioned at the 3′ end of the first donor nucleic acid sequence.

In some embodiments, the one or more engineered nucleases is an engineered meganuclease, a TALEN, a compact TALEN, a zinc finger nuclease, a CRISPR system nuclease, or a megaTAL. In certain embodiments, the one or more engineered nucleases is an engineered meganuclease.

In certain embodiments, the first engineered nuclease is not capable of binding and cleaving an endogenous nuclease recognition sequence normally present in the genome of a eukaryotic cell of interest. In some embodiments, the one or more engineered nucleases comprises a second engineered nuclease capable of binding and cleaving the endogenous nuclease recognition sequence.

In certain embodiments, the first engineered nuclease is capable of binding and cleaving the first nuclease recognition sequence and an endogenous nuclease recognition sequence normally present in the genome of a eukaryotic cell of interest. In some embodiments, the first nuclease recognition sequence is identical to the endogenous nuclease recognition sequence.

In certain embodiments, the endogenous nuclease recognition sequence is within a T cell receptor (TCR) alpha gene. In certain embodiments, the endogenous nuclease recognition sequence is within a TCR beta gene. In some embodiments, the endogenous nuclease recognition sequence is within a TCR alpha constant (TRAC) gene. In some embodiments, the endogenous nuclease recognition sequence is within a TCR beta constant (TRBC) gene. In particular embodiments, the first nuclease recognition sequence comprises SEQ ID NO: 1.

In certain embodiments, the one or more nucleic acids encoding the one or more engineered nucleases are mRNA. In some embodiments, the one or more nucleic acids encoding the one or more engineered nucleases are comprised within one or more nuclease adeno-associated viruses (AAVs).

In certain embodiments, the second donor nucleic acid sequence does not comprise a second nuclease recognition sequence. In some embodiments, the second donor nucleic acid does not comprise a 5′ portion of a nuclease recognition sequence that is capable of pairing with the 3′ portion of the first nuclease recognition sequence to generate a second nuclease recognition sequence. In some embodiments, the second donor nucleic acid does not comprise a 3′ portion of a nuclease recognition sequence that is capable of pairing with the 5′ portion of the first nuclease recognition sequence to generate a second nuclease recognition sequence.

In certain embodiments, the second donor nucleic acid sequence comprises a second nuclease recognition sequence. In some embodiments, the second donor nucleic acid sequence comprises a 5′ portion of a nuclease recognition sequence capable of pairing with the 3′ portion of the first nuclease recognition sequence to generate a second nuclease recognition sequence. In some embodiments, the second donor nucleic acid sequence comprises a 3′ portion of a nuclease recognition sequence capable of pairing with the 5′ portion of the first nuclease recognition sequence to generate a second nuclease recognition sequence.

In certain embodiments, the first engineered nuclease is not capable of binding and cleaving the second nuclease recognition sequence. In some embodiments, the second nuclease recognition sequence is not identical to the first nuclease recognition sequence. In certain embodiments, the one or more engineered nucleases comprises a third engineered nuclease capable of binding and cleaving the second nuclease recognition sequence.

In certain embodiments, the first engineered nuclease is capable of binding and cleaving the first nuclease recognition sequence, the second nuclease recognition sequence, and the endogenous nuclease recognition sequence. In some embodiments, the first nuclease recognition sequence, the second nuclease recognition sequence, and the endogenous nuclease recognition sequence are identical.

In certain embodiments, the first donor nucleic acid sequence comprises a first transgene which is expressed in the eukaryotic cell.

In certain embodiments, the first donor nucleic acid sequence comprises a first promoter that is operably linked to the first transgene. In some embodiments, the first donor nucleic acid sequence comprises a sequence capable of operably linking the first transgene to an endogenous promoter of the eukaryotic cell.

In certain embodiments, first donor nucleic acid sequence comprises, from 5′ to 3′, a first portion of the first transgene, a first untranslated sequence, the first recognition sequence, and the first homology region, wherein the second donor nucleic acid sequence comprises, from 5′ to 3′, the 5′ homology arm, a second untranslated sequence, and a second portion of the first transgene, wherein the 5′ homology arm comprises, from 5′ to 3′, a sequence having homology to at least a portion of the first transgene, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first portion of the first transgene, the 5′ portion of the first nuclease recognition sequence flanked by the first and the second untranslated sequence, and the second portion of the first transgene.

In certain embodiments, the first untranslated sequence is a first intron sequence comprising a splice donor sequence at its 5′ end, and the second untranslated sequence is a second intron sequence comprising a splice acceptor sequence at its 3′ end, wherein the splice donor sequence and the splice acceptor sequence are capable of being recognized by a splicing complex, and the first intron sequence, the 5′ portion of the first nuclease recognition sequence, and the second intron sequence are capable of being spliced from the first polynucleotide upon insertion of the second donor nucleic acid sequence into the first nuclease recognition sequence and expression of the first transgene.

In certain embodiments, the second donor nucleic acid sequence comprises a second transgene which is expressed in the eukaryotic cell.

In certain embodiments, the second donor nucleic acid sequence further comprises a second promoter which is operably linked to the second transgene.

In certain embodiments, the first donor nucleic acid sequence comprises, from 5′ to 3′, the first transgene, an IRES or 2A element, a first untranslated sequence, the first recognition sequence, and the first homology region, wherein the second donor nucleic acid sequence comprises, from 5′ to 3′, the 5′ homology arm, a second untranslated sequence, and a second transgene, wherein the 5′ homology arm comprises, from 5′ to 3′, a sequence having homology to at least a portion of the first transgene, a sequence having homology to the IRES or 2A element, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first transgene, the 2A or IRES element, the 5′ portion of the first nuclease recognition sequence flanked by the first and the second untranslated sequence, and the second transgene, such that the first transgene and the second transgene are operably linked to a single promoter.

In certain embodiments, the first donor nucleic acid sequence comprises, from 5′ to 3′, the first transgene, a first untranslated sequence, the first recognition sequence, and the first homology region, wherein the second donor nucleic acid sequence comprises, from 5′ to 3′, the 5′ homology arm, a second untranslated sequence, an IRES or 2A element, and a second transgene, wherein the 5′ homology arm comprises, from 5′ to 3′, a sequence having homology to at least a portion of the first transgene, a sequence having homology to the IRES or 2A element, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first transgene, the 2A or IRES element, the 5′ portion of the first nuclease recognition sequence flanked by the first and the second untranslated sequence, and the second transgene, such that the first transgene and the second transgene are operably linked to a single promoter.

In certain embodiments, the first untranslated sequence is a first intron sequence comprising a splice donor sequence at its 5′ end, and the second untranslated sequence is a second intron sequence comprising a splice acceptor sequence at its 3′ end, wherein the splice donor sequence and the splice acceptor sequence are capable of being recognized by a splicing complex, and the first intron sequence, the 5′ portion of the first nuclease recognition sequence, and the second intron sequence are capable of being spliced from the first polynucleotide upon insertion of the second donor nucleic acid sequence into the first nuclease recognition sequence and expression of the first transgene and the second transgene.

In certain embodiments, the first donor nucleic acid sequence comprises, from 5′ to 3′, the first transgene, the first recognition sequence, and the first homology region, wherein the second donor nucleic acid sequence comprises, from 5′ to 3′, the 5′ homology arm, a second promoter, and a second transgene operably linked to the second promoter, and wherein insertion of the second donor nucleic acid sequence into the first nuclease recognition sequence would generate a sequence comprising, from 5′ to 3′, the first transgene, the 5′ portion of the first nuclease recognition sequence, the second promoter, and the second transgene.

In certain embodiments, the first donor nucleic acid sequence comprises, from 5′ to 3′, the first transgene, a second promoter, and a first untranslated sequence, wherein the second donor nucleic acid sequence comprises, from 5′ to 3′, the 5′ homology arm and a second transgene, wherein the 5′ homology arm has homology to at least a portion of the first untranslated sequence and to the 5′ portion of the first nuclease recognition sequence, and wherein insertion of the second donor nucleic acid sequence into the first nuclease recognition sequence would generate a sequence comprising, from 5′ to 3′, the first transgene, the second promoter, the first untranslated sequence, the 5′ portion of the first nuclease recognition sequence, and the second transgene.

In certain embodiments, the first untranslated sequence is an intron sequence comprising a splice donor sequence at its 5′ end and a splice acceptor sequence at its 3′ end, wherein the splice donor sequence and the splice acceptor sequence are capable of being recognized by a splicing complex, and the intron sequence is capable of being spliced from the first polynucleotide upon insertion of the second donor nucleic acid sequence into the first nuclease recognition sequence and expression of the second transgene.

In certain embodiments, the first transgene encodes a chimeric antigen receptor. In some embodiments, the first transgene encodes an exogenous TCR. In some embodiments, the first transgene encodes an inhibitory nucleic acid. In some embodiments, the first transgene encodes a reporter protein. In some embodiments, the first transgene encodes a protein useful for purification of a eukaryotic cell of interest. In some embodiments, the first transgene encodes a therapeutic protein. In some embodiments, the first transgene encodes a suicide protein.

In certain embodiments, the second transgene encodes a chimeric antigen receptor. In some embodiments, the second transgene encodes an exogenous TCR. In some embodiments, the second transgene encodes an inhibitory nucleic acid. In some embodiments, the second transgene encodes a reporter protein. In some embodiments, the second transgene encodes a protein useful for purification of a eukaryotic cell of interest. In some embodiments, the second transgene encodes a therapeutic protein. In some embodiments, the second transgene encodes a suicide protein.

In certain embodiments, the inhibitory nucleic acid comprises an shRNA or a microRNA-adapted shRNA.

In certain embodiments, the first transgene encodes a protein that exceeds 5 kilobases in size.

In certain embodiments, the eukaryotic cell comprises the first polynucleotide in its genome. In some embodiments, the eukaryotic cell comprises the first polynucleotide in its genome within the endogenous nuclease recognition sequence.

In certain embodiments, the eukaryotic cell comprises a recombinant virus comprising the second polynucleotide.

In certain embodiments, the recombinant virus is a recombinant AAV. In some embodiments, the AAV vector has a serotype of AAV1, AAV2, AAV3, AAV4, AAV5, AAV6, AAV7, AAV8, AAV9, AAV10, or AAV11. In some embodiments, the recombinant AAV has a serotype of AAV6.

In certain embodiments, the second polynucleotide comprises only one D sequence. In some embodiments, the D sequence is positioned within a 5′ inverted terminal repeat (ITR). In some embodiments, the D sequence overlaps the 5′ ITR. In some embodiments, the D sequence is positioned 3′ downstream of the 5′ ITR and 5′ upstream of the 5′ homology arm. In some embodiments, the D sequence is positioned 5′ upstream of a 3′ ITR and 3′ downstream of the 3′ homology arm. In some embodiments, the D sequence overlaps the 3′ ITR. In some embodiments, the D sequence is positioned within the 3′ ITR.

In certain embodiments, the first polynucleotide is comprised within a first recombinant virus. In some embodiments, the first polynucleotide is comprised within a first lipid nanoparticle. In some embodiments, the second polynucleotide is comprised within a second recombinant virus. In some embodiments, the second polynucleotide is comprised within a second lipid nanoparticle.

In certain embodiments, the first heterologous nucleic acid sequence further comprises: (a) a 5′ homology arm that is homologous to a sequence 5′ upstream of the endogenous nuclease recognition sequence and to a 5′ portion of the endogenous nuclease recognition sequence; and (b) a 3′ homology arm that is homologous to a sequence 3′ downstream of the endogenous nuclease recognition sequence and to a 3′ portion of the endogenous nuclease recognition sequence; or wherein the first homology region is homologous to a sequence 3′ downstream of the endogenous nuclease recognition sequence; wherein the 5′ homology arm and the 3′ homology arm flank the first heterologous nucleic acid sequence.

In certain embodiments, the first polynucleotide is comprised within a first recombinant virus. In some embodiments, the first polynucleotide is comprised within a first lipid nanoparticle. In some embodiments, the second polynucleotide is comprised within a second recombinant virus. In some embodiments, the second polynucleotide is comprised within a second lipid nanoparticle.

In certain embodiments, the first heterologous nucleic acid sequence further comprises a 5′ homology arm that is homologous to a sequence 5′ upstream of the endogenous nuclease recognition sequence and to a 5′ portion of the endogenous nuclease recognition sequence, and wherein the first homology region is homologous to a sequence 3′ downstream of the endogenous nuclease recognition sequence.

In certain embodiments, the first recombinant virus is a first recombinant AAV. In some embodiments, the second recombinant virus is a second recombinant AAV. In certain embodiments, the first AAV vector and/or the second AAV vector has a serotype of AAV1, AAV2, AAV3, AAV4, AAV5, AAV6, AAV7, AAV8, AAV9, AAV10, or AAV11. In some embodiments, the first AAV vector and/or the second AAV vector has a serotype of AAV6. In certain embodiments, the first polynucleotide comprises only one D sequence. In certain embodiments, the second polynucleotide comprises only one D sequence.

In some embodiments, the D sequence comprised by the first polynucleotide is positioned within a 5′ inverted terminal repeat (ITR). In some embodiments, the D sequence comprised by the first polynucleotide overlaps the 5′ ITR. In some embodiments, the D sequence comprised by the first polynucleotide is positioned 3′ downstream of the 5′ ITR and 5′ upstream of the 5′ homology arm. In some embodiments, the D sequence comprised by the first polynucleotide is positioned 5′ upstream of a 3′ ITR and 3′ downstream of the 3′ homology arm. In some embodiments, the D sequence comprised by the first polynucleotide overlaps the 3′ ITR. In some embodiments, the D sequence comprised by the first polynucleotide is positioned within the 3′ ITR.

In some embodiments, the D sequence comprised by the second polynucleotide is positioned within a 5′ inverted terminal repeat (ITR). In some embodiments, the D sequence comprised by the second polynucleotide overlaps the 5′ ITR. In some embodiments, the D sequence comprised by the second polynucleotide is positioned 3′ downstream of the 5′ ITR and 5′ upstream of the 5′ homology arm. In some embodiments, the D sequence comprised by the second polynucleotide is positioned 5′ upstream of a 3′ ITR and 3′ downstream of the 3′ homology arm. In some embodiments, the D sequence comprised by the second polynucleotide overlaps the 3′ ITR. In some embodiments, the D sequence comprised by the second polynucleotide is positioned within the 3′ ITR.

In certain embodiments, the D sequence comprised by the first polynucleotide: (a) is positioned within a 5′ inverted terminal repeat (ITR); (b) overlaps the 5′ ITR; or (c) is positioned 3′ downstream of the 5′ ITR and 5′ upstream of the 5′ homology arm; and wherein the D sequence comprised by the second polynucleotide: (d) is positioned within a 5′ inverted terminal repeat (ITR); (e) overlaps the 5′ ITR; or (f) is positioned 3′ downstream of the 5′ ITR and 5′ upstream of the 5′ homology arm. Embodiments include any combination of any one of (a)-(c) with any one of (d)-(f).

In certain embodiments, the D sequence comprised by the first polynucleotide: (a) is positioned 5′ upstream of a 3′ ITR and 3′ downstream of the 3′ homology arm; (b) overlaps the 3′ ITR; or (c) is positioned within the 3′ ITR; and wherein the D sequence comprised by the second polynucleotide: (d) is positioned 5′ upstream of a 3′ ITR and 3′ downstream of the 3′ homology arm; (e) overlaps the 3′ ITR; or (f) is positioned within the 3′ ITR. Embodiments include any combination of any one of (a)-(c) with any one of (d)-(f).

In certain embodiments, the eukaryotic cell is mammalian cell. In some embodiments, the mammalian cell is human cell. In some embodiments, the human cell is a human immune cell. In some embodiments, the human immune cell is a human T cell or a human natural killer cell. In some embodiments, the human cell is an induced pluripotent stem cell (iPSC). In some embodiments, the eukaryotic cell is a plant cell.

In another aspect, the invention provides a population of eukaryotic cells comprising a plurality of a eukaryotic cell of the invention.

In some embodiments, at least about 20%, about 30%, about 40%, about 50%, about 55%, about 60%, about 65%, about 70%, about 75%, about 80%, about 85%, about 90%, about 95%, or up to 100% of cells in the population are a eukaryotic cell of the invention.

In another aspect, the invention provides a pharmaceutical composition comprising a pharmaceutically-acceptable carrier and a eukaryotic cell of the invention.

In another aspect, the invention provides a pharmaceutical composition comprising a pharmaceutically-acceptable carrier and a population of eukaryotic cells described herein.

In another aspect, the invention provides a method of immunotherapy for treating a cancer in a subject in need thereof, the method comprising administering to the subject an effective amount of a pharmaceutical composition described herein, wherein the eukaryotic cell is a genetically-modified human T cell, or a cell derived therefrom, or a genetically-modified NK cell, or a cell derived therefrom, and wherein the eukaryotic cell comprises a CAR or exogenous TCR, wherein the CAR or the exogenous TCR comprises an extracellular ligand-binding domain having specificity for a tumor-specific antigen.

In certain embodiments, the first donor nucleic acid sequence and/or the second donor nucleic acid sequence comprises a transgene encoding the CAR or the exogenous TCR.

In certain embodiments, the first donor nucleic acid sequence is inserted into the genome of the eukaryotic cell within a TCR alpha gene. In certain embodiments, the first donor nucleic acid sequence is inserted into the genome of the eukaryotic cell within a TCR beta gene. In certain embodiments, the first donor nucleic acid sequence is inserted into the genome of the eukaryotic cell within a TRAC gene. In certain embodiments, the first donor nucleic acid sequence is inserted into the genome of the eukaryotic cell within a TRBC gene.

In certain embodiments, the eukaryotic cell has no detectable cell-surface expression of an endogenous TCR (i.e., an alpha/beta TCR).

In certain embodiments, the cancer is selected from the group consisting of a cancer of carcinoma, lymphoma, sarcoma, blastomas, and leukemia. In some embodiments, the cancer is selected from the group consisting of a cancer of B-cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin's lymphoma. In some embodiments, the cancer of B-cell origin is selected from the group consisting of B-lineage acute lymphoblastic leukemia, B-cell chronic lymphocytic leukemia, B-cell non-Hodgkin's lymphoma, and multiple myeloma.

In another aspect, the invention provides a method for producing a genetically-modified eukaryotic cell, the method comprising introducing into a eukaryotic cell: (a) a first polynucleotide comprising a first heterologous nucleic acid sequence comprising: (i) a first donor nucleic acid sequence comprising a first nuclease recognition sequence for a first engineered nuclease; and (ii) a first homology region positioned 3′ downstream of the first nuclease recognition sequence; (b) a second polynucleotide comprising a second heterologous nucleic acid sequence comprising: (i) a 5′ homology arm having homology to at least a portion of the first donor nucleic acid sequence and to a 5′ portion of the first nuclease recognition sequence; (ii) a 3′ homology arm having homology to a 3′ portion of the first nuclease recognition sequence and to the first homology region; and (iii) a second donor nucleic acid sequence positioned between the 5′ homology arm and the 3′ homology arm; and (c) one or more engineered nucleases, or one or more nucleic acids encoding the one or more engineered nucleases, comprising the first engineered nuclease, wherein the one or more engineered nucleases are expressed in the eukaryotic cell and generate a first cleavage site at an endogenous nuclease recognition sequence in the genome of the eukaryotic cell, wherein the first donor nucleic acid sequence is inserted into the first cleavage site, wherein the one or more engineered nucleases generate a second cleavage site at the first nuclease recognition sequence, and wherein the second donor nucleic acid sequence is inserted into the second cleavage site.

In some embodiments, the first nuclease recognition sequence is positioned at the 3′ end of the first donor nucleic acid sequence.

In certain embodiments, the first polynucleotide and the second polynucleotide are introduced simultaneously into the eukaryotic cell. In some embodiments, the first polynucleotide and the second polynucleotide are introduced sequentially into the eukaryotic cell.

In certain embodiments, the second polynucleotide is introduced into the eukaryotic cell within at least 1 hour, 2 hours, 3 hours, 4 hours, 5 hours, 6 hours, 7 hours, 8 hours, 9 hours, 10 hours, 15 hours, 20 hours, or 24 hours of the first polynucleotide. In such embodiments, the first polynucleotide can be introduced prior to the second polynucleotide. In other such embodiments, the second polynucleotide can be introduced prior to the first polynucleotide.

In certain embodiments, the first polynucleotide, the second polynucleotide, and the one or more nucleases or nucleic acids encoding the one or more engineered nucleases are introduced simultaneously into the eukaryotic cell.

In certain embodiments, the one or more engineered nucleases, or nucleic acids encoding the one or more engineered nucleases, is introduced into the eukaryotic cell within at least 1 hour, 2 hours, 3 hours, 4 hours, 5 hours, 6 hours, 7 hours, 8 hours, 9 hours, 10 hours, 15 hours, 20 hours, or 24 hours of the first polynucleotide and the second polynucleotide.

In some embodiments, the one or more engineered nucleases is an engineered meganuclease, a TALEN, a compact TALEN, a zinc finger nuclease, a CRISPR system nuclease, or a megaTAL. In certain embodiments, the one or more engineered nucleases is an engineered meganuclease.

In certain embodiments, the first engineered nuclease is not capable of binding and cleaving an endogenous nuclease recognition sequence normally present in the genome of a eukaryotic cell of interest. In some embodiments, the one or more engineered nucleases comprises a second engineered nuclease capable of binding and cleaving the endogenous nuclease recognition sequence, wherein the second engineered nuclease generates the first cleavage site (i.e., the cleavage site in the gnome), and wherein the first engineered nuclease generates the second cleavage site (i.e., in the first polynucleotide).

In certain embodiments, the first polynucleotide further comprises: (a) a 5′ homology arm that is homologous to a sequence 5′ upstream of the endogenous nuclease recognition sequence and to a 5′ portion of the endogenous nuclease recognition sequence; and (b) a 3′ homology arm that is homologous to a sequence 3′ downstream of the endogenous nuclease recognition sequence and to a 3′ portion of the endogenous nuclease recognition sequence; wherein the 5′ homology arm and the 3′ homology arm flank the first heterologous nucleic acid sequence.

In certain embodiments, the first nuclease recognition sequence is identical to the endogenous nuclease recognition sequence.

In certain embodiments, the first engineered nuclease generates the first cleavage site and the second cleavage site.

In certain embodiments, the first polynucleotide further comprises a 5′ homology arm that is homologous to a sequence 5′ upstream of the endogenous nuclease recognition sequence and to a 5′ portion of the endogenous nuclease recognition sequence, and wherein the first homology region is homologous to a sequence 3′ downstream of the endogenous nuclease recognition sequence.

In certain embodiments, the endogenous nuclease recognition sequence is within a T cell receptor (TCR) alpha gene. In certain embodiments, the endogenous nuclease recognition sequence is within a TCR beta gene. In some embodiments, the endogenous nuclease recognition sequence is within a TCR alpha constant (TRAC) gene. In some embodiments, the endogenous nuclease recognition sequence is within a TCR beta constant (TRBC) gene. In particular embodiments, the first nuclease recognition sequence comprises SEQ ID NO: 1.

In certain embodiments, the one or more nucleic acids encoding the one or more engineered nucleases are mRNA. In some embodiments, the one or more nucleic acids encoding the one or more engineered nucleases are comprised within one or more nuclease adeno-associated viruses (AAVs).

In certain embodiments, the second donor nucleic acid sequence does not comprise a second nuclease recognition sequence. In some embodiments, the second donor nucleic acid does not comprise a 5′ portion of a nuclease recognition sequence that is capable of pairing with the 3′ portion of the first nuclease recognition sequence to generate a second nuclease recognition sequence. In some embodiments, the second donor nucleic acid does not comprise a 3′ portion of a nuclease recognition sequence that is capable of pairing with the 5′ portion of the first nuclease recognition sequence to generate a second nuclease recognition sequence.

In certain embodiments, the second donor nucleic acid sequence comprises a second nuclease recognition sequence. In some embodiments, the second donor nucleic acid sequence comprises a 5′ portion of a nuclease recognition sequence capable of pairing with the 3′ portion of the first nuclease recognition sequence to generate a second nuclease recognition sequence. In some embodiments, the second donor nucleic acid sequence comprises a 3′ portion of a nuclease recognition sequence capable of pairing with the 5′ portion of the first nuclease recognition sequence to generate a second nuclease recognition sequence.

In certain embodiments, the second nuclease recognition sequence is not identical to the first nuclease recognition sequence.

In certain embodiments, the one or more engineered nucleases comprises a third engineered nuclease capable of binding and cleaving the second nuclease recognition sequence, wherein the third engineered nuclease generates a third cleavage site in the second nuclease recognition sequence.

In certain embodiments, the first engineered nuclease is capable of binding and cleaving the first nuclease recognition sequence, the second nuclease recognition sequence, and the endogenous nuclease recognition sequence.

In certain embodiments, the first nuclease recognition sequence, the second nuclease recognition sequence, and the endogenous nuclease recognition sequence are identical.

In certain embodiments, the first donor nucleic acid sequence comprises a first transgene which is expressed in the eukaryotic cell.

In certain embodiments, the first donor nucleic acid sequence comprises a first promoter that is operably linked to the first transgene, or a sequence capable of operably linking the first transgene to an endogenous promoter of the eukaryotic cell.

In certain embodiments, first donor nucleic acid sequence comprises, from 5′ to 3′, a first portion of the first transgene, a first untranslated sequence, the first recognition sequence, and the first homology region, wherein the second donor nucleic acid sequence comprises, from 5′ to 3′, the 5′ homology arm, a second untranslated sequence, and a second portion of the first transgene, wherein the 5′ homology arm comprises, from 5′ to 3′, a sequence having homology to at least a portion of the first transgene, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first portion of the first transgene, the 5′ portion of the first nuclease recognition sequence flanked by the first and the second untranslated sequence, and the second portion of the first transgene.

In certain embodiments, the first untranslated sequence is a first intron sequence comprising a splice donor sequence at its 5′ end, and the second untranslated sequence is a second intron sequence comprising a splice acceptor sequence at its 3′ end, wherein the splice donor sequence and the splice acceptor sequence are capable of being recognized by a splicing complex, and the first intron sequence, the 5′ portion of the first nuclease recognition sequence, and the second intron sequence are capable of being spliced from the first polynucleotide upon insertion of the second donor nucleic acid sequence into the first nuclease recognition sequence and expression of the first transgene.

In certain embodiments, the second donor nucleic acid sequence comprises a second transgene which is expressed in the eukaryotic cell.

In certain embodiments, the second donor nucleic acid sequence further comprises a second promoter which is operably linked to the second transgene.

In certain embodiments, the first donor nucleic acid sequence comprises, from 5′ to 3′, the first transgene, an IRES or 2A element, a first untranslated sequence, the first recognition sequence, and the first homology region, wherein the second donor nucleic acid sequence comprises, from 5′ to 3′, the 5′ homology arm, a second untranslated sequence, and a second transgene, wherein the 5′ homology arm comprises, from 5′ to 3′, a sequence having homology to at least a portion of the first transgene, a sequence having homology to the IRES or 2A element, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first transgene, the 2A or IRES element, the 5′ portion of the first nuclease recognition sequence flanked by the first and the second untranslated sequence, and the second transgene, such that the first transgene and the second transgene are operably linked to a single promoter.

In certain embodiments, the first donor nucleic acid sequence comprises, from 5′ to 3′, the first transgene, a first untranslated sequence, the first recognition sequence, and the first homology region, wherein the second donor nucleic acid sequence comprises, from 5′ to 3′, the 5′ homology arm, a second untranslated sequence, an IRES or 2A element, and a second transgene, wherein the 5′ homology arm comprises, from 5′ to 3′, a sequence having homology to at least a portion of the first transgene, a sequence having homology to the IRES or 2A element, a sequence having homology to the first untranslated sequence, and a sequence having homology to a 5′ portion of the first nuclease recognition sequence, and wherein insertion of the second donor nucleic acid sequence into the first nuclease recognition sequence would generate a sequence comprising, from 5′ to 3′, the first transgene, the 2A or IRES element, the 5′ portion of the first nuclease recognition sequence flanked by the first and the second untranslated sequence, and the second transgene, such that the first transgene and the second transgene are operably linked to a single promoter.

In certain embodiments, the first untranslated sequence is a first intron sequence comprising a splice donor sequence at its 5′ end, and the second untranslated sequence is a second intron sequence comprising a splice acceptor sequence at its 3′ end, wherein the splice donor sequence and the splice acceptor sequence are capable of being recognized by a splicing complex, and the first intron sequence, the 5′ portion of the first nuclease recognition sequence, and the second intron sequence are capable of being spliced from the first polynucleotide upon insertion of the second donor nucleic acid sequence into the first nuclease recognition sequence and expression of the first transgene and the second transgene.

In certain embodiments, the first donor nucleic acid sequence comprises, from 5′ to 3′, the first transgene, the first recognition sequence, and the first homology region, wherein the second donor nucleic acid sequence comprises, from 5′ to 3′, the 5′ homology arm, a second promoter, and a second transgene operably linked to the second promoter, and wherein insertion of the second donor nucleic acid sequence into the first nuclease recognition sequence would generate a sequence comprising, from 5′ to 3′, the first transgene, the 5′ portion of the first nuclease recognition sequence, the second promoter, and the second transgene.

In certain embodiments, the first donor nucleic acid sequence comprises, from 5′ to 3′, the first transgene, a second promoter, and a first untranslated sequence, wherein the second donor nucleic acid sequence comprises, from 5′ to 3′, the 5′ homology arm and a second transgene, wherein the 5′ homology arm has homology to at least a portion of the first untranslated sequence and to the 5′ portion of the first nuclease recognition sequence, and wherein insertion of the second donor nucleic acid sequence into the first nuclease recognition sequence would generate a sequence comprising, from 5′ to 3′, the first transgene, the second promoter, the first untranslated sequence, the 5′ portion of the first nuclease recognition sequence, and the second transgene.

In certain embodiments, the first untranslated sequence is an intron sequence comprising a splice donor sequence at its 5′ end and a splice acceptor sequence at its 3′ end, wherein the splice donor sequence and the splice acceptor sequence are capable of being recognized by a splicing complex, and the intron sequence is capable of being spliced from the first polynucleotide upon insertion of the second donor nucleic acid sequence into the first nuclease recognition sequence and expression of the second transgene.

In certain embodiments, the first transgene encodes a chimeric antigen receptor. In some embodiments, the first transgene encodes an exogenous TCR. In some embodiments, the first transgene encodes an inhibitory nucleic acid. In some embodiments, the first transgene encodes a reporter protein. In some embodiments, the first transgene encodes a protein useful for purification of a eukaryotic cell of interest. In some embodiments, the first transgene encodes a therapeutic protein. In some embodiments, the first transgene encodes a suicide protein.

In certain embodiments, the second transgene encodes a chimeric antigen receptor. In some embodiments, the second transgene encodes an exogenous TCR. In some embodiments, the second transgene encodes an inhibitory nucleic acid. In some embodiments, the second transgene encodes a reporter protein. In some embodiments, the second transgene encodes a protein useful for purification of a eukaryotic cell of interest. In some embodiments, the second transgene encodes a therapeutic protein. In some embodiments, the second transgene encodes a suicide protein.

In certain embodiments, the inhibitory nucleic acid comprises an shRNA or a microRNA-adapted shRNA.

In certain embodiments, the first transgene encodes a protein that exceeds 5 kilobases in size.

In certain embodiments, the first polynucleotide is comprised within a first recombinant virus. In some embodiments, the first polynucleotide is comprised within a first lipid nanoparticle. In some embodiments, the second polynucleotide is comprised within a second recombinant virus. In some embodiments, the second polynucleotide is comprised within a second lipid nanoparticle.

In certain embodiments, the first recombinant virus is a first recombinant AAV. In some embodiments, the second recombinant virus is a second recombinant AAV. In certain embodiments, the first AAV vector and/or the second AAV vector has a serotype of AAV1, AAV2, AAV3, AAV4, AAV5, AAV6, AAV7, AAV8, AAV9, AAV10, or AAV11. In some embodiments, the first AAV vector and/or the second AAV vector has a serotype of AAV6.

In certain embodiments, the first polynucleotide comprises only one D sequence. In certain embodiments, the second polynucleotide comprises only one D sequence.

In some embodiments, the D sequence comprised by the first polynucleotide is positioned within a 5′ inverted terminal repeat (ITR). In some embodiments, the D sequence comprised by the first polynucleotide overlaps the 5′ ITR. In some embodiments, the D sequence comprised by the first polynucleotide is positioned 3′ downstream of the 5′ ITR and 5′ upstream of the 5′ homology arm. In some embodiments, the D sequence comprised by the first polynucleotide is positioned 5′ upstream of a 3′ ITR and 3′ downstream of the 3′ homology arm. In some embodiments, the D sequence comprised by the first polynucleotide overlaps the 3′ ITR. In some embodiments, the D sequence comprised by the first polynucleotide is positioned within the 3′ ITR.

In some embodiments, the D sequence comprised by the second polynucleotide is positioned within a 5′ inverted terminal repeat (ITR). In some embodiments, the D sequence comprised by the second polynucleotide overlaps the 5′ ITR. In some embodiments, the D sequence comprised by the second polynucleotide is positioned 3′ downstream of the 5′ ITR and 5′ upstream of the 5′ homology arm. In some embodiments, the D sequence comprised by the second polynucleotide is positioned 5′ upstream of a 3′ ITR and 3′ downstream of the 3′ homology arm. In some embodiments, the D sequence comprised by the second polynucleotide overlaps the 3′ ITR. In some embodiments, the D sequence comprised by the second polynucleotide is positioned within the 3′ ITR.

In certain embodiments, the D sequence comprised by the first polynucleotide: (a) is positioned within a 5′ inverted terminal repeat (ITR); (b) overlaps the 5′ ITR; or (c) is positioned 3′ downstream of the 5′ ITR and 5′ upstream of the 5′ homology arm; and wherein the D sequence comprised by the second polynucleotide: (d) is positioned within a 5′ inverted terminal repeat (ITR); (e) overlaps the 5′ ITR; or (f) is positioned 3′ downstream of the 5′ ITR and 5′ upstream of the 5′ homology arm. Embodiments include any combination of any one of (a)-(c) with any one of (d)-(f).

In certain embodiments, the D sequence comprised by the first polynucleotide: (a) is positioned 5′ upstream of a 3′ ITR and 3′ downstream of the 3′ homology arm; (b) overlaps the 3′ ITR; or (c) is positioned within the 3′ ITR; and wherein the D sequence comprised by the second polynucleotide: (d) is positioned 5′ upstream of a 3′ ITR and 3′ downstream of the 3′ homology arm; (e) overlaps the 3′ ITR; or (f) is positioned within the 3′ ITR. Embodiments include any combination of any one of (a)-(c) with any one of (d)-(f).

In certain embodiments, the eukaryotic cell is mammalian cell. In some embodiments, the mammalian cell is human cell. In some embodiments, the human cell is a human immune cell. In some embodiments, the human immune cell is a human T cell or a human natural killer cell. In some embodiments, the human cell is an induced pluripotent stem cell (iPSC). In some embodiments, the eukaryotic cell is a plant cell.

In another aspect, the invention provides a method for inserting a transgene into the genome of a target cell in vivo, the method comprising delivering to a target cell in a subject: (a) a first polynucleotide comprising a first heterologous nucleic acid sequence comprising: (i) a first donor nucleic acid sequence comprising, from 5′ to 3′, a first portion of the transgene, a first untranslated sequence, and a first nuclease recognition sequence for a first engineered nuclease; and (ii) a first homology region positioned 3′ downstream of the first nuclease recognition sequence; (b) a second polynucleotide comprising a second heterologous nucleic acid sequence comprising: (i) a 5′ homology arm having homology to at least a portion of the first donor nucleic acid sequence and to a 5′ portion of the first nuclease recognition sequence; (ii) a 3′ homology arm having homology to a 3′ portion of the first nuclease recognition sequence and the first homology region; and (iii) a second donor nucleic acid sequence positioned between the 5′ homology arm and the 3′ homology arm comprising, from 5′ to 3′, a second untranslated sequence and a second portion of the transgene; and (c) one or more nucleic acids encoding one or more engineered nucleases, wherein the one or more engineered nucleases comprise the first engineered nuclease; wherein the one or more engineered nucleases is expressed in the target cell and generate a first cleavage site at an endogenous nuclease recognition sequence normally present in the genome of the target cell, wherein the first donor nucleic acid sequence is inserted into the first cleavage site, wherein the one or more engineered nucleases generate a second cleavage site at the first nuclease recognition sequence, wherein the second donor nucleic acid sequence is inserted into the second cleavage site such that the genome comprises a sequence comprising, from 5′ to 3′, the first portion of the first transgene, the 5′ portion of the first nuclease recognition sequence flanked by the first and the second untranslated sequence, and the second portion of the first transgene, and wherein a full-length protein encoded by the transgene is expressed by the target cell.

In some embodiments, the first nuclease recognition sequence is positioned at the 3′ end of the first donor nucleic acid sequence.

In certain embodiments, the first untranslated sequence is a first intron sequence comprising a splice donor sequence at its 5′ end, and the second untranslated sequence is a second intron sequence comprising a splice acceptor sequence at its 3′ end, wherein the splice donor sequence and the splice acceptor sequence are capable of being recognized by a splicing complex, and the first intron sequence, the 5′ portion of the first nuclease recognition sequence, and the second intron sequence are capable of being spliced from the first polynucleotide upon insertion of the second donor nucleic acid sequence into the first nuclease recognition sequence and expression of the first transgene.

In certain embodiments, the transgene is at least 5 kilobases in size.

In certain embodiments, the first polynucleotide and the second polynucleotide are delivered to the target cell simultaneously. In some embodiments, the first polynucleotide and the second polynucleotide are delivered sequentially to the target cell.

In certain embodiments, the second polynucleotide is delivered to the target cell within at least 1 hour, 2 hours, 3 hours, 4 hours, 5 hours, 6 hours, 7 hours, 8 hours, 9 hours, 10 hours, 15 hours, 20 hours, or 24 hours of the first polynucleotide. In such embodiments, the first polynucleotide can be delivered prior to the second polynucleotide. In other such embodiments, the second polynucleotide can be delivered prior to the first polynucleotide.

In certain embodiments, the first polynucleotide, the second polynucleotide, and the one or more nucleases or nucleic acids encoding the one or more engineered nucleases are delivered simultaneously to the target cell.

In certain embodiments, the one or more engineered nucleases, or nucleic acids encoding the one or more engineered nucleases, is delivered to the target cell within at least 1 hour, 2 hours, 3 hours, 4 hours, 5 hours, 6 hours, 7 hours, 8 hours, 9 hours, 10 hours, 15 hours, 20 hours, or 24 hours of the first polynucleotide and the second polynucleotide.

In some embodiments, the one or more engineered nucleases is an engineered meganuclease, a TALEN, a compact TALEN, a zinc finger nuclease, a CRISPR system nuclease, or a megaTAL. In certain embodiments, the one or more engineered nucleases is an engineered meganuclease.

In certain embodiments, the first nuclease recognition sequence is identical to the endogenous nuclease recognition sequence.

In certain embodiments, the first polynucleotide further comprises: (a) a 5′ homology arm that is homologous to a sequence 5′ upstream of the endogenous nuclease recognition sequence and to a 5′ portion of the endogenous nuclease recognition sequence; and (b) a 3′ homology arm that is homologous to a sequence 3′ downstream of the endogenous nuclease recognition sequence and to a 3′ portion of the endogenous nuclease recognition sequence; wherein the 5′ homology arm and the 3′ homology arm flank the first heterologous nucleic acid sequence.

In certain embodiments, the first polynucleotide comprises a 5′ homology arm that is homologous to a sequence 5′ upstream of the endogenous nuclease recognition sequence and to a 5′ portion of the endogenous nuclease recognition sequence, and wherein the first homology region is homologous to a sequence 3′ downstream of the endogenous nuclease recognition sequence.

In certain embodiments, the endogenous nuclease recognition sequence is within a T cell receptor (TCR) alpha gene. In certain embodiments, the endogenous nuclease recognition sequence is within a TCR beta gene. In some embodiments, the endogenous nuclease recognition sequence is within a TCR alpha constant (TRAC) gene. In some embodiments, the endogenous nuclease recognition sequence is within a TCR beta constant (TRBC) gene. In particular embodiments, the first nuclease recognition sequence comprises SEQ ID NO: 1.

In certain embodiments, the one or more nucleic acids encoding the one or more engineered nucleases are mRNA. In some embodiments, the one or more nucleic acids encoding the one or more engineered nucleases are comprised within one or more nuclease adeno-associated viruses (AAVs).

In certain embodiments, the second donor nucleic acid sequence does not comprise a second nuclease recognition sequence. In some embodiments, the second donor nucleic acid does not comprise a 5′ portion of a nuclease recognition sequence that is capable of pairing with the 3′ portion of the first nuclease recognition sequence to generate a second nuclease recognition sequence. In some embodiments, the second donor nucleic acid does not comprise a 3′ portion of a nuclease recognition sequence that is capable of pairing with the 5′ portion of the first nuclease recognition sequence to generate a second nuclease recognition sequence.

In certain embodiments, the second donor nucleic acid sequence comprises a second nuclease recognition sequence. In some embodiments, the second donor nucleic acid sequence comprises a 5′ portion of a nuclease recognition sequence capable of pairing with the 3′ portion of the first nuclease recognition sequence to generate a second nuclease recognition sequence. In some embodiments, the second donor nucleic acid sequence comprises a 3′ portion of a nuclease recognition sequence capable of pairing with the 5′ portion of the first nuclease recognition sequence to generate a second nuclease recognition sequence.

In certain embodiments, the engineered nuclease does not have specificity for the second nuclease recognition sequence.

In certain embodiments, the second nuclease recognition sequence is not identical to the first nuclease recognition sequence.

In certain embodiments, the one or more engineered nucleases comprises a third engineered nuclease capable of binding and cleaving the second nuclease recognition sequence, wherein the third engineered nuclease generates a third cleavage site in the second nuclease recognition sequence.

In certain embodiments, the engineered nuclease is capable of binding and cleaving the first nuclease recognition sequence, the second nuclease recognition sequence, and the endogenous nuclease recognition sequence.

In certain embodiments, the first nuclease recognition sequence, the second nuclease recognition sequence, and the endogenous nuclease recognition sequence are identical.

In certain embodiments, the first donor nucleic acid sequence comprises a first promoter that is operably linked to the transgene, or a sequence that operably links the first transgene to an endogenous promoter of the target cell.

In certain embodiments, the transgene encodes a therapeutic protein.

In certain embodiments, the therapeutic protein is expressed at a clinically therapeutic level in the subject.

In certain embodiments, the first polynucleotide is comprised within a first recombinant AAV. In certain embodiments, the first polynucleotide is comprised within a first lipid nanoparticle. In certain embodiments, the second polynucleotide is comprised within a second recombinant AAV. In certain embodiments, the second polynucleotide is comprised within a second lipid nanoparticle.

In certain embodiments, the first recombinant AAV and/or the second recombinant AAV has a serotype of AAV1, AAV2, AAV3, AAV4, AAV5, AAV6, AAV7, AAV8, AAV9, AAV10, or AAV11. In certain embodiments, the first recombinant AAV and/or the second recombinant AAV has a serotype of AAV8.

In certain embodiments, the first polynucleotide comprises only one D sequence and the second polynucleotide comprises only one D sequence.

In some embodiments, the D sequence comprised by the first polynucleotide is positioned within a 5′ inverted terminal repeat (ITR). In some embodiments, the D sequence comprised by the first polynucleotide overlaps the 5′ ITR. In some embodiments, the D sequence comprised by the first polynucleotide is positioned 3′ downstream of the 5′ ITR and 5′ upstream of the 5′ homology arm. In some embodiments, the D sequence comprised by the first polynucleotide is positioned 5′ upstream of a 3′ ITR and 3′ downstream of the 3′ homology arm. In some embodiments, the D sequence comprised by the first polynucleotide overlaps the 3′ ITR. In some embodiments, the D sequence comprised by the first polynucleotide is positioned within the 3′ ITR.

In some embodiments, the D sequence comprised by the second polynucleotide is positioned within a 5′ inverted terminal repeat (ITR). In some embodiments, the D sequence comprised by the second polynucleotide overlaps the 5′ ITR. In some embodiments, the D sequence comprised by the second polynucleotide is positioned 3′ downstream of the 5′ ITR and 5′ upstream of the 5′ homology arm. In some embodiments, the D sequence comprised by the second polynucleotide is positioned 5′ upstream of a 3′ ITR and 3′ downstream of the 3′ homology arm. In some embodiments, the D sequence comprised by the second polynucleotide overlaps the 3′ ITR. In some embodiments, the D sequence comprised by the second polynucleotide is positioned within the 3′ ITR.

In certain embodiments, the D sequence comprised by the first polynucleotide: (a) is positioned within a 5′ inverted terminal repeat (ITR); (b) overlaps the 5′ ITR; or (c) is positioned 3′ downstream of the 5′ ITR and 5′ upstream of the 5′ homology arm; and wherein the D sequence comprised by the second polynucleotide: (d) is positioned within a 5′ inverted terminal repeat (ITR); (e) overlaps the 5′ ITR; or (f) is positioned 3′ downstream of the 5′ ITR and 5′ upstream of the 5′ homology arm. Embodiments include any combination of any one of (a)-(c) with any one of (d)-(f).

In certain embodiments, the D sequence comprised by the first polynucleotide: (a) is positioned 5′ upstream of a 3′ ITR and 3′ downstream of the 3′ homology arm; (b) overlaps the 3′ ITR; or (c) is positioned within the 3′ ITR; and wherein the D sequence comprised by the second polynucleotide: (d) is positioned 5′ upstream of a 3′ ITR and 3′ downstream of the 3′ homology arm; (e) overlaps the 3′ ITR; or (f) is positioned within the 3′ ITR. Embodiments include any combination of any one of (a)-(c) with any one of (d)-(f).

In certain embodiments, the target cell is human cell.

In particular embodiments of the compositions, eukaryotic cells, and methods described herein, the first polynucleotide of the composition comprises a first nucleic acid sequence comprising, from 5′ to 3′: a first donor nucleic acid sequence comprising at its 3′ end a first nuclease recognition sequence; and a first homology region. The second polynucleotide comprises a second nucleic acid sequence comprising, from 5′ to 3′: a 5′ homology region having homology to at least a portion of the first donor nucleic acid sequence of the first polynucleotide; a 5′ portion of the first nuclease recognition sequence; a second donor nucleic acid sequence comprising at its 3′ end a 5′ portion of the first nuclease recognition sequence; a 3′ portion of the first nuclease recognition sequence which is adjacent to the 5′ portion and generates a second nuclease recognition sequence which is identical to the first nuclease recognition sequence; and a 3′ homology region having homology to the first homology region of the first polynucleotide. See, for example FIG. 1B.

In particular embodiments of the compositions, eukaryotic cells, and methods described herein, the first polynucleotide of the composition comprises a first nucleic acid sequence comprising, from 5′ to 3′: a first donor nucleic acid sequence comprising at its 3′ end a first nuclease recognition sequence; and a first homology region. The second polynucleotide comprises a second nucleic acid sequence comprising, from 5′ to 3′: a 5′ homology region having homology to at least a portion of the first donor nucleic acid sequence of the first polynucleotide; a 5′ portion of the first nuclease recognition sequence; a second donor nucleic acid sequence comprising at its 3′ end a second nuclease recognition sequence; a 3′ portion of the first nuclease recognition sequence which is adjacent to the second nuclease recognition sequence; and a 3′ homology region having homology to the first homology region of the first polynucleotide. See, for example, FIG. 1C.

In particular embodiments of the compositions, eukaryotic cells, and methods described herein, the first polynucleotide of the composition comprises a first nucleic acid sequence comprising, from 5′ to 3′: a 5′ inverted terminal repeat (ITR); a 5′ homology region having homology to a sequence 5′ upstream of an endogenous nuclease recognition sequence; a 5′ portion of the endogenous nuclease recognition sequence; a first donor nucleic acid sequence comprising at its 3′ end a first nuclease recognition sequence which is identical to the endogenous nuclease recognition sequence; a 3′ homology region that is homologous to a sequence 3′ downstream of the endogenous recognition sequence; and a 3′ ITR. The second polynucleotide comprises a second nucleic acid sequence comprising, from 5′ to 3′: a 5′ ITR; a 5′ homology region having homology to at least a portion of the first donor nucleic acid sequence of the first polynucleotide; a 5′ portion of the first nuclease recognition sequence; a second donor nucleic acid sequence comprising at its 3′ end a 3′ portion of the first nuclease recognition sequence; a 3′ homology region having homology to the 3′ homology region of the first polynucleotide; and a 3′ ITR. See, for example, FIG. 2A. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a 5′ inverted terminal repeat (ITR); a 5′ homology region having homology to a sequence 5′ upstream of an endogenous nuclease recognition sequence; a 5′ portion of the endogenous nuclease recognition sequence; a first donor nucleic acid sequence comprising at its 3′ end a first nuclease recognition sequence which is different than the endogenous nuclease recognition sequence; a first homology region, a 3′ portion of the endogenous nuclease recognition sequence; a 3′ homology region that is homologous to a sequence 3′ downstream of the endogenous recognition sequence; and a 3′ ITR. The second polynucleotide comprises a second nucleic acid sequence comprising, from 5′ to 3′: a 5′ ITR; a 5′ homology region having homology to at least a portion of the first donor nucleic acid sequence of the first polynucleotide; a 5′ portion of the first nuclease recognition sequence; a second donor nucleic acid sequence comprising at its 3′ end a 3′ portion of the first nuclease recognition sequence; a 3′ homology region having homology to the first homology region of the first polynucleotide; and a 3′ ITR. See, for example, FIG. 2D. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a 5′ ITR; a 5′ homology region having homology to a sequence 5′ upstream of an endogenous nuclease recognition sequence; a 5′ portion of the endogenous nuclease recognition sequence; a first donor nucleic acid sequence comprising at its 3′ end a first nuclease recognition sequence which is identical to the endogenous nuclease recognition sequence; a 3′ homology region that is homologous to a sequence 3′ downstream of the endogenous recognition sequence; and a 3′ ITR. The second polynucleotide comprises a second nucleic acid sequence comprising, from 5′ to 3′: a 5′ ITR; a 5′ homology region having homology to at least a portion of the first donor nucleic acid sequence of the first polynucleotide; a 5′ portion of the first nuclease recognition sequence; a second donor nucleic acid sequence comprising at its 3′ end a 5′ portion of the first nuclease recognition sequence; a 3′ portion of the first nuclease recognition sequence which is adjacent to the 5′ portion and generates a second nuclease recognition sequence that is identical to the first nuclease recognition sequence; a 3′ homology region having homology to the 3′ homology region of the first polynucleotide; and a 3′ ITR. See, for example, FIG. 3A. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a 5′ ITR; a 5′ homology region having homology to a sequence 5′ upstream of an endogenous nuclease recognition sequence; a 5′ portion of the endogenous nuclease recognition sequence; a first donor nucleic acid sequence comprising at its 3′ end a first nuclease recognition sequence which is identical to the endogenous nuclease recognition sequence; a 3′ homology region that is homologous to a sequence 3′ downstream of the endogenous recognition sequence; and a 3′ ITR. The second polynucleotide comprises a second nucleic acid sequence comprising, from 5′ to 3′: a 5′ ITR; a 5′ homology region having homology to at least a portion of the first donor nucleic acid sequence of the first polynucleotide; a 5′ portion of the first nuclease recognition sequence; a second donor nucleic acid sequence comprising at its 3′ end a second nuclease recognition sequence that is different than the first nuclease recognition sequence; a 3′ portion of the first nuclease recognition sequence; a 3′ homology region having homology to the 3′ homology region of the first polynucleotide; and a 3′ ITR. See, for example, FIG. 3B. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a 5′ ITR; a 5′ homology region having homology to a sequence 5′ upstream of an endogenous nuclease recognition sequence; a 5′ portion of the endogenous nuclease recognition sequence; a first donor nucleic acid sequence comprising at its 3′ end a first nuclease recognition sequence which is different that the endogenous nuclease recognition sequence; a first homology region, a 3′ portion of the endogenous nuclease recognition sequence; a 3′ homology region that is homologous to a sequence 3′ downstream of the endogenous recognition sequence; and a 3′ ITR. The second polynucleotide comprises a second nucleic acid sequence comprising, from 5′ to 3′: a 5′ ITR; a 5′ homology region having homology to at least a portion of the first donor nucleic acid sequence of the first polynucleotide; a 5′ portion of the first nuclease recognition sequence; a second donor nucleic acid sequence comprising at its 3′ end a second nuclease recognition sequence that is different than the first nuclease recognition sequence; a 3′ portion of the first nuclease recognition sequence; a 3′ homology region having homology to the first homology region of the first polynucleotide; and a 3′ ITR. See, for example, FIG. 3C. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a first donor nucleic acid sequence comprising, from 5′ to 3′, a promoter at its 5′ end, a first portion of a transgene operably linked to the promoter, a first untranslated sequence (e.g., an intron) comprising a splice donor sequence at its 5′ end, and a first nuclease recognition sequence at its 3′ end; and a first homology region. The second polynucleotide comprises a second nucleic acid sequence comprising, from 5′ to 3′: a 5′ homology region having homology to at least a portion of the first donor nucleic acid sequence of the first polynucleotide; a first untranslated sequence identical to, or having a high degree of homology with, the first untranslated sequence of the first polynucleotide; a 5′ portion of the first nuclease recognition sequence; a second donor nucleic acid sequence comprising, from 5′ to 3′, a second untranslated sequence (e.g., and intron) comprising a splice acceptor sequence at its 3′ end, a second portion of the transgene, and a 5′ portion of the first nuclease recognition sequence; a 3′ portion of the first nuclease recognition sequence which is adjacent to the 5′ portion and generates a second nuclease recognition sequence which is identical to the first nuclease recognition sequence; and a 3′ homology region having homology to the first homology region of the first polynucleotide Following cleavage of the first nuclease recognition sequence by an engineered nuclease, insertion of the second donor nucleic acid sequence into the first polynucleotide results in a polynucleotide comprising, from 5′ to 3′, the promoter that is operably linked to the first portion of the transgene, the first untranslated sequence, the 5′ portion of the first recognition sequence, the second untranslated sequence, the second portion of the transgene, the 5′ portion of the second nuclease recognition sequence, the 3′ portion of the first nuclease recognition sequence, and the first homology region. In those embodiments wherein the first untranslated sequence comprises an intron comprising a splice donor sequence and the second untranslated sequence comprises an intron comprising a splice acceptor sequence, the two untranslated sequences and the 5′ portion of the first nuclease recognition sequence can be spliced out by a splicing complex, allowing for the promoter to be operably linked to the complete transgene. See, for example, FIG. 4A. In certain embodiments, the first polynucleotide further comprises a second promoter operably linked to another transgene, wherein the promoter and additional transgene are positioned 5′ upstream of the first promoter in the first donor nucleic acid sequence. See, for example, FIG. 4B. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a first donor nucleic acid sequence comprising, from 5′ to 3′, a promoter at its 5′ end, a first transgene operably linked to the promoter, and a first nuclease recognition sequence at its 3′ end; and a first homology region. The second polynucleotide comprises a second nucleic acid sequence comprising, from 5′ to 3′: a 5′ homology region having homology to at least a portion of the first donor nucleic acid sequence of the first polynucleotide; a 5′ portion of the first nuclease recognition sequence; a second donor nucleic acid sequence comprising, from 5′ to 3′, a second promoter, a second transgene operably linked to the second promoter, and a 5′ portion of the first nuclease recognition sequence; a 3′ portion of the first nuclease recognition sequence which is adjacent to the 5′ portion and generates a second nuclease recognition sequence which is identical to the first nuclease recognition sequence; and a 3′ homology region having homology to the first homology region of the first polynucleotide. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the 5′ portion of the first nuclease recognition sequence, the second promoter that is operably linked to the second transgene, the 5′ portion of the second nuclease recognition sequence, the 3′ portion of the first nuclease recognition sequence, and the first homology region. See, for example, FIG. 5A. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a first donor nucleic acid sequence comprising, from 5′ to 3′, a first promoter at its 5′ end, a first transgene operably linked to the first promoter, a second promoter, an untranslated sequence (e.g., an intron) comprising a splice donor sequence at its 5′ end and a splice acceptor sequence at its 3′ end, and a first nuclease recognition sequence at its 3′ end; and a first homology region. The second polynucleotide comprises a second nucleic acid sequence comprising, from 5′ to 3′: a 5′ homology region having homology to at least a portion of the untranslated sequence of the first polynucleotide; a 5′ portion of the first nuclease recognition sequence; a second donor nucleic acid sequence comprising, from 5′ to 3′, a second transgene and a 5′ portion of the first nuclease recognition sequence; a 3′ portion of the first nuclease recognition sequence which is adjacent to the 5′ portion and generates a second nuclease recognition sequence which is identical to the first nuclease recognition sequence; and a 3′ homology region having homology to the first homology region of the first polynucleotide. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the second promoter, the untranslated sequence, the 5′ portion of the first nuclease recognition sequence, the second transgene, the 5′ portion of the second nuclease recognition sequence, the 3′ portion of the first nuclease recognition sequence, and the first homology region. In those embodiments wherein the untranslated sequence comprises an intron comprising both a splice donor sequence and a splice acceptor sequence, the untranslated sequence can be spliced out by a splicing complex, allowing for operable linkage of the second promoter to the second transgene. See, for example, FIG. 5B. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a first donor nucleic acid sequence comprising, from 5′ to 3′, a promoter at its 5′ end, a first transgene operably linked to the promoter, a 2A or IRES sequence, a first untranslated sequence (e.g., an intron) comprising a splice donor sequence at its 5′ end, and a first nuclease recognition sequence at its 3′ end; and a first homology region. The second polynucleotide comprises a second nucleic acid sequence comprising, from 5′ to 3′: a 5′ homology region having homology to at least a portion of the first donor nucleic acid sequence of the first polynucleotide; a first untranslated sequence identical to, or having a high degree of homology with, the first untranslated sequence of the first polynucleotide; a 5′ portion of the first nuclease recognition sequence; a second donor nucleic acid sequence comprising, from 5′ to 3′, a second untranslated sequence (e.g., and intron) comprising a splice acceptor sequence at its 3′ end, a second transgene, and a 5′ portion of the first nuclease recognition sequence; a 3′ portion of the first nuclease recognition sequence which is adjacent to the 5′ portion and generates a second nuclease recognition sequence which is identical to the first nuclease recognition sequence; and a 3′ homology region having homology to the first homology region of the first polynucleotide. Following cleavage of the first nuclease recognition sequence by an engineered nuclease, insertion of the second donor nucleic acid sequence into the first polynucleotide results in a polynucleotide comprising, from 5′ to 3′, the promoter that is operably linked to the first transgene, the 2A sequence, the first untranslated sequence, the 5′ portion of the first recognition sequence, the second untranslated sequence, the second transgene, the 5′ portion of the second nuclease recognition sequence, the 3′ portion of the first nuclease recognition sequence, and the first homology region. In those embodiments wherein the first untranslated sequence comprises an intron comprising a splice donor sequence and the second untranslated sequence comprises an intron comprising a splice acceptor sequence, the two untranslated sequences and the 5′ portion of the first nuclease recognition sequence can be spliced out by a splicing complex, allowing for the promoter to be operably linked to both the first and the second transgene. See, for example, FIG. 6A. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

In particular embodiments of the compositions, eukaryotic cells, and methods described herein, the first polynucleotide of the composition comprises a first nucleic acid sequence comprising, from 5′ to 3′: a first donor nucleic acid sequence comprising, from 5′ to 3′, a promoter at its 5′ end, a first transgene operably linked to the promoter, a first untranslated sequence (e.g., an intron) comprising a splice donor sequence at its 5′ end, and a first nuclease recognition sequence at its 3′ end; and a first homology region. The second polynucleotide comprises a second nucleic acid sequence comprising, from 5′ to 3′: a 5′ homology region having homology to at least a portion of the first donor nucleic acid sequence of the first polynucleotide; a first untranslated sequence identical to, or has a high degree of homology with, the first untranslated sequence of the first polynucleotide; a 5′ portion of the first nuclease recognition sequence; a second donor nucleic acid sequence comprising, from 5′ to 3′, a second untranslated sequence (e.g., and intron) comprising a splice acceptor sequence at its 3′ end, a 2A or IRES sequence, a second transgene, and a 5′ portion of the first nuclease recognition sequence; a 3′ portion of the first nuclease recognition sequence which is adjacent to the 5′ portion and generates a second nuclease recognition sequence which is identical to the first nuclease recognition sequence; and a 3′ homology region having homology to the first homology region of the first polynucleotide. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the first untranslated sequence, the 5′ portion of the first nuclease recognition sequence, the second untranslated sequence, the 2A sequence, the second transgene, the 5′ portion of the second nuclease recognition sequence, the 3′ portion of the first nuclease recognition sequence, and the first homology region. In those embodiments wherein the first untranslated sequence comprises an intron comprising a splice donor sequence and the second untranslated sequence comprises an intron comprising a splice acceptor sequence, the two untranslated sequences and the 5′ portion of the first nuclease recognition sequence can be spliced out by a splicing complex, allowing for the first promoter to be operably linked to both the first and the second transgene. See, for example, FIG. 6B. In some such embodiments, each of the first and second polynucleotides comprise only one D sequence, wherein the D sequence is positioned at the 3′ end of the 5′ ITR, overlaps the 3′ end of the 5′ ITR, or is positioned adjacent to the 3′ end of the 5′ ITR of each polynucleotide. In other such embodiments, each of the first and second polynucleotides comprise only one D sequence, wherein the D sequence is positioned at the 5′ end of the 3′ ITR, overlaps the 5′ end of the 3′ ITR, or is positioned adjacent to the 5′ end of the 3′ ITR of each polynucleotide.

### BRIEF DESCRIPTION OF THE FIGURES

FIGS. 1A-1C provide non-limiting examples of presently disclosed compositions comprising two polynucleotides that can be utilized to insert a portion of the sequence of the second polynucleotide into the first polynucleotide through nuclease cleavage and homologous recombination.

FIG. 1A shows two polynucleotides, the first of which comprises, from 5′ to 3′, a first donor nucleic acid sequence comprising a nuclease recognition sequence for an engineered nuclease, and a first homology region. The second polynucleotide comprises, from 5′ to 3′, a 5′ homology arm having homology to at least a portion of the first donor nucleic acid sequence and to a 5′ portion of the nuclease recognition sequence, a second donor nucleic acid sequence, and a 3′ homology arm having homology to a 3′ portion of the nuclease recognition sequence and the first homology region. Following cleavage of the nuclease recognition sequence by an engineered nuclease, homologous recombination between the two polynucleotides results in an insertion of the second donor nucleic acid sequence into the nuclease recognition sequence.

FIG. 1B depicts two polynucleotides, the first of which comprises, from 5′ to 3′, a first donor nucleic acid sequence comprising a first nuclease recognition sequence for an engineered nuclease, and a first homology region. The second polynucleotide comprises, from 5′ to 3′, a 5′ homology arm having homology to at least a portion of the first donor nucleic acid sequence and to a 5′ portion of the first nuclease recognition sequence, a second donor nucleic acid sequence comprising a 5′ portion of a second nuclease recognition sequence that is identical to the first nuclease recognition sequence, and a 3′ homology arm having homology to the 3′ portion of the first nuclease recognition sequence and the first homology region. Following cleavage of the first nuclease recognition sequence by an engineered nuclease, homologous recombination between the two polynucleotides results in an insertion of the second donor nucleic acid sequence into the first nuclease recognition sequence and the resulting polynucleotide comprises a nuclease recognition sequence to allow for a subsequent insertion of a donor nucleic acid sequence from another polynucleotide.

FIG. 1C depicts two polynucleotides, the first of which comprises, from 5′ to 3′, a first donor nucleic sequence comprising a first nuclease recognition sequence for an engineered nuclease, and a first homology region. The second polynucleotide comprises, from 5′ to 3′, a 5′ homology arm having homology to at least a portion of the first donor nucleic acid sequence and to a 5′ portion of the first nuclease recognition sequence, a second donor nucleic acid sequence comprising a second nuclease recognition sequence, and a 3′ homology arm having homology to a 3′ portion of the first nuclease recognition sequence and the first homology region. In this example, the first and second nuclease recognition sequences are not identical. Following cleavage of the first nuclease recognition sequence by an engineered nuclease, homologous recombination between the two polynucleotides results in an insertion of the second donor nucleic acid sequence into the first nuclease recognition sequence and the resulting polynucleotide comprises the second nuclease recognition sequence to allow for a subsequent insertion of a donor nucleic acid sequence from another polynucleotide.

FIGS. 2A-2D provide non-limiting examples of presently disclosed compositions comprising two polynucleotides that can be utilized to recombine a portion of the first polynucleotide into a genome of a desired organism and to recombine a portion of the second polynucleotide into the first polynucleotide within the genome.

FIG. 2A shows two polynucleotides, the first of which comprises, from 5′ to 3′, a 5′ inverted terminal repeat (ITR), a 5′ homology arm that is homologous to a sequence 5′ upstream of an endogenous nuclease recognition sequence and to a 5′ portion of the endogenous nuclease recognition sequence, a first donor nucleic acid sequence comprising a first nuclease recognition sequence that is identical to the endogenous nuclease recognition sequence, a first homology region, and a 3′ ITR. The second polynucleotide comprises, from 5′ to 3′, a 5′ ITR, a 5′ homology arm having homology to at least a portion of the first donor nucleic acid sequence and to a 5′ portion of the first nuclease recognition sequence, a second donor nucleic acid sequence, a 3′ homology arm having homology to a 3′ portion of the first nuclease recognition sequence and the first homology region, and a 3′ ITR. Following cleavage of the endogenous nuclease recognition sequence and the first nuclease recognition sequence, homologous recombination between the first polynucleotide and the genomic region comprising the endogenous nuclease recognition sequence and between the first and second polynucleotide results in insertion of the first and second donor nucleic acid sequence into the genome within the endogenous nuclease recognition sequence.

FIGS. 2B and 2C depict the potential two-step insertion of the first and second donor nucleic acid sequences of the polynucleotides depicted in FIG. 2A into the genome. FIG. 2B depicts the cleavage of an endogenous nuclease recognition sequence by a nuclease and the subsequent insertion of a first polynucleotide comprising, from 5′ to 3′, a 5′ ITR, a 5′ homology arm that is homologous to a sequence 5′ upstream of the endogenous nuclease recognition sequence and to a 5′ portion of the endogenous nuclease recognition sequence, a first donor nucleic acid sequence comprising a first nuclease recognition sequence that is identical to the endogenous nuclease recognition sequence, a first homology region, and a 3′ ITR. FIG. 2C depicts the cleavage of the first nuclease recognition sequence within the first donor nucleic acid sequence that has previously been inserted into the genome (in FIG. 2B) within an endogenous nuclease recognition sequence and insertion of the second donor sequence from the second polynucleotide into the genome.

FIG. 2D shows two polynucleotides, the first of which comprises, from 5′ to 3′, a 5′ ITR, a 5′ homology arm that is homologous to a sequence 5′ upstream of an endogenous nuclease recognition sequence and to a 5′ portion of the endogenous nuclease recognition sequence, a first donor nucleic acid sequence comprising a first nuclease recognition sequence that is not identical to the endogenous nuclease recognition sequence, a first homology region, a 3′ homology arm that is homologous to a 3′ portion of the endogenous nuclease recognition sequence and a sequence 3′ downstream from the endogenous nuclease recognition sequence, and a 3′ ITR. The second polynucleotide comprises, from 5′ to 3′, a 5′ ITR, a 5′ homology arm having homology to at least a portion of the first donor nucleic acid sequence and to a 5′ portion of the first nuclease recognition sequence, a second donor nucleic acid sequence, a 3′ homology arm having homology to a 3′ portion of the first nuclease recognition sequence and the first homology region, and a 3′ ITR. Following cleavage of the endogenous nuclease recognition sequence by a first engineered nuclease and the cleavage of the first nuclease recognition sequence by a second engineered nuclease, homologous recombination between the first polynucleotide and the genomic region comprising the endogenous nuclease recognition sequence and between the first and second polynucleotide results in insertion of the first and second donor nucleic acid sequence into the genome within the endogenous nuclease recognition sequence.

FIGS. 3A-3C provide non-limiting examples of presently disclosed compositions comprising two polynucleotides that can be utilized to recombine a portion of the first polynucleotide into a genome of a desired organism and to recombine a portion of the second polynucleotide into the first polynucleotide within the genome. The second polynucleotide further comprises an additional nuclease recognition sequence that can be used for the insertion of an additional polynucleotide into the genome.

FIG. 3A depicts two polynucleotides and a genomic region comprising an endogenous nuclease recognition sequence. The first polynucleotide comprises, from 5′ to 3′, a 5′ ITR, a 5′ homology arm that is homologous to a sequence 5′ upstream of an endogenous nuclease recognition sequence and to a 5′ portion of the endogenous nuclease recognition sequence, a first donor nucleic acid sequence comprising a first nuclease recognition sequence that is identical to the endogenous nuclease recognition sequence, a first homology region, and a 3′ ITR. The second polynucleotide comprises, from 5′ to 3′, a 5′ ITR, a 5′ homology arm having homology to at least a portion of the first donor nucleic acid sequence and to a 5′ portion of the first nuclease recognition sequence, a second donor nucleic acid sequence comprising a 5′ portion of a second nuclease recognition sequence that is identical to the first nuclease recognition sequence and the endogenous nuclease recognition sequence, a 3′ homology arm having homology to a 3′ portion of the first nuclease recognition sequence and the first homology region, and a 3′ ITR. Following cleavage of the endogenous nuclease recognition sequence and the first nuclease recognition sequence, homologous recombination between the first polynucleotide and the genomic region comprising the endogenous nuclease recognition sequence and between the first and second polynucleotide results in insertion of the first and second donor nucleic acid sequence into the genome within the endogenous nuclease recognition sequence. The presence of a nuclease recognition sequence within the genome following the recombination and insertion allows for the potential insertion of additional exogenous sequence into the genome.

FIG. 3B depicts two polynucleotides and a genomic region comprising an endogenous nuclease recognition sequence. The first polynucleotide comprises, from 5′ to 3′, a 5′ ITR, a 5′ homology arm that is homologous to a sequence 5′ upstream of an endogenous nuclease recognition sequence and to a 5′ portion of the endogenous nuclease recognition sequence, a first donor nucleic acid sequence comprising a first nuclease recognition sequence that is identical to the endogenous nuclease recognition sequence, a first homology region, and a 3′ ITR. The second polynucleotide comprises, from 5′ to 3′, a 5′ ITR, a 5′ homology arm having homology to at least a portion of the first donor nucleic acid sequence and to a 5′ portion of the first nuclease recognition sequence, a second donor nucleic acid sequence comprising a second nuclease recognition sequence that is different from the first nuclease recognition sequence and the endogenous nuclease recognition sequence, a 3′ homology arm having homology to a 3′ portion of the first nuclease recognition sequence and the first homology region, and a 3′ ITR. Following cleavage of the endogenous nuclease recognition sequence and the first nuclease recognition sequence, homologous recombination between the first polynucleotide and the genomic region comprising the endogenous nuclease recognition sequence and between the first and second polynucleotide results in insertion of the first and second donor nucleic acid sequence into the genome within the endogenous nuclease recognition sequence. The presence of a nuclease recognition sequence within the genome following the recombination and insertion allows for the potential insertion of additional exogenous sequence into the genome following cleavage of the second nuclease recognition sequence by an engineered nuclease that is different from the engineered nuclease that cleaved the endogenous nuclease recognition sequence and the first nuclease recognition sequence.

FIG. 3C depicts two polynucleotides and a genomic region comprising an endogenous nuclease recognition sequence. The first polynucleotide comprises, from 5′ to 3′, a 5′ ITR, a 5′ homology arm that is homologous to a sequence 5′ upstream of an endogenous nuclease recognition sequence and to a 5′ portion of the endogenous nuclease recognition sequence, a first donor nucleic acid sequence comprising a first nuclease recognition sequence, a first homology region, a 3′ homology arm having homology to a 3′ portion of the endogenous nuclease recognition sequence and to a sequence 3′ downstream of the endogenous nuclease recognition sequence, and a 3′ ITR. The second polynucleotide comprises, from 5′ to 3′, a 5′ ITR, a 5′ homology arm having homology to at least a portion of the first donor nucleic acid sequence and to a 5′ portion of the first nuclease recognition sequence, a second donor nucleic acid sequence comprising a second nuclease recognition sequence, a 3′ homology arm having homology to a 3′ portion of the first nuclease recognition sequence and the first homology region, and a 3′ ITR. Each of the endogenous, the first, and the second nuclease recognition sequences are unique from each other and are recognized by three unique engineered nucleases. Following cleavage of the endogenous nuclease recognition sequence by a first engineered nuclease and the first nuclease recognition sequence by a second engineered nuclease, homologous recombination between the first polynucleotide and the genomic region comprising the endogenous nuclease recognition sequence and between the first and second polynucleotide results in insertion of the first and second donor nucleic acid sequence into the genome within the endogenous nuclease recognition sequence. The presence of a nuclease recognition sequence within the genome following the recombination and insertion allows for the potential insertion of additional exogenous sequence into the genome following cleavage of the second nuclease recognition sequence by a third engineered nuclease.

FIGS. 4A-4B provide non-limiting examples of presently disclosed compositions comprising two polynucleotides, each of which comprise a portion of a transgene that allows for the subsequent recombination of the two polynucleotides into a single polynucleotide comprising the full-length transgene.

FIG. 4A shows two polynucleotides, the first of which comprises, from 5′ to 3′, a first donor nucleic acid sequence comprising a promoter operably linked to a first portion of a transgene, a first untranslated sequence, and a first nuclease recognition sequence, and a first homology region. The second polynucleotide comprises, from 5′ to 3′, a 5′ homology arm having homology to at least a portion of the first transgene portion, the first untranslated sequence, and a 5′ portion of the first nuclease recognition sequence, a second donor nucleic acid sequence comprising a second untranslated sequence, a second portion of the transgene, and a 5′ portion of a second nuclease recognition sequence that is identical to the first nuclease recognition sequence, and a 3′ homology arm having homology to a 3′ portion of the first nuclease recognition sequence and the first homology region. Following cleavage of the first nuclease recognition sequence by an engineered nuclease, insertion of the second donor nucleic acid sequence into the first polynucleotide results in a polynucleotide comprising, from 5′ to 3′, the promoter that is operably linked to the first portion of the transgene, the first untranslated sequence, the 5′ portion of the first recognition sequence, the second untranslated sequence, the second portion of the transgene, the 5′ portion of the second nuclease recognition sequence, the 3′ portion of the first nuclease recognition sequence, and the first homology region. If the first untranslated sequence comprises an intron comprising a splice donor sequence and the second untranslated sequence comprises an intron comprising a splice acceptor sequence, the two untranslated sequences and the 5′ portion of the first nuclease recognition sequence can be spliced out by a splicing complex, allowing for the promoter to be operably linked to the complete transgene.

FIG. 4B depicts two polynucleotides, the first of which comprises, from 5′ to 3′, a first donor nucleic acid sequence comprising a first promoter operably linked to a first transgene, a second promoter operably linked to a first portion of a second transgene, a first untranslated sequence, and a first nuclease recognition sequence, and a first homology region. The second polynucleotide comprises, from 5′ to 3′, a 5′ homology arm having homology to at least a portion of the first portion of the second transgene, the first untranslated sequence, and a 5′ portion of the first nuclease recognition sequence, a second donor nucleic acid sequence comprising a second untranslated sequence, a second portion of the second transgene, and a 5′ portion of a second nuclease recognition sequence that is identical to the first nuclease recognition sequence, and a 3′ homology arm having homology to a 3′ portion of the first nuclease recognition sequence and the first homology region. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the second promoter that is operably linked to the first portion of the second transgene, the first untranslated sequence, the 5′ portion of the first nuclease recognition sequence, the second untranslated sequence, the second portion of the second transgene, the 5′ portion of the second nuclease recognition sequence, the 3′ portion of the first nuclease recognition sequence, and the first homology region. If the first untranslated sequence comprises an intron comprising a splice donor sequence and the second untranslated sequence comprises an intron comprising a splice acceptor sequence, the two untranslated sequences and the 5′ portion of the first nuclease recognition sequence can be spliced out by a splicing complex, allowing for the second promoter to be operably linked to the complete second transgene.

FIGS. 5A-5B provides non-limiting examples of presently disclosed compositions comprising two polynucleotides wherein the first polynucleotide comprises a first transgene and the second polynucleotide comprises a second transgene.

FIG. 5A shows two polynucleotides, the first of which comprises, from 5′ to 3′, a first donor nucleic acid sequence comprising a first promoter operably linked to a first transgene and a first nuclease recognition sequence, and a first homology region. The second polynucleotide comprises, from 5′ to 3′, a 5′ homology arm having homology to at least a portion of the first transgene and a 5′ portion of the first nuclease recognition sequence, a second donor nucleic acid sequence comprising a second promoter operably linked to a second transgene, and a 5′ portion of a second nuclease recognition sequence that is identical to the first nuclease recognition sequence, and a 3′ homology arm having homology to a 3′ portion of the first nuclease recognition sequence and the first homology region. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the 5′ portion of the first nuclease recognition sequence, the second promoter that is operably linked to the second transgene, the 5′ portion of the second nuclease recognition sequence, the 3′ portion of the first nuclease recognition sequence, and the first homology region.

FIG. 5B depicts two polynucleotides, the first of which comprises, from 5′ to 3′, a first donor nucleic acid sequence comprising a first promoter operably linked to a first transgene, a second promoter, an untranslated sequence, and a first nuclease recognition sequence, and a first homology region. The second polynucleotide comprises, from 5′ to 3′, a 5′ homology arm having homology to at least a portion of the untranslated sequence and a 5′ portion of the first nuclease recognition sequence, a second donor nucleic acid sequence comprising a second transgene and a 5′ portion of a second nuclease recognition sequence that is identical to the first nuclease recognition sequence, and a 3′ homology arm having homology to a 3′ portion of the first nuclease recognition sequence and the first homology region. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the second promoter, the untranslated sequence, the 5′ portion of the first nuclease recognition sequence, the second transgene, the 5′ portion of the second nuclease recognition sequence, the 3′ portion of the first nuclease recognition sequence, and the first homology region. If the untranslated sequence comprises an intron comprising both a splice donor sequence and a splice acceptor sequence, the untranslated sequence can be spliced out by a splicing complex, allowing for operable linkage of the second promoter to the second transgene.

FIGS. 6A-6B provides non-limiting examples of presently disclosed compositions comprising two polynucleotides wherein a single promoter present in the first polynucleotide is operably linked to both a first transgene and a second transgene following recombination of the two polynucleotides and insertion of the second donor nucleic acid sequence into the first polynucleotide.

FIG. 6A shows two polynucleotides, the first of which comprises, from 5′ to 3′, a first donor nucleic acid sequence comprising a promoter operably linked to a first transgene, a 2A sequence, a first untranslated sequence, and a first nuclease recognition sequence, and a first homology region. The second polynucleotide comprises, from 5′ to 3′, a 5′ homology arm having homology to at least the 2A sequence, the first untranslated sequence and a 5′ portion of the first nuclease recognition sequence, a second donor nucleic acid sequence comprising a second untranslated sequence, a second transgene, and a 5′ portion of a second nuclease recognition sequence that is identical to the first nuclease recognition sequence, and a 3′ homology arm having homology to a 3′ portion of the first nuclease recognition sequence and the first homology region. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the 2A sequence, the first untranslated sequence, the 5′ portion of the first recognition sequence, the second untranslated sequence, the second transgene, the 5′ portion of the second nuclease recognition sequence, the 3′ portion of the first nuclease recognition sequence, and the first homology region. If the first untranslated sequence comprises an intron comprising a splice donor sequence and the second untranslated sequence comprises an intron comprising a splice acceptor sequence, the two untranslated sequences and the 5′ portion of the first nuclease recognition sequence can be spliced out by a splicing complex, allowing for the first promoter to be operably linked to both the first and the second transgene.

FIG. 6B shows two polynucleotides, the first of which comprises, from 5′ to 3′, a first donor nucleic acid sequence comprising a promoter operably linked to a first transgene, a first untranslated sequence, and a first nuclease recognition sequence, and a first homology region. The second polynucleotide comprises, from 5′ to 3′, a 5′ homology arm having homology to at least a portion of the first transgene, the first untranslated sequence, and a 5′ portion of the first nuclease recognition sequence, a second donor nucleic acid sequence comprising a second untranslated sequence, a 2A sequence, a second transgene, and a 5′ portion of a second nuclease recognition sequence that is identical to the first nuclease recognition sequence, and a 3′ homology arm having homology to a 3′ portion of the first nuclease recognition sequence and the first homology region. Following cleavage of the first nuclease recognition sequence by an engineered nuclease, insertion of the second donor nucleic acid sequence into the first polynucleotide results in a polynucleotide comprising, from 5′ to 3′, the first promoter that is operably linked to the first transgene, the first untranslated sequence, the 5′ portion of the first nuclease recognition sequence, the second untranslated sequence, the 2A sequence, the second transgene, the 5′ portion of the second nuclease recognition sequence, the 3′ portion of the first nuclease recognition sequence, and the first homology region. If the first untranslated sequence comprises an intron comprising a splice donor sequence and the second untranslated sequence comprises an intron comprising a splice acceptor sequence, the two untranslated sequences and the 5′ portion of the first nuclease recognition sequence can be spliced out by a splicing complex, allowing for the first promoter to be operably linked to both the first and the second transgene.

FIG. 7 provides a map of the plasmid encoding the 7373 construct comprising the following elements from 5′ to 3′: a 5′ ITR: a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; an SV40 polyA sequence; an EF1-alpha promoter; an intron sequence containing a 5′ splice donor and 3′ splice acceptor; a full TRC 1-2 recognition sequence; a second homology region of 287 by and a 3′ ITR.

FIG. 8 provides a map of the plasmid encoding the 7374 construct comprising the following elements from 5′ to 3′: a 5′ ITR; a single D sequence; an intron sequence containing a 5′ splice donor and 3′ splice acceptor having homology to the intron sequence of construct 7373; the 5′ half-site of the TRC 1-2 recognition sequence; a GFP coding sequence; an SV40 polyA sequence; a full TRC 1-2 recognition sequence; a homology region having homology to the sequence 3′ downstream of the endogenous TRC 1-2 recognition sequence and a 3′ ITR.

FIG. 9 provides a map of the plasmid encoding the 7375 construct comprising the following elements from 5′ to 3′: a 5′ ITR; a single D sequence; an intron sequence containing a 5′ splice donor (not depicted) and 3′ splice acceptor having homology to the intron sequence of construct 7373; the 5′ half-site of the TRC 1-2 recognition sequence; a GFP coding sequence; an SV40 polyA sequence; a homology region having homology to the sequence 3′ downstream of the endogenous TRC 1-2 recognition sequence and a 3′ ITR.

FIG. 10 provides a map of the plasmid encoding the 73234 construct comprising the following elements from 5′ to 3′: a 5′ ITR; a single D sequence; an intron sequence containing a 5′ splice donor (not depicted) and 3′ splice acceptor having homology to the intron sequence of construct 7373; the 5′ half-site of the TRC 1-2 recognition sequence; a GFP coding sequence; an SV40 polyA sequence; a full HAO 1-2 recognition sequence; the 3′ half-site of the TRC1-2 recognition sequence; a homology region having 750 bp homology to the sequence 3′ downstream of the endogenous TRC 1-2 recognition sequence and a 3′ ITR.

FIG. 11 provides a map of the plasmid encoding the 73235 construct comprising the following elements from 5′ to 3′: a 5′ ITR, a single D sequence, a beta-2M-specific left homology arm, a 5′ portion of a beta-2M recognition sequence, a JeT promoter, a coding sequence for a CD19-specific chimeric antigen receptor, an EF1-alpha promoter, an intron sequence containing a 5′ splice donor and 3′ splice acceptor having homology to the intron sequence of construct 7373, a full TRC 1-2 recognition sequence, a homology region having 287 bp homology to the sequence 3′ downstream of the endogenous TRC 1-2 recognition sequence, the 3′ half-site of the beta-2M recognition sequence, and a 3′ ITR.

FIG. 12 provides a map of the plasmid encoding the 73236 construct comprising the following elements from 5′ to 3′: a 5′ ITR; a single D sequence; the 5′ 350 bp an intron sequence having homology to the intron sequence of construct 73237; the 5′ half-site of the TRC 1-2 recognition sequence; the 3′ 326 bp of the intron that contains a splice acceptor; the 3′ 477 bp of the GFP coding sequence; an SV40 polyA sequence; a full TRC 1-2 recognition sequence; a homology region of 750 bp having homology to the sequence 3′ downstream of the endogenous TRC 1-2 recognition sequence and a 3′ ITR.

FIG. 13 provides a map of the plasmid encoding the 73237 construct comprising the following elements from 5′ to 3′: a 5′ ITR; a single D sequence; a first homology region of 972 bp having homology to the sequence 5′ upstream of the endogenous TRC 1-2 recognition sequence; the 5′ half-site of the TRC 1-2 recognition sequence; an EF1-alpha promoter; the 5′ 237 bp of the GFP coding sequence; the 5′ 500 bp of the intron containing a 5′ splice donor; a full TRC 1-2 recognition sequence; a second homology region of 750 bp and a 3′ ITR.

FIG. 14 provides a map of the plasmid encoding the 73238 construct comprising the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a bGH polyA sequence; an EF1-alpha promoter; the 5′ 237 bp of the GFP coding sequence; the 5′ 500 bp of the intron containing a splice donor having homology to the intron sequence of construct 73236; a full TRC 1-2 recognition sequence; a second homology region of 750 bp and a 3′ ITR.

FIG. 15 provides a map of the plasmid encoding the 73245 construct comprising the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a bGH polyA sequence; a full TRC 1-2 recognition sequence; a second homology region of 750 bp; and a 3′ ITR.

FIG. 16 provides a map of the plasmid encoding the 73246 construct comprising the following elements from 5′ to 3′: a 5′ ITR; a single D sequence; the 3′ 365 bp of the CAR coding sequence (denoted as R) having homology to the CAR coding sequence in 73245; a bGH polyA sequence; the 5′ half-site of the TRC 1-2 recognition sequence; an EF1-alpha promoter; a GFP coding sequence; an SV40 polyA sequence; a full TRC 1-2 recognition sequence; a homology region of 750 bp having homology to the sequence 3′ downstream of the endogenous TRC 1-2 recognition sequence and a 3′ ITR.

FIG. 17 provides a map of the plasmid encoding the 73247 construct comprising the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a bGH polyA sequence; an EF1-alpha promoter; 463 bp of an intron sequence containing a splice acceptor and homology to the intron in 7374; a full TRC 1-2 recognition sequence; a second 750 bp homology region and a 3′ ITR.

FIG. 18 provides a map of the plasmid encoding the 73248 construct comprising the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a 2A element; the 5′ 500 bp of an intron sequence containing a splice donor; a full TRC 1-2 recognition sequence; a 750 bp second homology region; and a 3′ ITR.

FIG. 19 provides a map of the plasmid encoding the 73249 construct comprising the following elements from 5′ to 3′: a 5′ ITR; a single D sequence; the 3′ 365 bp of the CAR coding sequence (denoted as (R)) having homology to the CAR sequence in 73248; a P2A cleavage sequence; the 5′ 350 bp of an intron containing a splice donor; the 5′ half-site of the TRC 1-2 recognition sequence; the 3′ 326 bp of an intron containing a splice donor; a GFP coding sequence; an SV40 polyA sequence; a full TRC 1-2 recognition sequence; a homology region having 750 bp homology to the sequence 3′ downstream of the endogenous TRC 1-2 recognition sequence and a 3′ ITR.

FIG. 20 provides a map of the plasmid encoding the 73250 construct comprising the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; the 5′ 500 bp of an intron sequence containing a splice donor; a full TRC 1-2 recognition sequence; a second 750 bp homology region; and a 3′ ITR.

FIG. 21 provides a map of the plasmid encoding the 73251 construct comprising the following elements from 5′ to 3′: a 5′ ITR; a single D sequence; the 3′ 365 bp of a CAR (denoted as (R)) having homology to the CAR sequence in 73250; the 5′ 350 bp of an intron; the 5′ half-site of the TRC 1-2 recognition sequence; the 3′ 326 bp of an intron containing a splice acceptor; a 2A element; a GFP coding sequence; an SV40 polyA sequence; a full TRC 1-2 recognition sequence; a 750 bp homology region having homology to the sequence 3′ downstream of the endogenous TRC 1-2 recognition sequence and a 3′ ITR.

FIGS. 22A-22C show flow cytometry results obtained from primary T cells electroporated with a TRC 1-2L.1592 meganuclease in the absence of AAVs providing a first or second polynucleotide template for stacking. FIG. 22A shows staining for cell-surface CD3 expression (X-axis; as a measure of cell-surface TCR expression) and expression of a CD19 chimeric antigen receptor (CAR) (Y-axis). FIG. 22B shows staining for GFP-positive cells (X-axis) and CAR (Y-axis) within the population of CD3-negative/CAR-positive cells. FIG. 22C shows staining for cell-surface CD3 (X-axis) and GFP (Y-axis).

FIGS. 23A-23C show flow cytometry results obtained from primary T cells electroporated with a TRC 1-2L.1592 meganuclease and further transduced with an AAV comprising the 7373 construct. FIG. 23A shows staining for cell-surface CD3 expression (X-axis) and expression of a CD19 CAR (Y-axis). FIG. 23B shows staining for GFP-positive cells (X-axis) and CAR (Y-axis) within the population of CD3-negative/CAR-positive cells. FIG. 23C shows staining for cell-surface CD3 (X-axis) and GFP (Y-axis).

FIGS. 24A-24C show flow cytometry results obtained from primary T cells electroporated with a TRC 1-2L.1592 meganuclease and further transduced with an AAV comprising the 7374 construct. FIG. 24A shows staining for cell-surface CD3 expression (X-axis) and expression of a CD19 CAR (Y-axis). FIG. 24B shows staining for GFP-positive cells (X-axis) and CAR (Y-axis) within the population of CD3-negative/CAR-positive cells. FIG. 24C shows staining for cell-surface CD3 (X-axis) and GFP (Y-axis).

FIGS. 25A-25C show flow cytometry results obtained from primary T cells electroporated with a TRC 1-2L.1592 meganuclease and further transduced with an AAV comprising the 7375 construct. FIG. 25A shows staining for cell-surface CD3 expression (X-axis) and expression of a CD19 CAR (Y-axis). FIG. 25B shows staining for GFP-positive cells (X-axis) and CAR (Y-axis) within the population of CD3-negative/CAR-positive cells. FIG. 25C shows staining for cell-surface CD3 (X-axis) and GFP (Y-axis).

FIGS. 26A-26D show flow cytometry results obtained from primary T cells electroporated with a TRC 1-2L.1592 meganuclease and further transduced with a first AAV comprising the 7373 construct and a second AAV comprising the 7374 construct. FIG. 26A shows staining for cell-surface CD3 expression (X-axis) and expression of a CD19 CAR (Y-axis). FIG. 26B shows staining for GFP-positive cells (X-axis) and CAR (Y-axis) within the population of CD3-negative/CAR-positive cells. FIG. 26C shows staining for GFP (X-axis) and expression of the CAR (Y-axis) in cells within the CD3/CAR double-negative population. FIG. 26D shows staining for CD3 (X-axis) and GFP (Y-axis) in cells within the CD3/CAR double-negative population.

FIGS. 27A-27D show flow cytometry results obtained from primary T cells electroporated with a TRC 1-2L.2792 meganuclease and further transduced with a first AAV comprising the 7373 construct and a second AAV comprising the 7374 construct. FIG. 27A shows staining for cell-surface CD3 expression (X-axis) and expression of a CD19 CAR (Y-axis). FIG. 27B shows staining for GFP-positive cells (X-axis) and CAR (Y-axis) within the population of CD3-negative/CAR-positive cells. FIG. 27C shows staining for GFP (X-axis) and expression of the CAR (Y-axis) in cells within the CD3/CAR double-negative population. FIG. 27D shows staining for CD3 (X-axis) and GFP (Y-axis) in cells within the CD3/CAR double-negative population.

FIGS. 28A-28D show flow cytometry results obtained from primary T cells electroporated with a TRC 1-2L.1592 meganuclease and transduced with a first AAV comprising the 7373 construct, then further transduced with a second AAV comprising the 7374 construct 22 hours later. FIG. 28A shows staining for cell-surface CD3 expression (X-axis) and expression of a CD19 CAR (Y-axis). FIG. 28B shows staining for GFP-positive cells (X-axis) and CAR (Y-axis) within the population of CD3-negative/CAR-positive cells. FIG. 28C shows staining for GFP (X-axis) and expression of the CAR (Y-axis) in cells within the CD3/CAR double-negative population. FIG. 28D shows staining for CD3 (X-axis) and GFP (Y-axis) in cells within the CD3/CAR double-negative population.

FIGS. 29A-29D show flow cytometry results obtained from primary T cells electroporated with a TRC 1-2L.1592 meganuclease and transduced with a first AAV comprising the 7373 construct, then further transduced with a second AAV comprising the 7375 construct 22 hours later. FIG. 29A shows staining for cell-surface CD3 expression (X-axis) and expression of a CD19 CAR (Y-axis). FIG. 29B shows staining for GFP-positive cells (X-axis) and CAR (Y-axis) within the population of CD3-negative/CAR-positive cells. FIG. 29C shows staining for GFP (X-axis) and expression of the CAR (Y-axis) in cells within the CD3/CAR double-negative population. FIG. 29D shows staining for CD3 (X-axis) and GFP (Y-axis) in cells within the CD3/CAR double-negative population.

FIGS. 30A-30B show flow cytometry results obtained from primary T cells electroporated with a TRC 1-2L.1592 meganuclease in the absence of AAVs providing a first or second polynucleotide template for stacking. FIG. 30A shows staining for cell-surface CD3 expression (X-axis; as a measure of cell-surface TCR expression) and expression of a CD19 chimeric antigen receptor (CAR) (Y-axis). FIG. 30B shows staining for GFP-positive cells (X-axis) and CAR (Y-axis) within the population of CD3-negative/CAR-positive cells.

FIGS. 31A-31C show flow cytometry results obtained from primary T cells electroporated with a TRC 1-2L.1592 meganuclease and further transduced with an AAV comprising the 7373 construct. FIG. 31A shows staining for cell-surface CD3 expression (X-axis) and expression of a CD19 CAR (Y-axis). FIG. 31B shows staining for GFP-positive cells (X-axis) and CAR (Y-axis) within the population of CD3-negative/CAR-positive cells. FIG. 31C shows staining for cell-surface CAR (Y-axis) and GFP (X-axis) within the population of CD3-negative/CAR-negative cells.

FIGS. 32A-32D show flow cytometry results obtained from primary T cells electroporated with a TRC 1-2L.1592 meganuclease and further transduced with an AAV comprising the 7374 construct or the 73234 construct. FIG. 32A and FIG. 32C shows staining for cell-surface CD3 expression (X-axis) and expression of a CD19 CAR (Y-axis) for cells transduced with either the 7374 or 73234 construct, respectively. FIG. 32B and FIG. 32D shows staining for GFP-positive cells (X-axis) and CAR (Y-axis) within the population of CD3-negative/CAR-negative cells for cells transduced with either the 7374 or 73234 construct, respectively.

FIGS. 33A-33C show flow cytometry results obtained from primary T cells electroporated with a TRC 1-2L.1592 meganuclease and further transduced with a first AAV comprising the 7373 construct and a second AAV comprising the 7374 construct. FIG. 33A shows staining for cell-surface CD3 expression (X-axis) and expression of a CD19 CAR (Y-axis). FIG. 33B shows staining for GFP-positive cells (X-axis) and CAR (Y-axis) within the population of CD3-negative/CAR-positive cells. FIG. 33C shows staining for GFP (X-axis) and expression of the CAR (Y-axis) in cells within the CD3/CAR double-negative population.

FIGS. 34A-34C show flow cytometry results obtained from primary T cells electroporated with a TRC 1-2L.1592 meganuclease and further transduced with a first AAV comprising the 7373 construct and a second AAV comprising the 73234 construct. FIG. 34A shows staining for cell-surface CD3 expression (X-axis) and expression of a CD19 CAR (Y-axis). FIG. 34B shows staining for GFP-positive cells (X-axis) and CAR (Y-axis) within the population of CD3-negative/CAR-positive cells. FIG. 34C shows staining for GFP (X-axis) and expression of the CAR (Y-axis) in cells within the CD3/CAR double-negative population.

FIGS. 35A-35B show flow cytometry results obtained from primary T cells electroporated with a TRC 1-2L.1592 meganuclease in the absence of AAVs providing a first or second polynucleotide template for stacking. FIG. 35A shows staining for cell-surface CD3 expression (X-axis; as a measure of cell-surface TCR expression) and expression of a CD19 chimeric antigen receptor (CAR) (Y-axis). FIG. 35B shows staining for GFP-positive cells (X-axis) and CAR (Y-axis) within the population of CD3-negative/CAR-positive cells.

FIGS. 36A-36D show flow cytometry results obtained from primary T cells electroporated with a TRC 1-2L.1592 meganuclease and further transduced with an AAV comprising the 73236 construct or the 73237 construct. FIG. 36A and FIG. 36C shows staining for cell-surface CD3 expression (X-axis) and expression of a CD19 CAR (Y-axis) for cells transduced with either the 73236 or 73237 construct, respectively. FIG. 36B and FIG. 36D shows staining for GFP-positive cells (X-axis) and CAR (Y-axis) within the population of CD3-negative/CAR-negative cells for cells transduced with either the 73236 or 73237 construct, respectively.

FIGS. 37A-37C show flow cytometry results obtained from primary T cells electroporated with a TRC 1-2L.1592 meganuclease and further transduced with an AAV comprising the 73238 construct. FIG. 37A shows staining for cell-surface CD3 expression (X-axis) and expression of a CD19 CAR (Y-axis). FIG. 37B shows staining for GFP-positive cells (X-axis) and CAR (Y-axis) within the population of CD3-negative/CAR-positive cells. FIG. 37C shows staining for cell-surface CAR (Y-axis) and GFP (X-axis) within the population of CD3-negative/CAR-negative cells.

FIGS. 38A-38B show flow cytometry results obtained from primary T cells electroporated with a TRC 1-2L.1592 meganuclease and further transduced with a first AAV comprising the 73236 construct and a second AAV comprising the 73237 construct. FIG. 38A shows staining for cell-surface CD3 expression (X-axis) and expression of a CD19 CAR (Y-axis). FIG. 38B shows staining for GFP-positive cells (X-axis) and CAR (Y-axis) within the CD3/CAR double-negative population. Neither of the 73236 or 73237 constructs have a CAR expression cassette, therefore, staining for CAR expression was done only for consistency amongst other experiments.

FIGS. 39A-39C show flow cytometry results obtained from primary T cells electroporated with a TRC 1-2L.1592 meganuclease and further transduced with a first AAV comprising the 73236 construct and a second AAV comprising the 73237 construct. FIG. 39A shows staining for cell-surface CD3 expression (X-axis) and expression of a CD19 CAR (Y-axis). FIG. 39B shows staining for GFP-positive cells (X-axis) and CAR (Y-axis) within the population of CD3-negative/CAR-positive cells. FIG. 39C shows staining for GFP (X-axis) and expression of the CAR (Y-axis) in cells within the CD3/CAR double-negative population.

FIGS. 40A-40C show flow cytometry results obtained from primary T cells electroporated with a TRC 1-2L.1592 meganuclease and further transduced with an AAV comprising the 73245 construct. FIG. 40A shows staining for cell-surface CD3 expression (X-axis) and expression of a CD19 CAR (Y-axis). FIG. 40B shows staining for GFP-positive cells (X-axis) and CAR (Y-axis) within the population of CD3-negative/CAR-positive cells. FIG. 40C shows staining for cell-surface CAR (Y-axis) and GFP (X-axis) within the population of CD3-negative/CAR-negative cells.

FIGS. 41A-41B show flow cytometry results obtained from primary T cells electroporated with a TRC 1-2L.1592 meganuclease and further transduced with an AAV comprising the 73246 construct. FIG. 41A shows staining for cell-surface CD3 expression (X-axis) and expression of a CD19 CAR (Y-axis). FIG. 41B shows staining for cell-surface CAR (Y-axis) and GFP (X-axis) within the population of CD3-negative/CAR-negative cells.

FIGS. 42A-42C show shows flow cytometry results obtained from primary T cells electroporated with a TRC 1-2L.1592 meganuclease and further transduced with an AAV comprising the 73247 construct. FIG. 42A shows staining for cell-surface CD3 expression (X-axis) and expression of a CD19 CAR (Y-axis). FIG. 42B shows staining for GFP-positive cells (X-axis) and CAR (Y-axis) within the population of CD3-negative/CAR-positive cells. FIG. 42C shows staining for cell-surface CAR (Y-axis) and GFP (X-axis) within the population of CD3-negative/CAR-negative cells.

FIGS. 43A-43B show flow cytometry results obtained from primary T cells electroporated with a TRC 1-2L.1592 meganuclease and further transduced with an AAV comprising the 7374 construct. FIG. 43A shows staining for cell-surface CD3 expression (X-axis) and expression of a CD19 CAR (Y-axis). FIG. 43B shows staining for GFP-positive cells (X-axis) and CAR (Y-axis) within the population of CD3-negative/CAR-negative cells.

FIGS. 44A-44C show flow cytometry results obtained from primary T cells electroporated with a TRC 1-2L.1592 meganuclease and further transduced with a first AAV comprising the 73245 construct and a second AAV comprising the 73246 construct. FIG. 44A shows staining for cell-surface CD3 expression (X-axis) and expression of a CD19 CAR (Y-axis). FIG. 44B shows staining for GFP-positive cells (X-axis) and CAR (Y-axis) within the population of CD3-negative/CAR-positive cells. FIG. 44C shows staining for GFP (X-axis) and expression of the CAR (Y-axis) in cells within the CD3/CAR double-negative population.

FIGS. 45A-45C show flow cytometry results obtained from primary T cells electroporated with a TRC 1-2L.1592 meganuclease and further transduced with a first AAV comprising the 73247 construct and a second AAV comprising the 7374 construct. FIG. 45A shows staining for cell-surface CD3 expression (X-axis) and expression of a CD19 CAR (Y-axis). FIG. 45B shows staining for GFP-positive cells (X-axis) and CAR (Y-axis) within the population of CD3-negative/CAR-positive cells. FIG. 45C shows staining for GFP (X-axis) and expression of the CAR (Y-axis) in cells within the CD3/CAR double-negative population.

FIGS. 46A-46C show flow cytometry results obtained from primary T cells electroporated with a TRC 1-2L.1592 meganuclease and further transduced with an AAV comprising the 73248 construct. FIG. 46A shows staining for cell-surface CD3 expression (X-axis) and expression of a CD19 CAR (Y-axis). FIG. 46B shows staining for GFP-positive cells (X-axis) and CAR (Y-axis) within the population of CD3-negative/CAR-positive cells. FIG. 46C shows staining for cell-surface CAR (Y-axis) and GFP (X-axis) within the population of CD3-negative/CAR-negative cells.

FIGS. 47A-47B show flow cytometry results obtained from primary T cells electroporated with a TRC 1-2L.1592 meganuclease and further transduced with an AAV comprising the 73249 construct. FIG. 47A shows staining for cell-surface CD3 expression (X-axis) and expression of a CD19 CAR (Y-axis). FIG. 47B shows staining for GFP-positive cells (X-axis) and CAR (Y-axis) within the population of CD3-negative/CAR-negative cells.

FIGS. 48A-48C show flow cytometry results obtained from primary T cells electroporated with a TRC 1-2L.1592 meganuclease and further transduced with an AAV comprising the 73250 construct. FIG. 48A shows staining for cell-surface CD3 expression (X-axis) and expression of a CD19 CAR (Y-axis). FIG. 48B shows staining for GFP-positive cells (X-axis) and CAR (Y-axis) within the population of CD3-negative/CAR-positive cells. FIG. 48C shows staining for cell-surface CAR (Y-axis) and GFP (X-axis) within the population of CD3-negative/CAR-negative cells.

FIGS. 49A-49B show flow cytometry results obtained from primary T cells electroporated with a TRC 1-2L.1592 meganuclease and further transduced with an AAV comprising the 73251 construct. FIG. 49A shows staining for cell-surface CD3 expression (X-axis) and expression of a CD19 CAR (Y-axis). FIG. 49B shows staining for GFP-positive cells (X-axis) and CAR (Y-axis) within the population of CD3-negative/CAR-negative cells.

FIGS. 50A-50C show flow cytometry results obtained from primary T cells electroporated with a TRC 1-2L.1592 meganuclease and further transduced with a first AAV comprising the 73248 construct and a second AAV comprising the 73249 construct. FIG. 50A shows staining for cell-surface CD3 expression (X-axis) and expression of a CD19 CAR (Y-axis). FIG. 50B shows staining for GFP-positive cells (X-axis) and CAR (Y-axis) within the population of CD3-negative/CAR-positive cells. FIG. 50C shows staining for GFP (X-axis) and expression of the CAR (Y-axis) in cells within the CD3/CAR double-negative population.

FIGS. 51A-51C show flow cytometry results obtained from primary T cells electroporated with a TRC 1-2L.1592 meganuclease and further transduced with a first AAV comprising the 73250 construct and a second AAV comprising the 73251 construct. FIG. 51A shows staining for cell-surface CD3 expression (X-axis) and expression of a CD19 CAR (Y-axis). FIG. 51B shows staining for GFP-positive cells (X-axis) and CAR (Y-axis) within the population of CD3-negative/CAR-positive cells. FIG. 51C shows staining for GFP (X-axis) and expression of the CAR (Y-axis) in cells within the CD3/CAR double-negative population.

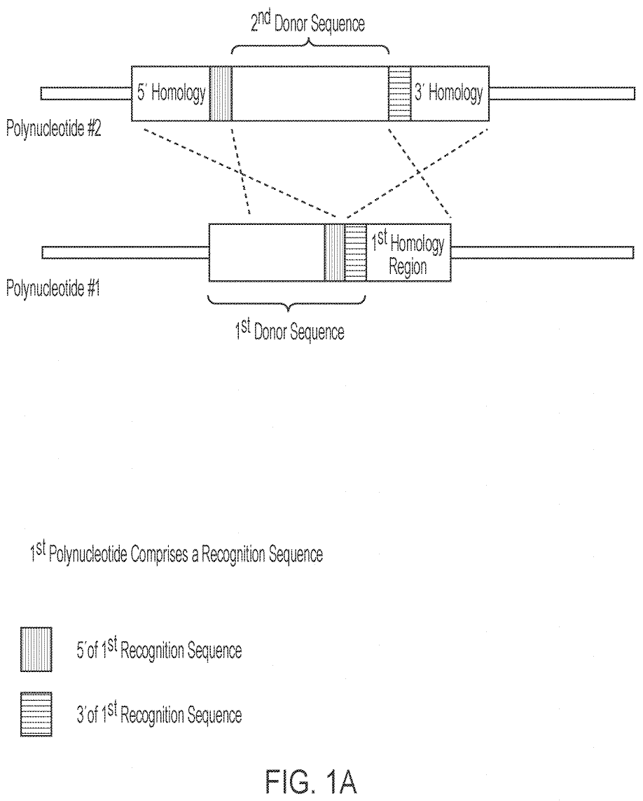

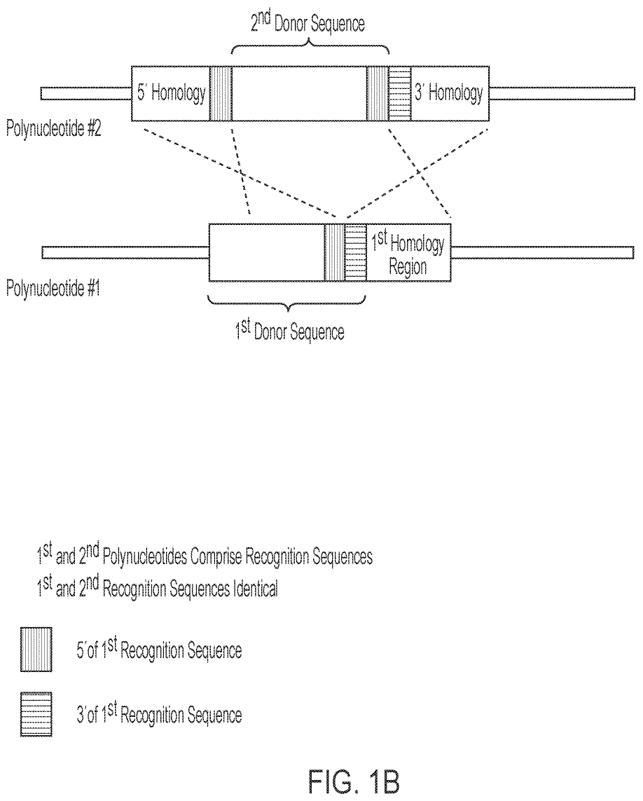

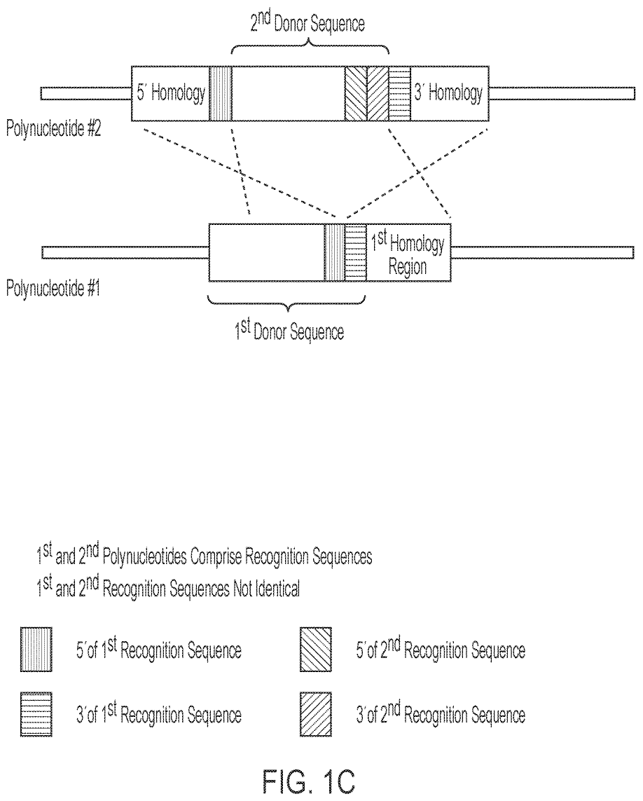

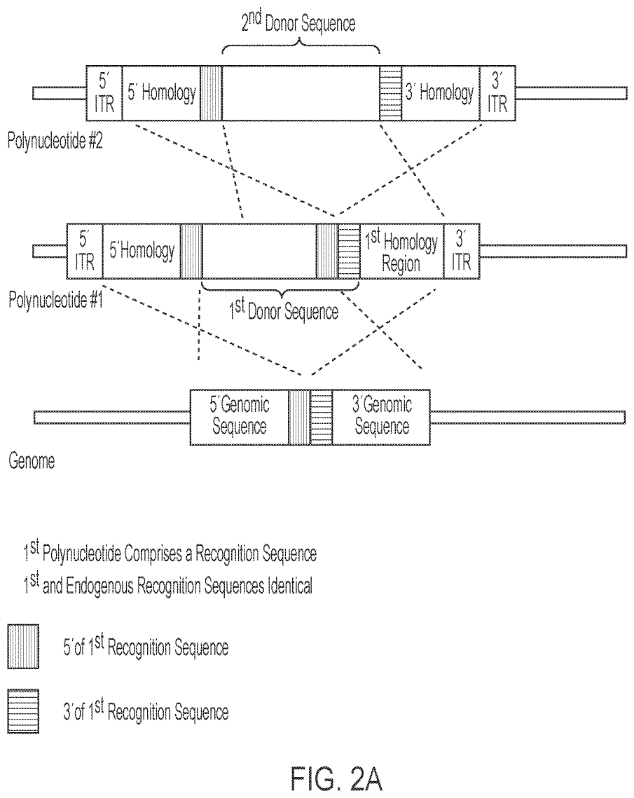

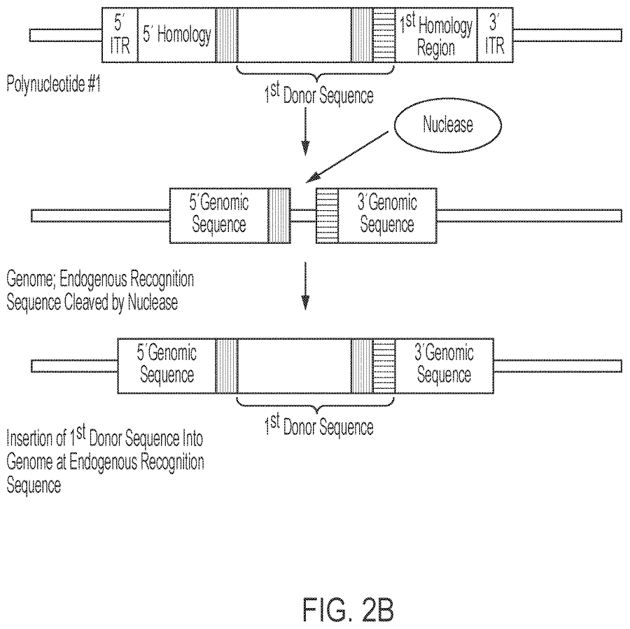

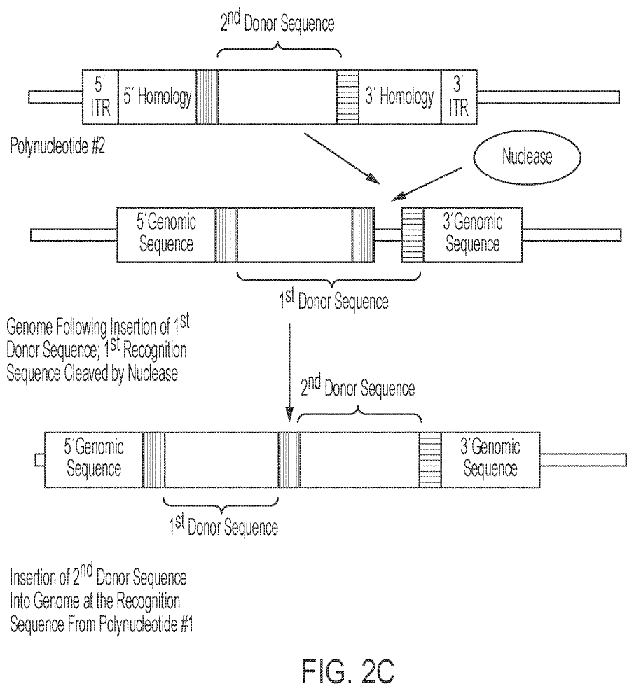

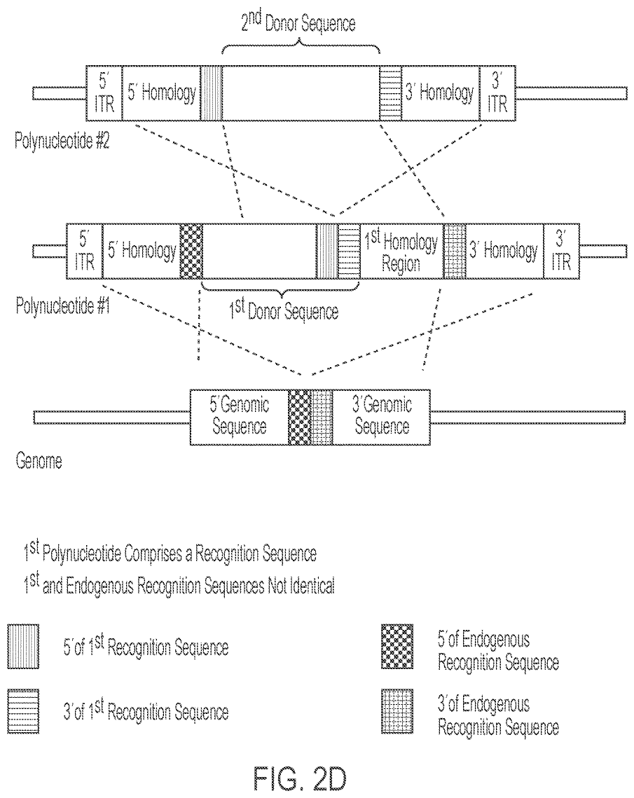

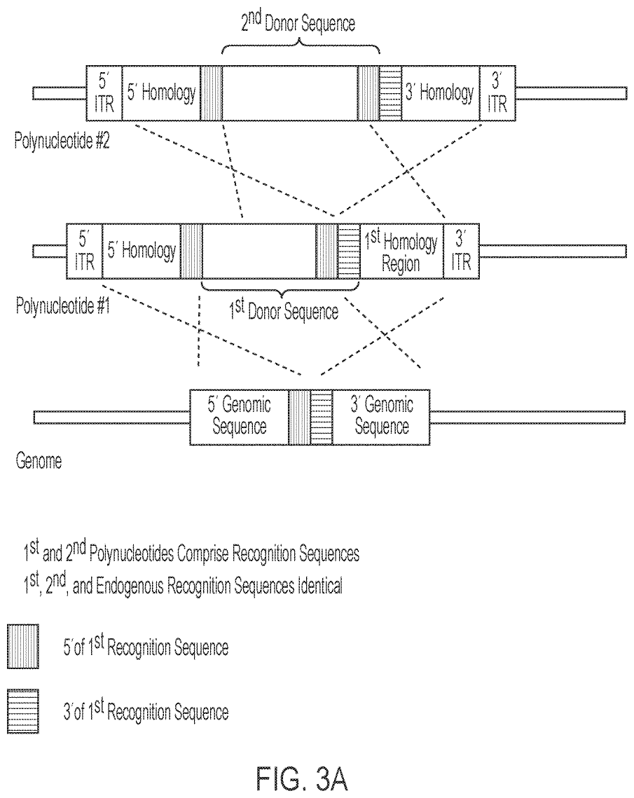

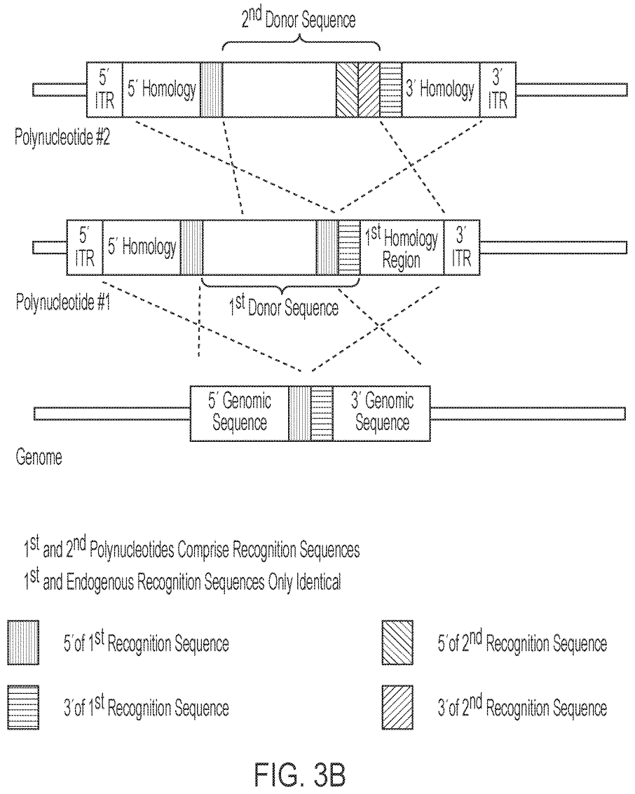

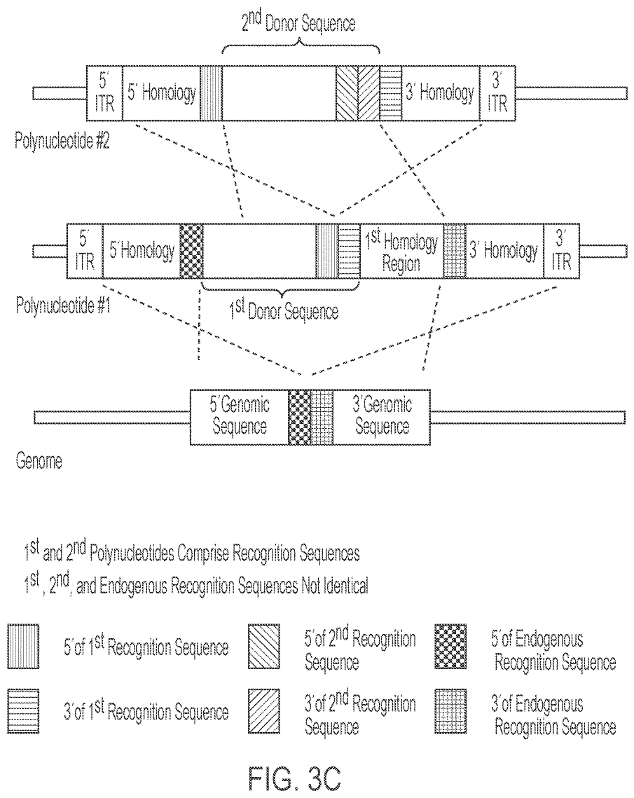

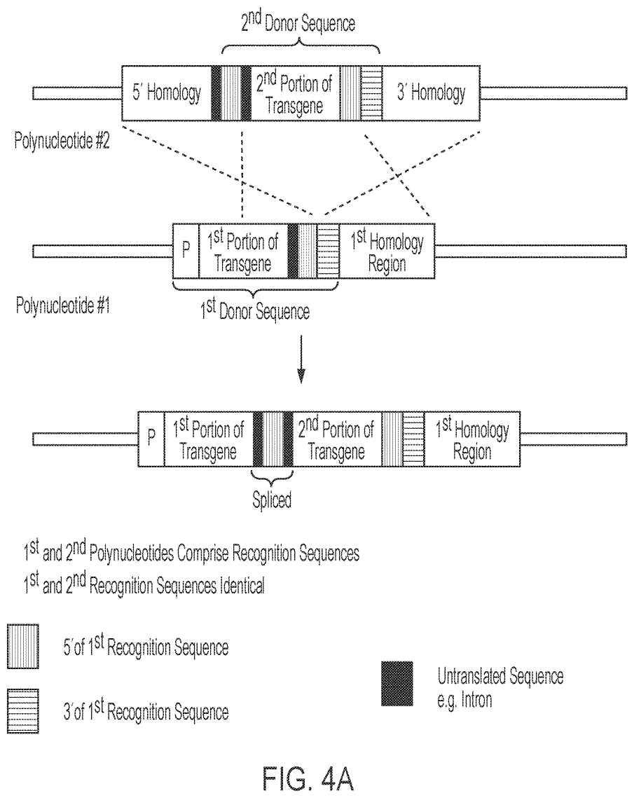

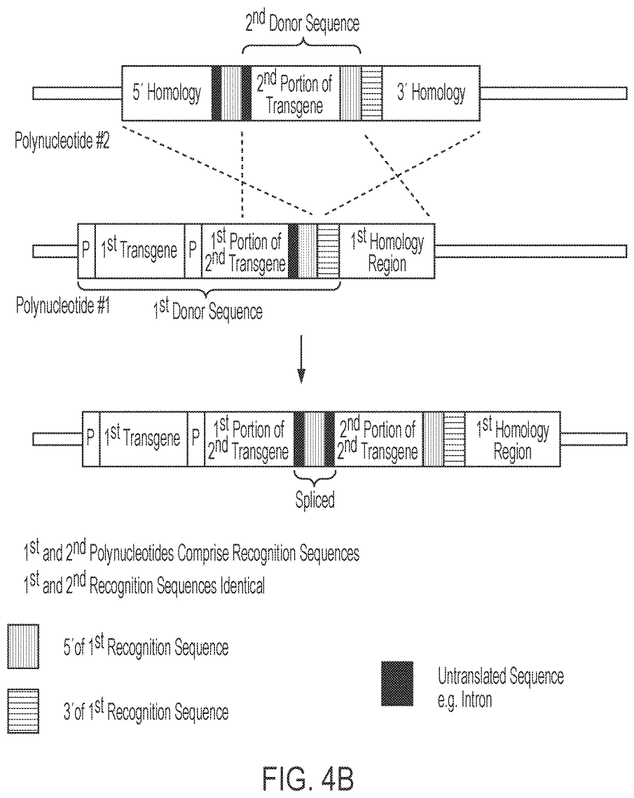

### BRIEF DESCRIPTION OF THE SEQUENCES

SEQ ID NO: 1 sets forth the amino acid sequence of the wild-type I-CreI homing endonuclease from Chlamydomonas reinhardtii.

SEQ ID NO: 2 sets forth the amino acid sequence of a LAGLIDADG motif.

SEQ ID NO: 3 sets forth the nucleic acid sequence of the 7373 construct beginning with the D sequence and ending prior to the 3′ ITR.

SEQ ID NO: 4 sets forth the nucleic acid sequence of the 7374 construct beginning with the D sequence and ending prior to the 3′ ITR.

SEQ ID NO: 5 sets forth the nucleic acid sequence of the 7375 construct beginning with the D sequence and ending prior to the 3′ ITR.

SEQ ID NO: 6 sets forth the nucleic acid sequence of the 73234 construct beginning with the D sequence and ending prior to the 3′ ITR.

SEQ ID NO: 7 sets forth the nucleic acid sequence of the 73235 construct beginning with the D sequence and ending prior to the 3′ ITR.

SEQ ID NO: 8 sets forth the nucleic acid sequence of the 73236 construct beginning with the D sequence and ending prior to the 3′ ITR.

SEQ ID NO: 9 sets forth the nucleic acid sequence of the 73237 construct beginning with the D sequence and ending prior to the 3′ ITR.

SEQ ID NO: 10 sets forth the nucleic acid sequence of the 73238 construct beginning with the D sequence and ending prior to the 3′ ITR.

SEQ ID NO: 11 sets forth the nucleic acid sequence of the 73245 construct beginning with the D sequence and ending prior to the 3′ ITR.

SEQ ID NO: 12 sets forth the nucleic acid sequence of the 73426 construct beginning with the D sequence and ending prior to the 3′ ITR.

SEQ ID NO: 13 sets forth the nucleic acid sequence of the 73247 construct beginning with the D sequence and ending prior to the 3′ ITR.

SEQ ID NO: 14 sets forth the nucleic acid sequence of the 73248 construct beginning with the D sequence and ending prior to the 3′ ITR.

SEQ ID NO: 15 sets forth the nucleic acid sequence of the 73249 construct beginning with the D sequence and ending prior to the 3′ ITR.

SEQ ID NO: 16 sets forth the nucleic acid sequence of the 73250 construct beginning with the D sequence and ending prior to the 3′ ITR.

SEQ ID NO: 17 sets forth the nucleic acid sequence of the 73251 construct beginning with the D sequence and ending prior to the 3′ ITR.

SEQ ID NO: 18 sets forth the nucleic acid sequence of a D sequence.

SEQ ID NO: 19 sets forth the nucleic acid sequence of the TRC 1-2 recognition sequence (sense strand).

SEQ ID NO: 20 sets forth the nucleic acid sequence of a 5′ portion of the TRC 1-2 recognition sequence (nucleotides 1-13).

SEQ ID NO: 21 sets forth the nucleic acid sequence of a 3′ portion of the TRC 1-2 recognition sequence (nucleotides 10-22).

SEQ ID NO: 22 sets forth the nucleic acid sequence of the B2M 13-14 recognition sequence (sense strand).

SEQ ID NO: 23 sets forth the nucleic acid sequence of a 5′ portion of the B2M 13-14 recognition sequence (nucleotides 1-13).

SEQ ID NO: 24 sets forth the nucleic acid sequence of a 3′ portion of the B2M 13-14 recognition sequence (nucleotides 10-22).

SEQ ID NO: 25 sets forth the nucleic acid sequence of the HAO 1-2 recognition sequence (sense strand).

SEQ ID NO: 26 sets forth the nucleic acid sequence encoding a 5′ portion of a GFP transgene.

SEQ ID NO: 27 sets forth the nucleic acid sequence encoding a 3′ portion of a GFP transgene.

SEQ ID NO: 28 sets forth the nucleic acid sequence encoding a green fluorescent protein (GFP) transgene.

SEQ ID NO: 29 sets forth the nucleic acid sequence encoding a chimeric antigen receptor (CAR).

SEQ ID NO: 30 sets forth the nucleic acid sequence of a BGH polyA signal.

SEQ ID NO: 31 sets forth the nucleic acid sequence of an sv40 polyA signal.

SEQ ID NO: 32 sets forth the nucleic acid sequence of an intron sequence comprising a 5′ splice donor sequence and a 3′ splice acceptor sequence.

SEQ ID NO: 33 sets forth the nucleic acid sequence of an intron sequence comprising a 5′ splice donor sequence.

SEQ ID NO: 34 sets forth the nucleic acid sequence of an intron sequence comprising a 3′ splice acceptor sequence.

SEQ ID NO: 35 sets forth the nucleic acid sequence of a splice donor sequence.

SEQ ID NO: 36 sets forth the nucleic acid sequence of a splice acceptor sequence.

SEQ ID NO: 37 sets forth the nucleic acid sequence of a JeT promoter.

SEQ ID NO: 38 sets forth the nucleic acid sequence of an EF1 alpha promoter.

SEQ ID NO: 39 sets forth the nucleic acid sequence of a 2A element.

SEQ ID NO: 40 sets forth the amino acid sequence of the TRC 1-2L.1592 meganuclease.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.

As used herein, the terms “polynucleotide” and “nucleic acid molecule” are used interchangeably, and refer to a polymer of nucleotide monomers. The polynucleotide can be comprised of ribonucleotides, deoxyribonucleotides, or a combination thereof. Such ribonucleotides and deoxyribonucleotides include both naturally occurring molecules and synthetic analogues. Polynucleotides can be single-stranded, double-stranded, or a combination thereof.

As used herein, the term “nucleic acid sequence” refers to the linear arrangement of nucleotides within a nucleic acid molecule.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

As used herein, the term “operably linked” is intended to mean a functional linkage between two or more elements. For example, an operable linkage between a nucleic acid sequence encoding a nuclease and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:3389-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “endogenous” in reference to a nucleotide sequence or protein is intended to mean a sequence or protein that is naturally comprised within or expressed by a cell.

As used herein, the terms “exogenous” or “heterologous” in reference to a nucleotide sequence or amino acid sequence are intended to mean a sequence that is purely synthetic, that originates from a foreign species, or, if from the same species, is substantially modified from its native form in composition and/or genomic locus by deliberate human intervention.

As used herein, the term “expression” refers to the transcription and/or translation of a particular nucleotide sequence driven by a promoter.

As used herein, the terms “expression vector,” “expression cassette,” and “expression construct” are used interchangeably herein and refer to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “promoter” or “regulatory sequence” refers to a nucleic acid sequence which is required for expression of a gene product operably linked to the promoter or regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, the term “polycistronic” mRNA refers to a single messenger RNA that comprises two or more coding sequences (i.e., cistrons) and encodes more than one protein. A polycistronic mRNA can comprise any element known in the art to allow for the translation of two or more genes from the same mRNA molecule including, but not limited to, an IRES element or a 2A element (e.g., a T2A element, a P2A element, an E2A element, and an F2A element).

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “homology arms” refer to nucleic acid sequences flanking the 5′ and 3′ ends of a nucleic acid molecule, which promote insertion of the nucleic acid molecule into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein, the term “homology region” refers to a nucleic acid sequence that has homology to a portion of a polynucleotide comprising a donor nucleic acid sequence intended for insertion within the polynucleotide. The presence of the homology region can thus facilitate the insertion of the donor nucleic acid sequence via homology directed repair.

As used herein, the term “high degree of homology” refers to two sequences that share sufficient homology as to promote homology recombination. For example, two sequences having a high degree of homology may share greater than 95% sequence identity, while retaining their functionality or elements responsible for their functionality.

As used herein, the terms “donor nucleic acid,” “template nucleic acid,” “donor template,” or “repair template” refer to a nucleic acid sequence that is desired to be inserted into a cleavage site within a cell's genome. Such template nucleic acids or donor templates can comprise, for example, a transgene, such as an exogenous transgene, which encodes a protein of interest (e.g., a CAR). The template nucleic acid or donor template can comprise 5′ and 3′ homology arms having homology to 5′ and 3′ sequences, respectively, that flank a cleavage site in the genome where insertion of the template is desired. Insertion can be accomplished, for example, by homology-directed repair (HDR).

As used herein, the term “stacking polynucleotide” refers to a polynucleotide that comprises a donor nucleic acid and the means (e.g., homology arms) for insertion of the donor nucleic acid into a heterologous polynucleotide or genome. In certain embodiments, the stacking polynucleotide also comprises the means (e.g., nuclease recognition sequence or a portion of a nuclease recognition sequence that could be paired with the remainder of the nuclease recognition sequence upon insertion) for the sequential insertion of a second donor nucleic acid into the first donor nucleic acid sequence.

As used herein, the term “insertion” refers to the addition of a heterologous nucleic acid into a DNA sequence, such as a plasmid, viral vector, or genome. The insertion can be random or targeted. The insertion site can be created by cleavage with a nuclease (double-stranded or single-stranded breaks creating staggered or blunt ends) and the insertion can occur through any mechanism, including but not limited to non-homologous end joining (NHEJ), homology-directed repair (HDR), and microhomology-mediated end joining (MMEJ).

As used herein, the term “transgene” refers to a nucleic acid molecule that encodes a polypeptide or RNA that is heterologous to the vector sequences flanking the coding sequence or is intended for transfer or has been transferred to a non-native cell or genomic locus.

As used herein, the phrase “a portion of a transgene” refers to a fragment of a transgene that does not include the entire coding sequence. A portion of a transgene may be, for example, about 1%, about 5%, about 10%, about 20%, about 30%, about 40%, about 50%, about 60%, about 70%, about 80%, about 90%, or about 95% of the coding sequence.

As used herein, the term “inhibitory nucleic acid” refers to a nucleic acid molecule that reduces or inhibits the expression of at least one gene through RNA interference (RNAi). Non-limiting examples of inhibitory nucleic acids include double-stranded RNA (dsRNA), small interfering RNA (siRNA), antisense RNA, microRNA (miRNA), short hairpin RNA (shRNA), and microRNA-adapted shRNA (shRNAmiR).

As used herein, the terms “shRNA” or “short hairpin RNA” refers to an artificial RNA molecule comprising a hairpin that can be used to silence gene expression via RNA interference.

As used herein, the terms “shRNAmiR” and “microRNA-adapted shRNA” refer to shRNA sequences embedded within a microRNA scaffold. A shRNAmiR molecule mimics naturally-occurring pri-miRNA molecules in that they comprise a hairpin flanked by sequences necessary for efficient processing and can be processed by the Drosha enzyme into pre-miRNAs, exported into the cytoplasm, and cleaved by Dicer, after which the mature miRNA can enter the RISC. The microRNA scaffold can be derived from naturally-occurring microRNA, pre-miRNAs, or pri-miRNAs or variants thereof. In some embodiments, the shRNA sequences which the shRNAmiR is based upon is of a different length from miRNAs (which are 22 nucleotides long) and the miRNA scaffold must therefore be modified in order to accommodate the longer or shorter shRNA sequence length. The shRNAmiR molecules used in the presently disclosed compositions and methods can comprise in the 5′ to 3′ direction: (a) a 5′ miR scaffold domain; (b) a 5′ miR basal stem domain; (c) a passenger strand; (d) a miR loop domain; (e) a guide strand; (f) a 3′ miR basal stem domain; and (g) a 3′ miR scaffold domain.

As used herein, the term “reporter protein” refers to a protein that confers to a cell expressing it a property that is detectable or measurable. Reporter proteins can be used as a selectable marker. Non-limiting examples of reporter proteins include fluorescent proteins, luciferase, β-galactosidase, and various proteins that confer antibiotic resistance.

As used herein, the term “protein useful for purification” refers to a protein that can be utilized in order to select or isolate the biological complex bound to the protein, or a cell expressing the protein. Such proteins or tags include but are not limited to biotin, myc, maltose binding protein (MBP), and glutathione-S-transferase (GST), or a cell-surface protein that can be detected with a specific antibody.

As used herein, the term “therapeutic protein” refers to a protein that can effect beneficial or desirable biological and/or clinical results. The therapeutic protein may be effective in treating or preventing a pathological condition.

As used herein, the term “suicide protein” refers to a protein the expression of which provokes cell death and allows for selective destruction of the cells expressing the protein in vitro or in vivo.

As used herein, the term “untranslated sequence” refers to a nucleotide sequence that is not translated from an mRNA into a protein sequence. The term “untranslated sequence” can refer to the untranslated region of an mRNA or a nucleotide sequence that encodes the untranslated region of an mRNA.

As used herein, the term “intron sequence” refers to a nucleotide sequence within a gene or transgene that is removed by RNA splicing during maturation of the final RNA product. The term “intron” refers to both the nucleotide sequence encoding an mRNA and the corresponding sequence in RNA transcripts. Sequences that are joined together in the final mature RNA after RNA splicing are exons.

As used herein, the term “splice donor sequence” refers to a nucleotide sequence at the 5′ end of an intron that, together with the splice acceptor sequence, mediates the splicing of the intervening intronic sequence. Suitable sequences for the 5′ splice donor site used in RNA splicing are well known in the art. See, e.g., Moore, et al., 1993, The RNA World, Cold Spring Harbor Laboratory Press, p. 303-358. The splice donor sequence comprises a dinucleotide at its 5′ end. GU is the conserved dinucleotide sequence recognized by the major spliceosome and AU is the conserved dinucleotide sequence recognized by the minor spliceosome.

As used herein, the term “splice acceptor sequence” refers to a nucleotide sequence at the 3′ end of an intron that, together with the splice donor sequence, mediates the splicing of the intervening intronic sequence. Suitable sequences for the 3′ splice acceptor region, as well as the branchpoint sequence, used in RNA splicing are well known in the art. See, e.g., Moore, et al., 1993, The RNA World, Cold Spring Harbor Laboratory Press, p. 303-358. The splice acceptor sequence comprises a dinucleotide at its 3′ end. AG is the conserved dinucleotide sequence recognized by the major spliceosome and AC is the conserved dinucleotide sequence recognized by the minor spliceosome. Upstream or 5′ of the dinucleotide sequence can also be a region high in pyrimidines or a polypyrimidine tract. In addition to the dinucleotide sequence, and optionally a polypyrimidine tract, the splice acceptor sequence may also comprise a branchpoint sequence further upstream from the polypyrimidine tract which includes an adenine nucleotide that is involved in lariat formation.

As used herein, the term “splicing complex” refers to any ribonucleoprotein spliceosome that is capable of splicing introns from an RNA transcript. The major spliceosome comprises U1, U2, U4, U5, and U6 small nuclear ribonucleoproteins (snRNPs), as well as other proteins including U2 small nuclear RNA auxiliary factor 1 (U2AF35), U2AF2 (U2AF65), and SF1. The major spliceosome splices introns comprising GU at the 5′ splice donor site and AG at the 3′ splice acceptor site. The minor spliceosome is responsible for splicing introns with more rare splice site sequences. The minor spliceosome comprises U5 snRNP, U11, U12, U4atac, and U6atac. Most naturally occurring introns that are recognized by the minor spliceosome either comprise an AT donor sequence and AC acceptor sequence or a GT donor sequence and AG acceptor sequence, although other sequences can function as splice donor and acceptor sequences for the minor spliceosome. See, e.g., Turunen et al. (2013) Wiley Interdiscip Rev RNA 4(1):61-76).

As used herein, the term “flanked” in relation to a particular nucleic acid sequence refers to a sequence (i.e., flanking sequence) being on each side of the particular nucleic acid sequence. In general, the flanking sequences are directly adjacent to the particular nucleic acid sequence. The flanking sequence on the 5′ end of the particular nucleic acid sequence can be identical or different from the flanking sequence on its 3′ end.

As used herein, the term “substantially purified cell” refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cells that have been separated from the cells with which they are naturally associated in their natural state. In some aspects, the cells are cultured in vitro. In other aspects, the cells are not cultured in vitro.

As used herein, the term “introduce” refers to contacting a host cell with an exogenous nucleic acid in such a manner that the nucleic acid gains access to the interior of the host cell.

As used herein, the terms “transfected” or “transformed” or “transduced” or “nucleofected” refer to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the term “simultaneous” in relation to introducing polynucleotides into a host cell refers to the provision of two or more polynucleotides at the same time (i.e., within a single transfection, transformation, or transduction procedure) to a cell or group of cells such that the two or more polynucleotides gain entry into the host cell.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the presently disclosed isolated nucleotides or nucleic acid sequences. In some embodiments, a “vector” also refers to a viral vector, which is derived from a virus. Viral vectors can include, without limitation, retroviral vectors, lentiviral vectors, adenoviral vectors, and adeno-associated viral vectors (AAV).

As used herein, the term “adeno-associated virus” or “adeno-associated viral vector” or “AAV” or “AAV vector” are used interchangeably and refer to viruses and vectors derived from the Parvoviridae family. Adeno-associated viruses can infect both dividing and quiescent cells. Adeno-associated viral vectors of any serotype can be used in the presently disclosed methods and compositions, as well as variants thereof. As used herein, the term “serotype” refers to a distinct variant within a species of virus that is determined based on the viral cell surface antigens. Known serotypes of AAV include, for example, AAV1, AAV2, AAV3, AAV4, AAV5, AAV6, AAV7, AAV8, AAV9, AAV10, and AAV11 (Weitzman and Linden (2011) In Snyder and Moullier Adeno-associated virus methods and protocols. Totowa, N.J.: Humana Press). Other serotypes of AAV are known, and a person of skill in the art could determine their usefulness in the present invention. Generally, adeno-associated viruses comprise a transgene or portion thereof that is flanked by parvoviral or AAV inverted terminal repeat sequences (ITRs). Such AAV vectors can be replicated and packaged into infectious viral particles when present in a host cell that is expressing AAV replication (rep) and capsid (cap) gene products.

As used herein, the term “nuclease adeno-associated virus” or “nuclease AAV” refers to an AAV comprising a transgene encoding a nuclease.

As used herein, the term “inverted terminal repeat” or “ITR” refers to regions found at the 5′ and 3′ termini of the AAV genome. The ITRs are about 145 nt each that flank a transgene or portion thereof within an adeno-associated viral vector. The ITRs are self-complementary and are organized so that an energetically stable intramolecular duplex forming a T-shaped hairpin may be formed. These hairpin structures function as an origin for viral DNA replication, serving as primers for the cellular DNA polymerase complex. The ITRs also aid in concatamer formation in the nucleus and integration into the genome. Sequences of AAV-associated ITRs are known in the art and disclosed by Yan et al., J. Virol. 79(1):364-379 (2005). ITR sequences that find use herein may be full length, wild-type AAV ITRs or fragments thereof that retain functional capability, or may be sequence variants of full-length, wild-type AAV ITRs that are capable of functioning in cis as origins of replication. AAV ITRs useful in the presently disclosed methods and compositions may derive from any known AAV serotype.

As used herein, the term “D sequence” refers to D-sequence, a stretch of nucleotides, which in some cases can be 20 nucleotides in length, that are associated with an AAV inverted terminal repeat but do not play a role in hairpin formation. The D sequence has been shown to play a role in the life cycle of AAV in a number of ways. For example, the D sequence acts as the packaging signal for AAV. Further, the first 10 nucleotides of a D sequence have been shown to be necessary for AAV DNA replication (see, Kwon et al., Human Gene Therapy (2020), Vol. 31 (9-10): 565-574).

As used herein, the term “lipid nanoparticle” refers to a lipid composition having a typically spherical structure with an average diameter between 10 and 1000 nanometers. In some formulations, lipid nanoparticles can comprise at least one cationic lipid, at least one non-cationic lipid, and at least one conjugated lipid. Lipid nanoparticles known in the art that are suitable for encapsulating nucleic acids, such as mRNA, are contemplated for use in the invention.

As used herein, the terms “nuclease” and “endonuclease” are used interchangeably to refer to naturally-occurring or engineered enzymes, which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the term “5′ portion” when referring to a nuclease recognition sequence is intended to mean the nucleotides of the recognition sequence that are 5′ upstream of a cleavage site generated by an engineered nuclease. Similarly, the term “3′ portion” when referring to a nuclease recognition sequence is intended to mean the nucleotides of the recognition sequence that are 3′ downstream of a cleavage site generated by an engineered nuclease. By way of example, where an engineered nuclease, such as a CRISPR/Cas9 nuclease system, generates a blunt end cleavage site within a recognition sequence, the 5′ portion of the recognition sequence comprises the nucleotides of the sequence that are 5′ upstream of the cleavage site, while the 3′ portion of the recognition sequence comprises the nucleotides of the sequence that are 3′ downstream of the cleavage site. For nucleases that generate 5′ or 3′ overhangs (e.g., engineered meganucleases), each 5′ portion or 3′ portion can, in some examples, include only the nucleotides that are 5′ upstream or 3′ downstream of the cleavage site, respectively.

In other examples, each 5′ portion or 3′ portion can include the nucleotides that are 5′ upstream or 3′ downstream of the cleavage site, respectively, as well as the nucleotides of the 5′ or 3′ overhang. By way of example, an I-CreI-derived engineered meganuclease generates a cleavage site between nucleotide positions 13 and 14 of its 22 base pair recognition sequence, wherein positions 9-13 represent the 4 base pair 3′ overhang. Thus, in some examples, the 5′ portion of the sequence can comprise the nucleotides of positions 1-13, and the 3′ portion of the sequence can comprise the nucleotides of positions 14-22. In other examples, the 5′ portion of the sequence can comprise the nucleotides of positions 1-13, and the 3′ portion of the sequence can comprise the nucleotides of positions 10-22, which includes the 4 base pair overhang and the 9 base pairs of the 3′ half site. In some cases, the inclusion or exclusion of the overhang nucleotides as components of a homology arm may be used to affect homology directed repair. It is understood that in examples wherein a 5′ portion of a recognition sequence and a 3′ portion of a recognition sequence are positioned adjacent to one another for the purpose of generating a complete recognition sequence, only the portion that normally includes the overhang base pairs will comprise the overhang nucleotides.

As used herein, “capable of pairing with” when referring to a nuclease recognition sequence refers to the ability of a portion of a nuclease recognition sequence to align with a second portion of the nuclease recognition sequence when the two portions are directly adjacent to one another in order to form the functional nuclease recognition sequence that can be recognized and cleaved by a nuclease. By way of example, for an I-CreI-derived engineered meganuclease recognition sequence, a 5′ portion of the sequence comprising the nucleotides of positions 1-13 is capable of pairing with a 3′ portion of the sequence comprising the nucleotides of positions 14-22, in order to form the functional recognition sequence.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the terms “target site” or “target sequence” refers to a region of the chromosomal DNA of a cell comprising a recognition sequence for a nuclease.

As used herein, the term “meganuclease” refers to an endonuclease that binds double-stranded DNA at a recognition sequence that is greater than 12 base pairs. In some embodiments, the recognition sequence for a meganuclease used in the presently disclosed methods and compositions is 22 base pairs. A meganuclease can be an endonuclease that is derived from I-CreI (SEQ ID NO: 1), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases used in the presently disclosed methods and compositions are substantially non-toxic when expressed in the targeted cells as described herein such that cells can be transfected and maintained at 37° C. without observing deleterious effects on cell viability or significant reductions in meganuclease cleavage activity.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, Si nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, Endl, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the term “megaTAL” refers to a single-chain endonuclease comprising a transcription activator-like effector (TALE) DNA binding domain with an engineered, sequence-specific homing endonuclease.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, typeV enzyme, such as Cpfl. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, 51 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the term “treatment”, “treating”, or “treating a subject” refers to the administration of a pharmaceutical composition disclosed herein, comprising a population of eukaryotic cells (e.g., CAR T cells), to a subject having a disease, disorder or condition. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, a partial or complete reduction in the number of cancer cells present in the subject, and remission or improved prognosis. In some aspects, treatment includes the administration of a lymphodepletion regimen to reduce endogenous lymphocytes in the subject for immunotherapy.

As used herein, the term “effective amount,” “therapeutically effective amount,” “or “clinically therapeutic level” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In specific embodiments, the therapeutic amount or level in a subject of a therapeutic compound (e.g., protein) reduces at least one symptom of a disease in a subject. In those embodiments wherein the disease is a cancer, a therapeutically effective level of the therapeutic compound (e.g., protein) reduces the level of proliferation or metastasis of cancer, causes a partial or full response or remission of cancer, or reduces at least one symptom of cancer in a subject.

As used herein, the term “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

As used herein, the term “prophylaxis” means the prevention of or protective treatment for a disease or disease state.

As used herein, the term “reduced” refers to any reduction in the symptoms or severity of a disease or any reduction in the proliferation or number of cancerous cells. In either case, such a reduction may be up to 5%, 10%, 20%, 30%, 40%, 50%, 60%, 70%, 80%, 90%, 95%, or up to 100%. Accordingly, the term “reduced” encompasses both a partial reduction and a complete reduction of a disease state.

As used herein, the term “response,” “complete response,” “complete response with incomplete blood count recovery,” “refractory disease,” “partial response,” “disease progression” or “progressive disease,” “refractory disease,” “relapse” or “relapsed disease” each refer to assessments of disease state and response in subjects following treatment according to the methods disclosed herein.

As used herein, the term “cancer” should be understood to encompass any neoplastic disease (whether invasive or metastatic) which is characterized by abnormal and uncontrolled cell division causing malignant growth or tumor.

As used herein, the term “carcinoma” refers to a malignant growth made up of epithelial cells.

As used herein, the term “blastoma” refers to a type of cancer that is caused by malignancies in precursor cells or blasts (immature or embryonic tissue).

As used herein, the term “leukemia” refers to malignancies of the hematopoietic organs/systems and is generally characterized by an abnormal proliferation and development of leukocytes and their precursors in the blood and bone marrow.

As used herein, the term “lymphoma” refers to a group of blood cell tumors that develop from lymphocytes.

As used herein, the term “melanoma” refers to a tumor arising from the melanocytic system of the skin and other organs.

As used herein, the term “sarcoma” refers to a tumor which is made up of a substance like the embryonic connective tissue and is generally composed of closely packed cells embedded in a fibrillary, heterogeneous, or homogeneous substance.

As used herein, the term “cancer of B-cell origin” refers to any blood cancer that affects immature and/or mature B lymphocytes.

As used herein, the term “B-lineage acute lymphoblastic leukemia” or “B-lineage ALL” refers to a cancer of the lymphoid line of blood cells characterized by the development of large numbers of immature B lymphocytes.

As used herein, the term “B-cell non-Hodgkin's lymphoma” or “B-cell NHL” refers to a group of blood cancers that includes all types of B-cell lymphomas except Hodgkin's lymphomas.

As used herein, the term “B-cell chronic lymphocytic leukemia” or “B-cell CLL” refers to a type of non-Hodgkin's lymphoma cancer characterized by the clonal proliferation and accumulation of neoplastic B lymphocytes in the blood and bone marrow.

As used herein, the term “multiple myeloma” refers to a cancer affecting plasma cells. As used herein, the terms “tumor associated antigen” or “tumor antigen” or “hyperproliferative disorder antigen” or “antigen associated with a hyperproliferative disorder” refers to antigens that are common to specific hyperproliferative disorders. In certain aspects, the hyperproliferative disorder antigens useful in the presently disclosed methods and compositions are derived from, cancers including but not limited to primary or metastatic melanoma, thymoma, lymphoma, sarcoma, lung cancer, liver cancer, NHL, leukemias, uterine cancer, cervical cancer, bladder cancer, kidney cancer and adenocarcinomas such as breast cancer, prostate cancer, ovarian cancer, pancreatic cancer, and the like.

As used herein, the terms “antigen” or “Ag” refers to a molecule that is capable of being bound specifically by an antibody, or otherwise provokes an immune response. This immune response may involve either antibody production, or the activation of specific immunologically-competent cells, or both.

As used herein, the terms “anti-tumor activity” or “anti-tumor effect” refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the genetically-modified cells of the present disclosure in prevention of the occurrence of tumor in the first place.

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety, a transmembrane domain, and an intracellular domain, wherein the intracellular domain comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcγ receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain. As used herein, the term “chimeric antigen receptor T cell” or “CAR T cell” refers to a human T cell modified to comprise a transgene encoding a CAR, wherein the CAR is expressed on the cell surface of the T cell.

As used herein, the term “detectable cell surface expression of an endogenous TCR” refers to the ability to detect one or more components of the TCR complex (e.g., an alpha/beta TCR complex) on the cell surface of a T cell (e.g., a CAR T cell), or a population of T cells (e.g., CAR T cells) described herein, using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in MacLeod et al. (2017) Molecular Therapy 25(4): 949-961.

As used herein, the terms “exogenous T cell receptor” or “exogenous TCR” refer to a TCR whose sequence is introduced into the genome of an immune effector cell (e.g., a human T cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest.

As used herein, the term “target cell” refers to a cell into which it is desired to introduce a transgene. The target cell can be any type of cell, including but not limited to, an immune cell.

As used herein, the term “immune cell” refers to any cell that is part of the immune system (innate and/or adaptive) and is of hematopoietic origin. Non-limiting examples of immune cells include lymphocytes, B cells, T cells, monocytes, macrophages, dendritic cells, granulocytes, megakaryocytes, monocytes, macrophages, natural killer cells, myeloid-derived suppressor cells, innate lymphoid cells, platelets, red blood cells, thymocytes, leukocytes, neutrophils, mast cells, eosinophils, basophils, and granulocytes.

As used herein, the terms “T cell” and “T lymphocyte” are used interchangeably herein and refer to a white blood cell of the lymphocyte subtype that expresses T cell receptors on the cell membrane. T cells develop in the thymus gland and include both CD8+ T cells and CD4+ T cells, as well as natural killer T cells, memory T cells, gamma delta T cells, and any other lymphocytic cell that expresses a T cell receptor.

As used herein, the terms “B cell” and “B lymphocyte” are used interchangeably herein and refer to a white blood cell of the lymphocyte subtype that expresses B cell receptors on the cell membrane, through which the cells bind specific antigens and initiate an antibody response.

As used herein, the term “macrophage” refers to a white blood cell of the immune system that engulfs cells, cellular debris, foreign substances, microbes, cancer cells, and the like through phagocytosis.

As used herein, the term “induced pluripotent stem cell” or “iPSC” refers to a stem cell that is generated by reprogramming a somatic cell by expressing or inducing expression of a combination of factors (herein referred to as reprogramming factors). iPSCs can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, and Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell. iPSCs are similar in properties to embryonic stem cells in that iPSCs can proliferate and differentiate into various types of cells.

As used herein, the terms “human natural killer cell” or “human NK cell” or “natural killer cell” or “NK cell” refers to a type of cytotoxic lymphocyte critical to the innate immune system. The role NK cells play is analogous to that of cytotoxic T-cells in the vertebrate adaptive immune response. NK cells provide rapid responses to virally infected cells and respond to tumor formation, acting at around 3 days after infection. Human NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, the term “human T cell” or “isolated human T cell” refers to a T cell isolated from a human donor. In some cases, the human donor is not the subject treated according to the method (i.e., the T cells are allogeneic), but instead a healthy human donor. In some cases, the human donor is the subject treated according to the method. T cells, and cells derived therefrom, can include, for example, isolated T cells that have not been passaged in culture, or T cells that have been passaged and maintained under cell culture conditions without immortalization.

As used herein, the term “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” or “TRAC” refers to a coding sequence of the T cell receptor alpha gene. The TCR alpha constant region includes the wild-type sequence, and functional variants thereof, identified by NCBI Gene ID NO. 28755.

As used herein, the term “T cell receptor beta gene” or “TCR beta gene” refers to the locus in a T cell which encodes the T cell receptor beta subunit. The T cell receptor beta gene can refer to NCBI Gene ID number 6957.

As used herein, the term “T cell receptor beta constant region” or “TCR beta constant region” refers to a coding sequence of the T cell receptor beta gene. The TCR beta constant region includes the wild-type sequence, and functional variants thereof, identified by NCBI Gene ID No. 28639.

### 2.1 Principle of the Invention

The present invention provides for sequential stacking of donor nucleic acids into the genome of a cell to allow for the introduction of relatively long nucleic acid sequences. For example, the presently disclosed compositions and methods can be used to introduce into a genome a nucleic acid sequence that exceeds the packaging capacity of a single adeno-associated virus (AAV). In various examples, the nucleic acid sequence can encode a single transgene, or multiple transgenes. In examples wherein a single transgene is utilized, the present invention can allow for a first polynucleotide (e.g., virus) to provide a first portion of the transgene, and a second polynucleotide to provide a second portion of the transgene, such that the entire transgene is properly assembled when the second polynucleotide is incorporated into the first polynucleotide. In those examples wherein the first and second polynucleotides are AAVs, the size of the transgene can exceed the packaging capacity of a single AAV. The presently disclosed methods and compositions thus allow for sequential stacking of nucleic acid sequences into a single genomic locus, which can occur in a single-step process through the simultaneous introduction of the multiple polynucleotides into a cell along with a nuclease that generates the necessary cleavage sites. Furthermore, the strategic use of intron sequences that can be spliced out of the genome, as described herein, allows for the reliable removal of 5′ or 3′ portions of the nuclease recognition sequences that may otherwise remain in the genome after insertion of the polynucleotides and potentially interfere with transgene expression. Moreover, the deliberate use of a single D sequence positioned on the same end (either 5′ or 3′) of each polynucleotide is designed to generate AAVs comprising a genome synthesized from only a sense strand, or only an antisense strand, of each construct, thus reducing the possibility that a mixture of sense and antisense sequence-derived AAVs will recombine when introduced into a target cell and negatively impact proper insertion of the polynucleotides in the genome.

This technology is not only applicable to cell therapy and gene therapy but can also be useful in the development of cell lines and animal models. By way of example, the presently disclosed methods and compositions can be used to generate, in a single-step, chimeric antigen receptor (CAR) T cells that express multiple CARs, proteins useful for purification or safety switches, or nucleic acids useful for inhibiting endogenous protein synthesis (e.g., shRNAs, shRNAmiRs). Using the claimed invention, the coding sequences for all of these elements could be incorporated into the same locus in the genome (e.g., the T cell receptor alpha constant (TRAC) locus) using a single nuclease that advantageously generates only one cleavage site in the genome.

### 2.2 Stacking Polynucleotides

The present invention provides compositions comprising at least one engineered nuclease, or a nucleic acid encoding an engineered nuclease, and at least a first and a second polynucleotide that can be recombined with each other following cleavage by the engineered nuclease. These compositions can be used to stack multiple nucleic acid sequences (i.e., donor nucleic acid sequences) into a single genomic locus within a cell.

Generally, a first polynucleotide described herein comprises a number of elements necessary to allow for insertion of a first donor sequence into the genome of a cell within an endogenous nuclease recognition sequence. Such elements can include, for example, sequences that are homologous to genomic regions upstream and downstream of the nuclease cleavage site, including the 5′ and 3′ portions of the nuclease recognition sequence. The first polynucleotide further comprises its own nuclease recognition sequence that may be the same, or different, than the endogenous nuclease recognition sequence. Cleavage of this recognition sequence by a nuclease, and additional elements present in the first polynucleotide, promote the insertion of a second donor sequence from the second polynucleotide into the first donor sequence (i.e., stacking). By arranging the elements and homology regions of the first polynucleotide and the second polynucleotide in the various combinations described herein, this method can be used for a number of purposes, and primarily to insert large nucleic acid sequences into a single genomic locus that would not otherwise fit into a single viral vector. In some examples, a single transgene, such as a gene that is larger than the carrying capacity of a single viral vector, can be split between the first polynucleotide and the second polynucleotide, such that a complete coding sequence is formed after insertion of the first donor sequence into the genome, and insertion of the second donor sequence into the first. This is particularly advantageous when the transgene size exceeds the capacity of the method of introducing the transgene into the genome, such as those transgenes that exceed the packaging capacity of a viral vector, such as an adeno-associated viral (AAV) vector that has a packaging capacity of approximately 4.7 kb. In other examples, when it is desired to insert multiple transgenes at a single genomic locus, the individual transgenes can be split between the first and second polynucleotides and, ultimately, be inserted into a single locus in a manner that allows for expression of each gene.

Described herein are a number of configurations of the first and second polynucleotide that can be utilized with the invention. In some configurations, only the first polynucleotide comprises a nuclease recognition sequence. In such examples, additional polynucleotides cannot be stacked into the second polynucleotide. In other embodiments, however, the second polynucleotide also comprises a nuclease recognition sequence, allowing for the insertion of yet further donor sequences. In some cases, the nuclease recognition sequence of the first polynucleotide and, if present, the nuclease recognition sequence of the second polynucleotide, are identical to an endogenous nuclease recognition sequence at a locus where insertion is desired. In this scenario, a single engineered nuclease can be utilized to cleave the endogenous recognition sequence and the recognition sequences in the polynucleotides in order to promote the stacking process. In other examples where the endogenous nuclease recognition sequence and the recognition sequences in the polynucleotides differ, two or more engineered nucleases may be required for cleavage and stacking of the donor sequences. Various embodiments of such combinations are described herein.

Also described herein are configurations wherein a single large transgene can be inserted into the genome, or wherein multiple smaller transgenes that are collectively too large for a single viral vector can be inserted at a single locus. In the case of a single large transgene, configurations are described wherein a first portion of the transgene is encoded by the first polynucleotide and the second portion of the transgene is encoded by the second polynucleotide. Elements of each polynucleotide, and cleavage by an engineered nuclease, allows for assembly of the first and second donor sequences in the genome such that the full-length transgene can be expressed. In such examples, the first and/or second polynucleotide can comprise additional transgenes that are also expressed. In the case of multiple smaller transgenes, these can be split between the two polynucleotides as previously discussed. The elements of the polynucleotides, and cleavage by the engineered nuclease, allows for each transgene to be inserted into the same genomic locus in a manner that allows for them to each be expressed. Examples of such configurations, and their associated elements, are described herein.

A number of elements in each of the first and second polynucleotides enable the stacked insertion of the first and second donor sequences into the genome, and expression of any transgenes encoded therein. For example, the first polynucleotide can comprise one or more regulatory sequences, such as promoters, that are operably linked to a full-length transgene encoded by the first polynucleotide, or to the first portion of a transgene encoded by the first polynucleotide. In other examples, the first polynucleotide can comprise elements that are capable of operably linking a transgene or portion of a transgene to an endogenous promoter following insertion of the donor sequences into the genome. In some cases, the first promoter can comprise multiple promoters, wherein a first promoter is operably linked to a first transgene encoded by the first polynucleotide, and a second promoter that will be operably linked to a second transgene encoded on the second polynucleotide after insertion of the sequences into the genome. In further examples, the second polynucleotide can comprise one or more promoters that are operably linked to one or more transgenes encoded by the second polynucleotide. In certain examples, the elements of the first and second polynucleotide allow for multiple transgenes present on the first and/or second polynucleotides to be operably linked to a single promoter. Such elements can include, for example, a 2A or IRES sequence that is appropriately positioned on the first polynucleotide or the second polynucleotide, such that each transgene is operably linked to the same promoter (e.g., an endogenous promoter or exogenous promoter). Examples of such configurations, and their associated elements, are described herein.

The invention further includes the deliberate use of untranslated sequences, particularly intron sequences, in order to reliably prevent potential expression of, or interference by, any remaining portion of a nuclease recognition sequence that remains in the donor sequences after they have stacked, and which could be expressed as polypeptides along with the desired transgenes. The untranslated sequence can be any sequence which is not ultimately translated into an amino acid sequence. This includes introns that are spliced from the transcript prior to translation. In some examples, such untranslated sequences (e.g., introns) are appropriately positioned such that they ultimately flank a portion of a nuclease recognition sequence in the genome. The presence of splice donor and splice acceptor sequences in the flanking sequences allows for the portion of the nuclease recognition sequence to be reliably and advantageously spliced out during expression of the insert. An untranslated sequence (e.g., an intron) can also be utilized, as described herein, in some cases where a promoter is present on the first polynucleotide, and it is desired for a transgene on the second polynucleotide to be operably linked to that promoter. Examples of such configurations, and their associated elements, are described herein.

Additionally, in some examples of the invention described herein, the first polynucleotide and the second polynucleotide each comprise only one D sequence. In such examples, the first and second polynucleotides comprise a single D sequence at the same end; i.e., the D sequence is positioned within, near, or overlapping the 5′ ITR in both polynucleotides, or is positioned within, near, or overlapping the 3′ ITR in both polynucleotides. The use of a single D sequence, positioned on the same end of each polynucleotide, is particularly advantageous in the context of the present invention where different viral vectors having multiple areas of homology are introduced into the same cell. If two D sequences were present on each polynucleotide, or if single D sequences were present on different ends of the polynucleotides, the result would be a mixture of viral vectors synthesized from the sense strand of each construct, and viral vectors synthesized from the antisense strand of each construct. The presence of both sense and antisense strand-derived viral vectors, with multiple areas of homology, within the same cell could give rise to unwanted recombination events between the viral vectors, negatively impacting the proper assembly of the donor sequences in the genome. Thus, by only using a single D sequence in each polynucleotide, and ensuring that each D sequence is positioned on the same end in each polynucleotide, the present invention can advantageously avoid such unwanted recombination events. Examples of such configurations, and their associated elements, are described herein.

The configurations described herein also rely on appropriate use of homology regions, homology arms, and portions of the nuclease recognition sequences in order to promote insertion of the first donor sequence into the genome, and subsequent insertion of the second donor sequence into the first. Examples of such configurations, and their associated elements, are described herein.

In addition to the elements described herein, the first and second polynucleotides can comprise intervening nucleotides present between the elements, at a few enough number such that they do not interfere with homologous recombination events or expression of any encoded transgenes. In certain embodiments, there are fewer than 50, fewer than 40, fewer than 30, fewer than 20, fewer than 10, fewer than 5 or less nucleotides intervening between the recited elements.

### 2.3 Donor Nucleic Acids and Transgenes

The present invention involves the insertion of donor nucleic acids into a heterologous polynucleotide or a genomic locus. Donor nucleic acids can comprise any type of nucleic acid sequence, including but not limited to transgenes or portions thereof, regulatory sequences involved in the control of transcription or translation of transgenes, and nuclease recognition sequences. In particular embodiments of the presently disclosed compositions and methods, one or more transgenes are inserted within a heterologous polynucleotide or genomic locus. The transgene can encode for any type of RNA or protein.

In certain embodiments, the transgene that is desired to be inserted into a genomic locus is relatively long such that its length exceeds the packaging capacity of a single AAV (i.e., approximately 4.7 kb). Thus, in some embodiments, the transgene is longer than 4.7 kb, longer than 4.8 kb, longer than 4.9 kb, longer than 5 kb, longer than 6 kb, longer than 7 kb, longer than 8 kb, longer than 9 kb, longer than 10 kb, or longer. In those embodiments wherein the transgene exceeds the packaging capacity of an AAV, multiple stacking polynucleotides can be used to deliver portions of the transgene that can be inserted sequentially into the genome. Thus, in some embodiments, each stacking polynucleotide comprises a portion of the transgene, wherein each portion is about 4.7 kb or shorter in length.

Non-limiting examples of transgenes include those that encode a chimeric antigen receptor (CAR), an exogenous TCR, an inhibitory nucleic acid (e.g., dsRNA, siRNA, antisense RNA, miRNA, shRNA, shRNAmiR), a reporter protein, a protein useful for the purification of a cell of interest, a therapeutic protein, or a suicide protein.

Generally, a CAR of the present disclosure will comprise at least an extracellular domain, a transmembrane domain, and an intracellular domain. In some embodiments, the extracellular domain comprises a target-specific binding element otherwise referred to as an extracellular ligand-binding domain or moiety. In some embodiments, the intracellular domain, or cytoplasmic domain, comprises at least one co-stimulatory domain and one or more signaling domains.

A CAR useful in the invention comprises an extracellular ligand-binding domain. The choice of ligand-binding domain depends upon the type and number of ligands that define the surface of a target cell. For example, the ligand-binding domain may be chosen to recognize a ligand that acts as a cell surface marker on target cells associated with a particular disease state. Thus, some examples of cell surface markers that may act as ligands for the ligand-binding domain in a CAR can include those associated with viruses, bacterial and parasitic infections, autoimmune disease, and cancer cells. In some embodiments, a CAR is engineered to target a cancer-specific antigen of interest by way of engineering a desired ligand-binding moiety that specifically binds to an antigen on a cancer (i.e., tumor) cell. In the context of the present disclosure, “cancer antigen,” tumor antigen,” “cancer-specific antigen,” or “tumor-specific antigen” refer to antigens that are common to specific hyperproliferative disorders such as cancer.

In some embodiments, the extracellular ligand-binding domain of the CAR is specific for any antigen or epitope of interest, particularly any cancer antigen or epitope of interest. As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79B, IL1RAP, glypican 3 (GPC3), CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, insulin growth factor (IGFl)-l, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the Al domain of tenascin-C (TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), CS1, or a virus-specific surface antigen such as an HIV-specific antigen (such as HIV gp120); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

In certain embodiments, the ligand-binding domain of the CAR is an scFv. In some such embodiments, the scFv comprises a heavy chain variable (VH) domain and a light chain variable (VL) domain from a monoclonal antibody having specificity for a cancer cell antigen.

A CAR further comprises a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcγ receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain.

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular signaling domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

In other embodiments, a transgene described herein is an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest.

In various embodiments, the CARs and exogenous TCRs described herein have specificity for cancer cell antigens. Such cancers can include, without limitation, carcinoma, lymphoma, sarcoma, blastomas, leukemia, cancers of B cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin lymphoma. In specific embodiments, cancers and disorders include but are not limited to pre-B ALL (pediatric indication), adult ALL, mantle cell lymphoma, diffuse large B cell lymphoma, salvage post allogenic bone marrow transplantation, and the like. These cancers can be treated using a combination of CARs that target, for example, CD19, CD20, CD22, and/or ROR1. In some non-limiting examples, a genetically-modified immune cell or population thereof of the present disclosure targets carcinomas, lymphomas, sarcomas, melanomas, blastomas, leukemias, and germ cell tumors, including but not limited to cancers of B-cell origin, neuroblastoma, osteosarcoma, prostate cancer, renal cell carcinoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, multiple myeloma, and B-cell non-Hodgkin lymphoma. In some examples, cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

In some embodiments, modified eukaryotic cells of the present invention comprise an inactivated TCR alpha gene and/or an inactivated TCR beta gene. Inactivation of the TCR alpha gene and/or TCR beta gene to generate the CAR T cells of the present invention occurs in at least one or both alleles where the TCR alpha gene and/or TCR beta gene is being expressed. Accordingly, inactivation of one or both genes prevents expression of the endogenous TCR alpha chain or the endogenous TCR beta chain protein. Expression of these proteins is required for assembly of the endogenous alpha/beta TCR on the cell surface. Thus, inactivation of the TCR alpha gene and/or the TCR beta gene results in CAR T cells that have no detectable cell surface expression of the endogenous alpha/beta TCR. The endogenous alpha/beta TCR incorporates CD3. Therefore, cells with an inactivated TCR alpha gene and/or TCR beta chain can have no detectable cell surface expression of CD3. In particular embodiments, the inactivated gene is a TCR alpha constant region (TRAC) gene.

In some examples, the TCR alpha gene, the TRAC gene, or the TCR beta gene is inactivated by insertion of the polynucleotides described herein. Such insertion disrupts expression of the endogenous TCR alpha chain or TCR beta chain and, therefore, prevents assembly of an endogenous alpha/beta TCR on the T cell surface. In some examples, the polynucleotides of the invention are inserted into the TRAC gene. In a particular example, the polynucleotides are inserted into the TRAC gene at an engineered meganuclease recognition sequence comprising SEQ ID NO: 19. In particular examples, the polynucleotides of the invention are inserted into SEQ ID NO: 19 between nucleotide positions 13 and 14.

In other embodiments, the transgene of the presently disclosed compositions and methods encodes an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest.

In yet other embodiments, the transgene of the presently disclosed compositions and methods is a suicide gene, the expression of which can be inducible such that upon induction, cell death results, which allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

In certain embodiments, the presently disclosed compositions and methods involve the insertion of one or more transgenes that encode an inhibitory nucleic acid into a heterologous polynucleotide or genomic locus. The inhibitory nucleic acid can be any nucleic acid that reduces or inhibits the expression of at least one protein through RNA interference (RNAi). In some embodiments, the inhibitory RNA is a short hairpin RNA (shRNA) or microRNA-adapted shRNA (shRNAmiR) such as those described in U.S. Provisional Application Nos. 62/828,794, 62/843,804, 62/900,126, 62/930,905, and 63/000,774, each of which is incorporated by reference in its entirety. In particular embodiments, the shRNA or shRNAmiR reduces or inhibits the expression of at least one target protein selected from the group consisting of beta-2 microglobulin, CS1, transforming growth factor-beta receptor 2 (TGFBR2), Cbl proto-oncogene B (CBL-B), CD52, a TCR alpha gene, a TCR alpha constant region gene, CD7, glucocorticoid receptor (GR), deoxycytidine kinase (DCK), nuclear receptor subfamily 2 group F member 6 (NR2F6), cytotoxic T-lymphocyte-associated protein 4 (CTLA-4), and C-C chemokine receptor type 5 (CCR5).

In other embodiments, the presently disclosed compositions and methods involve the insertion into a heterologous polynucleotide or genome of one or more transgenes that encode a reporter protein. Reporter proteins can facilitate the identification and selection of expressing cells from the population of cells sought to be transfected or infected or cells that have donor nucleic acid(s) inserted into the genome. Useful reporter proteins include, for example, antibiotic-resistance genes, fluorescent marker genes, luciferase, and β-galactosidase.

In still other embodiments, the presently disclosed compositions and methods involve the insertion into a heterologous polynucleotide or genome of one or more transgenes that encode a protein useful for purification of a cell expressing the protein. Such proteins or tags are known in the art and include but are not limited to biotin, myc, maltose binding protein (MBP), and glutathione-S-transferase (GST), or a cell-surface protein that can be detected with a specific antibody.

In certain embodiments, the transgene can encode for a protein fused to a tag or epitope useful for detection or purification. For example, in order to assess the expression of a CAR or an exogenous T cell receptor in a genetically-modified cell, a CAR coding sequence may include a QBend10 epitope and/or EGFR epitope, which allows for detection using an anti-CD34 antibody and/or an anti-EGFR antibody (see, WO2011/056894, WO2013/153391, and WO2019/070856 each of which is incorporated by reference herein in its entirety). In other embodiments, sequences encoding a nuclease or a transgene can include at least one nuclear localization signal. Examples of nuclear localization signals are known in the art (see, e.g., Lange et al., J. Biol. Chem., 2007, 282:5101-5105).

In yet other embodiments, one or more transgenes that are inserted into a heterologous polynucleotide or genome comprise a therapeutic protein that effects beneficial or desirable biological and/or clinical results in the subject expressing it.

### 2.4 Nucleases

The presently disclosed compositions and methods utilize nucleases to cleave nuclease recognition sequences in order to allow the sequential insertion of donor nucleic acid sequences into a genomic locus. Non-limiting examples of nucleases useful in the present invention include zinc finger nucleases (ZFNs), transcription activator-like effector nucleases (TALENs), megaTALs, and CRISPR systems (e.g., Osborn et al. (2016), Molecular Therapy 24(3): 570-581; Eyquem et al. (2017), Nature 543: 113-117; U.S. Pat. No. 8,956,828; U.S. Publication No. US2014/0301990; U.S. Publication No. US2012/0321667).

In particular embodiments, engineered nucleases are used for the sequential stacking of donor nucleic acid sequences. Any engineered nuclease can be used for targeted insertion of the donor template, including an engineered meganuclease, a zinc finger nuclease, a TALEN, a compact TALEN, a CRISPR system nuclease, or a megaTAL.

For example, zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). In those embodiments wherein a CRISPR system is used for insertion of a donor nucleic acid sequence into a heterologous polynucleotide or genomic locus, the CRISPR system comprises two components: (1) a CRISPR nuclease; and (2) a short “guide RNA” comprising a ˜20 nucleotide targeting sequence that directs the nuclease to a location of interest in the genome or on a polynucleotide. The CRISPR system may also comprise a tracrRNA. By expressing multiple guide RNAs in the same cell, each having a different targeting sequence, it is possible to target DNA breaks simultaneously to multiple sites in the genome. The presently disclosed compositions and methods utilizing a CRISPR system may comprise a CRISPR nuclease and the guide RNA(s) or nucleic acids encoding the CRISPR nuclease and/or the guide RNA(s).

Engineered meganucleases that bind double-stranded DNA at a recognition sequence that is greater than 12 base pairs can be used for the presently disclosed methods. A meganuclease can be an endonuclease that is derived from I-CreI and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker.

Nucleases referred to as megaTALs are single-chain endonucleases comprising a transcription activator-like effector (TALE) DNA binding domain with an engineered, sequence-specific homing endonuclease.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. For example, nuclease-mediated insertion using engineered single-chain meganucleases has been disclosed in International Publication Nos. WO 2017/062439, WO 2017/062451, and WO/2019200122. Nuclease-mediated insertion of donor nucleic acid sequences can also be accomplished using an engineered single-chain meganuclease comprising SEQ ID NO: 40 (i.e., TRC 1-2L.1592).

In those embodiments wherein the donor nucleic acid sequences are being inserted into a human T cell receptor alpha constant (TRAC) gene, nucleases that cleave DNA within the TRAC gene are used. The specific use of engineered meganucleases for cleaving DNA targets in the human TRAC gene has also been previously disclosed. For example, International Publication No. WO 2014/191527, which disclosed variants of the I-OnuI meganuclease that were engineered to target a recognition sequence within exon 1 of the TCR alpha constant region gene. Moreover, in International Publication Nos. WO 2017/062439, WO 2017/062451 and WO2019/200122, Applicants disclosed engineered meganucleases which have specificity for recognition sequences in exon 1 of the TCR alpha constant region gene. These included “TRC 1-2 meganucleases” which have specificity for the TRC 1-2 recognition sequence (SEQ ID NO: 19) in exon 1 of the TRAC gene. The '439, '451, and '122 publications also disclosed methods for targeted insertion of a CAR coding sequence or an exogenous TCR coding sequence into a cleavage site in the TCR alpha constant region gene.

The presently disclosed compositions and methods can utilize purified nuclease proteins, or nucleic acids encoding nucleases. These can be delivered into cells to cleave genomic DNA or a polynucleotide by a variety of different mechanisms known in the art, including those further detailed elsewhere herein. In those embodiments wherein a CRISPR/cas nuclease is utilized, a ribonucleoprotein complex comprising the CRISPR nuclease and guide RNA(s) can be introduced into a cell.

### 2.5 Variant Polynucleotides and Polypeptides

The present invention encompasses variants of the polypeptide and polynucleotide sequences described herein. As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein (e.g., nuclease activity for an engineered nuclease). Such variants may result, for example, from human manipulation. Biologically active variants of polypeptides described herein will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, as determined by sequence alignment programs and parameters described elsewhere herein. A biologically active variant of a polypeptide may differ from that polypeptide or subunit by as few as about 1-40 amino acid residues, as few as about 1-20, as few as about 1-10, as few as about 5, as few as 4, 3, 2, or even 1 amino acid residue.

The polypeptides may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a polypeptide or RNA. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide (e.g., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its biological activity.

### 2.6 Methods of Introducing Polynucleotides and Nucleases into Cells

The present invention provides methods for producing genetically-modified cells by introducing into cells polynucleotides useful for stacking donor nucleic acids within a particular genomic locus, as well as nucleases or nucleic acids encoding the same that mediate the targeted insertion of the donor nucleic acids.

Thus, provided herein are vectors comprising the stacking polynucleotide(s) and/or the nucleic acid(s) encoding nuclease(s) molecules of the present disclosure. In some embodiments, the stacking polynucleotide(s) and/or the nucleic acid(s) encoding nuclease(s) are cloned into a vector including, but not limited to a plasmid, a phagemid, a phage derivative, an animal virus, or a cosmid. Vectors of particular interest include expression vectors, replication vectors, probe generation vectors, sequencing vectors, and viral vectors.

If the nuclease genes are delivered in DNA form (e.g. plasmid) and/or via a viral vector (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a preferred embodiment, nuclease genes are operably linked to a promoter that drives gene expression preferentially in the target cell (e.g., a T cell).

The stacking polynucleotide(s) may also comprise promoters and/or other regulatory sequences that are operably linked to the transgene(s) and regulate the expression thereof or will become operably linked to the transgene(s) following insertion of the donor nucleic acid molecule(s). Various promoters can be used to drive the expression of the transgene(s). One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Synthetic promoters are also contemplated as part of the present disclosure. For example, in particular embodiments, the promoter is a JeT promoter (see, WO/2002/012514).

In some embodiments, the promoters are selected based on the desired outcome. It is recognized that different applications can be enhanced by the use of different promoters in the expression cassettes to modulate the timing, location and/or level of expression of the polynucleotides disclosed herein. Such expression constructs may also contain, if desired, a promoter regulatory region (e.g., one conferring inducible, constitutive, environmentally- or developmentally-regulated, or cell- or tissue-specific/selective expression), a transcription initiation start site, translation initiation sites (e.g., Kozak sequences), a ribosome binding site, an RNA processing signal, a transcription termination site, a polyadenylation signal, and/or origins of replication.

The stacking polynucleotide(s) and nuclease(s) or nucleic acids encoding the same can be introduced into a cell by any method known in the art.

In some embodiments, mRNA encoding the engineered nuclease(s) is delivered to the cell because this reduces the likelihood that the gene encoding the nuclease will integrate into the genome of the cell. The mRNA encoding a nuclease can be produced using methods known in the art such as in vitro transcription. In some embodiments, the mRNA comprises a modified 5′ cap. Such modified 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, Calif.), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element. The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In particular embodiments, nucleoside modifications can include a modification of uridine to pseudouridine, and/or a modification of uridine to N1-methyl pseudouridine.

In other embodiments, the stacking polynucleotide(s) and/or nucleic acids encoding nuclease(s) are introduced into a cell using a linearized DNA template. Such linearized DNA templates can be produced by methods known in the art. For example, a plasmid DNA can be digested by one or more restriction enzymes such that the circular plasmid DNA is linearized prior to being introduced into a cell.

In some embodiments, the stacking polynucleotide(s) and/or nucleic acids encoding nuclease(s) are delivered using recombinant viruses, and particularly using adeno-associated viruses (AAVs) (reviewed in Vannucci, et al. (2013 New Microbiol. 36:1-22). Viral vector technology is well known in the art and is described, for example, in Sambrook et al. (2001, Molecular Cloning: A Laboratory Manual, Cold Spring Harbor Laboratory, New York), and in other virology and molecular biology manuals. In general, a suitable vector contains an origin of replication functional in at least one organism, convenient restriction endonuclease sites, and one or more selectable markers, (e.g., WO 01/96584; WO 01/29058; and U.S. Pat. No. 6,326,193).

Recombinant AAVs useful in the invention can have any serotype that allows for transduction of the virus into a target cell type, expression of the nuclease gene, insertion of the first and second polynucleotides into the genome according to the invention, and expression of any transgenes encoded thereby. In some embodiments, the AAV vector has a serotype of AAV1, AAV2, AAV3, AAV4, AAV5, AAV6, AAV7, AAV8, AAV9, AAV10, or AAV11. Other AAV serotypes are known in the art can be selected for use by those of skill in the art. In some embodiments, the recombinant AAV has a serotype of AAV6. In some embodiments, the recombinant AAV has a serotype of AAV8. Recombinant AAV vectors can be single-stranded AAV vectors. AAV vectors can also be self-complementary such that they do not require second-strand DNA synthesis in the host cell (McCarty, et al. (2001) Gene Ther. 8:1248-54). The viral vector can comprise a 5′ and/or a 3′ AAV inverted terminal repeat (ITR) upstream and/or downstream of the sequence encoding the nuclease or the donor nucleic acid sequences in stacking polynucleotides.

Recombinant AAV vectors are typically produced in mammalian cell lines such as HEK-293. Because the viral cap and rep genes are removed from the vector to prevent its self-replication to make room for transgene(s) to be delivered, it is necessary to provide these in trans in the packaging cell line. In addition, it is necessary to provide the “helper” (e.g. adenoviral) components necessary to support replication (Cots et al. (2013), Curr. Gene Ther. 13(5): 370-81). Frequently, recombinant AAV vectors are produced using a triple-transfection in which a cell line is transfected with a first plasmid encoding the “helper” components, a second plasmid comprising the cap and rep genes, and a third plasmid comprising the viral inverted terminal repeats (ITRs) containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient.

Because recombinant AAV particles are typically produced (manufactured) in cells, precautions must be taken in practicing the current invention to ensure that the engineered nuclease(s) are not expressed in the packaging cells. Because the viral genomes of the invention may comprise a recognition sequence for the nuclease(s), any nuclease expressed in the packaging cell line may be capable of cleaving the viral genome before it can be packaged into viral particles. This will result in reduced packaging efficiency and/or the packaging of fragmented genomes. Several approaches can be used to prevent nuclease expression in the packaging cells.

The nuclease(s) can be placed under the control of a tissue-specific promoter that is not active in the packaging cells. For example, if a viral vector is developed for delivery of nuclease gene(s) to muscle tissue, a muscle-specific promoter can be used. Examples of muscle-specific promoters include C5-12 (Liu, et al. (2004) Hum Gene Ther. 15:783-92), the muscle-specific creatine kinase (MCK) promoter (Yuasa, et al. (2002) Gene Ther. 9:1576-88), or the smooth muscle 22 (SM22) promoter (Haase, et al. (2013) BMC Biotechnol. 13:49-54). Examples of CNS (neuron)-specific promoters include the NSE, Synapsin, and MeCP2 promoters (Lentz, et al. (2012) Neurobiol Dis. 48:179-88). Examples of liver-specific promoters include albumin promoters (such as Palb), human α1-antitrypsin (such as Pa1AT), and hemopexin (such as Phpx) (Kramer et al., (2003) Mol. Therapy 7:375-85), hybrid liver-specific promoter (hepatic locus control region from ApoE gene (ApoE-HCR) and a liver-specific alpha1-antitrypsin promoter), human thyroxine binding globulin (TBG) promoter, and apolipoprotein A-II promoter. Examples of eye-specific promoters include opsin, and corneal epithelium-specific K12 promoters (Martin et al. (2002) Methods (28): 267-75) (Tong et al., (2007) J Gene Med, 9:956-66). These promoters, or other tissue-specific promoters known in the art, are not highly-active in HEK-293 cells and, thus, will not be expected to yield significant levels of nuclease gene expression in packaging cells when incorporated into viral vectors of the present invention. Similarly, the viral vectors of the present invention contemplate the use of other cell lines with the use of incompatible tissue specific promoters (i.e., the well-known HeLa cell line (human epithelial cell) and using the liver-specific hemopexin promoter). Other examples of tissue specific promoters include: synovial sarcomas PDZD4 (cerebellum), C6 (liver), ASB5 (muscle), PPP1R12B (heart), SLC5A12 (kidney), cholesterol regulation APOM (liver), ADPRHL1 (heart), and monogenic malformation syndromes TP73L (muscle). (Jacox et al., (2010), PLoS One v.5(8):e12274).

Alternatively, the vector can be packaged in cells from a different species in which the nuclease(s) are not likely to be expressed. For example, viral particles can be produced in microbial, insect, or plant cells using mammalian promoters, such as the well-known cytomegalovirus- or SV40 virus-early promoters, which are not active in the non-mammalian packaging cells. In a particular embodiment, viral particles are produced in insect cells using the baculovirus system as described by Gao, et al. (Gao et al. (2007), J. Biotechnol. 131(2):138-43). A nuclease under the control of a mammalian promoter is unlikely to be expressed in these cells (Airenne et al. (2013), Mol. Ther. 21(4):739-49). Moreover, insect cells utilize different mRNA splicing motifs than mammalian cells. Thus, it is possible to incorporate a mammalian intron, such as the human growth hormone (HGH) intron or the SV40 large T antigen intron, into the coding sequence of a nuclease. Because these introns are not spliced efficiently from pre-mRNA transcripts in insect cells, insect cells will not express a functional nuclease and will package the full-length genome. In contrast, mammalian cells to which the resulting recombinant AAV particles are delivered will properly splice the pre-mRNA and will express functional nuclease protein. Haifeng Chen has reported the use of the HGH and SV40 large T antigen introns to attenuate expression of the toxic proteins barnase and diphtheria toxin fragment A in insect packaging cells, enabling the production of recombinant AAV vectors carrying these toxin genes (Chen, H (2012) Mol Ther Nucleic Acids. 1(11): e57).

The nuclease gene(s) can be operably linked to an inducible promoter such that a small-molecule inducer is required for nuclease expression. Examples of inducible promoters include the Tet-On system (Clontech; Chen et al. (2015), BMC Biotechnol. 15(1):4)) and the RheoSwitch system (Intrexon; Sowa et al. (2011), Spine, 36(10): E623-8). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). Practicing the current invention using such ligand-inducible transcription activators includes: 1) placing the nuclease gene(s) under the control of a promoter that responds to the corresponding transcription factor, the nuclease gene(s) having (a) binding site(s) for the transcription factor; and 2) including the gene encoding the transcription factor in the packaged viral genome. The latter step is necessary because the nuclease(s) will not be expressed in the target cells or tissues following recombinant AAV delivery if the transcription activator is not also provided to the same cells. The transcription activator then induces nuclease gene expression only in cells or tissues that are treated with the cognate small-molecule activator. This approach is advantageous because it enables nuclease gene expression to be regulated in a spatio-temporal manner by selecting when and to which tissues the small-molecule inducer is delivered. However, the requirement to include the inducer in the viral genome, which has significantly limited carrying capacity, creates a drawback to this approach.

In another particular embodiment, recombinant AAV particles are produced in a mammalian cell line that expresses a transcription repressor that prevents expression of the nuclease(s). Transcription repressors are known in the art and include the Tet-Repressor, the Lac-Repressor, the Cro repressor, and the Lambda-repressor. Many nuclear hormone receptors such as the ecdysone receptor also act as transcription repressors in the absence of their cognate hormone ligand. To practice the current invention, packaging cells are transfected/transduced with a vector encoding a transcription repressor and the nuclease gene(s) in the viral genome (packaging vector) is operably linked to a promoter that is modified to comprise binding sites for the repressor such that the repressor silences the promoter. The gene encoding the transcription repressor can be placed in a variety of positions. It can be encoded on a separate vector; it can be incorporated into the packaging vector outside of the ITR sequences; it can be incorporated into the cap/rep vector or the adenoviral helper vector; or it can be stably integrated into the genome of the packaging cell such that it is expressed constitutively. Methods to modify common mammalian promoters to incorporate transcription repressor sites are known in the art. For example, Chang and Roninson modified the strong, constitutive CMV and RSV promoters to comprise operators for the Lac repressor and showed that gene expression from the modified promoters was greatly attenuated in cells expressing the repressor (Chang and Roninson (1996), Gene 183:137-42). The use of a non-human transcription repressor ensures that transcription of the nuclease gene(s) will be repressed only in the packaging cells expressing the repressor and not in target cells or tissues transduced with the resulting recombinant AAV vector.

In other embodiments, the nuclease protein(s) or nucleic acid(s) encoding the nuclease(s) and/or the stacking polynucleotide(s), are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, engineered nucleases or nucleic acids encoding nucleases and/or stacking polynucleotide(s) are coupled covalently or non-covalently to an antibody that recognizes a specific cell-surface receptor expressed on target cells such that the nuclease protein(s) or nucleic acid(s) encoding the nuclease(s) and/or stacking polynucleotide(s) binds to and are internalized by the target cells. Alternatively, the nuclease protein(s) or nucleic acid(s) encoding the nuclease(s) and/or stacking polynucleotide(s) can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

Nuclease proteins, nucleic acids encoding nuclease proteins, and/or stacking polynucleotides described herein can be introduced into cells, or into particular tissues, by various means other than viral deliver. In some embodiments, nuclease protein(s) or nucleic acid(s) encoding nuclease(s) and/or stacking polynucleotide(s) are encapsulated within biodegradable hydrogels for injection or implantation within the desired region of a tissue. Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, nuclease protein(s) or nucleic acid(s) encoding the nuclease(s) and/or stacking polynucleotide(s) are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins nucleic acids can be attached to or encapsulated with the nanoparticle core. This increases the copy number of the protein or nucleic acid that is delivered to each cell and, so, increases the intracellular expression of each nuclease to maximize the likelihood that the nuclease recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). In some embodiments, the nuclease protein(s) or nucleic acid(s) encoding the nuclease(s) and/or stacking polynucleotide(s) are encapsulated within a lipid nanoparticle. Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the nuclease protein(s) or nucleic acid(s) encoding the nuclease(s) and/or stacking polynucleotide(s) are encapsulated within liposomes or complexed using cationic lipids (see, e.g., LIPOFECTAMINE™, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, nuclease protein(s) or nucleic acid(s) encoding the nuclease(s) and/or stacking polynucleotide(s) are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease protein(s) or nucleic acid(s) encoding the nuclease(s) and/or stacking polynucleotide(s) are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease protein(s) or nucleic acid(s) encoding the nuclease(s) and/or stacking polynucleotide(s) are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, nuclease protein(s) or nucleic acid(s) encoding the nuclease(s) and/or stacking polynucleotide(s) are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and release of the nuclease and/or polynucleotides.

In different alternatives of the invention, the two or more stacking polynucleotides can be introduced simultaneously or sequentially into a cell. In certain embodiments, one stacking polynucleotide described herein is introduced into the cell within at least 1 hour, 2 hours, 3 hours, 4 hours, 5 hours, 6 hours, 7 hours, 8 hours, 9 hours, 10 hours, 12 hours, 15 hours, 20 hours, 24 hours, 2 days, 3 days, or more of the other stacking polynucleotide.

The nuclease, or nucleic acid encoding the nuclease, can similarly be introduced to the cell simultaneously or sequentially with the first, second, and/or subsequent stacking polynucleotides. In some embodiments, the nuclease, or nucleic acid encoding the nuclease, is introduced within at least 1 hour, 2 hours, 3 hours, 4 hours, 5 hours, 6 hours, 7 hours, 8 hours, 9 hours, 10 hours, 12 hours, 15 hours, 20 hours, 24 hours, 2 days, 3 days, or more of the first, second, and/or subsequent stacking polynucleotides.

### 2.7 Genetically-Modified Cells

The invention provides cells comprising stacking polynucleotide(s) and one or more nucleases or nucleic acids encoding such nucleases. Also provided are genetically-modified cells wherein one or more donor nucleic acids from one or more stacking polynucleotides have been inserted into the genome, and populations thereof and methods for producing the same. The donor nucleic acid sequence(s) of the stacking polynucleotide(s) can be inserted into any genomic locus that comprises an endogenous nuclease recognition site. In some embodiments, the donor nucleic acid sequence(s) are inserted into a gene of interest. In some of these embodiments, the insertion of the donor nucleic acid sequence(s) into a gene of interest results in the disruption of that gene such that the gene product is not expressed or the gene product is not functional. In particular embodiments, the gene of interest is a TCR alpha gene or a TCR beta gene. In some of these embodiments, the gene of interest is a TCR alpha constant (TRAC) gene or a TCR beta constant (TRBC) gene.

In general, the cells that are genetically-modified using the presently disclosed compositions and methods are eukaryotic cells. In some embodiments, the cells are mammalian cells, including but not limited to nonhuman primates, mice, rats, rabbits, cats, dogs, and humans. In particular embodiments, the cells provided herein are immune cells or cells derived therefrom and populations thereof and methods for producing the same are provided. In some embodiments, the cells of the presently disclosed compositions and methods are human immune cells or cells derived therefrom. In some embodiments, the immune cells are T cells, or cells derived therefrom. In other embodiments, the immune cells are natural killer (NK) cells, or cells derived therefrom. In still other embodiments, the immune cells are B cells, or cells derived therefrom. In yet other embodiments, the immune cells are monocyte or macrophage cells or cells derived therefrom. In certain embodiments, the immune cells are induced pluripotent stem cells (iPSCs) or cells derived therefrom.

Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines, NK cell lines, B cell lines, monocyte cells lines, macrophage cell, or iPSC cell lines available in the art may be used. In some embodiments of the present disclosure, immune cells are obtained from a unit of blood collected from a subject using any number of techniques known to the skilled artisan. In one embodiment, cells from the circulating blood of an individual are obtained by apheresis.

In some embodiments, the cell of the present invention is an induced pluripotent stem cell or cells derived therefrom, such as iPSCs that have been differentiated into a particular cell type. iPSCs are stem cells that are generated by the reprogramming of somatic cells by expressing or inducing expression of a combination of factors (i.e., reprogramming factors). iPSCs can be generated using fetal, postnatal, newborn, juvenile, or adult somatic cells. Somatic cell types that can be reprogrammed into iPSCs include but are not limited to fibroblast, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, the somatic cell that is reprogrammed into an iPSC can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell. When reprogrammed cells are used for therapeutic purposes, it is desirable, but not required, to use somatic cells isolated from the patient being treated. For example, somatic cells involved in diseases, and somatic cells participating in therapeutic treatment of diseases and the like can be used.

The obtained somatic cells can be reprogrammed into iPSCs using any method known in the art. Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. The efficiency of reprogramming (i.e., the number of reprogrammed cells) derived from a population of starting cells can be enhanced by the addition of various small molecules as shown by Shi, Y., et al (2008) Cell—Stem Cell 2:525-528, Huangfu, D., et al (2008) Nature Biotechnology 26(7):795-797, and Marson, A., et al (2008) Cell—Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; Slc2a3; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; CD9; Dnmt3L; Sox15; Stat3; Grb2; β-catenin; and Bmi. The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells. In some embodiments, a method for selecting the reprogrammed cells from a heterogeneous population comprising reprogrammed cells and somatic cells from which they were derived or generated from can be performed by any known means. For example, a drug resistance gene or the like, such as a selectable marker gene can be used to isolate the reprogrammed cells using the selectable marker as an index.

In some embodiments, the genetically-modified cells express CARs or exogenous TCRs that have specificity for cancer cell antigens. Such cancers can include, without limitation, those cancers described elsewhere herein.

T cells modified by the present invention may require activation prior to introduction of a nuclease and/or stacking polynucleotide(s). For example, T cells can be contacted with anti-CD3 and anti-CD28 antibodies that are soluble or conjugated to a support (e.g., beads) for a period of time sufficient to activate the cells.

The invention provides a population of cells that have been genetically-modified using the presently disclosed methods and compositions. In various embodiments of the invention, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified cell as described herein. In a particular example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population express a CAR or exogenous TCR and have an inactivated TCR alpha and/or beta gene.

### 2.8 Pharmaceutical Compositions

In one aspect of the invention, the present disclosure provides a pharmaceutical composition comprising a pharmaceutically acceptable carrier and a composition described herein, wherein the composition comprises the stacking polynucleotide(s) and one or more nucleases or nucleic acids encoding nucleases described herein.

In another aspect, the present disclosure provides a pharmaceutical composition comprising a pharmaceutically acceptable carrier and a cell of the invention (e.g., genetically-modified cell) or population thereof. The cell or population thereof can be delivered to a target tissue.

Pharmaceutical compositions of the invention can be useful for treating a subject having a disease by modifying a gene associated with the disease or providing a transgene that when expressed, treats the disease. In other embodiments, the pharmaceutical composition comprises genetically-modified immune cells that express a CAR or exogenous TCR.

The present disclosure also provides compositions comprising stacking polynucleotide(s) and nuclease(s) or nucleic acid(s) encoding the same, as well as cells (e.g., genetically-modified cells), or populations thereof, described herein for use as a medicament. The present disclosure further provides the use of compositions comprising stacking polynucleotide(s) and nuclease(s) or nucleic acid(s) encoding the same, as well as cells (e.g., genetically-modified cells), or populations thereof, described herein in the manufacture of a medicament for treating a disease in a subject in need thereof. In one such aspect, a medicament comprising genetically-modified immune cells expressing a CAR or exogenous TCR is useful for cancer immunotherapy in subjects in need thereof.

Such pharmaceutical compositions can be prepared in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, stacking polynucleotide(s) and nuclease(s) or nucleic acid(s) encoding the same, or cells (e.g., genetically-modified cells), or populations thereof, described herein are typically admixed with a pharmaceutically acceptable carrier, and the resulting composition is administered to a subject. The carrier must be acceptable in the sense of being compatible with any other ingredients in the formulation and must not be deleterious to the subject. In some embodiments, pharmaceutical compositions of the invention can further comprise one or more additional agents or biological molecules useful in the treatment of a disease in the subject. In additional embodiments, pharmaceutical compositions of the invention can further include biological molecules, such as cytokines (e.g., IL-2, IL-7, IL-15, and/or IL-21), which may promote in vivo cell proliferation and engraftment of genetically-modified cells. Likewise, the additional agent(s) and/or biological molecule(s) can be co-administered as a separate composition.

In particular embodiments of the invention, the pharmaceutical composition comprises viral vectors comprising stacking polynucleotide(s) and/or nucleic acid(s) encoding nuclease(s) described herein. Such vectors are known in the art and include, for example, recombinant AAVs. In some embodiments, the viral vectors are injected directly into target tissues. In alternative embodiments, the viral vectors are delivered systemically via the circulatory system. It is known in the art that different AAV vectors tend to localize to different cells or tissues, and one of skill in the art can select an appropriate AAV serotype depending on the target cell or target tissue.

In particular embodiments of the invention, the pharmaceutical composition comprises one or more stacking polynucleotide, nuclease, or nucleic acid encoding a nuclease described herein, formulated within lipid nanoparticles.

The selection of cationic lipids, non-cationic lipids and/or lipid conjugates which comprise the lipid nanoparticle, as well as the relative molar ratio of such lipids to each other, is based upon the characteristics of the selected lipid(s), the nature of the intended target cells, and the characteristics of the nuclease or nucleic acid to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method of the invention can be prepared by various techniques which are presently known in the art. Nucleic acid-lipid particles and their method of preparation are disclosed in, for example, U.S. Patent Publication Nos. 20040142025 and 20070042031, the disclosures of which are herein incorporated by reference in their entirety for all purposes.

Selection of the appropriate size of lipid nanoparticles must take into consideration the site of the target cell and the application for which the lipid nanoparticles is being made. Generally, the lipid nanoparticles will have a size within the range of about 25 to about 500 nm. In some embodiments, the lipid nanoparticles have a size from about 50 nm to about 300 nm or from about 60 nm to about 120 nm. The size of the lipid nanoparticles may be determined by quasi-electric light scattering (QELS) as described in Bloomfield, Ann. Rev. Biophys. Bioeng., 10:421-450 (1981), incorporated herein by reference. A variety of methods are known in the art for producing a population of lipid nanoparticles of particular size ranges, for example, sonication or homogenization. One such method is described in U.S. Pat. No. 4,737,323, incorporated herein by reference.

Some lipid nanoparticles contemplated for use in the invention comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino)butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOB A), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid comprises from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid comprises from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain particular embodiments, the phospholipid is DPPC, DSPC, or mixtures thereof.

In some embodiments, the non-cationic lipid (e.g., one or more phospholipids and/or cholesterol) comprises from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-di lauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′(1,2-dimyristoyl-3-propanoxy)-carboxamido-3′,6′-dioxaoctanyl]carbamoyl-ω-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition comprises amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are charged more and all anionic charge carriers lose their charge.

Cationic compounds useful for amphoteric liposomes include those cationic compounds previously described herein above. Without limitation, strongly cationic compounds can include, for example: DC-Chol 3-β-[N—(N′,N′-dimethylmethane) carbamoyl] cholesterol, TC-Chol 3-β-[N—(N′, N′, N′-trimethylaminoethane) carbamoyl cholesterol, BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylarnide, DOTMA (1,2-dioleoyloxypropyl)-N,N,N-trimethylamronium chloride) (Lipofectin®), DORIE 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18)2Gly+N,N-dioctadecylamido-glycine, CTAB cetyltrimethylarnmonium bromide, CpyC cetylpyridinium chloride, DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Some particular examples are PEG-modified diacylglycerols and dialkylglycerols.

In some embodiments, the neutral lipids comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

### 2.9 Methods of Administering Genetically-Modified Cells

The method of the invention comprises administering to a subject a pharmaceutical composition comprising stacking polynucleotide(s) and nuclease(s) or nucleic acid(s) encoding such nucleases, or cells comprising the same, or genetically-modified cells that have been modified using the presently disclosed compositions and methods, or populations of cells disclosed herein. In some embodiments, the invention comprises administering a population of genetically-modified cells, wherein the population comprises a plurality of immune cells expressing a CAR or an exogenous TCR (e.g., CAR T cells). For example, the pharmaceutical composition administered to the subject can comprise an effective dose of immune cells expressing a CAR or an exogenous TCR (e.g., CAR T cells) for treatment of a cancer and administration of the genetically-modified immune cells of the invention represent an immunotherapy. The administered CAR T cells are able to reduce the proliferation, reduce the number, or kill target cells in the recipient. Unlike antibody therapies, genetically-modified cells of the present disclosure are able to replicate and expand in vivo, resulting in long-term persistence that can lead to sustained control of a disease.

When an “effective amount” or “therapeutic amount” is indicated, the precise amount to be administered can be determined by a physician with consideration of individual differences in age, weight, tumor size (if present), extent of infection or metastasis, and condition of the patient (subject). In some embodiments, a pharmaceutical composition comprising the genetically-modified immune cells or populations thereof described herein is administered at a dosage of 10⁴ to 10⁹ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regimen for a particular patient can readily be determined by one skilled in the art of medicine by monitoring the patient for signs of disease and adjusting the treatment accordingly.

Examples of possible routes of administration of pharmaceutical compositions described herein include parenteral, (e.g., intravenous (IV), intramuscular (IM), intradermal, subcutaneous (SC), or infusion) administration. Moreover, the administration may be by continuous infusion or by single or multiple boluses. In specific embodiments, one or both of the agents is infused over a period of less than about 12 hours, less than about 10 hours, less than about 8 hours, less than about 6 hours, less than about 4 hours, less than about 3 hours, less than about 2 hours, or less than about 1 hour. In still other embodiments, the infusion occurs slowly at first and then is increased over time.

In some embodiments, pharmaceutical compositions of the invention can be useful for treating any disease state that can be targeted by adoptive immunotherapy, and particularly T cell adoptive immunotherapy. In a particular embodiment, pharmaceutical compositions and medicaments of the invention are useful in the treatment of cancer. In some embodiments, a pharmaceutical composition of the present disclosure comprises immune cells comprising a CAR or exogenous TCR targeting a cancer cell antigen (i.e., an antigen expressed on the surface of a cancer cell) for the purpose of treating cancer. Such cancers can include, without limitation, those cancers described elsewhere herein.

In some embodiments, the administration of pharmaceutical compositions of the present disclosure reduces at least one symptom of a target disease or condition. For example, administration of genetically-modified cells of the present disclosure can reduce at least one symptom of a cancer, such as cancers of B-cell origin. Symptoms of cancers are well known in the art and can be determined by known techniques.

In some of these embodiments wherein cancer is treated, the subject can be further administered an additional therapeutic agent or treatment, including, but not limited to gene therapy, radiation, surgery, or a chemotherapeutic agent(s) (i.e., chemotherapy).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Design of Constructs for AAV Stacking

To demonstrate an example of an AAV stacking approach, three different constructs were engineered to generate first and second AAV vectors. These vectors were designed to be inserted at a recognition sequence within the T cell receptor alpha constant (TRAC) gene at a recognition sequence referred to as TRC 1-2 (SEQ ID NO: 1). The TRC 1-2 recognition sequence is incorporated into the first AAV vector and in one of the second AAV vectors. By utilizing a recognition sequence in the AAV vectors that is identical to the recognition sequence in the genome, a single engineered meganuclease, referred to as TRC 1-2L.1592, can be expressed in the cell to generate the necessary double-stranded breaks in the genome and the constructs. Further, the AAVs utilized in these experiments are single-stranded AAVs, which should prevent cleavage of the AAVs by the nuclease and increase persistence of the AAVs and efficiency of the system.

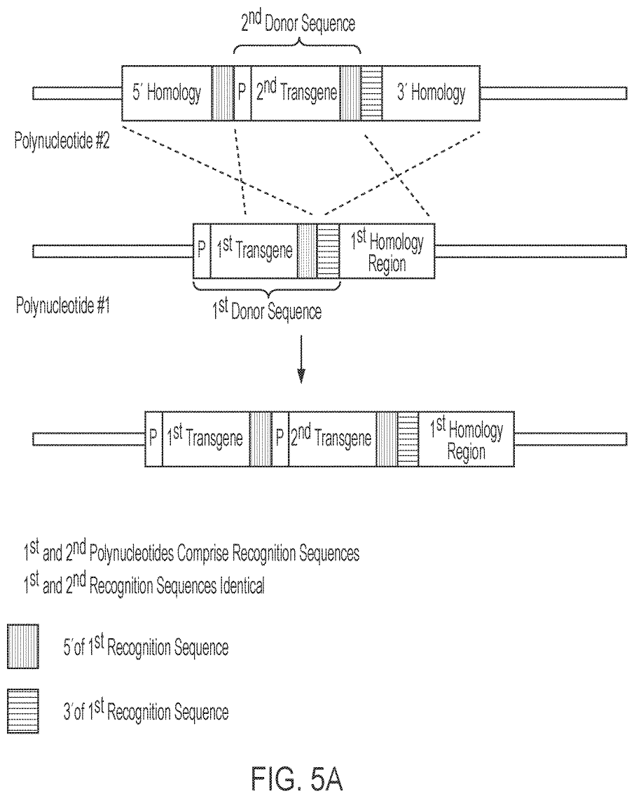

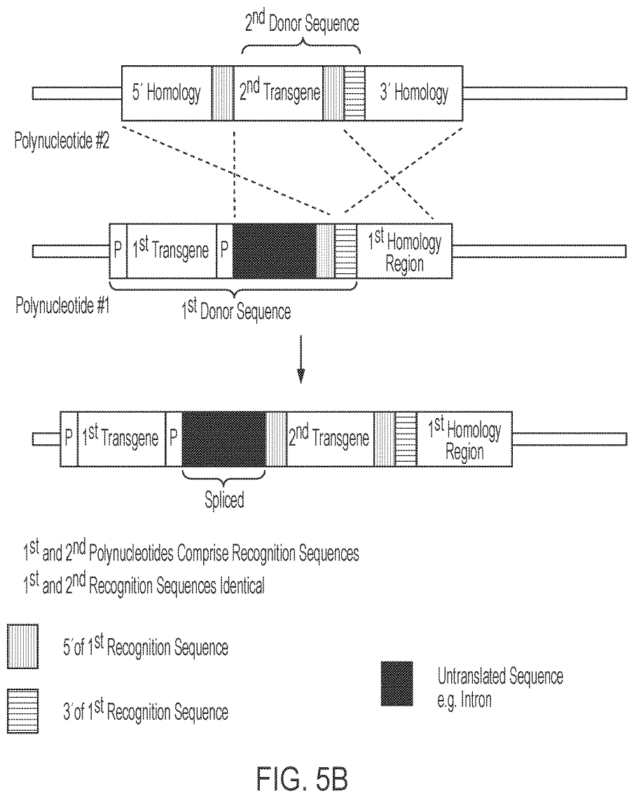

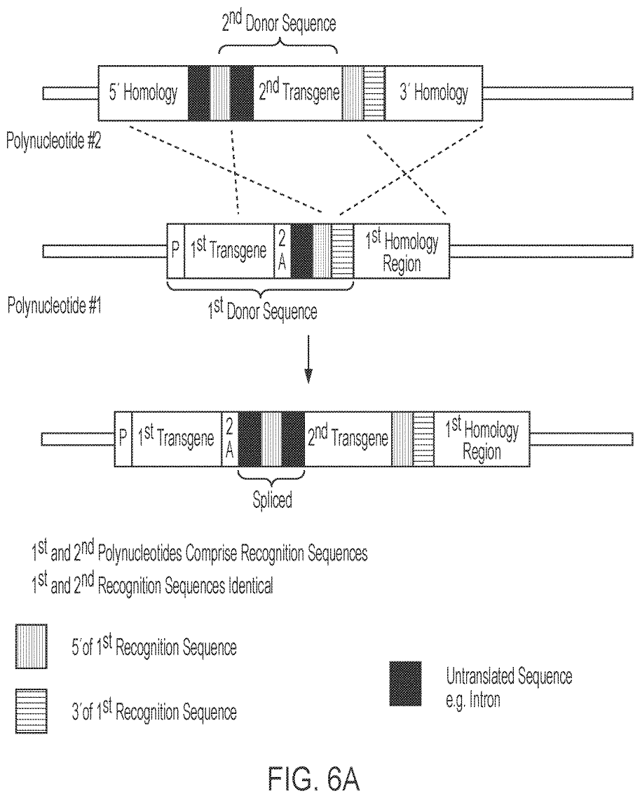

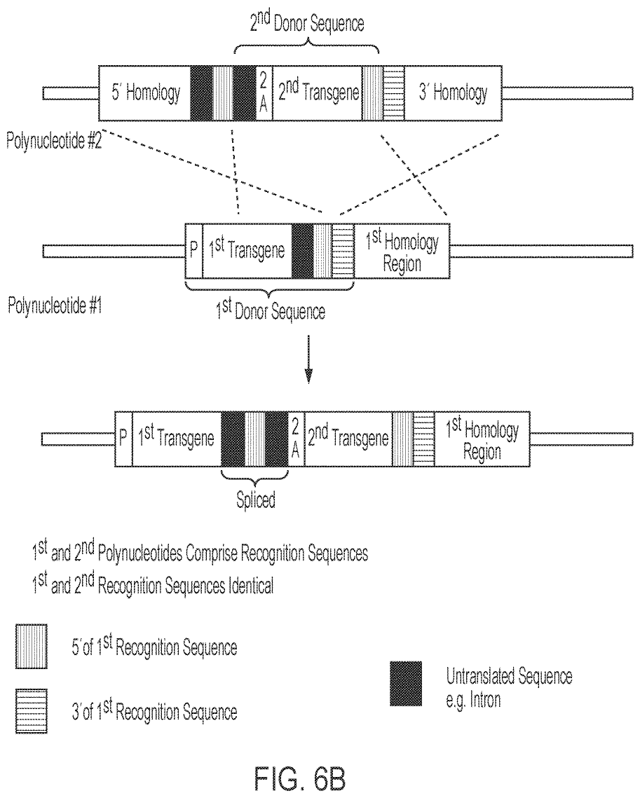

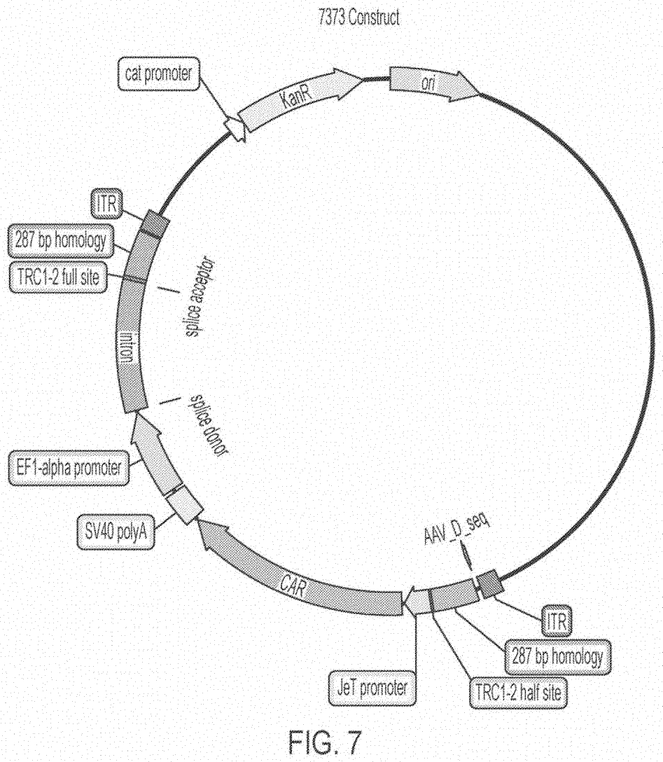

The first AAV vector, referred to as 7373 (FIG. 7; SEQ ID NO: 3), comprises a single D sequence, a 287 bp TRAC-specific left homology arm, a 5′ portion of the TRC 1-2 recognition sequence, a JeT promoter, a coding sequence for a CD19-specific chimeric antigen receptor, an EF1-alpha promoter, an 826 bp intron, a full TRC 1-2 recognition sequence, and a 287 bp TRAC-specific right homology arm. These vectors are designed such that a first promoter (i.e., the JeT promoter) drives expression of the CAR, and the second promoter (i.e., the EF1-alpha promoter) will be able to drive expression of a second transgene on the second AAV once stacking has occurred. The inclusion of the intron sequence provides a 5′ homology region for the second AAV vector. Further, the intron sequence comprises a splice donor sequence and a splice acceptor sequence, allowing for the intron sequence to be spliced out when it is incorporated into the genome. This architecture is illustrated in FIG. 5B.

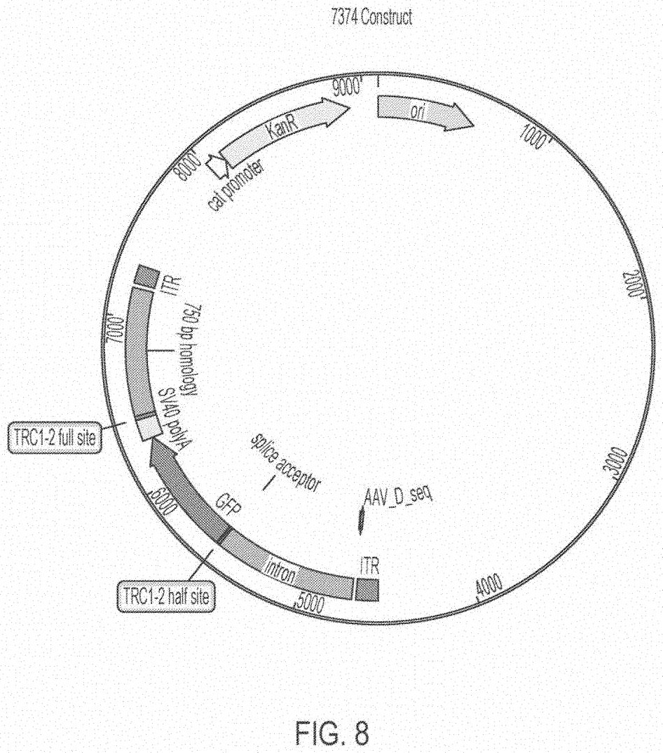

The second AAV vector, referred to as 7374 (FIG. 8; SEQ ID NO: 4), contains a single D sequence, an 826 bp intron having homology to the intron sequence in 7373, a 5′ portion of the TRC 1-2 recognition sequence, a 714 bp coding sequence for GFP ORF with an SV40 polyA, a full TRC 1-2 recognition sequence (shown as two adjacent TRC 1-2 half sites), and a 750 bp homology arm having homology to the right homology arm of 7373. Inclusion of the full TRC 1-2 recognition sequence allows for the option of stacking an additional AAV vector into 7374.

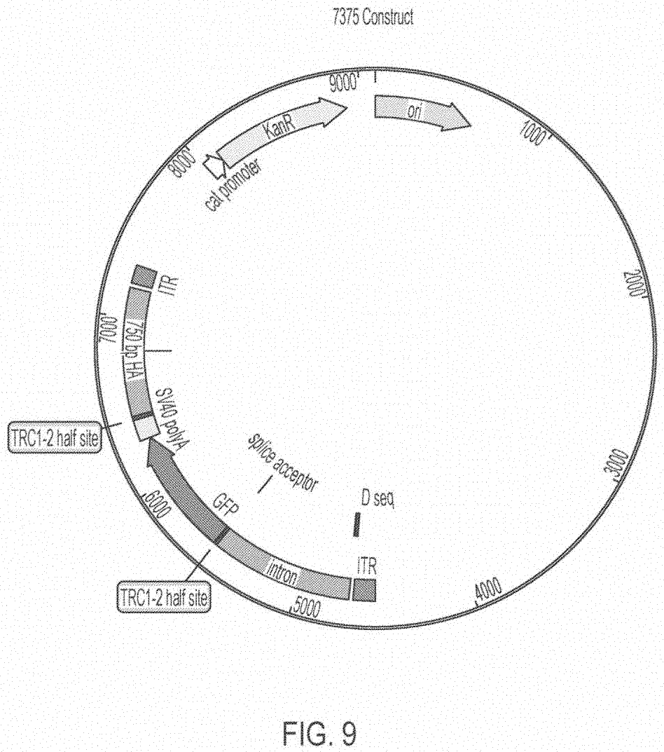

The third AAV vector, referred to as 7375 (FIG. 9; SEQ ID NO: 5), is identical to 7374 but does not comprise a full TRC 1-2 recognition sequence following the GFP ORF and SV40 polyA. Rather, 7375 only comprises the 3′ portion of the TRC 1-2 recognition sequence after the GFP ORF and SV40 polyA to serve as part of the homology arm. By omitting the 5′ portion of the TRC 1-2 recognition sequence, this “locks” the system and does not allow for any further AAVs to be stacked. Also, this prevents the 7375 construct from being cleaved by the TRC 1-2L.1592 nuclease once it has been introduced into the genome.

In principle, the TRC 1-2L.1592 meganuclease should generate a double-stranded break within the TRC 1-2 recognition sequence in the genome, allowing insertion of the 7373 template and additional TRC 1-2 recognition sequence. The CAR can be expressed from this template, but GFP will not be expressed using the EF1-alpha promoter unless the 7374 or 7375 templates are successfully stacked into the 7373 donor sequence.

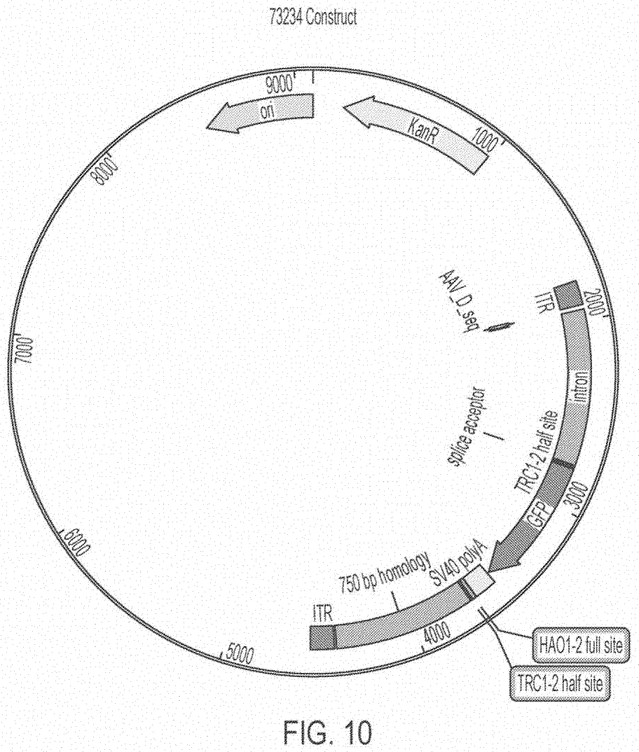

The fourth AAV vector, referred to as 73234 (FIG. 10; SEQ ID NO: 6) comprises the following elements from 5′ to 3′: a 5′ ITR; a single D sequence; an intron sequence containing a 5′ splice donor and 3′ splice acceptor having homology to the intron sequence of construct 7373; the 5′ half-site of the TRC 1-2 recognition sequence; a GFP coding sequence; an SV40 polyA sequence; a full HAO 1-2 recognition sequence; the 3′ half-site of the TRC1-2 recognition sequence; a homology region having 750 bp homology to the sequence 3′ downstream of the endogenous TRC 1-2 recognition sequence and a 3′ ITR.

The fifth AAV vector, referred to as 73235 (FIG. 11; SEQ ID NO: 7) comprises the following elements from 5′ to 3′: a 5′ ITR, a single D sequence, a beta-2M-specific left homology arm, a 5′ portion of a beta-2M recognition sequence, a JeT promoter, a coding sequence for a CD19-specific chimeric antigen receptor, an EF1-alpha promoter, an intron sequence containing a 5′ splice donor and 3′ splice acceptor having homology to the intron sequence of construct 7373, a full TRC 1-2 recognition sequence, a homology region having 287 bp homology to the sequence 3′ downstream of the endogenous TRC 1-2 recognition sequence, the 3′ half-site of the beta-2M recognition sequence, and a 3′ ITR.

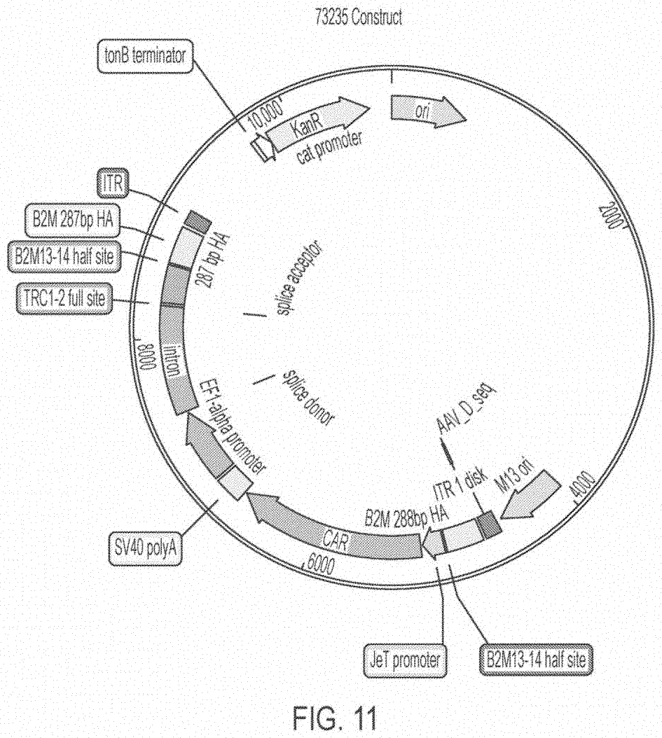

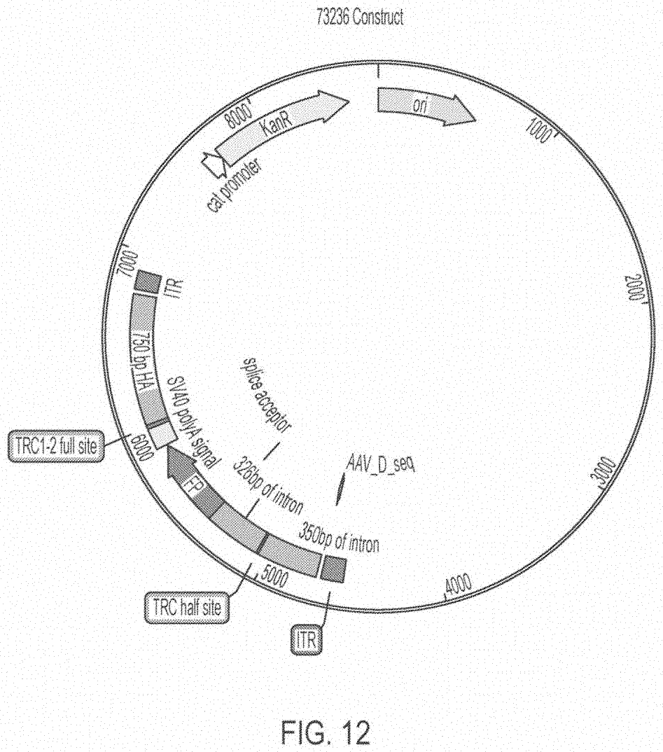

The sixth AAV vector, referred to as construct 73236 (FIG. 12; SEQ ID NO: 8) comprises the following elements from 5′ to 3′: a 5′ ITR; a single D sequence; the 5′ 350 bp an intron sequence having homology to the intron sequence of construct 73237; the 5′ half-site of the TRC 1-2 recognition sequence; the 3′ 326 bp of the intron that contains a splice acceptor; the 3′ 477 bp of the GFP coding sequence; an SV40 polyA sequence; a full TRC 1-2 recognition sequence; a homology region of 750 bp having homology to the sequence 3′ downstream of the endogenous TRC 1-2 recognition sequence and a 3′ ITR. When stacked with construct 73237 the 5′ and 3′ portions of the transgene are separated by an intronic sequence, which is spliced out resulting in expression of a transgene (e.g., GFP) from a promoter. The additional presence of a 3′ full TRC 1-2 recognition sequence allows for additional stacking to occur.

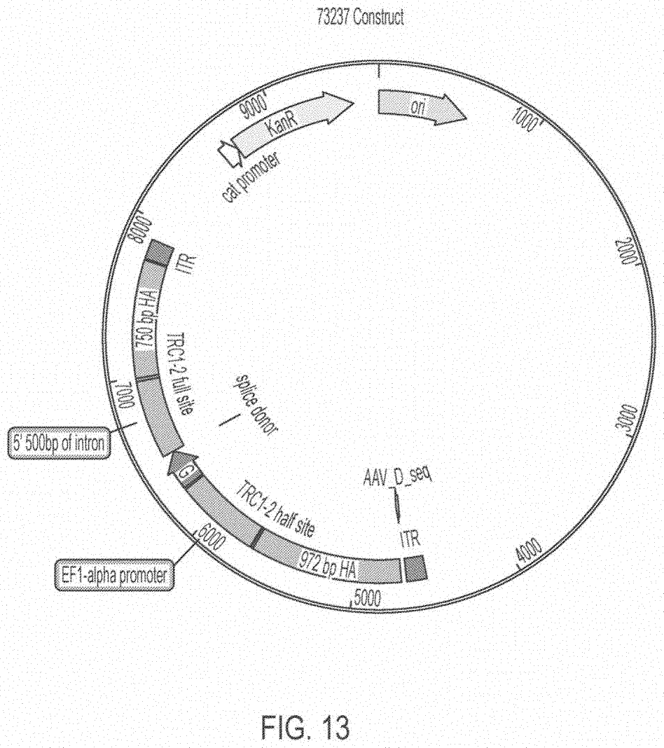

The seventh construct 73237 (FIG. 13; SEQ ID NO: 9) comprises the following elements from 5′ to 3′: a 5′ ITR; a single D sequence; a first homology region of 972 bp having homology to the sequence 5′ upstream of the endogenous TRC 1-2 recognition sequence; the 5′ half-site of the TRC 1-2 recognition sequence; an EF1-alpha promoter; the 5′ 237 bp of the GFP coding sequence; the 5′ 500 bp of the intron containing a 5′ splice donor; a full TRC 1-2 recognition sequence; a second homology region of 750 bp and a 3′ ITR. μ

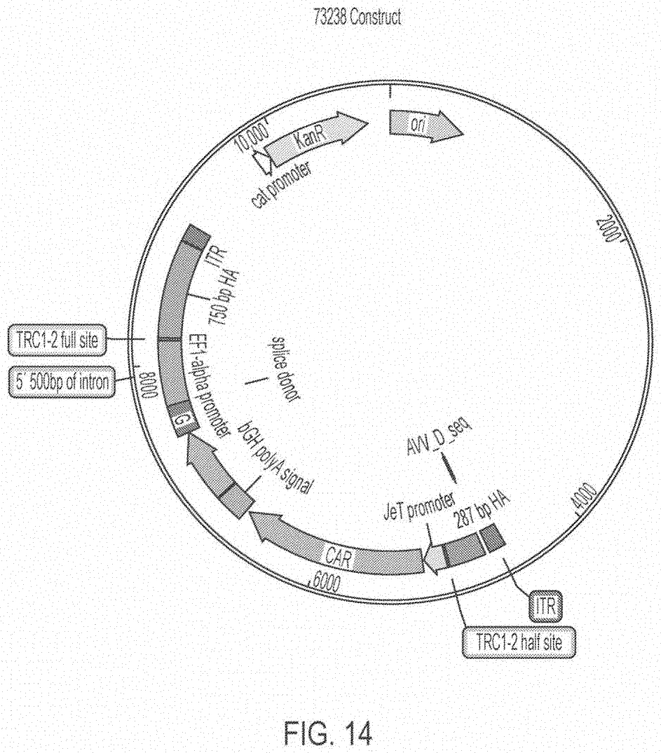

The eight construct 73238 (FIG. 14; SEQ ID NO: 10) comprises the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a bGH polyA sequence; an EF1-alpha promoter; the 5′ 237 bp of the GFP coding sequence; the 5′ 500 bp of the intron containing a splice donor having homology to the intron sequence of construct 73236; a full TRC 1-2 recognition sequence; a second homology region of 750 bp and a 3′ ITR. When stacked with construct 73236 two transgenes can be expressed from two different promoters. The intron within the second transgene is spliced out resulting in its expression (e.g., the first transgene being a CD19 specific CAR and the second transgene being GFP). The additional presence of a 3′ full TRC 1-2 recognition sequence allows for additional stacking to occur.

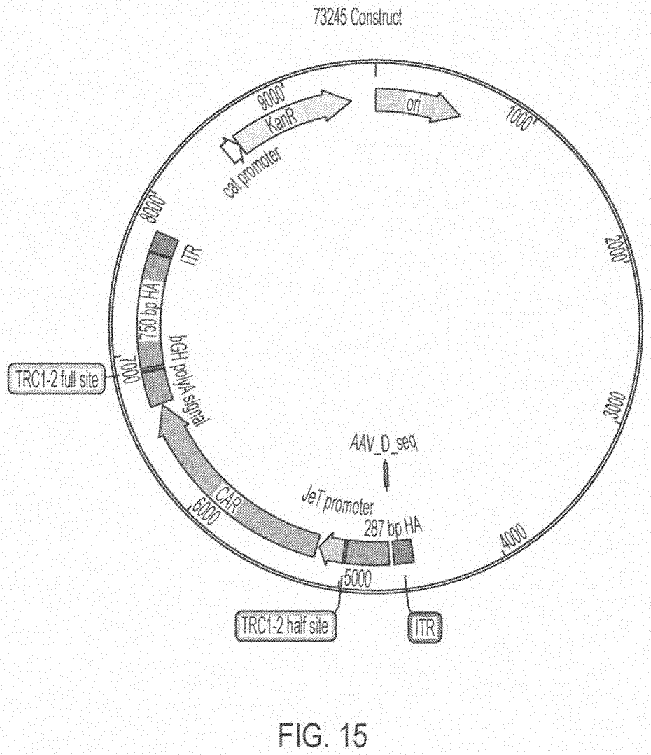

The ninth construct 73245 (FIG. 15; SEQ ID NO: 11) comprises the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a bGH polyA sequence; a full TRC 1-2 recognition sequence; a second homology region of 750 bp; and a 3′ ITR.

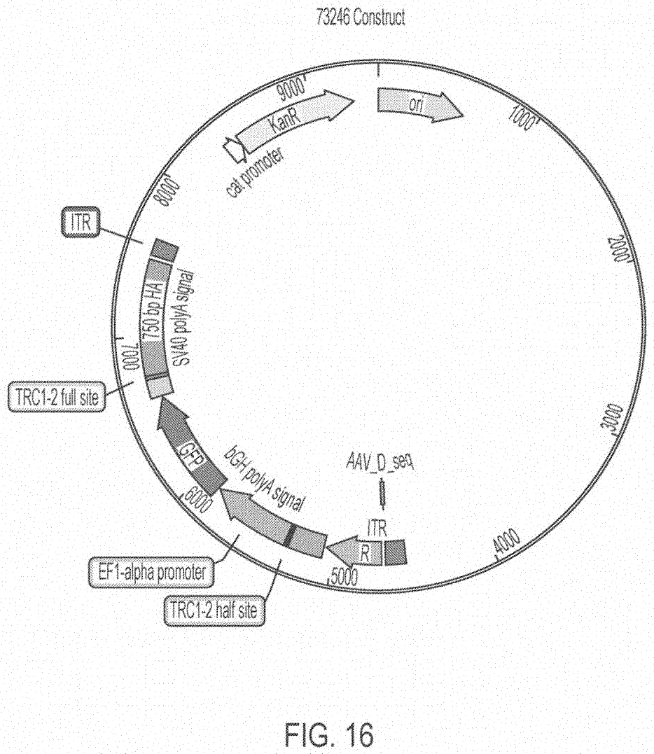

The tenth construct 73246 (FIG. 16; SEQ ID NO: 12) comprises the following elements from 5′ to 3′: a 5′ ITR; a single D sequence; the 3′ 365 bp of the CAR coding sequence having homology to the CAR coding sequence in 73245; a bGH polyA sequence; the 5′ half-site of the TRC 1-2 recognition sequence; an EF1-alpha promoter; a GFP coding sequence; an SV40 polyA sequence; a full TRC 1-2 recognition sequence; a homology region of 750 bp having homology to the sequence 3′ downstream of the endogenous TRC 1-2 recognition sequence and a 3′ ITR. When stacked with construct 73245, two different transgenes can be expressed from two different promoters. Relying on the homology arm of 73246 being homologous to a 3′ portion of the 1^{st} transgene (e.g., a CD19 specific CAR) of construct 73245 eliminates the intronic sequence and splicing. The additional presence of a 3′ full TRC 1-2 recognition sequence allows for additional stacking to occur.

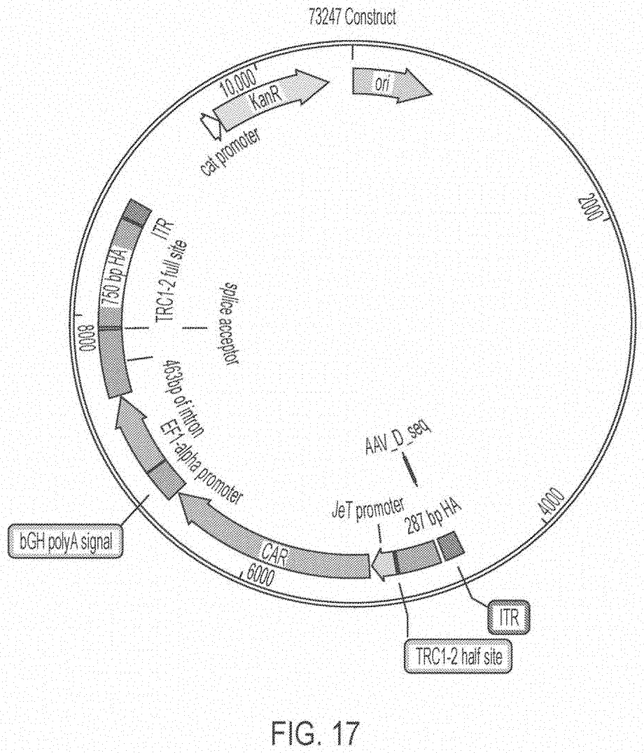

The eleventh construct 73247 (FIG. 17; SEQ ID NO: 13) comprises the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a bGH polyA sequence; an EF1-alpha promoter; 463 bp of an intron sequence containing a splice acceptor and homology to the intron in 7374; a full TRC 1-2 recognition sequence; a second 750 bp homology region and a 3′ ITR. When stacked with construct 7374, two different transgenes can be expressed from two different promoters. The intron between the second promoter and second transgene is spliced out. The additional presence of a 3′ full TRC 1-2 recognition sequence allows for additional stacking to occur.

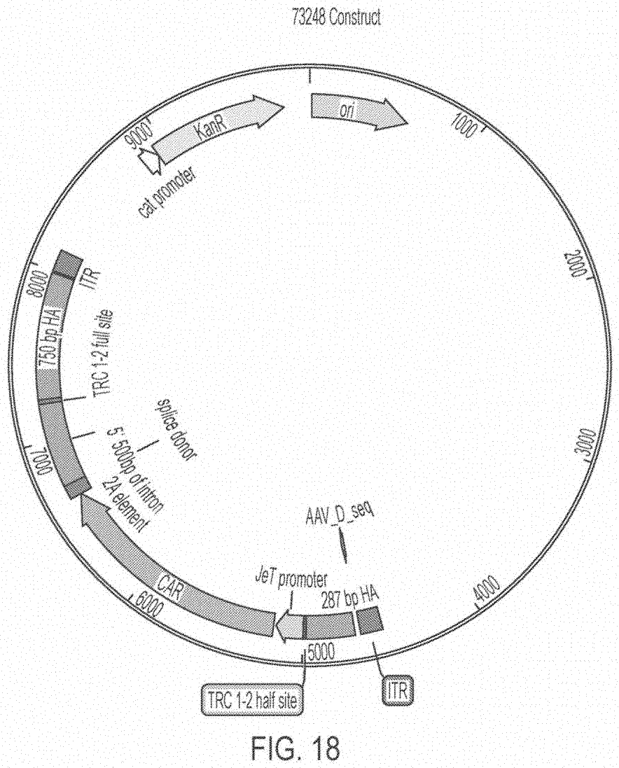

The twelfth construct 73248 (FIG. 18; SEQ ID NO: 14) comprises the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; a 2A element; the 5′ 500 bp of an intron sequence containing a splice donor; a full TRC 1-2 recognition sequence; a 750 bp second homology region; and a 3′ ITR.

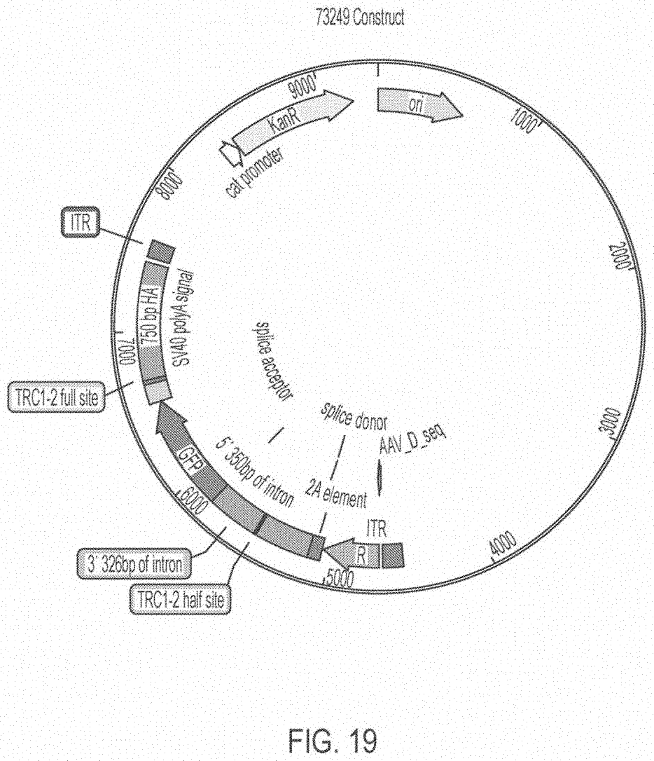

The thirteenth construct 73249 (FIG. 19; SEQ ID NO: 15) comprises the following elements from 5′ to 3′: a 5′ ITR; a single D sequence; the 3′ 365 bp of the CAR coding sequence having homology to the CAR sequence in 73248; a P2A cleavage sequence; the 5′ 350 bp of an intron containing a splice donor; the 5′ half-site of the TRC 1-2 recognition sequence; the 3′ 326 bp of an intron containing a splice donor; a GFP coding sequence; an SV40 polyA sequence; a full TRC 1-2 recognition sequence; a homology region having 750 bp homology to the sequence 3′ downstream of the endogenous TRC 1-2 recognition sequence and a 3′ ITR. When stacked with construct 73248, two different transgenes can be expressed from a single promoters owing to the presence of a P2A cleavage sequence. The intron located 3′ of the P2A sequence is spliced out. Lastly, the additional presence of a 3′ full TRC 1-2 recognition sequence allows for additional stacking to occur.

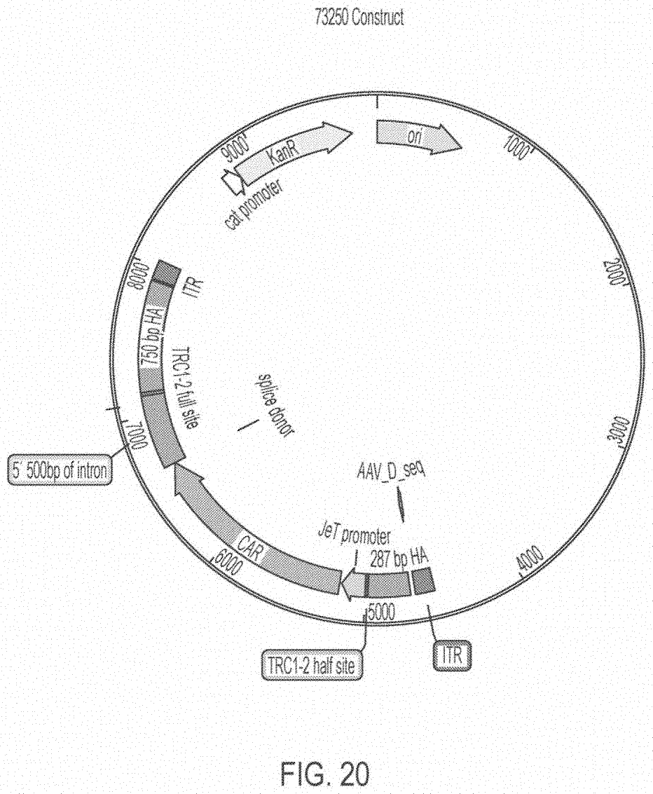

The fourteenth construct 73250 (FIG. 20; SEQ ID NO: 16) comprises the following elements from 5′ to 3′: a 5′ ITR; a single D sequence; a first homology region of 287 bp having homology to the sequence 5′ upstream of the endogenous TRC 1-2 recognition sequence; the 5′ half-site of the TRC 1-2 recognition sequence; a JeT promoter; a CAR coding sequence; the 5′ 500 bp of an intron sequence containing a splice donor; a full TRC 1-2 recognition sequence; a second 750 bp homology region; and a 3′ ITR.

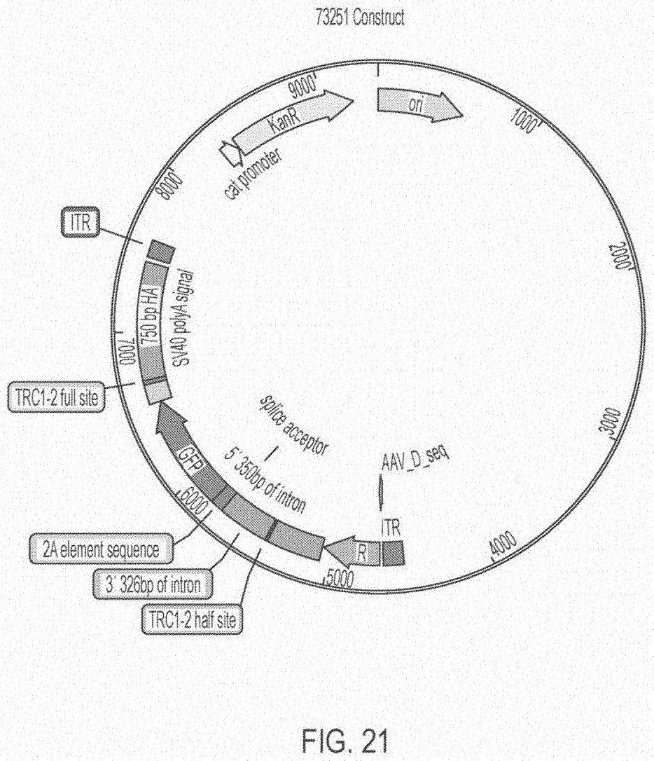

Construct 73251 (FIG. 21; SEQ ID NO: 17) comprises the following elements from 5′ to 3′: a 5′ ITR; a single D sequence; the 3′ 365 bp of a CAR having homology to the CAR sequence in 73250; the 5′ 350 bp of an intron; the 5′ half-site of the TRC 1-2 recognition sequence; the 3′ 326 bp of an intron containing a splice acceptor; a 2A element; a GFP coding sequence; an SV40 polyA sequence; a full TRC 1-2 recognition sequence; a 750 bp homology region having homology to the sequence 3′ downstream of the endogenous TRC 1-2 recognition sequence and a 3′ ITR. When stacked with construct 73248, two different transgenes can be expressed from a single promoter owing to the presence of a P2A cleavage sequence. The intron located 5′ of the P2A sequence is spliced out. Lastly, the additional presence of a 3′ full TRC 1-2 recognition sequence allows for additional stacking to occur.

### Example 2

### Insertion of CAR and GFP Coding Sequences in Primary T Cells by AAV Stacking

These experiments were performed to test the concept of AAV stacking in the context of manufacturing CAR T cells.

### Materials and Methods

In these experiments, CD3+ T cells were isolated and stimulated for 72 hours using Immunocult and human interleukin-2 (IL-2). Post-stimulation, viable cell numbers were enumerated, and cells were prepped for electroporation with mRNA encoding the TRC 1-2L.1592 nuclease by washing in room temperature PBS. After washing, cells were re-suspended in Lonza P3 buffer supplemented with 1 μg of nuclease mRNA per 1e6 viable cells as per the manufacturer's recommendations. Cells were electroporated with the Lonza 4D unit and subsequently rested in the electroporation cuvette for 5 minutes after which serum free cell culture medium containing 30 ng/ml IL-2 was added at a volume four times that of the P3 buffer volume. Samples were then rested for an additional 10 minutes and transferred to a tube containing additional cell culture medium with IL-2.

AAVs were prepared using the 7373, 7374, and 7375 constructs previously described. To calculate the volume of AAV needed for each experimental condition, viable cell numbers post-electroporation were enumerated, and cells were divided into 1×10⁶ viable cell aliquots for each experimental condition. AAV vectors were added to appropriate wells at a multiplicity of infection (MOI) of 2×10⁴ vg/viable cell, with viral vectors 7373 and 7374, or 7373 and 7375, added to separate experimental wells at equal MOIs. Cells electroporated with the TRC 1-2L.1592 TRC.1592 nuclease only and wells transduced individually with each of the three experimental vectors, acted as controls. In a separate subset of wells, electroporated T cells were initially transduced with 7373, followed by addition of either 7374 or 7375 at 22 hours post-electroporation. All experimental conditions were cultured in serum-free culture medium with 30 ng/ml IL-2 for a total of 28 hours, after which culture medium was exchanged for one containing 30 ng/ml IL-2 and 5% fetal bovine serum (FBS). Cells were added to cell culture plates at a concentration of 1×10⁶ viable cells/ml.

To assess the frequency of targeted insertion of both AAV vectors into gene-edited T cells, samples were grown in FBS and IL-2 supplemented medium for 72 hours. Viable cells in each experimental condition were then counted and 2.5×10⁵ viable cells/condition were transferred to a 96-well round bottom plate. Cells were spun down at 1350 RPM for 5 minutes, supernatants were decanted, and samples were subsequently washed with 200 μl of PBS/well. To prepare samples for flow cytometric analysis, cells were stained with 100 μl of PBS/well containing an antibody cocktail of human anti-CD3 PE and anti-FMC63 (CAR) Alexa467. Samples were mixed and allowed to incubate for 15 minutes at room temperature while covered. After the incubation period, samples were spun down and washed as before with room temperature PBS. Samples were re-suspended in 100 μl of PBS/well and run on a BD Cytoflex for data collection.

### Results

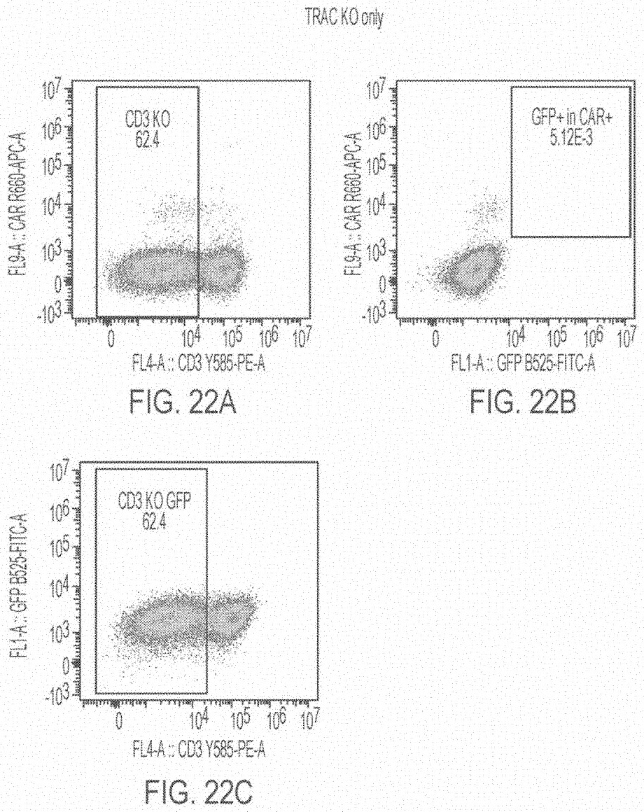

To serve as a control, T cells were electroporated with TRC 1-2L.1592 nuclease only (FIG. 22). To evaluate the efficiency of nuclease editing at the TRAC locus, cells were stained for loss of cell surface CD3 expression. FIG. 10A shows that over 60% of electroporated cells were devoid of CD3 cell surface expression. Importantly, cells treated with only nuclease failed to express the CD19 CAR (FIGS. 22A and 22B) or green fluorescent protein (GFP) (FIGS. 22B and 22C), showing that AAV transduction is required for transgene expression.

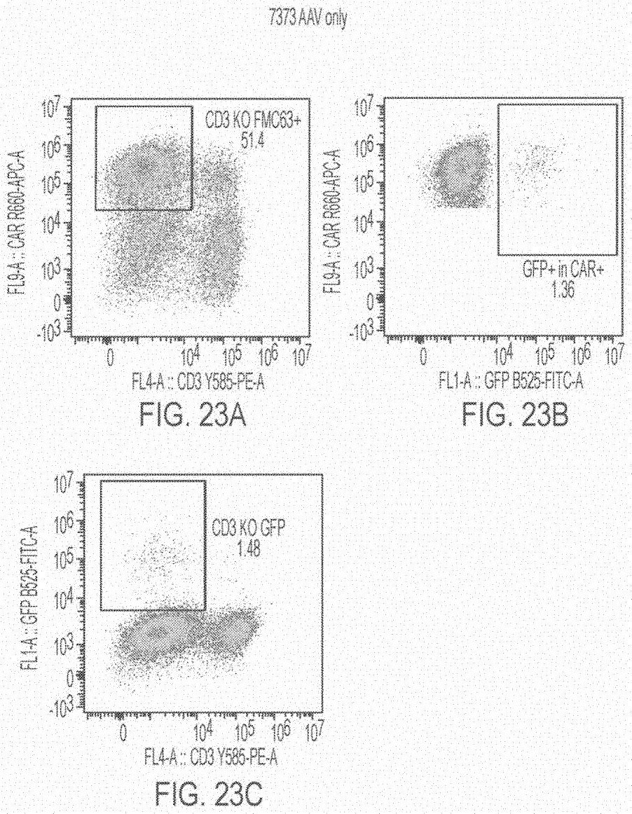

T cells that were electroporated with nuclease and subsequently transduced with AAV vector 7373, whose cargo contains a CD19 CAR coding sequence, driven by a JeT promoter, should allow for incorporation and expression of the CD19 CAR and disruption of the TRAC locus. FIG. 23A shows that over 50% of cells are CD3⁻CAR⁺, demonstrating successful incorporation of the 7373 template DNA into the edited TRAC locus. In addition, when gating on CD3⁻CAR⁺ cells, only background levels of GFP⁺ cells can be seen (FIGS. 23B and 23C).

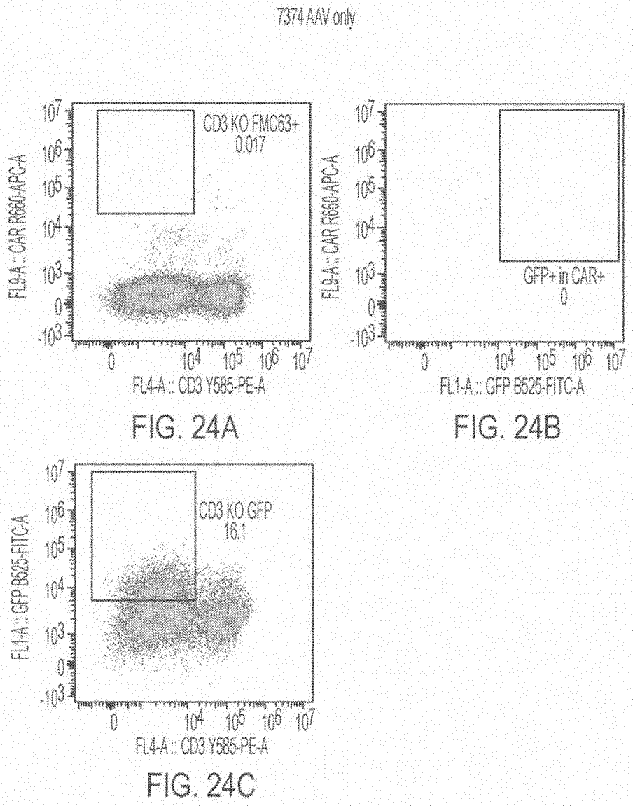

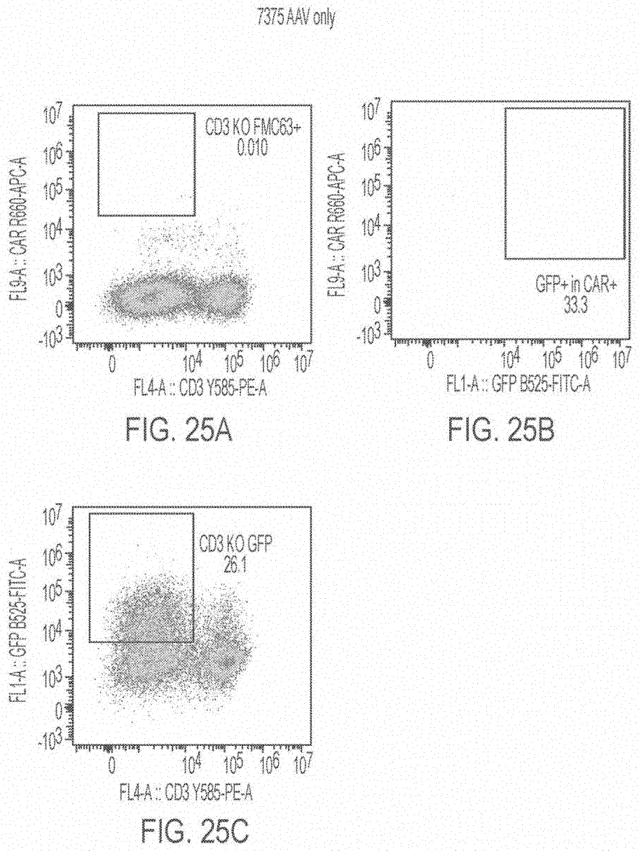

Representative flow cytometric data in T cells transduced with the AAV vector 7374 (FIG. 24) or 7375 (FIG. 25) after electroporation with nuclease mRNA demonstrates that the ability to “stack” the DNA of a second AAV is dependent on the presence of the DNA from a first AAV (e.g., 7373) being incorporated into the edited gene locus. Therefore, genes delivered by the second AAV should not be detected if the first AAV vector is not also transduced. As expected, cells transduced with AAV vector 7374 or 7375 failed to express the CAR (FIG. 24A and FIG. 24A). Interestingly, only a low level of transient GFP expression can be detected in T cells transduced with either of these viral vectors, with a slightly higher frequency of GFP⁺ cells detected in CD3⁻ compared to CD3⁺ T cells in each condition. Of note, GFP expression in T cells from these conditions continued to dissipate over time, supporting the notion that detectable GFP at this time point was not a result of insertion into the edited gene locus.

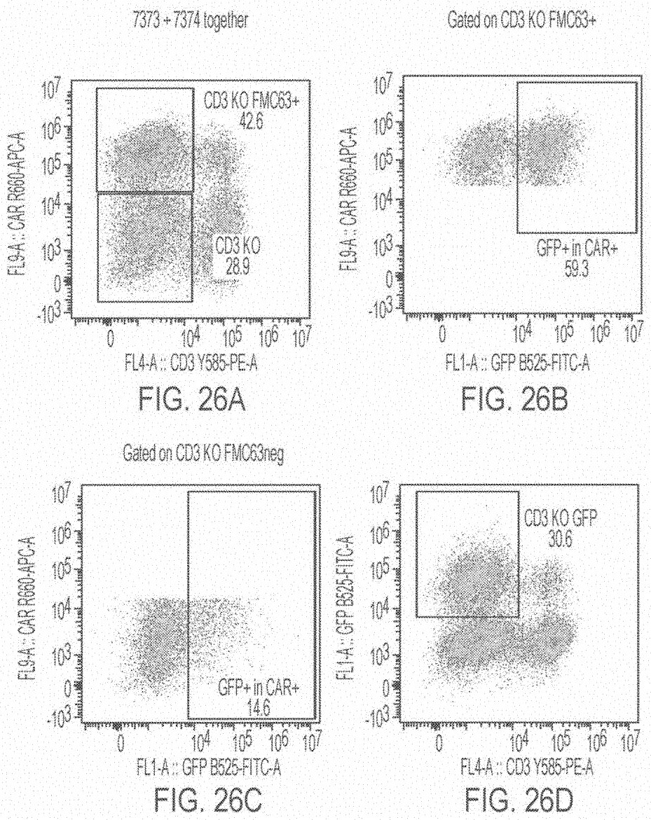

Co-transduction with AAV vectors 7373 and 7374 after electroporation with nuclease mRNA should result in the incorporation and expression of the CD19 CAR, disruption of the TRAC locus, and incorporation and expression of GFP. FIG. 26A shows that over 42% of total T cells are CD3⁻CAR⁺, demonstrating successful integration of DNA delivered by 7373 into the edited gene locus. To determine if the DNA from AAV vector 7374 was also incorporated (i.e., stacked) into the targeted site, CD3⁻CAR⁺ cells were gated on and analyzed for the frequency of detectable GFP⁺ cells. FIG. 26B shows that about 60% of CD3⁻CAR⁺ cells co-expressed GFP in this experimental condition. To determine if GFP expression correlated with CAR expression, the frequency of GFP⁺ cells was also evaluated in CD3⁻CAR⁻ cells (FIGS. 26C and 26D). Importantly, GFP frequency was much lower in this population compared to CAR⁺ cells.

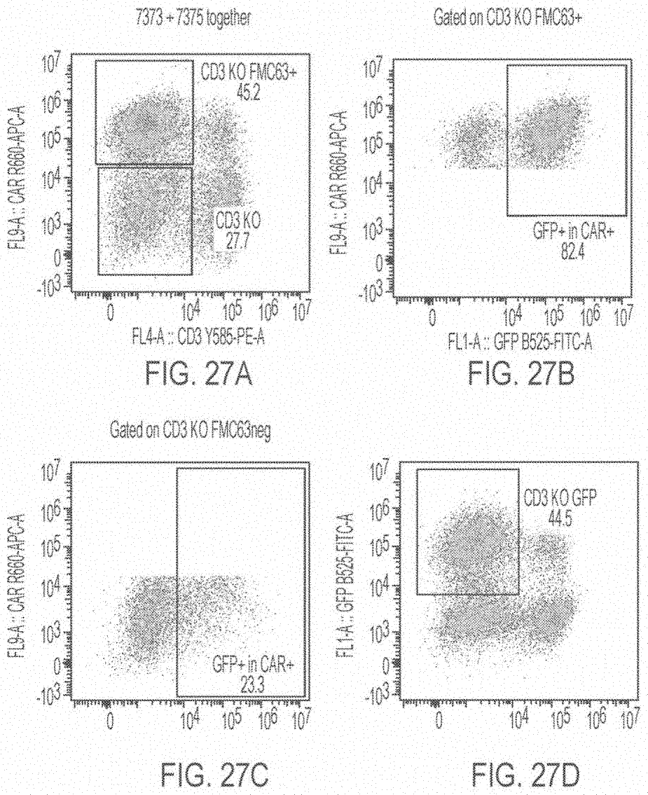

FIG. 27 shows results from T cells transduced with the 7373 and 7375 AAVs, and analyzed for expression of the CAR and co-expression of GFP in edited CAR⁺ cell populations. Notably, the 7375 AAV lacks the full TRC 1-2 recognition sequence that is present in the 7374 AAV vector. Interestingly, co-transduction with 7375 showed a higher frequency of GFP⁺ cells in the CD3⁻CAR⁺ cell population when compared to 7374, with a frequency over about 82% (FIGS. 27A and 27B). Furthermore, as with 7374, GFP delivered by the AAV vector 7375 was dependent on the presence of the CAR (FIGS. 27C and 27D).

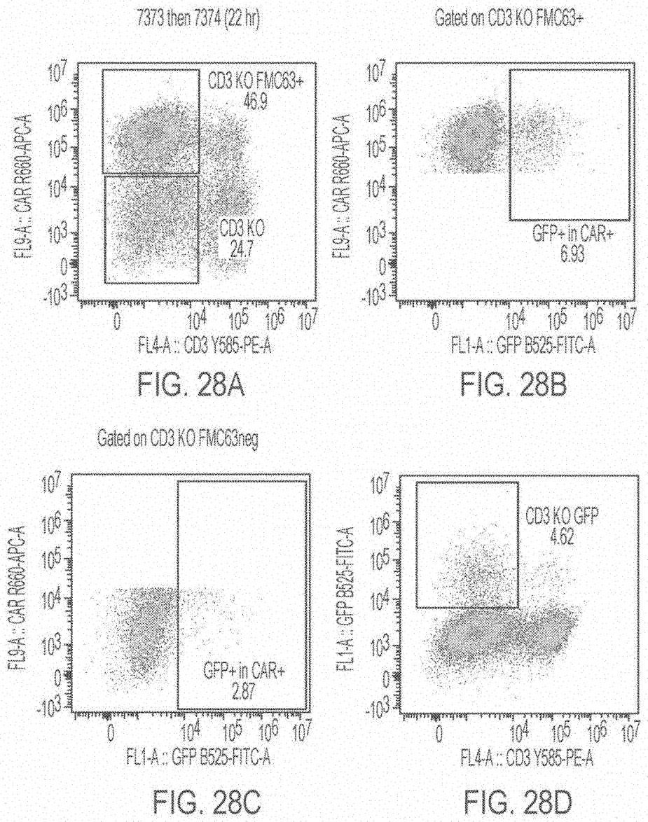

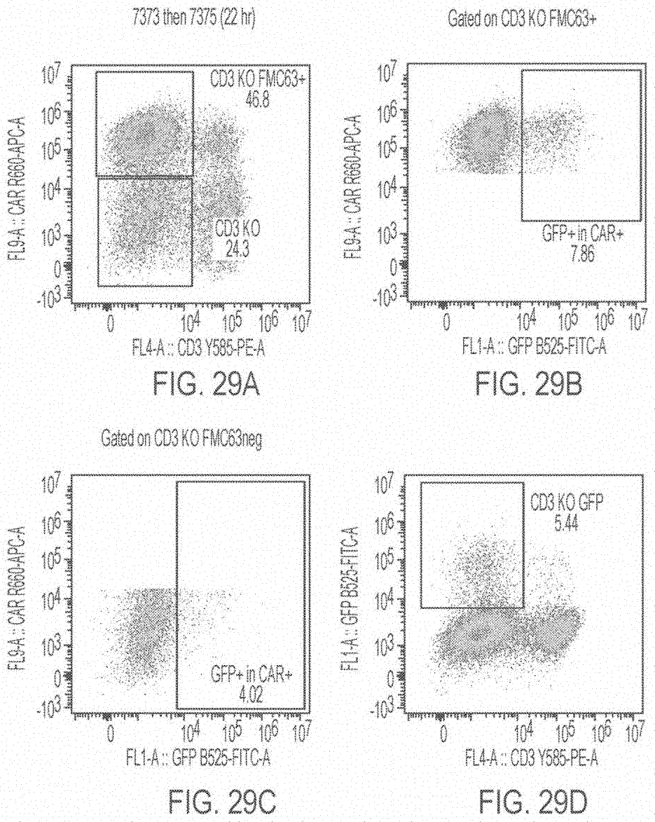

To determine if staggering the transduction of the AAV vectors was superior to co-transduction, T cells were first electroporated and transduced with 7373, followed by addition of 7374 or 7375 22 hours later. In FIG. 28 (7373 and 7374) and FIG. 29 (7373 and 7375), analyzed cells showed a similar frequency of CD3⁻CAR⁺ cells compared to conditions in which AAV vectors were co-administered. Collectively, this shows that the presence of a second AAV does not hinder the function of 7373 at early or late time points (FIGS. 28A and 29A). Of interest, while GFP expression correlated with CAR expression in treated T cells, the overall frequency of GFP⁺ cells was significantly lower compared to conditions where AAV vectors were co-administered (FIGS. 28B and 29B). GFP expression from the 7374 or 7375 AAV vector continued to be dependent on the presence of the CAR (FIGS. 28C and 28D, and FIGS. 29C and 29D).

### Conclusions

The ability to stack DNA delivered by multiple AAV vectors into a targeted site was successful based on the high frequency of GFP⁺ cells specifically in the CD3⁻CAR⁺ cell populations. This result was in stark contrast to the low and transient expression of GFP found in T cells transduced with 7374 or 7375 alone and in the absence of 7373.

Collectively, these results support the hypothesis that the DNA delivered by the first AAV vector, 7373, needs to be present for the DNA from the second AAV vector, either 7374 or 7375, to be stably incorporated. Furthermore, removal of the nuclease recognition sequence in the downstream AAV vector resulted in a higher frequency of GFP⁺CAR⁺ cells, as reflected in the results for cells co-transduced with 7373 and 7375. Presumably, continual nuclease activity on the 3′ nuclease recognition site in 7374 without an adequate downstream repair template present would result in increased DNA damage, thereby lowering the overall frequency of T cells containing stacked DNA.

Lastly, staggering the addition of AAV vectors resulted in significant reductions in CAR⁺GFP⁺ cells compared to T cells that were co-transduced with both viral vectors immediately after electroporation with nuclease. This is likely related to the half-life of the nuclease, with the amount of nuclease decreasing significantly by 24 hours post-electroporation. Overall, these experiments provide proof-of-concept for the AAV stacking methodology and provides a mechanism by which to increase the amount of DNA that can be successfully incorporated into a targeted genomic location during gene editing.

### Example 3

### Insertion of CAR and GFP Coding Sequences in Primary T Cells by AAV Stacking where the Promoter Driving GFP Expression is Provided on the AAV Containing CAR

These experiments were performed to test the concept of AAV stacking in the context of manufacturing CAR T cells.

### Materials and Methods

CD3+ T cells were isolated, stimulated, prepped, electroporated with mRNA encoding the TRC 1-2L.1592 nuclease and resuspended as described in example 2.

AAVs were prepared using the 7373, 7374 and 73234 constructs previously described. To calculate the volume of AAV needed for each experimental condition, viable cell numbers post-electroporation were enumerated, and cells were divided into 1×10⁶ viable cell aliquots for each experimental condition. AAV vectors were added to appropriate wells at a multiplicity of infection (MOI) of 2×10⁴ vg/viable cell, with viral vectors 7373 and 7374, or 7373 and 73234, or added to separate experimental wells at equal MOIs. Cells electroporated with the TRC 1-2L.1592 nuclease only, and wells transduced individually with each of the three experimental vectors, which acted as controls. All experimental conditions were cultured in serum-free culture medium with 30 ng/ml IL-2 for a total of 28 hours, after which culture medium was exchanged for one containing 30 ng/ml IL-2 and 5% fetal bovine serum (FBS). Cells were added to cell culture plates at a concentration of 1×10⁶ viable cells/ml.

To assess the frequency of targeted insertion of both AAV vectors into gene-edited T cells, samples were grown in FBS and IL-2 supplemented medium for 72 hours. Viable cells in each experimental condition were then counted and 2.5×10⁵ viable cells/condition were transferred to a 96-well round bottom plate. Cells were spun down at 1350 RPM for 5 minutes, supernatants were decanted, and samples were subsequently washed with 200 μl of PBS/well. To prepare samples for flow cytometric analysis, cells were stained with 100 μl of PBS/well containing an antibody cocktail of human anti-CD3 PE and anti-FMC63 (CAR) Alexa467. Samples were mixed and allowed to incubate for 15 minutes at room temperature while covered. After the incubation period, samples were spun down and washed as before with room temperature PBS. Samples were re-suspended in 100 μl of PBS/well and run on a BD Cytoflex for data collection.

### Results

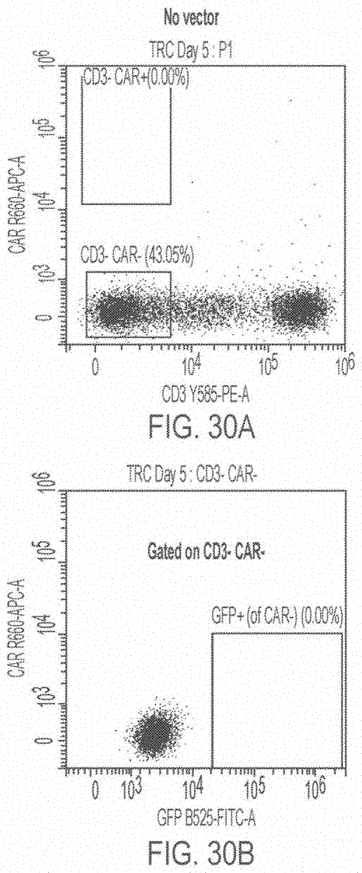

To serve as a control, T cells were electroporated with TRC 1-2L.1592 nuclease only (FIG. 30). To evaluate the efficiency of nuclease editing at the TRAC locus, cells were stained for loss of cell surface CD3 expression. FIG. 30A shows that over 43% of electroporated cells were devoid of CD3 cell surface expression. Importantly, cells treated with only nuclease failed to express the CD19 CAR (FIG. 30A) or green fluorescent protein (GFP) (FIG. 30B), showing that AAV transduction is required for transgene expression.

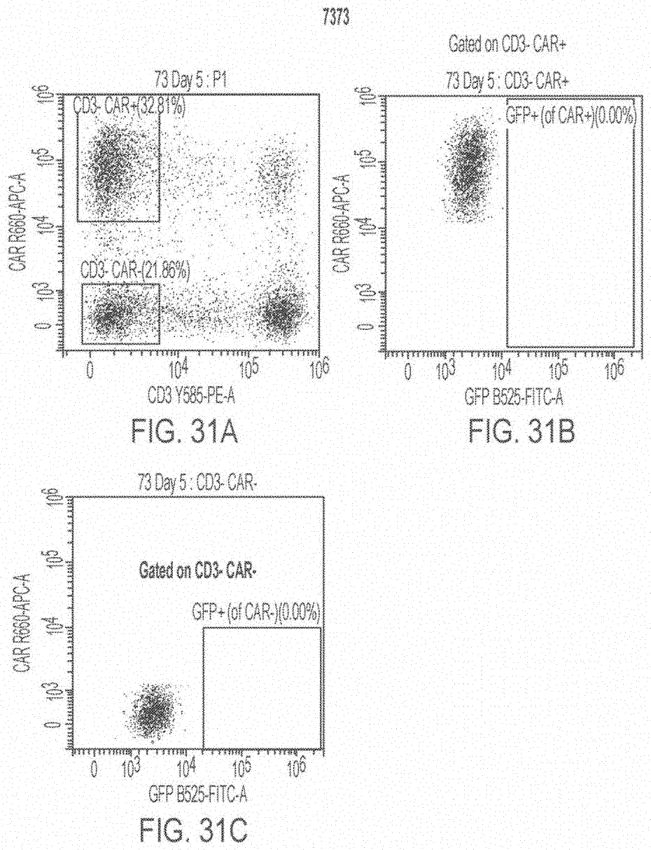

T cells that were electroporated with nuclease and subsequently transduced with each AAV vector, 7373 (FIG. 7), whose cargo contains a CD19 CAR coding sequence, driven by a JeT promoter, should allow for incorporation and expression of the CD19 CAR and disruption of the TRAC locus. FIG. 31 shows that over 32% of cells are CD3⁻CAR⁺, demonstrating successful incorporation of the 7373 template DNA into the edited TRAC locus. In addition, when gating on CD3⁻CAR⁺ cells, GFP⁺ cells were not detected (FIG. 31B).

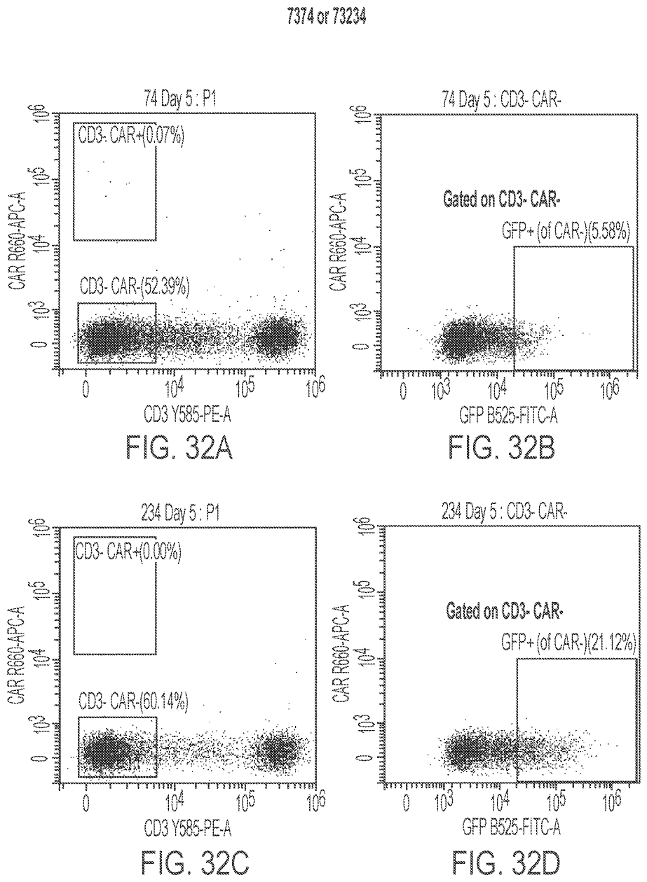

Representative flow cytometric data in T cells transduced with the AAV vector 7374 (FIG. 9) or 73234 (FIG. 10) after electroporation with nuclease mRNA demonstrates that the ability to “stack” the DNA of a second AAV is dependent on the presence of the DNA from a first AAV (e.g., 7373) being incorporated into the edited gene locus. 7374 and 73234, whose cargo contains the GFP coding sequence but lacks the EF1-alpha promoter to drive gene expression should show little to no GFP expression unless the 7373 AAV is first stably incorporated into the genome. Therefore, genes delivered by the second AAV should not be detected if the first AAV vector is not also transduced. As expected, cells transduced with AAV vector 7374 or 73234 failed to express the CAR (FIG. 32A and FIG. 32C). Low levels of leaky GFP expression were detected in T cells transduced with either the 7374 or 73234 AAV vectors (FIG. 32B and FIG. 32D).

Co-transduction with AAV vectors 7373 and 7374 after electroporation with nuclease mRNA should result in the incorporation and expression of the CD19 CAR, disruption of the TRAC locus, and incorporation and expression of GFP.

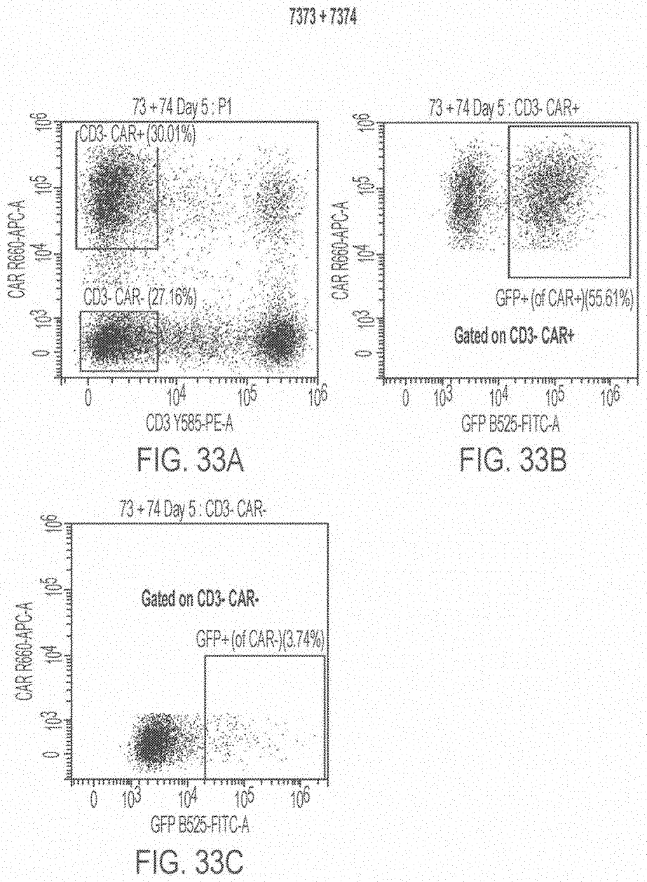

FIG. 33A shows that over 30% of total T cells are CD3⁻CAR⁺, demonstrating successful integration of DNA delivered by 7373 into the edited gene locus. To determine if the DNA from AAV vector 7374 was also incorporated (i.e., stacked) into the targeted site, CD3⁻CAR⁺ cells were gated on and analyzed for the frequency of detectable GFP⁺ cells. FIG. 33B shows that about 55% of CD3⁻CAR⁺ cells co-expressed GFP in this experimental condition. To determine if GFP expression correlated with CAR expression, the frequency of GFP⁺ cells was also evaluated in CD3⁻CAR⁻ cells (FIG. 33C). Importantly, GFP frequency was much lower in this population compared to CAR⁺ cells.

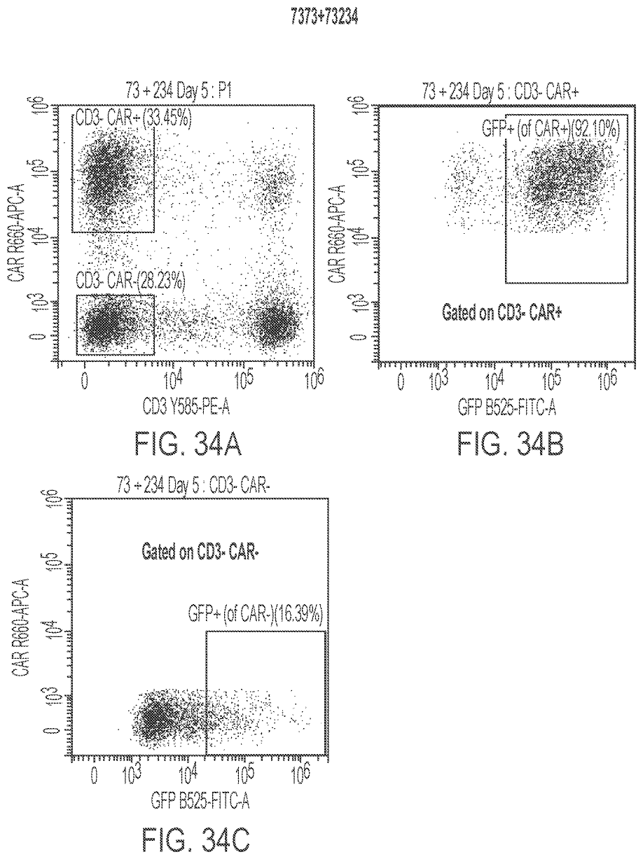

FIG. 34 shows results from T cells transduced with the 7373 and 73234 AAVs, and analyzed for expression of the CAR and co-expression of GFP in edited CAR⁺ cell populations. Notably, the 73234 AAV lacks the full TRC 1-2 recognition sequence that is present in the 7374 AAV vector. Interestingly, co-transduction with 73234 showed a higher frequency of GFP⁺ cells in the CD3⁻CAR⁺ cell population when compared to 7374, with a frequency over about 92% (FIGS. 33B and 34B). Furthermore, as with 7374, much lower levels of GFP expression were observed in cells lacking the 7373 vector containing the CAR (FIGS. 33C and 34C).

### Conclusions

The ability to stack DNA delivered by multiple AAV vectors into a targeted site was successful based on the high frequency of GFP⁺ cells specifically in the CD3⁻CAR⁺ cell populations. This result was in stark contrast to the low and transient expression of GFP found in T cells transduced with 7374 or 73234 alone and in the absence of 7373.

Collectively, these results support the hypothesis that the DNA delivered by the first AAV vector, 7373, needs to be present to drive GFP expression from the DNA from the second AAV vector. This is because both 7374 and 73234 lack the promoter present on 7373 that is necessary to drive GFP expression. Furthermore, removal of the nuclease recognition sequence in the downstream AAV vector resulted in a higher frequency of GFP⁺CAR⁺ cells, as reflected in the results for cells co-transduced with 7373 and 73234. Presumably, continual nuclease activity on the 3′ nuclease recognition site in 7374 without an adequate downstream repair template present may result in increased DNA damage, thereby lowering the overall frequency of T cells containing stacked DNA.

### Example 4

### Insertion of GFP Coding Sequences in Primary T Cells by AAV Stacking where the 5′ Portion of the GFP Gene is Provided on One AAV and the 3′ Portion is Provided on the Second AAV, Both Separated by an Internal Intron

### Materials and Methods

CD3+ T cells were isolated, stimulated, prepped, electroporated with mRNA encoding the TRC 1-2L.1592 nuclease and resuspended as described in example 2.

AAVs were prepared using the 73237 and 73236 constructs previously described. The volume of AAV needed for each experimental condition was calculated as described previously. AAV vectors were added to appropriate wells at a multiplicity of infection (MOI) of 2×10⁴ vg/viable cell, with viral vectors 73237 and 73236, and added to separate experimental wells at equal MOIs. Cells electroporated with the TRC 1-2L.1592 nuclease only, and wells transduced individually with each of the two experimental vectors, acted as controls. All experimental conditions were cultured as previously described in example 2.

To assess the frequency of targeted insertion of both AAV vectors into gene-edited T cells, samples were grown in FBS and IL-2 supplemented medium for 72 hours. Viable cells in each experimental condition were then counted, pelleted, resuspended, stained with an antibody cocktail of human anti-CD3 PE and anti-FMC63 (CAR) Alexa467 and subjected to FLOW cytometry as described in example 2.

### Results

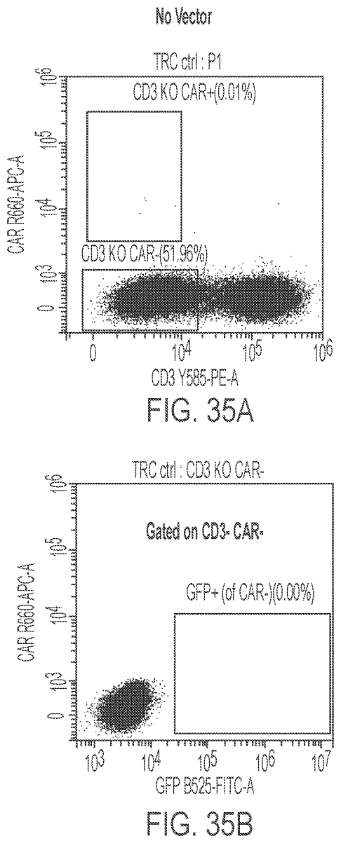

To serve as a control, T cells were electroporated with TRC 1-2L.1592 nuclease only (FIG. 35). To evaluate the efficiency of nuclease editing at the TRAC locus, cells were stained for loss of cell surface CD3 expression. FIG. 35A shows that over 51% of electroporated cells were devoid of CD3 cell surface expression. Importantly, cells treated with only nuclease failed to express green fluorescent protein (GFP) (FIG. 35B), showing that AAV transduction is required for GFP transgene expression.

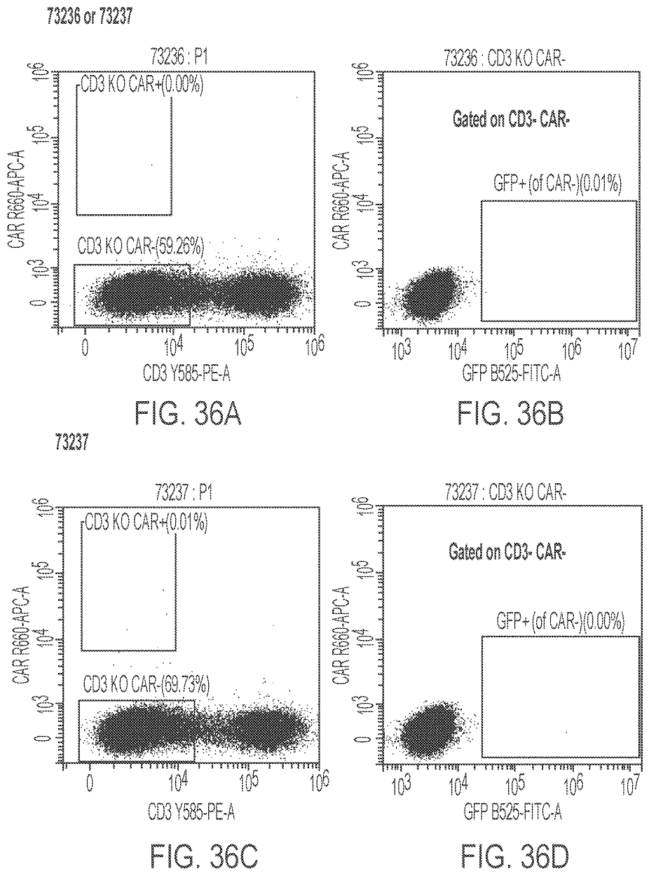

T cells that were electroporated with nuclease and subsequently transduced with AAV vector 73237, whose cargo contains an EF1-alpha promoter, 237 bp of the 5′ GFP coding sequence, followed by part of the intron containing a splice donor, results in disruption of the TRAC locus, similarly to results seen with nuclease alone (FIG. 35A and FIG. 36C)

Representative flow cytometric data in T cells transduced with the AAV vector 73237 (FIG. 13) or 73236 (FIG. 12) after electroporation with nuclease mRNA demonstrates the necessity to “stack” the DNA of a second AAV into the edited gene locus to obtain GFP expression. In this example, both AAVs must be incorporated into the TRAC locus, supplying two portions of GFP, separated by a full intron with a splice donor and a splice acceptor. Successful transcriptional splicing after insertion of both AAVs is required for expression of the GFP. As expected, cells transduced with AAV vector 73237 or 73236 alone failed to express the GFP (FIG. 36B and FIG. 36D).

Co-transduction with AAV vectors 73237 and 73236 after electroporation with nuclease mRNA should result in the disruption of the TRAC locus, and incorporation and expression of GFP.

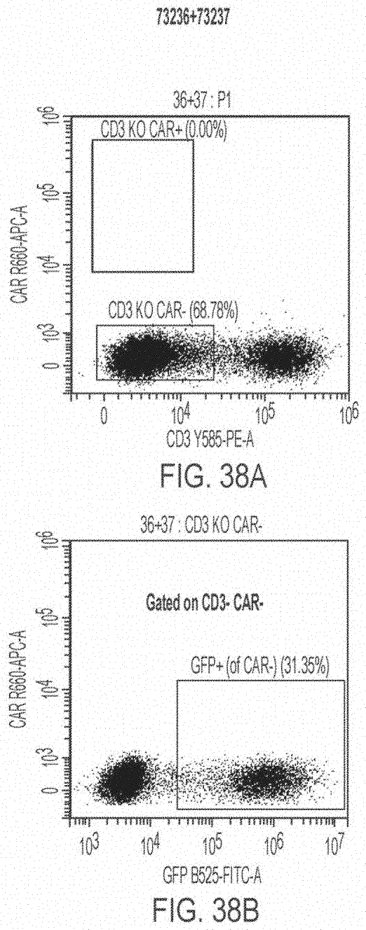

FIG. 38A shows that over 68% of total T cells are CD3⁻, demonstrating nuclease editing or integration of DNA delivered by 73237 into the edited TRAC locus. To determine if the DNA from AAV vector 73236 was also incorporated (i.e., stacked) into the targeted site, CD3⁻GFP⁺ cells were gated on and analyzed for the frequency of detectable GFP⁺ cells. FIG. 38B shows that 31.35% of CD3⁻ cells are GFP⁺. Expression of a CAR construct would not be expected since neither of the 73236 or 73237 vectors contain a CAR expression construct. CAR staining was done to be consistent with other experiments described herein.

### Conclusions

The ability to stack DNA delivered by multiple AAV vectors into a targeted site was successful based on the frequency of GFP⁺ cells specifically in the CD3⁻ cell populations. This result contrasts the lack of expression of GFP found in T cells transduced with 73237 or 73236 alone.

These results support the hypothesis that the DNA delivered by both AAV vectors must to be present and stably incorporated for expression of the full-length transgene. In addition, by separating two parts of the transgene on two independent AAVs, any leaky expression of GFP from the transgene being located on a single AAV was eliminated.

### Example 5

### Insertion of CAR and GFP Coding Sequences in Primary T Cells by AAV Stacking where the CAR is Provided on the First AAV and GFP is Provided on the Second AAV

### Materials and Methods

CD3+ T cells were isolated, stimulated, prepped, electroporated with mRNA encoding the TRC 1-2L.1592 nuclease and resuspended as described in example 2.

AAVs were prepared using the 73238 and 73236 constructs previously described. The volume of AAV needed for each experimental condition was calculated as described previously. AAV vectors were added to appropriate wells at a multiplicity of infection (MOI) of 2×10⁴ vg/viable cell, with viral vectors 73238 and 73236, and added to separate experimental wells at equal MOIs. Cells electroporated with the TRC 1-2L.1592 nuclease only, and wells transduced individually with each of the two experimental vectors, acted as controls. All experimental conditions were cultured as previously described in example 2.

To assess the frequency of targeted insertion of both AAV vectors into gene-edited T cells, samples were grown in FBS and IL-2 supplemented medium for 72 hours. Viable cells in each experimental condition were then counted, pelleted, resuspended, stained with an antibody cocktail of human anti-CD3 PE and anti-FMC63 (CAR) Alexa467 and subjected to FLOW cytometry as described in example 2.

### Results

To serve as a control, T cells were electroporated with TRC 1-2L.1592 nuclease only (FIG. 35). To evaluate the efficiency of nuclease editing at the TRAC locus, cells were stained for loss of cell surface CD3 expression. FIG. 35A shows that over 51% of electroporated cells were devoid of CD3 cell surface expression. Importantly, cells treated with only nuclease failed to express green fluorescent protein (GFP) (FIG. 35B), showing that AAV transduction is required for GFP transgene expression.

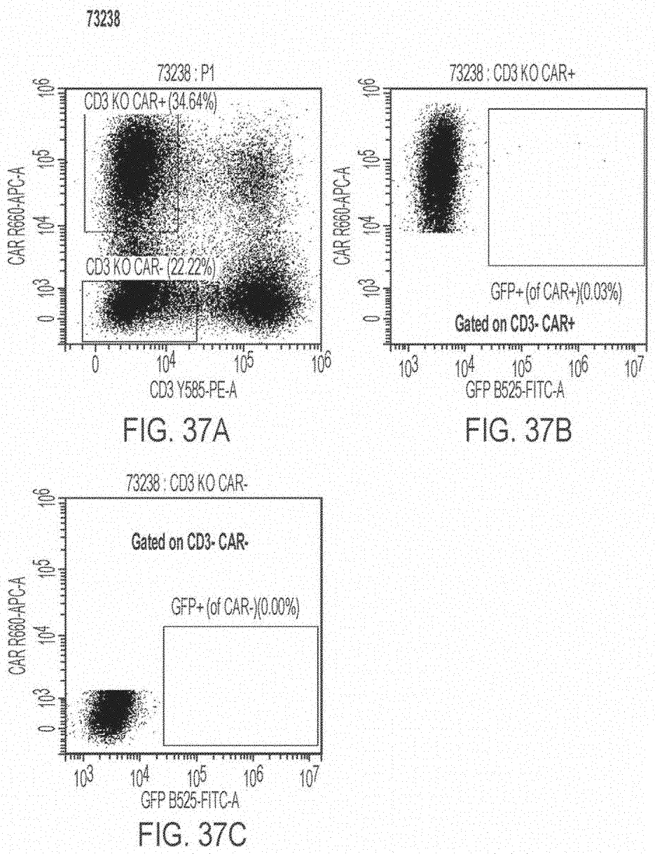

T cells that were electroporated with nuclease and subsequently transduced with AAV vector 73238, whose cargo contains a CD19 CAR coding sequence, driven by a JeT promoter, and the 5′ 237 bp of GFP sequence driven by an EF1-alpha promoter followed by part of an intron containing a splice donor which should allow for incorporation and expression of the CD19 CAR and disruption of the TRAC locus. FIG. 37A shows that over 34% of cells are CD3⁻CAR⁺, demonstrating successful incorporation of the 73238 template DNA into the edited TRAC locus. In addition, when gating on CD3⁻CAR⁺ cells, only background levels of GFP cells can be seen (FIG. 37B).

Representative flow cytometric data in T cells transduced with the AAV vector 73238 (FIG. 14) or 73236 (FIG. 12) after electroporation with nuclease mRNA demonstrates the necessity to “stack” the DNA of a second AAV into the edited gene locus to obtain GFP expression. The 73238 AAV supplies the full CAR coding sequence and one portion of the GFP, while the 73236 supplies only the second portion of the GFP. Accordingly, in this example, both AAVs must be incorporated into the TRAC locus, supplying two portions of GFP, separated by a full intron with a splice donor and a splice acceptor. Successful transcriptional splicing after insertion of both AAVs is required for expression of the GFP. As expected, cells transduced with AAV vector 73238 or 73236 alone failed to express the GFP (FIG. 37B and FIG. 36B).

Co-transduction with AAV vectors 73238 and 73236 after electroporation with nuclease mRNA should result in the incorporation and expression of the CD19 CAR, disruption of the TRAC locus, and incorporation and expression of GFP.

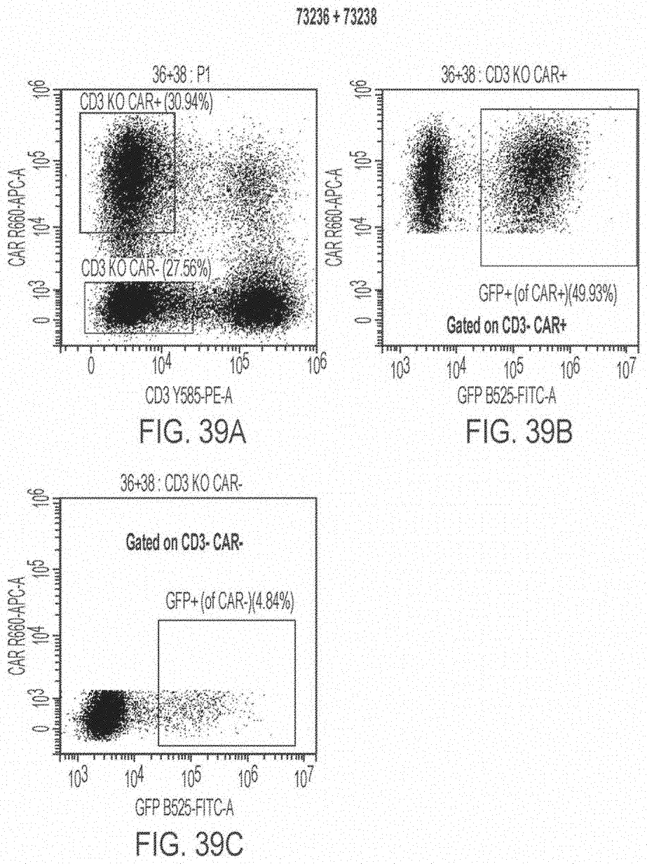

FIG. 39 shows that over 30% of total T cells are CD3⁻CAR⁺, demonstrating successful integration of DNA delivered by 73238 into the edited gene locus. To determine if the DNA from AAV vector 73236 was also incorporated (i.e., stacked) into the targeted site, CD3⁻CAR⁺ cells were gated on and analyzed for the frequency of detectable GFP⁺ cells. FIG. 39B shows that about 50% of CD3⁻CAR⁺ cells co-expressed GFP in this experimental condition. To determine if GFP expression correlated with CAR expression, the frequency of GFP⁺ cells was also evaluated in CD3⁻CAR⁻ cells (FIG. 39C). Importantly, GFP frequency was much lower in this population compared to CAR⁺ cells.

### Conclusions

The ability to stack DNA delivered by multiple AAV vectors into a targeted site was successful based on the high frequency of GFP⁺ cells specifically in the CD3⁻CAR⁺ cell populations. In this example, the 3′ 326 bp of the intron containing a splice acceptor and the 3′ 477 bp of the GFP coding sequence are supplied by the second AAV, 73236. Stable integration of both AAVs, 73238 and 73236, and successful transcriptional splicing is required for expression of GFP.

Collectively, these results support the hypothesis that the DNA delivered by the first AAV vector, 73238, needs to be present for the DNA from the second AAV vector, or 73236, to be stably incorporated and result in GFP expression. Consistent with example 4, separating two parts of the second transgene on two independent AAVs with an intron prevented any leaky expression of GFP.

### Example 6

### Insertion of CAR and GFP Coding Sequences in Primary T Cells by AAV Stacking where the Promoter Driving GFP Expression is Provided on the AAV Containing CAR

### Materials and Methods

CD3+ T cells were isolated, stimulated, prepped, electroporated with mRNA encoding the TRC 1-2L.1592 nuclease and resuspended as described in example 2.

AAVs were prepared using the 73245 and 73246 constructs previously described. The volume of AAV needed for each experimental condition was calculated as described previously. AAV vectors were added to appropriate wells at a multiplicity of infection (MOI) of 2×10⁴ vg/viable cell, with viral vectors 73245 and 73246, and added to separate experimental wells at equal MOIs. Cells electroporated with the TRC 1-2L.1592 nuclease only, and wells transduced individually with each of the two experimental vectors, acted as controls. All experimental conditions were cultured as previously described in example 2.

To assess the frequency of targeted insertion of both AAV vectors into gene-edited T cells, samples were grown in FBS and IL-2 supplemented medium for 72 hours. Viable cells in each experimental condition were then counted, pelleted, resuspended, stained with an antibody cocktail of human anti-CD3 PE and anti-FMC63 (CAR) Alexa467 and subjected to FLOW cytometry as described in example 2.

### Results

To serve as a control, T cells were electroporated with TRC 1-2L.1592 nuclease only (FIG. 35). To evaluate the efficiency of nuclease editing at the TRAC locus, cells were stained for loss of cell surface CD3 expression. FIG. 35A shows that over 51% of electroporated cells were devoid of CD3 cell surface expression. Importantly, cells treated with only nuclease failed to express green fluorescent protein (GFP) (FIG. 35B), showing that AAV transduction is required for GFP transgene expression.

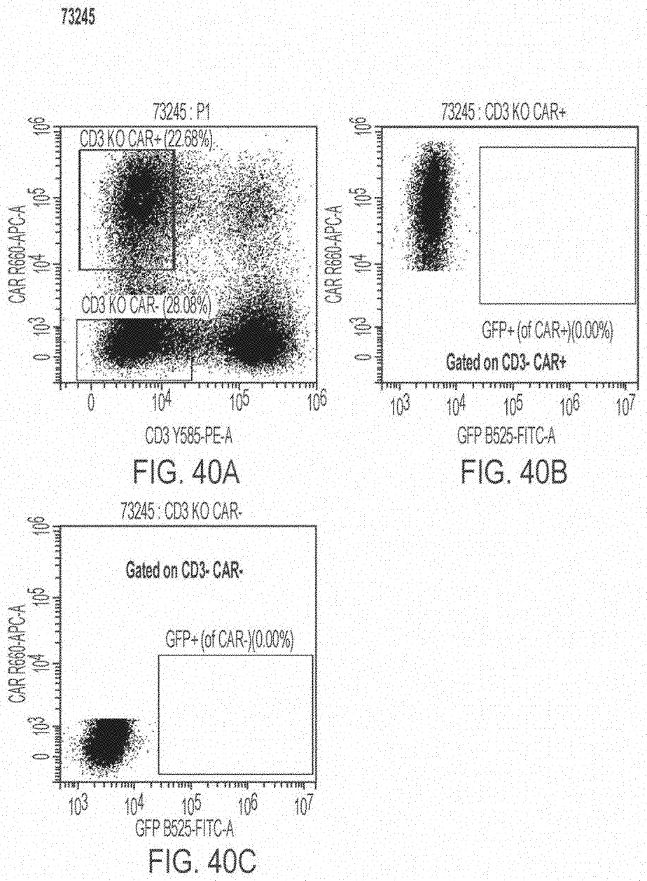

T cells that were electroporated with nuclease and subsequently transduced with AAV vector, 73245, whose cargo contains a CD19 CAR coding sequence, driven by a JeT promoter, should allow for incorporation and expression of the CD19 CAR and disruption of the TRAC locus. FIG. 40A shows that over 22% of cells are CD3⁻CAR⁺, demonstrating successful incorporation of the 73245 template DNA into the edited TRAC locus. In addition, when gating on CD3⁻CAR⁺ cells, no GFP⁺ cells can be seen (FIG. 40B).

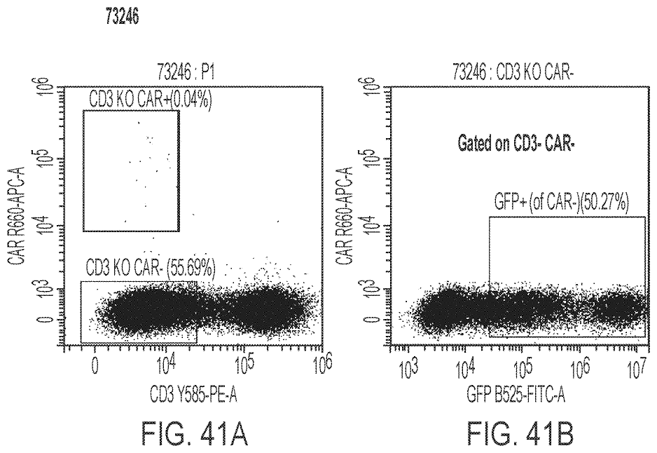

Representative flow cytometric data in T cells transduced with the AAV vector 73245 (FIG. 15) or 73246 (FIG. 16) after electroporation with nuclease mRNA is provided in FIG. 40 and FIG. 41, respectively. Each of these AAVs are capable of expressing their transgenes without elements provided by the stacking of the AAVs. 73246, whose cargo contains the GFP coding sequence driven by the EF1-alpha promoter, shows GFP expression roughly equivalent to the percentage of CAR⁻ cells (FIG. 41). As expected, because there is no CAR coding sequence present, cells transduced with AAV vector 73246 failed to express the CAR (FIG. 41A).

Co-transduction with AAV vectors 73245 and 73246 after electroporation with nuclease mRNA should result in the incorporation and expression of the CD19 CAR, disruption of the TRAC locus, and incorporation and expression of GFP.

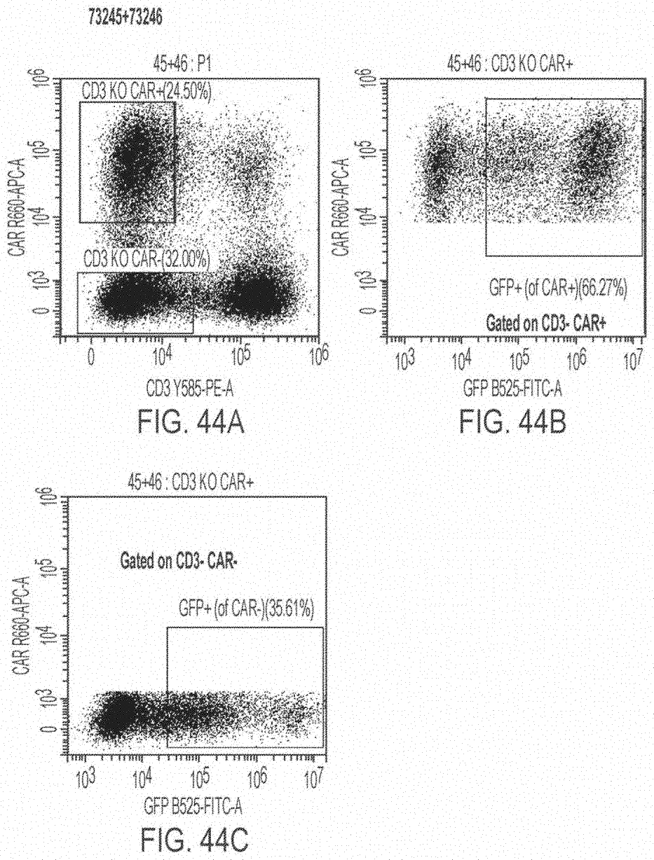

FIG. 44 shows that over 24% of total T cells are CD3⁻CAR⁺, demonstrating successful integration of DNA delivered by 73245 into the edited gene locus. To determine if these cells are also GFP+, CD3⁻CAR⁺ cells were gated on and analyzed for the frequency of detectable GFP cells. Because 73246 contains all the necessary elements for GFP expression, it is not possible, to definitively determine by flow cytometry in the absence of genetic sequencing if the GFP expression in the CD3⁻CAR⁺ cells is due to incorporation of 73246 into the edited gene locus or merely because of every edited cell containing an AAV expressing GFP. FIG. 44B shows that about 66% of CD3⁻CAR⁺ cells co-expressed GFP in this experimental condition. To determine if GFP expression correlated with CAR expression, the frequency of GFP⁺ cells was also evaluated in CD3⁻CAR⁻ cells (FIG. 44C). Importantly, GFP frequency was approximately half in this population compared to CAR⁺ cells suggesting that it is likely some percentage of the CAR⁺GFP⁺ is due to stacking.

### Conclusions

In this example where the expression of the transgene from either ‘stacking” AAV is not contingent on incorporation of both AAVs into the edited gene locus, stacking of each AAV individually is speculative without DNA sequence to confirm insertion.

Collectively, these results support the hypothesis that the DNA delivered by the first AAV vector, 73245, needs to be present for the DNA from the second AAV vector, 73246, to be stacked and stably incorporated.

### Example 7

### Insertion of CAR and GFP Coding Sequences in Primary T Cells by AAV Stacking where the Promoter Driving GFP Expression Followed by an Intron is Provided on the AAV Containing CAR

### Materials and Methods

CD3+ T cells were isolated, stimulated, prepped, electroporated with mRNA encoding the TRC 1-2L.1592 nuclease and resuspended as described in example 2.

AAVs were prepared using the 73247 and 7374 constructs previously described. The volume of AAV needed for each experimental condition was calculated as described previously. AAV vectors were added to appropriate wells at a multiplicity of infection (MOI) of 2×10⁴ vg/viable cell, with viral vectors 73247 and 7374, and added to separate experimental wells at equal MOIs. Cells electroporated with the TRC 1-2 L.1592 nuclease only, and wells transduced individually with each of the two experimental vectors, acted as controls. All experimental conditions were cultured as previously described in example 2.

To assess the frequency of targeted insertion of both AAV vectors into gene-edited T cells, samples were grown in FBS and IL-2 supplemented medium for 72 hours. Viable cells in each experimental condition were then counted, pelleted, resuspended, stained with an antibody cocktail of human anti-CD3 PE and anti-FMC63 (CAR) Alexa467 and subjected to FLOW cytometry as described in example 2.

### Results

To serve as a control, T cells were electroporated with TRC 1-2L.1592 nuclease only (FIG. 35). To evaluate the efficiency of nuclease editing at the TRAC locus, cells were stained for loss of cell surface CD3 expression. FIG. 35A shows that over 51% of electroporated cells were devoid of CD3 cell surface expression. Importantly, cells treated with only nuclease failed to express green fluorescent protein (GFP) (FIG. 35B), showing that AAV transduction is required for GFP transgene expression.

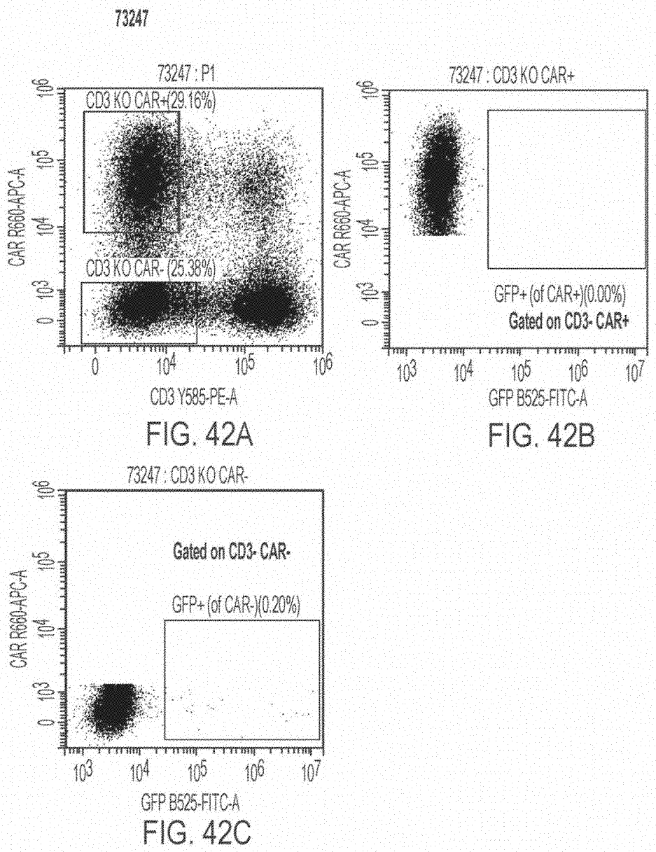

T cells that were electroporated with nuclease and subsequently transduced with each AAV vector, 73247, whose cargo contains a CD19 CAR coding sequence, driven by a JeT promoter, should allow for incorporation and expression of the CD19 CAR and disruption of the TRAC locus. FIG. 42A shows that over 29% of cells are CD3⁻CAR⁺, demonstrating successful incorporation of the 73247 template DNA into the edited TRAC locus. In addition, when gating on CD3⁻CAR⁺ cells, GFP⁺ cells were not detected (FIG. 42B).

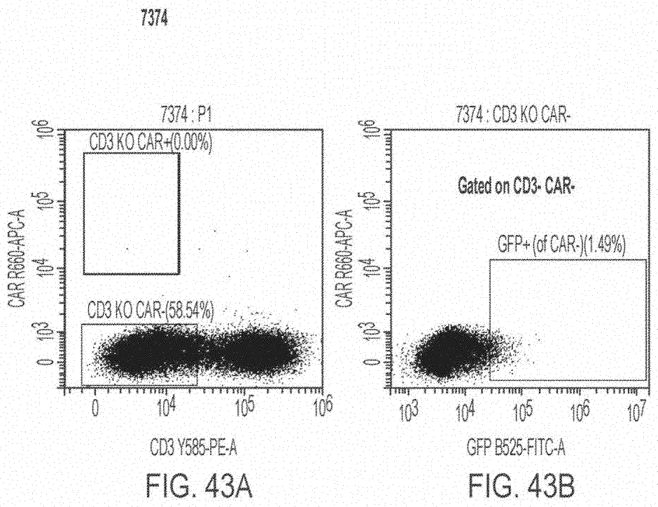

Representative flow cytometric data in T cells transduced with the AAV vector 7374 (FIG. 8) after electroporation with nuclease mRNA demonstrates that the ability to “stack” the DNA of a second AAV is dependent on the presence of the DNA from a first AAV (e.g., 73247) being incorporated into the edited gene locus. 7374, whose cargo contains the GFP coding sequence but lacks the EF1-alpha promoter to drive gene expression, does not show GFP expression unless the 73247 AAV is first stably incorporated into the genome. Therefore, genes delivered by the second AAV should not be detected if the first AAV vector is not also transduced. As expected, cells transduced with AAV vector 7374 failed to express the CAR (FIG. 43A). Only a low level of leaky and transient GFP expression can be detected in T cells transduced with the 7374 vector (FIG. 43B).

Co-transduction with AAV vectors 73247 and 7374 after electroporation with nuclease mRNA should result in the incorporation and expression of the CD19 CAR, disruption of the TRAC locus, and incorporation and expression of GFP.

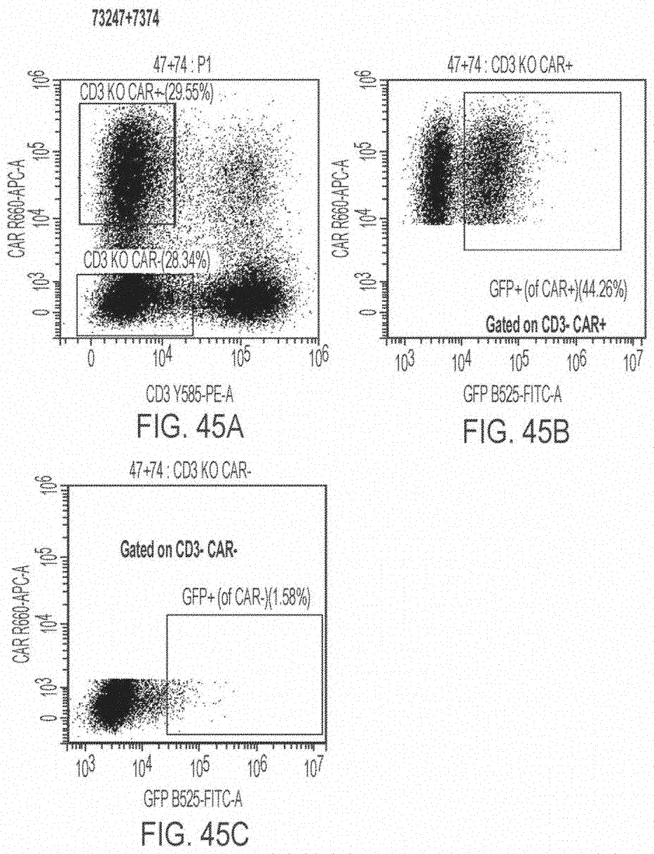

FIG. 45 shows that approximately 30% of total T cells are CD3⁻CAR⁺, demonstrating successful integration of DNA delivered by 73247 into the edited gene locus. To determine if the DNA from AAV vector 7374 was also incorporated (i.e., stacked) into the targeted site, CD3⁻CAR⁺ cells were gated on and analyzed for the frequency of detectable GFP⁺ cells. FIG. 45B shows that approximately 44% of CD3⁻CAR⁺ cells co-expressed GFP in this experimental condition. To determine if GFP expression correlated with CAR expression, the frequency of GFP⁺ cells was also evaluated in CD3⁻CAR⁻ cells (FIG. 45C). Importantly, GFP frequency was much lower in this population compared to CAR⁺ cells.

### Conclusions

The ability to stack DNA delivered by multiple AAV vectors into a targeted site was successful based on the high frequency of GFP⁺ cells specifically in the CD3⁻CAR⁺ cell populations. This result was in stark contrast to the low and transient expression of GFP found in T cells transduced with 7374 alone and in the absence of 73247.

Collectively, these results support the hypothesis that the DNA delivered by the first AAV vector, 73247, needs to be present and stable incorporated for the DNA from the second AAV vector, 7374, which lacks the promoter present on 73247 that is necessary to drive GFP expression. Furthermore, transcriptional splicing of the intron between the EF1-alpha promoter and the GFP gene needs to be successful for GFP expression to occur.

### Example 8

### Insertion of CAR and GFP Coding Sequences in Primary T Cells by AAV Stacking where the GFP Expression is Linked to CAR Expression by a 2A Element Preceding an Intron

### Materials and Methods

CD3+ T cells were isolated, stimulated, prepped, electroporated with mRNA encoding the TRC 1-2L.1592 nuclease and resuspended as described in example 2.

AAVs were prepared using the 73248 and 73249 constructs previously described. The volume of AAV needed for each experimental condition was calculated as described previously. AAV vectors were added to appropriate wells at a multiplicity of infection (MOI) of 2×10⁴ vg/viable cell, with viral vectors 73248 and 73249, and added to separate experimental wells at equal MOIs. Cells electroporated with the TRC1-2 L.1592 nuclease only, and wells transduced individually with each of the two experimental vectors, acted as controls. All experimental conditions were cultured as previously described in example 2.

To assess the frequency of targeted insertion of both AAV vectors into gene-edited T cells, samples were grown in FBS and IL-2 supplemented medium for 72 hours. Viable cells in each experimental condition were then counted, pelleted, resuspended, stained with an antibody cocktail of human anti-CD3 PE and anti-FMC63 (CAR) Alexa467 and subjected to FLOW cytometry as described in example 2.

### Results

To serve as a control, T cells were electroporated with TRC 1-2L.1592 nuclease only (FIG. 35). To evaluate the efficiency of nuclease editing at the TRAC locus, cells were stained for loss of cell surface CD3 expression. FIG. 35A shows that over 51% of electroporated cells were devoid of CD3 cell surface expression. Importantly, cells treated with only nuclease failed to express green fluorescent protein (GFP) (FIG. 35B), showing that AAV transduction is required for GFP transgene expression.

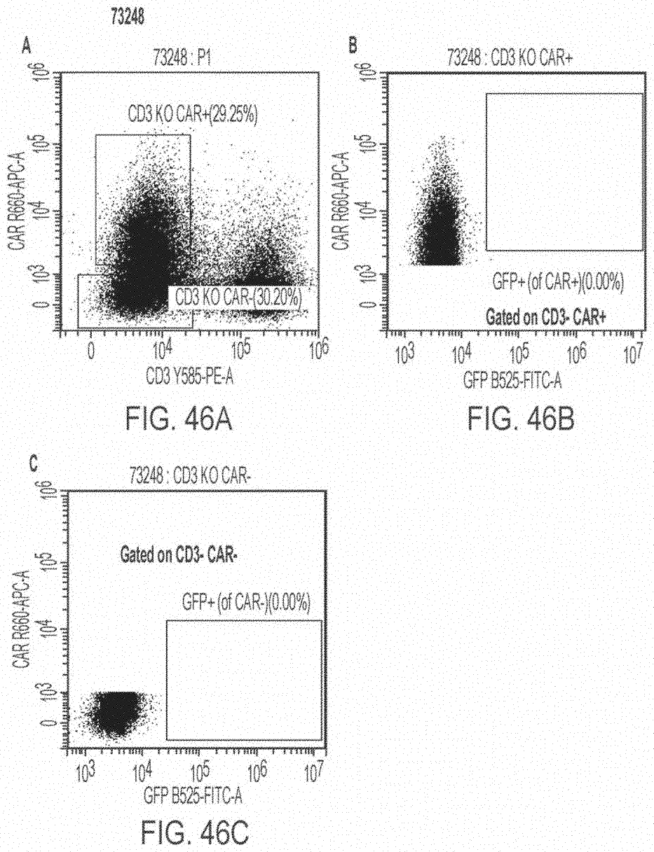

T cells that were electroporated with nuclease and subsequently transduced with each AAV vector, 73248 (FIG. 18), whose cargo contains a JeT promoter diving a CD19 CAR coding sequence, followed by a 2A element and an intron, should allow for incorporation and expression of the CD19 CAR and disruption of the TRAC locus. FIG. 46A shows that over 29% of cells are CD3⁻CAR⁺, demonstrating successful incorporation of the 73248 template DNA into the edited TRAC locus. In addition, when gating on CD3⁻CAR⁺ cells, GFP⁺ cells are not detected (FIG. 46B).

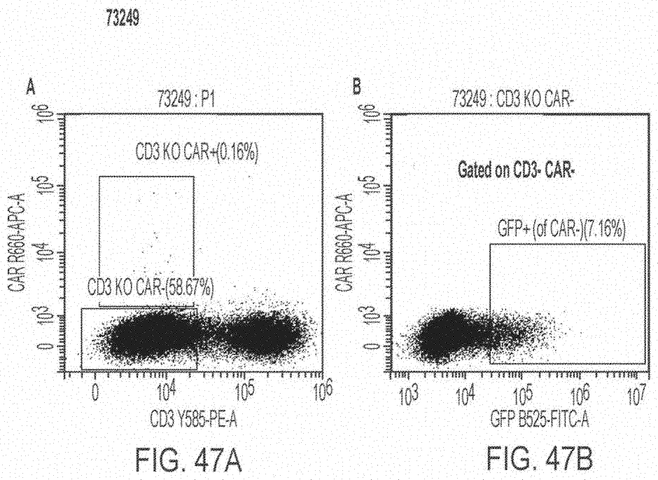

Representative flow cytometric data in T cells transduced with the AAV vector 73249 (FIG. 19) after electroporation with nuclease mRNA demonstrates that the ability to “stack” the DNA of a second AAV is dependent on the presence of the DNA from a first AAV (e.g., 73248) being incorporated into the edited gene locus. 73249 whose cargo contains the GFP coding sequence but lacks the EF1-alpha promoter to drive gene expression does not show GFP expression unless the 73248 AAV is first stably incorporated into the genome. Therefore, genes delivered by the second AAV should not be detected if the first AAV vector is not also transduced. As expected, cells transduced with AAV vector 73249 failed to express the CAR (FIG. 47A). Only a low level of leaky and transient GFP expression can be detected in T cells transduced with this viral vector.

Co-transduction with AAV vectors 73248 and 73249 after electroporation with nuclease mRNA should result in the incorporation and expression of the CD19 CAR, disruption of the TRAC locus, and incorporation and expression of GFP.

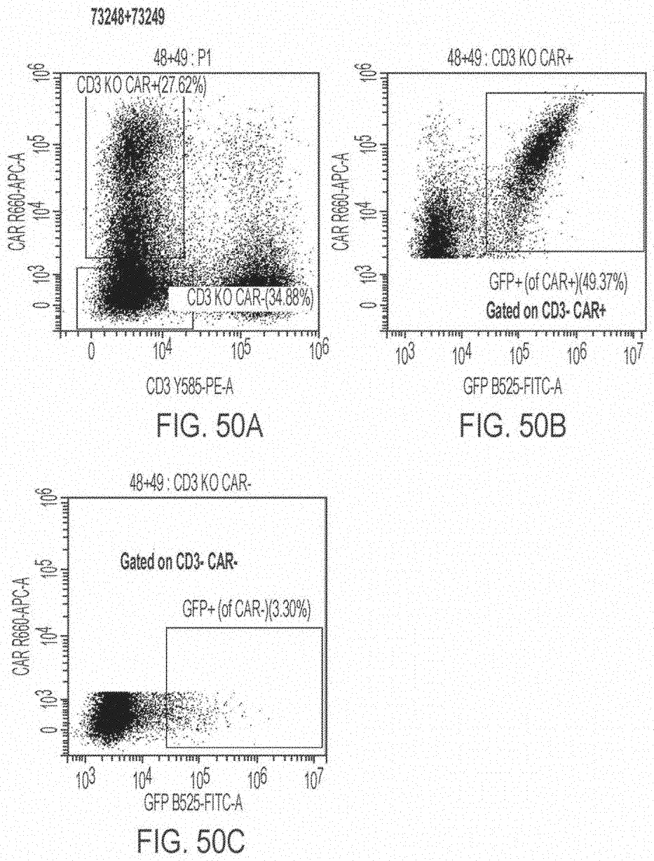

FIG. 50 shows that over 27% of total T cells are CD3⁻CAR⁺, demonstrating successful integration of DNA delivered by 73248 into the edited gene locus. To determine if the DNA from AAV vector 73249 was also incorporated (i.e., stacked) into the targeted site, CD3⁻CAR⁺ cells were gated on and analyzed for the frequency of detectable GFP⁺ cells. FIG. 50B shows that about 49% of CD3⁻CAR⁺ cells co-expressed GFP in this experimental condition. To determine if GFP expression correlated with CAR expression, the frequency of GFP⁺ cells was also evaluated in CD3⁻CAR⁻ cells (FIG. 50C). Importantly, GFP frequency was much lower in this population compared to CAR⁺ cells.

### Conclusions

The ability to stack DNA delivered by multiple AAV vectors into a targeted site was successful based on the high frequency of GFP⁺ cells specifically in the CD3⁻CAR⁺ cell populations. This result was in stark contrast to the low and transient expression of GFP found in T cells transduced with 73249 alone and in the absence of 73248.

Collectively, these results support the hypothesis that the DNA delivered by the first AAV vector, 73248, needs to be present for the DNA from the second AAV vector, 73249, to be expressed. Further, stable incorporation and expression of the second transgene GFP is dependent on expression of CAR and cleavage at the 2A element.

### Example 9

### Insertion of CAR and GFP Coding Sequences in Primary T Cells by AAV Stacking where the GFP Expression is Linked to CAR Expression by a 2A Element Preceded by an Intron

### Materials and Methods

CD3+ T cells were isolated, stimulated, prepped, electroporated with mRNA encoding the TRC 1-2L.1592 nuclease and resuspended as described in example 2.

AAVs were prepared using the 73250 and 73251 constructs previously described. The volume of AAV needed for each experimental condition was calculated as described previously. AAV vectors were added to appropriate wells at a multiplicity of infection (MOI) of 2×10⁴ vg/viable cell, with viral vectors 73250 and 73251, and added to separate experimental wells at equal MOIs. Cells electroporated with the TRC 1-2L.1592 nuclease only, and wells transduced individually with each of the two experimental vectors, acted as controls. All experimental conditions were cultured as previously described in example 2.

To assess the frequency of targeted insertion of both AAV vectors into gene-edited T cells, samples were grown in FBS and IL-2 supplemented medium for 72 hours. Viable cells in each experimental condition were then counted, pelleted, resuspended, stained with an antibody cocktail of human anti-CD3 PE and anti-FMC63 (CAR) Alexa467 and subjected to FLOW cytometry as described in example 2.

### Results

To serve as a control, T cells were electroporated with TRC 1-2L.1592 nuclease only (FIG. 35). To evaluate the efficiency of nuclease editing at the TRAC locus, cells were stained for loss of cell surface CD3 expression. FIG. 35A shows that over 51% of electroporated cells were devoid of CD3 cell surface expression. Importantly, cells treated with only nuclease failed to express green fluorescent protein (GFP) (FIG. 35B), showing that AAV transduction is required for GFP transgene expression.

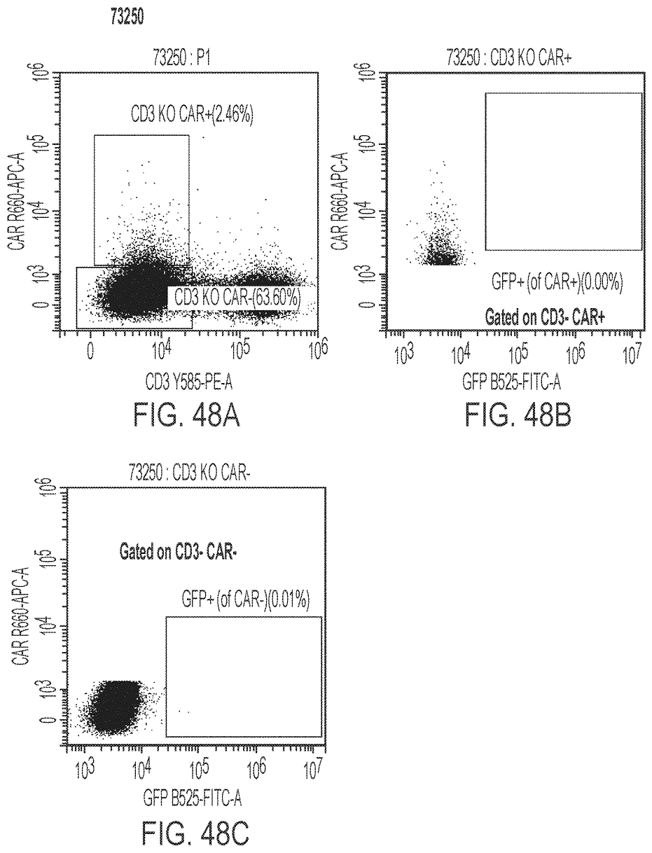

T cells that were electroporated with nuclease and subsequently transduced with each AAV vector, 73250 (FIG. 20), whose cargo contains a JeT promoter diving a CD19 CAR coding sequence but lacking a stop codon, should allow for incorporation and expression of the CD19 CAR and disruption of the TRAC locus. FIG. 48A shows that only about 2% of cells are CD3⁻CAR⁺. Low expression could be accounted for due to the lack of termination of the CAR coding sequence provided by this AAV alone. Gating on CD3⁻CAR⁺ cells, no GFP⁺ cells are detected (FIG. 48B).

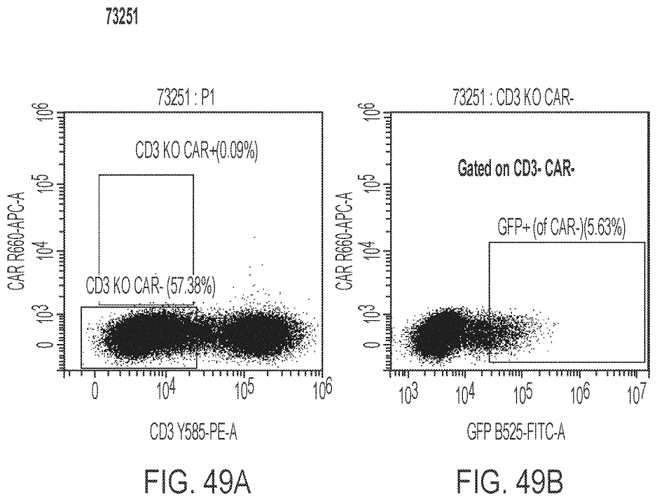

Representative flow cytometric data in T cells transduced with the AAV vector 73251 (FIG. 21) after electroporation with nuclease mRNA demonstrates that the ability to “stack” the DNA of a second AAV is dependent on the presence of the DNA from a first AAV (e.g., 73250) being incorporated into the edited gene locus. 73249 whose cargo contains an intron followed by a 2A element in frame with the GFP coding sequence but lacking the EF1-alpha promoter to drive gene expression should not show GFP expression unless the 73250 AAV is first stably incorporated into the genome. Therefore, genes delivered by the second AAV should not be detected if the first AAV vector is not also transduced. As expected, cells transduced with AAV vector 73251 failed to express the CAR (FIG. 49A). Only a low level of transient GFP expression can be detected in T cells transduced with this viral vector (FIG. 49B).

Co-transduction with AAV vectors 73250 and 73251 after electroporation with nuclease mRNA should result in the incorporation and expression of the CD19 CAR, disruption of the TRAC locus, and incorporation and expression of GFP.

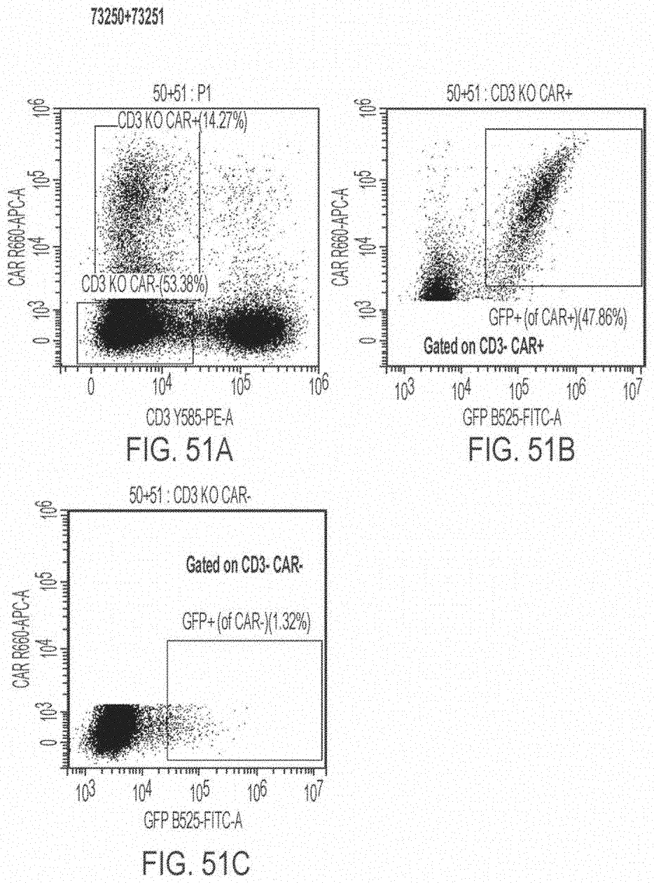

FIG. 51 shows that about 14% of total T cells are CD3⁻CAR⁺, demonstrating successful integration of DNA delivered by 73250 into the edited gene locus. To determine if the DNA from AAV vector 73251 was also incorporated (i.e., stacked) into the targeted site, CD3⁻CAR⁺ cells were gated on and analyzed for the frequency of detectable GFP⁺ cells. FIG. 51B shows that about 48% of CD3⁻CAR⁺ cells co-expressed GFP in this experimental condition. To determine if GFP expression correlated with CAR expression, the frequency of GFP⁺ cells was also evaluated in CD3⁻CAR⁻ cells (FIG. 51C). Importantly, GFP frequency was much lower in this population compared to CAR⁺ cells.

### Conclusions

The ability to stack DNA delivered by multiple AAV vectors into a targeted site was successful based on the high frequency of GFP⁺ cells specifically in the CD3⁻CAR⁺ cell populations. This result was in stark contrast to the low and transient expression of GFP found in T cells transduced with 73250 alone and in the absence of 73251.

Collectively, these results support the hypothesis that the DNA delivered by the first AAV vector, 73250, needs to be present for the DNA from the second AAV vector, 73251, to be expressed. Further, stable incorporation and expression of the second transgene GFP is dependent on expression of CAR and cleavage at the 2A element.

What is claimed is:

1. A composition comprising:
(a) a first polynucleotide comprising a first nucleic acid sequence comprising:
(i) a first donor nucleic acid sequence comprising a first nuclease recognition sequence for a first engineered nuclease; and
(ii) a first homology region positioned 3′ downstream of said first nuclease recognition sequence;
(b) a second polynucleotide comprising a second nucleic acid sequence comprising:
(i) a 5′ homology arm having homology to at least a portion of said first donor nucleic acid sequence and to a 5′ portion of said first nuclease recognition sequence;
(ii) a 3′ homology arm having homology to a 3′ portion of said first nuclease recognition sequence and to said first homology region; and
(iii) a second donor nucleic acid sequence positioned between said 5′ homology arm and said 3′ homology arm; and
(c) one or more engineered nucleases, or one or more nucleic acids encoding said one or more engineered nucleases, comprising said first engineered nuclease.

2. The composition of claim 1, wherein said first nuclease recognition sequence is positioned at the 3′ end of said first donor nucleic acid sequence.

3. The composition of claim 1 or 2, wherein said one or more engineered nucleases is an engineered meganuclease, a TALEN, a compact TALEN, a zinc finger nuclease, a CRISPR system nuclease, or a megaTAL.

4. The composition of any one of claims 1-3, wherein said one or more engineered nucleases is an engineered meganuclease.

5. The composition of any one of claims 1-4, wherein said first engineered nuclease is capable of binding and cleaving said first nuclease recognition sequence and an endogenous nuclease recognition sequence normally present in the genome of a eukaryotic cell of interest.

6. The composition of claim 5, wherein said first nuclease recognition sequence is identical to said endogenous nuclease recognition sequence.

7. The composition of claim 5 or 6, wherein said endogenous nuclease recognition sequence is within a T cell receptor (TCR) alpha gene or a TCR beta gene.

8. The composition of claim 7, wherein said endogenous nuclease recognition sequence is within a TCR alpha constant (TRAC) gene or TCR beta constant (TRBC) gene.

9. The composition of any one of claims 1-8, wherein said first nuclease recognition sequence comprises SEQ ID NO: 1.

10. The composition of any one of claims 1-9, wherein said one or more nucleic acids encoding said one or more engineered nucleases are mRNA, or wherein said one or more nucleic acids encoding said one or more engineered nucleases are comprised within one or more nuclease adeno-associated viruses (AAVs).

11. The composition of any one of claims 1-10, wherein said second donor nucleic acid sequence does not comprise a second nuclease recognition sequence, or does not comprise a 5′ portion of a recognition sequence that is capable of pairing with said 3′ portion of said first nuclease recognition sequence to generate a second nuclease recognition sequence, or does not comprise a 3′ portion of a nuclease recognition sequence that is capable of pairing with said 5′ portion of said first nuclease recognition sequence to generate a second nuclease recognition sequence.

12. The composition of any one of claims 1-10, wherein said second donor nucleic acid sequence comprises a second nuclease recognition sequence, or comprises a 5′ portion of a nuclease recognition sequence capable of pairing with said 3′ portion of said first nuclease recognition sequence to generate a second nuclease recognition sequence, or comprises a 3′ portion of a nuclease recognition sequence capable of pairing with said 5′ portion of said first nuclease recognition sequence to generate a second nuclease recognition sequence.

13. The composition of claim 12, wherein said first engineered nuclease is capable of binding and cleaving said first nuclease recognition sequence, said second nuclease recognition sequence, and said endogenous nuclease recognition sequence.

14. The composition of claim 12 or 13, wherein said first nuclease recognition sequence, said second nuclease recognition sequence, and said endogenous nuclease recognition sequence are identical.

15. The composition of any one of claims 1-14, wherein said first donor nucleic acid sequence comprises a first transgene.

16. The composition of claim 15, wherein said first donor nucleic acid sequence comprises a first promoter that is operably linked to said first transgene, or a sequence capable of operably linking said first transgene to an endogenous promoter.

17. The composition of claim 15 or 16, wherein said first donor nucleic acid sequence comprises, from 5′ to 3′, a first portion of said first transgene, a first untranslated sequence, said first recognition sequence, and said first homology region,
and wherein said second donor nucleic acid sequence comprises, from 5′ to 3′, said 5′ homology arm, a second untranslated sequence, and a second portion of said first transgene,
wherein said 5′ homology arm comprises, from 5′ to 3′, a sequence having homology to at least a portion of said first transgene, a sequence having homology to said first untranslated sequence, and a sequence having homology to a 5′ portion of said first nuclease recognition sequence,
wherein insertion of said second donor nucleic acid sequence into said first nuclease recognition sequence would generate a sequence comprising, from 5′ to 3′, said first portion of said first transgene, said 5′ portion of said first nuclease recognition sequence flanked by said first and said second untranslated sequence, and said second portion of said first transgene.

18. The composition of claim 17, wherein said first untranslated sequence is a first intron sequence comprising a splice donor sequence at its 5′ end, and said second untranslated sequence is a second intron sequence comprising a splice acceptor sequence at its 3′ end, wherein said splice donor sequence and said splice acceptor sequence are capable of being recognized by a splicing complex, and said first intron sequence, said 5′ portion of said first nuclease recognition sequence, and said second intron sequence are capable of being spliced from said first polynucleotide upon insertion of said second donor nucleic acid sequence into said first nuclease recognition sequence and expression of said first transgene.

19. The composition of any one of claims 15-18, wherein said second donor nucleic acid sequence comprises a second transgene.

20. The composition of claim 19, wherein said second donor nucleic acid sequence further comprises a second promoter which is operably linked to said second transgene.

21. The composition of claim 15 or 16, wherein said first donor nucleic acid sequence comprises, from 5′ to 3′, said first transgene, an IRES or 2A element, a first untranslated sequence, said first recognition sequence, and said first homology region,
and wherein said second donor nucleic acid sequence comprises, from 5′ to 3′, said 5′ homology arm, a second untranslated sequence, and a second transgene,
wherein said 5′ homology arm comprises, from 5′ to 3′, a sequence having homology to at least a portion of said first transgene, a sequence having homology to said IRES or 2A element, a sequence having homology to said first untranslated sequence, and a sequence having homology to a 5′ portion of said first nuclease recognition sequence,
wherein insertion of said second donor nucleic acid sequence into said first nuclease recognition sequence would generate a sequence comprising, from 5′ to 3′, said first transgene, said 2A or IRES element, said 5′ portion of said first nuclease recognition sequence flanked by said first and said second untranslated sequence, and said second transgene, such that said first transgene and said second transgene are operably linked to a single promoter.

22. The composition of claim 15 or 16, wherein said first donor nucleic acid sequence comprises, from 5′ to 3′, said first transgene, a first untranslated sequence, said first recognition sequence, and said first homology region,
and wherein said second donor nucleic acid sequence comprises, from 5′ to 3′, said 5′ homology arm, a second untranslated sequence, an IRES or 2A element, and a second transgene,
wherein said 5′ homology arm comprises, from 5′ to 3′, a sequence having homology to at least a portion of said first transgene, a sequence having homology to said IRES or 2A element, a sequence having homology to said first untranslated sequence, and a sequence having homology to a 5′ portion of said first nuclease recognition sequence,
wherein insertion of said second donor nucleic acid sequence into said first nuclease recognition sequence would generate a sequence comprising, from 5′ to 3′, said first transgene, said 2A or IRES element, said 5′ portion of said first nuclease recognition sequence flanked by said first and said second untranslated sequence, and said second transgene, such that said first transgene and said second transgene are operably linked to a single promoter.

23. The composition of claim 21 or 22, wherein said first untranslated sequence is a first intron sequence comprising a splice donor sequence at its 5′ end, and said second untranslated sequence is a second intron sequence comprising a splice acceptor sequence at its 3′ end, wherein said splice donor sequence and said splice acceptor sequence are capable of being recognized by a splicing complex, and said first intron sequence, said 5′ portion of said first nuclease recognition sequence, and said second intron sequence are capable of being spliced from said first polynucleotide upon insertion of said second donor nucleic acid sequence into said first nuclease recognition sequence and expression of said first transgene and said second transgene.

24. The composition of claim 15 or 16, wherein said first donor nucleic acid sequence comprises, from 5′ to 3′, said first transgene, said first recognition sequence, and said first homology region,
and wherein said second donor nucleic acid sequence comprises, from 5′ to 3′, said 5′ homology arm, a second promoter, and a second transgene operably linked to said second promoter,
wherein insertion of said second donor nucleic acid sequence into said first nuclease recognition sequence would generate a sequence comprising, from 5′ to 3′, said first transgene, said 5′ portion of said first nuclease recognition sequence, said second promoter, and said second transgene.

25. The composition of claim 15 or 16, wherein said first donor nucleic acid sequence comprises, from 5′ to 3′, said first transgene, a second promoter, and a first untranslated sequence,
and wherein said second donor nucleic acid sequence comprises, from 5′ to 3′, said 5′ homology arm and a second transgene,
wherein said 5′ homology arm has homology to at least a portion of said first untranslated sequence and to said 5′ portion of said first nuclease recognition sequence,
wherein insertion of said second donor nucleic acid sequence into said first nuclease recognition sequence would generate a sequence comprising, from 5′ to 3′, said first transgene, said second promoter, said first untranslated sequence, said 5′ portion of said first nuclease recognition sequence, and said second transgene.

26. The composition of claim 25, wherein said first untranslated sequence is an intron sequence comprising a splice donor sequence at its 5′ end and a splice acceptor sequence at its 3′ end, wherein said splice donor sequence and said splice acceptor sequence are capable of being recognized by a splicing complex, and said intron sequence is capable of being spliced from said first polynucleotide upon insertion of said second donor nucleic acid sequence into said first nuclease recognition sequence and expression of said second transgene.

27. The composition of any one of claims 15-26, wherein said first transgene encodes:
(a) a chimeric antigen receptor;
(b) an exogenous TCR;
(c) an inhibitory nucleic acid;
(d) a reporter protein;
(e) a protein useful for purification of a eukaryotic cell of interest;
(f) a therapeutic protein; or
(g) a suicide protein.

28. The composition of any one of claims 19-26, wherein said first transgene and/or said second transgene encodes:
(a) a chimeric antigen receptor;
(b) an exogenous TCR;
(c) an inhibitory nucleic acid;
(d) a reporter protein;
(e) a protein useful for purification of a eukaryotic cell of interest;
(f) a therapeutic protein; or
(g) a suicide protein.

29. The composition of claim 28, wherein said inhibitory nucleic acid comprises an shRNA or a microRNA-adapted shRNA.

30. The composition of any one of claims 15-20 and 27-29, wherein said first transgene encodes a protein that exceeds 5 kilobases in size.

31. The composition of any one of claims 1-30, wherein said second polynucleotide is comprised within a recombinant virus or a lipid nanoparticle.

32. The composition of claim 31, wherein said recombinant virus is a recombinant adeno-associated virus (AAV).

33. The composition of claim 32, wherein said AAV vector has a serotype of AAV1, AAV2, AAV3, AAV4, AAV5, AAV6, AAV7, AAV8, AAV9, AAV10, or AAV11.

34. The composition of claim 32 or 33, wherein said recombinant AAV has a serotype of AAV6.

35. The composition of any one of claims 32-34, wherein said second polynucleotide comprises only one D sequence.

36. The composition of claim 35, wherein said D sequence:
(a) is positioned within a 5′ inverted terminal repeat (ITR);
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

37. The composition of any one of claims 1-30, wherein said first polynucleotide is comprised within a first recombinant virus or a first lipid nanoparticle, and/or said second polynucleotide is comprised within a second recombinant virus or a second lipid nanoparticle.

38. The composition of any one of claims 5-37, wherein said first heterologous nucleic acid sequence further comprises:
(a) a 5′ homology arm that is homologous to a sequence 5′ upstream of said endogenous nuclease recognition sequence and to a 5′ portion of said endogenous nuclease recognition sequence; and
(b) a 3′ homology arm that is homologous to a sequence 3′ downstream of said endogenous nuclease recognition sequence and to a 3′ portion of said endogenous nuclease recognition sequence; or wherein said first homology region is homologous to a sequence 3′ downstream of said endogenous nuclease recognition sequence;
wherein said 5′ homology arm and said 3′ homology arm flank said first heterologous nucleic acid sequence.

39. The composition of any one of claims 5-38, wherein said first polynucleotide is comprised within a first recombinant virus or a first lipid nanoparticle, and/or said second polynucleotide is comprised within a second recombinant virus or a second lipid nanoparticle.

40. The composition of claim 39, wherein said first heterologous nucleic acid sequence further comprises a 5′ homology arm that is homologous to a sequence 5′ upstream of said endogenous nuclease recognition sequence and to a 5′ portion of said endogenous nuclease recognition sequence, and wherein said first homology region is homologous to a sequence 3′ downstream of said endogenous nuclease recognition sequence.

41. The composition of any one of claims 37-40, wherein said first recombinant virus is a first recombinant AAV, and/or wherein said second recombinant virus is a second recombinant AAV.

42. The composition of claim 41, wherein said first AAV vector and/or said second AAV vector has a serotype of AAV1, AAV2, AAV3, AAV4, AAV5, AAV6, AAV7, AAV8, AAV9, AAV10, or AAV11.

43. The composition of claim 41 or 42, wherein said first AAV vector and/or said second AAV vector has a serotype of AAV6.

44. The composition of any one of claims 41-43, wherein said first polynucleotide comprises only one D sequence, and wherein said second polynucleotide comprises only one D sequence.

45. The composition of claim 44, wherein said D sequence comprised by said first polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

46. The composition of claim 44 or 45, wherein said D sequence comprised by said second polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

47. The composition of any one of claims 44-46, wherein said D sequence comprised by said first polynucleotide:
(a) is positioned within a 5′ ITR;
(b) overlaps said 5′ ITR; or
(c) is positioned 3′ downstream of said 5′ ITR and 5′ upstream of said 5′ homology arm;
and wherein said D sequence comprised by said second polynucleotide:
(d) is positioned within a 5′ ITR;
(e) overlaps said 5′ ITR; or
(f) is positioned 3′ downstream of said 5′ ITR and 5′ upstream of said 5′ homology arm.

48. The composition of any one of claims 44-46, wherein said D sequence comprised by said first polynucleotide:
(a) is positioned 5′ upstream of a 3′ ITR and 3′ downstream of said 3′ homology arm;
(b) overlaps said 3′ ITR; or
(c) is positioned within said 3′ ITR;
and wherein said D sequence comprised by said second polynucleotide:
(d) is positioned 5′ upstream of a 3′ ITR and 3′ downstream of said 3′ homology arm;
(e) overlaps said 3′ ITR; or
(f) is positioned within said 3′ ITR.

49. The composition of any one of claims 1-48, wherein said composition is a eukaryotic cell.

50. A eukaryotic cell comprising:
(a) a first polynucleotide comprising a first heterologous nucleic acid sequence comprising:
(i) a first donor nucleic acid sequence comprising a first nuclease recognition sequence for a first engineered nuclease; and
(ii) a first homology region positioned 3′ downstream of said first nuclease recognition sequence;
(b) a second polynucleotide comprising a second heterologous nucleic acid sequence comprising:
(i) a 5′ homology arm having homology to at least a portion of said first donor nucleic acid sequence and to a 5′ portion of said first nuclease recognition sequence;
(ii) a 3′ homology arm having homology to a 3′ portion of said first nuclease recognition sequence and to said first homology region; and
(iii) a second donor nucleic acid sequence positioned between said 5′ homology arm and said 3′ homology arm; and
(c) one or more engineered nucleases, or one or more nucleic acids encoding said one or more engineered nucleases, comprising said first engineered nuclease.

51. The eukaryotic cell of claim 50, wherein said one or more engineered nucleases is an engineered meganuclease, a TALEN, a compact TALEN, a zinc finger nuclease, a CRISPR system nuclease, or a megaTAL.

52. The eukaryotic cell of claim 50 or 51, wherein said one or more engineered nucleases is an engineered meganuclease.

53. The eukaryotic cell of any one of claims 50-52, wherein said first engineered nuclease is capable of binding and cleaving said first nuclease recognition sequence and an endogenous nuclease recognition sequence normally present in the genome of a eukaryotic cell of interest.

54. The eukaryotic cell of claim 53, wherein said first nuclease recognition sequence is identical to said endogenous nuclease recognition sequence.

55. The eukaryotic cell of claim 53 or 54, wherein said endogenous nuclease recognition sequence is within a T cell receptor (TCR) alpha gene or a TCR beta gene.

56. The eukaryotic cell of claim 55, wherein said endogenous nuclease recognition sequence is within a TCR alpha constant (TRAC) gene or TCR beta constant (TRBC) gene.

57. The eukaryotic cell of any one of claims 50-56, wherein said first nuclease recognition sequence comprises SEQ ID NO: 1.

58. The eukaryotic cell of any one of claims 50-57, wherein said one or more nucleic acids encoding said one or more engineered nucleases are mRNA, or wherein said one or more nucleic acids encoding said one or more engineered nucleases are comprised within one or more nuclease adeno-associated viruses (AAVs).

59. The eukaryotic cell of any one of claims 50-58, wherein said second donor nucleic acid sequence does not comprise a second nuclease recognition sequence, or does not comprise a 5′ portion of a nuclease recognition sequence that is capable of pairing with said 3′ portion of said first nuclease recognition sequence to generate a second nuclease recognition sequence, or does not comprise a 3′ portion of a nuclease recognition sequence that is capable of pairing with said 5′ portion of said first nuclease recognition sequence to generate a second nuclease recognition sequence.

60. The eukaryotic cell of any one of claims 50-58, wherein said second donor nucleic acid sequence comprises a second nuclease recognition sequence, or comprises a 5′ portion of a nuclease recognition sequence capable of pairing with said 3′ portion of said first nuclease recognition sequence to generate a second nuclease recognition sequence, or comprises a 3′ portion of a nuclease recognition sequence capable of pairing with said 5′ portion of said first nuclease recognition sequence to generate a second nuclease recognition sequence.

61. The eukaryotic cell of claim 60, wherein said first engineered nuclease is capable of binding and cleaving said first nuclease recognition sequence, said second nuclease recognition sequence, and said endogenous nuclease recognition sequence.

62. The eukaryotic cell of claim 60 or 61, wherein said first nuclease recognition sequence, said second nuclease recognition sequence, and said endogenous nuclease recognition sequence are identical.

63. The eukaryotic cell of any one of claims 50-62, wherein said first donor nucleic acid sequence comprises a first transgene which is expressed in said eukaryotic cell.

64. The eukaryotic cell of claim 63, wherein said first donor nucleic acid sequence comprises a first promoter that is operably linked to said first transgene, or a sequence capable of operably linking said first transgene to an endogenous promoter of said eukaryotic cell.

65. The eukaryotic cell of claim 63 or 64, wherein said first donor nucleic acid sequence comprises, from 5′ to 3′, a first portion of said first transgene, a first untranslated sequence, said first recognition sequence, and said first homology region,
wherein said second donor nucleic acid sequence comprises, from 5′ to 3′, said 5′ homology arm, a second untranslated sequence, and a second portion of said first transgene,
wherein said 5′ homology arm comprises, from 5′ to 3′, a sequence having homology to at least a portion of said first transgene, a sequence having homology to said first untranslated sequence, and a sequence having homology to a 5′ portion of said first nuclease recognition sequence,
and wherein insertion of said second donor nucleic acid sequence into said first nuclease recognition sequence would generate a sequence comprising, from 5′ to 3′, said first portion of said first transgene, said 5′ portion of said first nuclease recognition sequence flanked by said first and said second untranslated sequence, and said second portion of said first transgene.

66. The eukaryotic cell of claim 65, wherein said first untranslated sequence is a first intron sequence comprising a splice donor sequence at its 5′ end, and said second untranslated sequence is a second intron sequence comprising a splice acceptor sequence at its 3′ end, wherein said splice donor sequence and said splice acceptor sequence are capable of being recognized by a splicing complex, and said first intron sequence, said 5′ portion of said first nuclease recognition sequence, and said second intron sequence are capable of being spliced from said first polynucleotide upon insertion of said second donor nucleic acid sequence into said first nuclease recognition sequence and expression of said first transgene.

67. The eukaryotic cell of any one of claims 63-66, wherein said second donor nucleic acid sequence comprises a second transgene which is expressed in said eukaryotic cell.

68. The eukaryotic cell of claim 67, wherein said second donor nucleic acid sequence further comprises a second promoter which is operably linked to said second transgene.

69. The eukaryotic cell of claim 63 or 64, wherein said first donor nucleic acid sequence comprises, from 5′ to 3′, said first transgene, an IRES or 2A element, a first untranslated sequence, said first recognition sequence, and said first homology region,
wherein said second donor nucleic acid sequence comprises, from 5′ to 3′, said 5′ homology arm, a second untranslated sequence, and a second transgene,
wherein said 5′ homology arm comprises, from 5′ to 3′, a sequence having homology to at least a portion of said first transgene, a sequence having homology to said IRES or 2A element, a sequence having homology to said first untranslated sequence, and a sequence having homology to a 5′ portion of said first nuclease recognition sequence,
and wherein insertion of said second donor nucleic acid sequence into said first nuclease recognition sequence would generate a sequence comprising, from 5′ to 3′, said first transgene, said 2A or IRES element, said 5′ portion of said first nuclease recognition sequence flanked by said first and said second untranslated sequence, and said second transgene, such that said first transgene and said second transgene are operably linked to a single promoter.

70. The eukaryotic cell of claim 63 or 64, wherein said first donor nucleic acid sequence comprises, from 5′ to 3′, said first transgene, a first untranslated sequence, said first recognition sequence, and said first homology region,
wherein said second donor nucleic acid sequence comprises, from 5′ to 3′, said 5′ homology arm, a second untranslated sequence, an IRES or 2A element, and a second transgene,
wherein said 5′ homology arm comprises, from 5′ to 3′, a sequence having homology to at least a portion of said first transgene, a sequence having homology to said IRES or 2A element, a sequence having homology to said first untranslated sequence, and a sequence having homology to a 5′ portion of said first nuclease recognition sequence,
wherein insertion of said second donor nucleic acid sequence into said first nuclease recognition sequence would generate a sequence comprising, from 5′ to 3′, said first transgene, said 2A or IRES element, said 5′ portion of said first nuclease recognition sequence flanked by said first and said second untranslated sequence, and said second transgene, such that said first transgene and said second transgene are operably linked to a single promoter.

71. The eukaryotic cell of claim 69 or 70, wherein said first untranslated sequence is a first intron sequence comprising a splice donor sequence at its 5′ end, and said second untranslated sequence is a second intron sequence comprising a splice acceptor sequence at its 3′ end, wherein said splice donor sequence and said splice acceptor sequence are capable of being recognized by a splicing complex, and said first intron sequence, said 5′ portion of said first nuclease recognition sequence, and said second intron sequence are capable of being spliced from said first polynucleotide upon insertion of said second donor nucleic acid sequence into said first nuclease recognition sequence and expression of said first transgene and said second transgene.

72. The eukaryotic cell of claim 63 or 64, wherein said first donor nucleic acid sequence comprises, from 5′ to 3′, said first transgene, said first recognition sequence, and said first homology region,
wherein said second donor nucleic acid sequence comprises, from 5′ to 3′, said 5′ homology arm, a second promoter, and a second transgene operably linked to said second promoter,
wherein insertion of said second donor nucleic acid sequence into said first nuclease recognition sequence would generate a sequence comprising, from 5′ to 3′, said first transgene, said 5′ portion of said first nuclease recognition sequence, said second promoter, and said second transgene.

73. The eukaryotic cell of claim 63 or 64, wherein said first donor nucleic acid sequence comprises, from 5′ to 3′, said first transgene, a second promoter, and a first untranslated sequence,
wherein said second donor nucleic acid sequence comprises, from 5′ to 3′, said 5′ homology arm and a second transgene,
wherein said 5′ homology arm has homology to at least a portion of said first untranslated sequence and to said 5′ portion of said first nuclease recognition sequence,
wherein insertion of said second donor nucleic acid sequence into said first nuclease recognition sequence would generate a sequence comprising, from 5′ to 3′, said first transgene, said second promoter, said first untranslated sequence, said 5′ portion of said first nuclease recognition sequence, and said second transgene.

74. The eukaryotic cell of claim 73, wherein said first untranslated sequence is an intron sequence comprising a splice donor sequence at its 5′ end and a splice acceptor sequence at its 3′ end, wherein said splice donor sequence and said splice acceptor sequence are capable of being recognized by a splicing complex, and said intron sequence is capable of being spliced from said first polynucleotide upon insertion of said second donor nucleic acid sequence into said first nuclease recognition sequence and expression of said second transgene.

75. The eukaryotic cell of any one of claims 50-74, wherein said first transgene encodes:
(a) a chimeric antigen receptor;
(b) an exogenous TCR;
(c) an inhibitory nucleic acid;
(d) a reporter protein;
(e) a protein useful for purification of said eukaryotic cell;
(f) a therapeutic protein; or
(g) a suicide protein.

76. The eukaryotic cell of any one of claims 67-74, wherein said first transgene and/or said second transgene encodes:
(a) a chimeric antigen receptor;
(b) an exogenous TCR;
(c) an inhibitory nucleic acid;
(d) a reporter protein;
(e) a protein useful for purification of said eukaryotic cell;
(f) a therapeutic protein; or
(g) a suicide protein.

77. The eukaryotic cell of claim 75 or 76, wherein said inhibitory nucleic acid comprises an shRNA or microRNA-adapted shRNA.

78. The eukaryotic cell of any one of claims 50-68 and 75-77, wherein said first transgene encodes a protein that exceeds 5 kilobases in size.

79. The eukaryotic cell of any one of claims 50-78, wherein said eukaryotic cell comprises said first polynucleotide in its genome.

80. The eukaryotic cell of any one of claims 53-79, wherein said eukaryotic cell comprises said first polynucleotide in its genome within said endogenous nuclease recognition sequence.

81. The eukaryotic cell of claim 79 or 80, wherein said eukaryotic cell comprises a recombinant virus comprising said second polynucleotide.

82. The eukaryotic cell of claim 81, wherein said recombinant virus is a recombinant AAV.

83. The eukaryotic cell of claim 82, wherein said recombinant AAV has a serotype of AAV1, AAV2, AAV3, AAV4, AAV5, AAV6, AAV7, AAV8, AAV9, AAV10, or AAV11.

84. The eukaryotic cell of claim 82 or 83, wherein said recombinant AAV has a serotype of AAV6.

85. The eukaryotic cell of any one of claims 82-84, wherein said second polynucleotide comprises only one D sequence.

86. The eukaryotic cell of claim 85, wherein said D sequence:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

87. The eukaryotic cell of any one of claims 50-78, wherein said first polynucleotide is comprised within a first recombinant virus or a first lipid nanoparticle, and/or said second polynucleotide is comprised within a second recombinant virus or a second lipid nanoparticle.

88. The eukaryotic cell of anyone of claims 50-87, wherein said first heterologous nucleic acid sequence further comprises:
(a) a 5′ homology arm that is homologous to a sequence 5′ upstream of said endogenous nuclease recognition sequence and to a 5′ portion of said endogenous nuclease recognition sequence; and
(b) a 3′ homology arm that is homologous to a sequence 3′ downstream of said endogenous nuclease recognition sequence and to a 3′ portion of said endogenous nuclease recognition sequence; or wherein said first homology region is homologous to a sequence 3′ downstream of said endogenous nuclease recognition sequence;
wherein said 5′ homology arm and said 3′ homology arm flank said first heterologous nucleic acid sequence.

89. The eukaryotic cell of any one of claims 53-80, wherein said first polynucleotide is comprised within a first recombinant virus or a first lipid nanoparticle, and/or said second polynucleotide is comprised within a second recombinant virus or a second lipid nanoparticle.

90. The eukaryotic cell of claim 89, wherein said first heterologous nucleic acid sequence further comprises a 5′ homology arm that is homologous to a sequence 5′ upstream of said endogenous nuclease recognition sequence and to a 5′ portion of said endogenous nuclease recognition sequence, and wherein said first homology region is homologous to a sequence 3′ downstream of said endogenous nuclease recognition sequence.

91. The eukaryotic cell of any one of claims 87-90, wherein said first recombinant virus is a first recombinant AAV, and/or wherein said second recombinant virus is a second recombinant AAV.

92. The eukaryotic cell of claim 91, wherein said first AAV vector and/or said second AAV vector has a serotype of AAV1, AAV2, AAV3, AAV4, AAV5, AAV6, AAV7, AAV8, AAV9, AAV10, or AAV11.

93. The eukaryotic cell of claim 91 or 92, wherein said first polynucleotide comprises only one D sequence, and wherein said second polynucleotide comprises only one D sequence.

94. The eukaryotic cell of claim 93, wherein said D sequence comprised by said first polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

95. The eukaryotic cell of claim 93 or 94, wherein said D sequence comprised by said second polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

96. The eukaryotic cell of any one of claims 93-95, wherein said D sequence comprised by said first polynucleotide:
(a) is positioned within a 5′ ITR;
(b) overlaps said 5′ ITR; or
(c) is positioned 3′ downstream of said 5′ ITR and 5′ upstream of said 5′ homology arm;
and wherein said D sequence comprised by said second polynucleotide:
(d) is positioned within a 5′ ITR;
(e) overlaps said 5′ ITR; or
(f) is positioned 3′ downstream of said 5′ ITR and 5′ upstream of said 5′ homology arm.

97. The eukaryotic cell of any one of claims 93-95, wherein said D sequence comprised by said first polynucleotide:
(a) is positioned 5′ upstream of a 3′ ITR and 3′ downstream of said 3′ homology arm;
(b) overlaps said 3′ ITR; or
(c) is positioned within said 3′ ITR;
and wherein said D sequence comprised by said second polynucleotide:
(d) is positioned 5′ upstream of a 3′ ITR and 3′ downstream of said 3′ homology arm;
(e) overlaps said 3′ ITR; or
(f) is positioned within said 3′ ITR.

98. The eukaryotic cell of any one of claims 50-97, wherein said eukaryotic cell is a eukaryotic cell.

99. A population of eukaryotic cells comprising a plurality of said eukaryotic cells of any one of claims 50-98.

100. The population of eukaryotic cells of claim 99, wherein at least about 20%, about 30%, about 40%, about 50%, about 55%, about 60%, about 65%, about 70%, about 75%, about 80%, about 85%, about 90%, about 95%, or up to 100% of cells in said population are said eukaryotic cell of any one of claims 50-98.

101. A pharmaceutical composition comprising a pharmaceutically-acceptable carrier and said eukaryotic cell of any one of claims 50-98.

102. A pharmaceutical composition comprising a pharmaceutically-acceptable carrier and said population of eukaryotic cells of claim 99 or 100.

103. A method of immunotherapy for treating a cancer in a subject in need thereof, said method comprising administering to said subject an effective amount of said pharmaceutical composition of claim 101 or 102, wherein said eukaryotic cell is a genetically-modified human T cell, or a cell derived therefrom, or a genetically-modified NK cell, or a cell derived therefrom, and wherein said eukaryotic cell comprises a CAR or exogenous TCR, wherein said CAR or said exogenous TCR comprises an extracellular ligand-binding domain having specificity for a tumor-specific antigen.

104. The method of claim 103, wherein said first donor nucleic acid sequence and/or said second donor nucleic acid sequence comprises a transgene encoding said CAR or said exogenous TCR.

105. The method of claim 103 or 104, wherein said first donor nucleic acid sequence is inserted into the genome of said eukaryotic cell within a TCR alpha gene or a TCR beta gene.

106. The method of claim 105, wherein said first donor nucleic acid sequence is inserted into the genome of said eukaryotic cell within a TRAC gene or TRBC gene.

107. The method of any one of claims 103-106, wherein said eukaryotic cell has no detectable cell-surface expression of an endogenous TCR.

108. The method of any one of claims 103-107, wherein said cancer is selected from the group consisting of a cancer of carcinoma, lymphoma, sarcoma, blastomas, and leukemia.

109. The method of any one of claims 103-108, wherein said cancer is selected from the group consisting of a cancer of B-cell origin, breast cancer, gastric cancer, neuroblastoma, osteosarcoma, lung cancer, melanoma, prostate cancer, colon cancer, renal cell carcinoma, ovarian cancer, rhabdomyo sarcoma, leukemia, and Hodgkin's lymphoma.

110. The method of claim 109, wherein said cancer of B-cell origin is selected from the group consisting of B-lineage acute lymphoblastic leukemia, B-cell chronic lymphocytic leukemia, B-cell non-Hodgkin's lymphoma, and multiple myeloma.

111. A method for producing a genetically-modified eukaryotic cell, said method comprising introducing into a eukaryotic cell:
(a) a first polynucleotide comprising a first heterologous nucleic acid sequence comprising:
(i) a first donor nucleic acid sequence comprising a first nuclease recognition sequence for a first engineered nuclease; and
(ii) a first homology region positioned 3′ downstream of said first nuclease recognition sequence;
(b) a second polynucleotide comprising a second heterologous nucleic acid sequence comprising:
(i) a 5′ homology arm having homology to at least a portion of said first donor nucleic acid sequence and to a 5′ portion of said first nuclease recognition sequence;
(ii) a 3′ homology arm having homology to a 3′ portion of said first nuclease recognition sequence and to said first homology region; and
(iii) a second donor nucleic acid sequence positioned between said 5′ homology arm and said 3′ homology arm; and
(c) one or more engineered nucleases, or one or more nucleic acids encoding said one or more engineered nucleases, comprising said first engineered nuclease,
wherein said one or more engineered nucleases are expressed in said eukaryotic cell and generate a first cleavage site at an endogenous nuclease recognition sequence in the genome of said eukaryotic cell,
wherein said first donor nucleic acid sequence is inserted into said first cleavage site,
wherein said one or more engineered nucleases generate a second cleavage site at said first nuclease recognition sequence,
and wherein said second donor nucleic acid sequence is inserted into said second cleavage site.

112. The method of claim 111, wherein said first polynucleotide and said second polynucleotide are introduced simultaneously into said eukaryotic cell.

113. The method of claim 111 or 112, wherein said one or more nucleic acids encoding said one or more engineered nucleases is introduced into said eukaryotic cell within at least 1 hour, 2 hours, 3 hours, 4 hours, 5 hours, 6 hours, 7 hours, 8 hours, 9 hours, 10 hours, 15 hours, 20 hours, or 24 hours of said first polynucleotide and said second polynucleotide.

114. The method of claim 111, wherein said first polynucleotide and said second polynucleotide are introduced sequentially into said eukaryotic cell.

115. The method of claim 114, wherein said second polynucleotide is introduced into said eukaryotic cell within at least 1 hour, 2 hours, 3 hours, 4 hours, 5 hours, 6 hours, 7 hours, 8 hours, 9 hours, 10 hours, 15 hours, 20 hours, or 24 hours of said first polynucleotide.

116. The method of claim 111 or 112, wherein said first polynucleotide, said second polynucleotide, and said one or more nucleic acids encoding said one or more engineered nucleases are introduced simultaneously into said eukaryotic cell.

117. The method of any one of claims 111-116, wherein said one or more engineered nucleases is an engineered meganuclease, a TALEN, a compact TALEN, a zinc finger nuclease, a CRISPR system nuclease, or a megaTAL.

118. The method of any one of claims 111-117, wherein said one or more engineered nucleases is an engineered meganuclease.

119. The method of any one of claims 111-118, wherein said first polynucleotide further comprises:
(a) a 5′ homology arm that is homologous to a sequence 5′ upstream of said endogenous nuclease recognition sequence and to a 5′ portion of said endogenous nuclease recognition sequence; and
(b) a 3′ homology arm that is homologous to a sequence 3′ downstream of said endogenous nuclease recognition sequence and to a 3′ portion of said endogenous nuclease recognition sequence;
wherein said 5′ homology arm and said 3′ homology arm flank said first heterologous nucleic acid sequence.

120. The method of any one of claims 111-119, wherein said first nuclease recognition sequence is identical to said endogenous nuclease recognition sequence.

121. The method of claim 120, wherein the first engineered nuclease generates said first cleavage site and said second cleavage site.

122. The method of any one of claims 111-121, wherein said first polynucleotide further comprises a 5′ homology arm that is homologous to a sequence 5′ upstream of said endogenous nuclease recognition sequence and to a 5′ portion of said endogenous nuclease recognition sequence, and wherein said first homology region is homologous to a sequence 3′ downstream of said endogenous nuclease recognition sequence.

123. The method of any one of claims 111-122, wherein said endogenous nuclease recognition sequence is within a T cell receptor (TCR) alpha gene or a TCR beta gene.

124. The method of any one of claims 111-123, wherein said endogenous nuclease recognition sequence is within a TCR alpha constant (TRAC) gene or TCR beta constant (TRBC) gene.

125. The method of any one of claims 111-124, wherein said first nuclease recognition sequence comprises SEQ ID NO: 1.

126. The method of any one of claims 111-125, wherein said one or more nucleic acids encoding said one or more engineered nucleases are mRNA, or wherein said one or more nucleic acids encoding said one or more engineered nucleases are comprised within one or more nuclease AAVs.

127. The method of any one of claims 111-126, wherein said second donor nucleic acid sequence does not comprise a second nuclease recognition sequence, or does not comprise a 5′ portion of a nuclease recognition sequence that is capable of pairing with said 3′ portion of said first nuclease recognition sequence to generate a second nuclease recognition sequence, or does not comprise a 3′ portion of a nuclease recognition sequence that is capable of pairing with said 5′ portion of said first nuclease recognition sequence to generate a second nuclease recognition sequence.

128. The method of any one of claims 111-126, wherein said second donor nucleic acid sequence comprises a second nuclease recognition sequence, or comprises a 5′ portion of a nuclease recognition sequence capable of pairing with said 3′ portion of said first nuclease recognition sequence to generate a second nuclease recognition sequence, or comprises a 3′ portion of a nuclease recognition sequence capable of pairing with said 5′ portion of said first nuclease recognition sequence to generate a second nuclease recognition sequence.

129. The method of claim 128, wherein said first engineered nuclease is capable of binding and cleaving said first nuclease recognition sequence, said second nuclease recognition sequence, and said endogenous nuclease recognition sequence.

130. The method of claim 128 or 129, wherein said first nuclease recognition sequence, said second nuclease recognition sequence, and said endogenous nuclease recognition sequence are identical.

131. The method of any one of claims 111-130, wherein said first donor nucleic acid sequence comprises a first transgene which is expressed in said eukaryotic cell.

132. The method of claim 131, wherein said first donor nucleic acid sequence comprises a first promoter that is operably linked to said first transgene, or a sequence capable of operably linking said first transgene to an endogenous promoter of said eukaryotic cell.

133. The method of claim 131 or 132, wherein said first donor nucleic acid sequence comprises, from 5′ to 3′, a first portion of said first transgene, a first untranslated sequence, said first recognition sequence, and said first homology region,
wherein said second donor nucleic acid sequence comprises, from 5′ to 3′, said 5′ homology arm, a second untranslated sequence, and a second portion of said first transgene,
wherein said 5′ homology arm comprises, from 5′ to 3′, a sequence having homology to at least a portion of said first transgene, a sequence having homology to said first untranslated sequence, and a sequence having homology to a 5′ portion of said first nuclease recognition sequence,
and wherein insertion of said second donor nucleic acid sequence into said first nuclease recognition sequence would generate a sequence comprising, from 5′ to 3′, said first portion of said first transgene, said 5′ portion of said first nuclease recognition sequence flanked by said first and said second untranslated sequence, and said second portion of said first transgene.

134. The method of claim 133, wherein said first untranslated sequence is a first intron sequence comprising a splice donor sequence at its 5′ end, and said second untranslated sequence is a second intron sequence comprising a splice acceptor sequence at its 3′ end, wherein said splice donor sequence and said splice acceptor sequence are capable of being recognized by a splicing complex, and said first intron sequence, said 5′ portion of said first nuclease recognition sequence, and said second intron sequence are capable of being spliced from said first polynucleotide upon insertion of said second donor nucleic acid sequence into said first nuclease recognition sequence and expression of said first transgene.

135. The method of any one of claims 131-134, wherein said second donor nucleic acid sequence comprises a second transgene which is expressed in said eukaryotic cell.

136. The method of claim 135, wherein said second donor nucleic acid sequence further comprises a second promoter which is operably linked to said second transgene.

137. The method of claim 131 or 132, wherein said first donor nucleic acid sequence comprises, from 5′ to 3′, said first transgene, an IRES or 2A element, a first untranslated sequence, said first recognition sequence, and said first homology region,
wherein said second donor nucleic acid sequence comprises, from 5′ to 3′, said 5′ homology arm, a second untranslated sequence, and a second transgene,
wherein said 5′ homology arm comprises, from 5′ to 3′, a sequence having homology to at least a portion of said first transgene, a sequence having homology to said IRES or 2A element, a sequence having homology to said first untranslated sequence, and a sequence having homology to a 5′ portion of said first nuclease recognition sequence,
and wherein insertion of said second donor nucleic acid sequence into said first nuclease recognition sequence would generate a sequence comprising, from 5′ to 3′, said first transgene, said 2A or IRES element, said 5′ portion of said first nuclease recognition sequence flanked by said first and said second untranslated sequence, and said second transgene, such that said first transgene and said second transgene are operably linked to a single promoter.

138. The method of claim 131 or 132, wherein said first donor nucleic acid sequence comprises, from 5′ to 3′, said first transgene, a first untranslated sequence, said first recognition sequence, and said first homology region,
wherein said second donor nucleic acid sequence comprises, from 5′ to 3′, said 5′ homology arm, a second untranslated sequence, an IRES or 2A element, and a second transgene,
wherein said 5′ homology arm comprises, from 5′ to 3′, a sequence having homology to at least a portion of said first transgene, a sequence having homology to said IRES or 2A element, a sequence having homology to said first untranslated sequence, and a sequence having homology to a 5′ portion of said first nuclease recognition sequence,
and wherein insertion of said second donor nucleic acid sequence into said first nuclease recognition sequence would generate a sequence comprising, from 5′ to 3′, said first transgene, said 2A or IRES element, said 5′ portion of said first nuclease recognition sequence flanked by said first and said second untranslated sequence, and said second transgene, such that said first transgene and said second transgene are operably linked to a single promoter.

139. The method of claim 137 or 138, wherein said first untranslated sequence is a first intron sequence comprising a splice donor sequence at its 5′ end, and said second untranslated sequence is a second intron sequence comprising a splice acceptor sequence at its 3′ end, wherein said splice donor sequence and said splice acceptor sequence are capable of being recognized by a splicing complex, and said first intron sequence, said 5′ portion of said first nuclease recognition sequence, and said second intron sequence are capable of being spliced from said first polynucleotide upon insertion of said second donor nucleic acid sequence into said first nuclease recognition sequence and expression of said first transgene and said second transgene.

140. The method of claim 131 or 132, wherein said first donor nucleic acid sequence comprises, from 5′ to 3′, said first transgene, said first recognition sequence, and said first homology region,
wherein said second donor nucleic acid sequence comprises, from 5′ to 3′, said 5′ homology arm, a second promoter, and a second transgene operably linked to a second promoter,
and wherein insertion of said second donor nucleic acid sequence into said first nuclease recognition sequence would generate a sequence comprising, from 5′ to 3′, said first transgene, said 5′ portion of said first nuclease recognition sequence, said second promoter, and said second transgene.

141. The method of claim 131 or 132, wherein said first donor nucleic acid sequence comprises, from 5′ to 3′, said first transgene, a second promoter, and a first untranslated sequence,
wherein said second donor nucleic acid sequence comprises, from 5′ to 3′, said 5′ homology arm and a second transgene,
wherein said 5′ homology arm has homology to at least a portion of said first untranslated sequence and to said 5′ portion of said first nuclease recognition sequence,
and wherein insertion of said second donor nucleic acid sequence into said first nuclease recognition sequence would generate a sequence comprising, from 5′ to 3′, said first transgene, said second promoter, said first untranslated sequence, said 5′ portion of said first nuclease recognition sequence, and said second transgene.

142. The method of claim 141, wherein said first untranslated sequence is an intron sequence comprising a splice donor sequence at its 5′ end and a splice acceptor sequence at its 3′ end, wherein said splice donor sequence and said splice acceptor sequence are capable of being recognized by a splicing complex, and said intron sequence is capable of being spliced from said first polynucleotide upon insertion of said second donor nucleic acid sequence into said first nuclease recognition sequence and expression of said second transgene.

143. The method of any one of claims 111-142, wherein said first transgene encodes:
(a) a chimeric antigen receptor;
(b) an exogenous TCR;
(c) an inhibitory nucleic acid;
(d) a reporter protein;
(e) a protein useful for purification of said genetically-modified eukaryotic cell;
(f) a therapeutic protein; or
(g) a suicide protein.

144. The method of any one of claims 135-143, wherein said first transgene and/or said second transgene encodes:
(a) a chimeric antigen receptor;
(b) an exogenous TCR;
(c) an inhibitory nucleic acid;
(d) a reporter protein;
(e) a protein useful for purification of said genetically-modified eukaryotic cell;
(f) a therapeutic protein; or
(g) a suicide protein.

145. The method of claim 143 or 144, wherein said inhibitory nucleic acid comprises an shRNA or microRNA-adapted shRNA.

146. The method of any one of claims 111-136 and 143-145, wherein said first transgene encodes a protein that exceeds 5 kilobases in size.

147. The method of any one of claims 111-146, wherein said first polynucleotide is comprised within a first recombinant virus or a first lipid nanoparticle, and/or said second polynucleotide is comprised within a second recombinant virus or a second lipid nanoparticle.

148. The method of claim 147, wherein said first recombinant virus is a first AAV, and/or wherein said second recombinant virus is a second AAV.

149. The method of claim 147, wherein said first AAV vector and/or said second AAV vector has a serotype of AAV1, AAV2, AAV3, AAV4, AAV5, AAV6, AAV7, AAV8, AAV9, AAV10, or AAV11.

150. The method of claim 148 or 149, wherein said first AAV vector and/or said second AAV vector has a serotype of AAV6.

151. The method of any one of claims 148-150, wherein said first polynucleotide comprises only one D sequence, and wherein said second polynucleotide comprises only one D sequence.

152. The method of claim 151, wherein said D sequence comprised by said first polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

153. The method of claim 151 or 152, wherein said D sequence comprised by said second polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

154. The method of any one of claims 151-153, wherein said D sequence comprised by said first polynucleotide:
(a) is positioned within a 5′ ITR;
(b) overlaps said 5′ ITR; or
(c) is positioned 3′ downstream of said 5′ ITR and 5′ upstream of said 5′ homology arm;
and wherein said D sequence comprised by said second polynucleotide:
(d) is positioned within a 5′ ITR;
(e) overlaps said 5′ ITR; or
(f) is positioned 3′ downstream of said 5′ ITR and 5′ upstream of said 5′ homology arm.

155. The method of any one of claims 151-153, wherein said D sequence comprised by said first polynucleotide:
(a) is positioned 5′ upstream of a 3′ ITR and 3′ downstream of said 3′ homology arm;
(b) overlaps said 3′ ITR; or
(c) is positioned within said 3′ ITR;
and wherein said D sequence comprised by said second polynucleotide:
(d) is positioned 5′ upstream of a 3′ ITR and 3′ downstream of said 3′ homology arm;
(e) overlaps said 3′ ITR; or
(f) is positioned within said 3′ ITR.

156. A method for inserting a transgene into the genome of a target cell in vivo, said method comprising delivering to a target cell in a subject:
(a) a first polynucleotide comprising a first heterologous nucleic acid sequence comprising:
(i) a first donor nucleic acid sequence comprising, from 5′ to 3′, a first portion of said transgene, a first untranslated sequence, and a first nuclease recognition sequence for a first engineered nuclease; and
(ii) a first homology region positioned 3′ downstream of said first nuclease recognition sequence;
(b) a second polynucleotide comprising a second heterologous nucleic acid sequence comprising:
(i) a 5′ homology arm having homology to at least a portion of said first donor nucleic acid sequence and to a 5′ portion of said first nuclease recognition sequence;
(ii) a 3′ homology arm having homology to a 3′ portion of said first nuclease recognition sequence and said first homology region; and
(iii) a second donor nucleic acid sequence positioned between said 5′ homology arm and said 3′ homology arm comprising, from 5′ to 3′, a second untranslated sequence and a second portion of said transgene; and
(c) one or more nucleic acids encoding one or more engineered nucleases, wherein said one or more engineered nucleases comprise said first engineered nuclease;
wherein said one or more engineered nucleases is expressed in said target cell and generate a first cleavage site at an endogenous nuclease recognition sequence normally present in the genome of said target cell,
wherein said first donor nucleic acid sequence is inserted into said first cleavage site,
wherein said one or more engineered nucleases generate a second cleavage site at said first nuclease recognition sequence,
wherein said second donor nucleic acid sequence is inserted into said second cleavage site such that the genome comprises a sequence comprising, from 5′ to 3′, said first portion of said first transgene, said 5′ portion of said first nuclease recognition sequence flanked by said first and said second untranslated sequence, and said second portion of said first transgene,
and wherein a full-length protein encoded by said transgene is expressed by said target cell.

157. The method of claim 156, wherein said first untranslated sequence is a first intron sequence comprising a splice donor sequence at its 5′ end, and said second untranslated sequence is a second intron sequence comprising a splice acceptor sequence at its 3′ end, wherein said splice donor sequence and said splice acceptor sequence are capable of being recognized by a splicing complex, and said first intron sequence, said 5′ portion of said first nuclease recognition sequence, and said second intron sequence are capable of being spliced from said first polynucleotide upon insertion of said second donor nucleic acid sequence into said first nuclease recognition sequence and expression of said first transgene.

158. The method of claim 156 or 157, wherein said transgene is at least 5 kilobases in size.

159. The method of any one of claims 156-158, wherein said first polynucleotide and said second polynucleotide are delivered simultaneously to said target cell.

160. The method of any one of claims 156-159, wherein said one or more nucleic acids encoding said one or more engineered nucleases is delivered to said target cell within at least 1 hour, 2 hours, 3 hours, 4 hours, 5 hours, 6 hours, 7 hours, 8 hours, 9 hours, 10 hours, 15 hours, 20 hours, or 24 hours of said first polynucleotide and said second polynucleotide.

161. The method of any one of claims 156-158, wherein said first polynucleotide and said second polynucleotide are delivered sequentially to said target cell.

162. The method of claim 161, wherein said second polynucleotide is delivered to said target cell within at least 1 hour, 2 hours, 3 hours, 4 hours, 5 hours, 6 hours, 7 hours, 8 hours, 9 hours, 10 hours, 15 hours, 20 hours, or 24 hours of said first polynucleotide.

163. The method of any one of claims 156-158, wherein said first polynucleotide, said second polynucleotide, and said one or more nucleic acids encoding said one or more engineered nucleases are delivered simultaneously to said target cell.

164. The method of any one of claims 156-163, wherein said one or more engineered nucleases is an engineered meganuclease, a TALEN, a compact TALEN, a zinc finger nuclease, a CRISPR system nuclease, or a megaTAL.

165. The method of any one of claims 156-163, wherein said one or more engineered nucleases is an engineered meganuclease.

166. The method of any one of claims 156-165, wherein said first polynucleotide further comprises:
(a) a 5′ homology arm that is homologous to a sequence 5′ upstream of said endogenous nuclease recognition sequence and to a 5′ portion of said endogenous nuclease recognition sequence; and
(b) a 3′ homology arm that is homologous to a sequence 3′ downstream of said endogenous nuclease recognition sequence and to a 3′ portion of said endogenous nuclease recognition sequence;
wherein said 5′ homology arm and said 3′ homology arm flank said first heterologous nucleic acid sequence.

167. The method of any one of claims 156-166, wherein said first nuclease recognition sequence is identical to said endogenous nuclease recognition sequence.

168. The method of any one of claims 156-165 and 167, wherein said first polynucleotide comprises a 5′ homology arm that is homologous to a sequence 5′ upstream of said endogenous nuclease recognition sequence and to a 5′ portion of said endogenous nuclease recognition sequence, and wherein said first homology region is homologous to a sequence 3′ downstream of said endogenous nuclease recognition sequence.

169. The method of any one of claims 156-168, wherein said endogenous nuclease recognition sequence is within a T cell receptor (TCR) alpha gene or a TCR beta gene.

170. The method of any one of claims 156-169, wherein said endogenous nuclease recognition sequence is within a TCR alpha constant (TRAC) gene or TCR beta constant (TRBC) gene.

171. The method of any one of claims 156-170, wherein said first nuclease recognition sequence comprises SEQ ID NO: 1.

172. The method of any one of claims 156-171, wherein said one or more nucleic acids encoding said one or more engineered nucleases are mRNA, or wherein said one or more nucleic acids encoding said one or more engineered nucleases are comprised within one or more nuclease AAVs.

173. The method of any one of claims 156-172, wherein said second donor nucleic acid sequence does not comprise a second nuclease recognition sequence, or does not comprise a 5′ portion of a nuclease recognition sequence capable of pairing with said 3′ portion of said first nuclease recognition sequence to generate a second nuclease recognition sequence, or does not comprise a 3′ portion of a nuclease recognition sequence capable of pairing with said 5′ portion of said first nuclease recognition sequence to generate a second nuclease recognition sequence.

174. The method of any one of claims 156-172, wherein said second donor nucleic acid sequence comprises a second nuclease recognition sequence, or comprises a 5′ portion of a nuclease recognition sequence capable of pairing with said 3′ portion of said first nuclease recognition sequence to generate a second nuclease recognition sequence, or comprises a 3′ portion of a nuclease recognition sequence capable of pairing with said 5′ portion of said first nuclease recognition sequence to generate a second nuclease recognition sequence.

175. The method of claim 174, wherein said engineered nuclease is capable of binding and cleaving said first nuclease recognition sequence, said second nuclease recognition sequence, and said endogenous nuclease recognition sequence.

176. The method of claim 174 or 175, wherein said first nuclease recognition sequence, said second nuclease recognition sequence, and said endogenous nuclease recognition sequence are identical.

177. The method of any one of claims 156-176, wherein said first donor nucleic acid sequence comprises a first promoter that is operably linked to said transgene, or a sequence that operably links said first transgene to an endogenous promoter of said target cell.

178. The method of any one of claims 156-177, wherein said transgene encodes a therapeutic protein.

179. The method of claim 178, wherein said therapeutic protein is expressed at a clinically therapeutic level in said subject.

180. The method of any one of claims 156-179, wherein said first polynucleotide is comprised within a first recombinant AAV or a first lipid nanoparticle, and/or said second polynucleotide is comprised within a second recombinant AAV or a second lipid nanoparticle.

181. The method of claim 180, wherein said first recombinant AAV and/or said second recombinant AAV has a serotype of AAV1, AAV2, AAV3, AAV4, AAV5, AAV6, AAV7, AAV8, AAV9, AAV10, or AAV11.

182. The method of claim 180 or 181, wherein said first recombinant AAV and/or said second recombinant AAV has a serotype of AAV8.

183. The method of any one of claims 180-181, wherein said first polynucleotide comprises only one D sequence, and wherein said second polynucleotide comprises only one D sequence.

184. The method of claim 183, wherein said D sequence comprised by said first polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

185. The method of claim 183 or 184, wherein said D sequence comprised by said second polynucleotide:
(a) is positioned within a 5′ ITR;
(b) overlaps said 5′ ITR;
(c) is positioned 3′ downstream of said 5′ ITR and 5′ upstream of said 5′ homology arm;
(d) is positioned 5′ upstream of a 3′ ITR and 3′ downstream of said 3′ homology arm;
(e) overlaps said 3′ ITR; or
(f) is positioned within said 3′ ITR.

186. The method of any one of claims 183-185, wherein said D sequence comprised by said first polynucleotide:
(a) is positioned within a 5′ ITR;
(b) overlaps said 5′ ITR; or
(c) is positioned 3′ downstream of said 5′ ITR and 5′ upstream of said 5′ homology arm;
and wherein said D sequence comprised by said second polynucleotide:
(d) is positioned within a 5′ ITR;
(e) overlaps said 5′ ITR; or
(f) is positioned 3′ downstream of said 5′ ITR and 5′ upstream of said 5′ homology arm.

187. The method of any one of claims 183-185, wherein said D sequence comprised by said first polynucleotide:
(a) is positioned 5′ upstream of a 3′ ITR and 3′ downstream of said 3′ homology arm;
(b) overlaps said 3′ ITR; or
(c) is positioned within said 3′ ITR;
and wherein said D sequence comprised by said second polynucleotide:
(d) is positioned 5′ upstream of a 3′ ITR and 3′ downstream of said 3′ homology arm;
(e) overlaps said 3′ ITR; or
(f) is positioned within said 3′ ITR.

188. The method of any one of claims 156-187, wherein said target cell is a mammalian cell.

189. The method of claim 188, wherein said mammalian cell is human cell.


SEQUENCE LISTING (40 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 9 aa, Chlamydomonas reinhardtii
LAGLIDADG

SEQ ID NO: 3 - DNA, 3925 bp, Artificial Sequence
  Synthesized
ctccatcactaggggttcctacgcgtagatctcgagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaactagtgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgaggtaccagcggccgcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaactggtactagtacgctagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagagacccgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcaggcctggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggaggaattgatgga

SEQ ID NO: 4 - DNA, 2519 bp, Artificial Sequence
  Synthesized
ctccatcactaggggttcctacgcgtagtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcagcctggcctggagcaagccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaacttgtttattgcagcttataatggttacaaataaagcaatagcatcacaaatttcacaaataaagcatttttttcactgcattctagttgtggtttgtccaaactcatcaatgtatcttaatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatggagttggccactccctct

SEQ ID NO: 5 - DNA, 2512 bp, Artificial Sequence
  Synthesized
ctccatcactaggggttcctacgcgtagtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcaggcctggcctggagcaagccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaacttgtttattgcagcttataatggttacaaataaagcaatagcatcacaaatttcacaaataaagcatttttttcactgcattctagttgtggtttgtccaaactcatcaatgtatcttaacgcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatggagttggccactccctct

SEQ ID NO: 6 - DNA, 2518 bp, Artificial Sequence
  Synthesized
ctccatcactaggggttcctacgcgtagtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcaggcctggcctggagcaagccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaacttgtttattgcagcttataatggttacaaataaagcaatagcatcacaaatttcacaaataaagcatttttttcactgcattctagttgtggtttgtccaaactcatcaatgtatcttaacaagacattggtgaggaaaaatcgcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatgga

SEQ ID NO: 7 - DNA, 4224 bp, Artificial Sequence
  Synthesized
ctccatcactaggggttcctaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtacgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgaggtaccagcggccgcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaactggtactagtacgctagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagagacccgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcaggcctggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggaggtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatcaattgatggagttggccactccctct

SEQ ID NO: 8 - DNA, 2124 bp, Artificial Sequence
  Synthesized
ctccatcactaggggttcctacgcgtagaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacatggcctggagcaagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaacttgtttattgcagcttataatggttacaaataaagcaatagcatcacaaatttcacaaataaagcatttttttcactgcattctagttgtggtttgtccaaactcatcaatgtatcttaatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatggagttggccactccctct

SEQ ID NO: 9 - DNA, 3100 bp, Artificial Sequence
  Synthesized
ctccatcactaggggttcctacgcgtagatctcatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaagctagcggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaccggtgccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaaggtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatgga

SEQ ID NO: 10 - DNA, 4299 bp, Artificial Sequence
  Synthesized
ctccatcactaggggttcctacgcgtagatctcgagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaactagtgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgaggtaccagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagcggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaccggtgccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaaggtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatgga

SEQ ID NO: 11 - DNA, 3006 bp, Artificial Sequence
  Synthesized
ctccatcactaggggttcctacgcgtagatctcgagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaactagtgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgaggtaccagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagctggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatgga

SEQ ID NO: 12 - DNA, 2864 bp, Artificial Sequence
  Synthesized
ctccatcactaggggttcctacgcgtacgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgaggtaccagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagctggcctggagcaagctagcggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaccggtgccaccatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagtcgacaacttgtttattgcagcttataatggttacaaataaagcaatagcatcacaaatttcacaaataaagcatttttttcactgcattctagttgtggtttgtccaaactcatcaatgtatcttaatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatggagttggccactccctct

SEQ ID NO: 13 - DNA, 4024 bp, Artificial Sequence
  Synthesized
ctccatcactaggggttcctacgcgtagatctcgagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaactagtgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgaggtaccagcggccgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggctagcggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaccggtgccacctgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcagtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatgga

SEQ ID NO: 14 - DNA, 3338 bp, Artificial Sequence
  Synthesized
ctccatcactaggggttcctacgcgtagatctcgagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaactagtgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgggccaagagatctggaagcggcgccacaaatttctcactgctgaaacaggccggcgacgtggaagagaaccctggacctgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatgga

SEQ ID NO: 15 - DNA, 2807 bp, Artificial Sequence
  Synthesized
ctccatcactaggggttcctacgcgtacgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatgggccaagagatctggaagcggcgccacaaatttctcactgctgaaacaggccggcgacgtggaagagaaccctggacctgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacatggcctggagcaagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcagatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaacttgtttattgcagcttataatggttacaaataaagcaatagcatcacaaatttcacaaataaagcatttttttcactgcattctagttgtggtttgtccaaactcatcaatgtatcttaatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatggagttggccactccctct

SEQ ID NO: 16 - DNA, 3257 bp, Artificial Sequence
  Synthesized
ctccatcactaggggttcctacgcgtagatctcgagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaactagtgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgttccggaaagccaccatggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgagtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatgga

SEQ ID NO: 17 - DNA, 2810 bp, Artificial Sequence
  Synthesized
ctccatcactaggggttcctacgcgtacgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgagaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacatggcctggagcaagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcagcgggccaagagatctggaagcggcgccacaaatttctcactgctgaaacaggccggcgacgtggaagagaaccctggacctatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagtcgacaacttgtttattgcagcttataatggttacaaataaagcaatagcatcacaaatttcacaaataaagcatttttttcactgcattctagttgtggtttgtccaaactcatcaatgtatcttaatggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccaattgatggagttggccactccctct

SEQ ID NO: 18 - DNA, 20 bp, Artificial Sequence
  Synthesized
ctccatcactaggggttcct

SEQ ID NO: 19 - DNA, 22 bp, Homo sapiens
tggcctggagcaacaaatctga

SEQ ID NO: 20 - DNA, 13 bp, Homo sapiens
tggcctggagcaa

SEQ ID NO: 21 - DNA, 13 bp, Homo sapiens
gcaacaaatctga

SEQ ID NO: 22 - DNA, 22 bp, Homo sapiens
ctatctcttgtactacactgaa

SEQ ID NO: 23 - DNA, 13 bp, Homo sapiens
ctatctcttgtac

SEQ ID NO: 24 - DNA, 13 bp, Homo sapiens
gtactacactgaa

SEQ ID NO: 25 - DNA, 22 bp, Homo sapiens
aagacattggtgaggaaaaatc

SEQ ID NO: 26 - DNA, 237 bp, Artificial Sequence
  Synthesized
atgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaag

SEQ ID NO: 27 - DNA, 477 bp, Artificial Sequence
  Synthesized
cagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 28 - DNA, 713 bp, Artificial Sequence
  Synthesized
atgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcaaaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaa

SEQ ID NO: 29 - DNA, 1458 bp, Artificial Sequence
  Synthesized
atggcgctcccagtgacagccttacttttacctctggcgttattattgcacgcggctcgtcctgacatacagatgactcagactacctcttccctatctgcttctttaggcgaccgagtaacaatatcttgccgggccagccaggacatctcaaaatacttaaactggtatcagcagaagccggacggaacagttaagttgctcatttaccacacgtcgagattacactcaggcgttcctagccgattttcgggttccggttccggtacggactacagcctgacaatcagtaaccttgagcaggaggacatcgccacctacttctgtcagcagggcaacacgctcccgtacacattcggtgggggaactaagctggagattaccggaggcggtggcagcggtggcggcggcagcgggggtggcggctcggaggtcaagttacaggagagcggaccgggcttggtcgcacctagccagagcctctcagtcacgtgcactgtgtctggagtcagtctcccagactacggggtatcatggatacgacagccgcctagaaagggcttagagtggctgggggttatctggggaagtgaaaccacatactacaactcagctctcaagagccgcctcaccatcattaaggacaacagtaagtcgcaggttttcttaaagatgaactctctccagactgacgacaccgctatttactactgcgcgaagcactactactacggcgggagttacgcaatggactactggggtcagggcacttctgtgaccgtatccagcactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 30 - DNA, 225 bp, Artificial Sequence
  Synthesized
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 31 - DNA, 122 bp, Artificial Sequence
  Synthesized
aacttgtttattgcagcttataatggttacaaataaagcaatagcatcacaaatttcacaaataaagcatttttttcactgcattctagttgtggtttgtccaaactcatcaatgtatctta

SEQ ID NO: 32 - DNA, 826 bp, Artificial Sequence
  Synthesized
gtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcag

SEQ ID NO: 33 - DNA, 350 bp, Artificial Sequence
  Synthesized
gaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaaca

SEQ ID NO: 34 - DNA, 326 bp, Artificial Sequence
  Synthesized
gtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaagcttggagggatccgtagtctgagcagtactcgttgctgccgcgcgcgccaccagacataatagctgacagactaacagactgttcctttccatgggtcttttctgcag

SEQ ID NO: 35 - DNA, 2 bp, Artificial Sequence
  Synthesized
gt

SEQ ID NO: 36 - DNA, 2 bp, Artificial Sequence
  Synthesized
ag

SEQ ID NO: 37 - DNA, 164 bp, Artificial Sequence
  Synthesized
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 38 - DNA, 544 bp, Artificial Sequence
  Synthesized
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 39 - DNA, 81 bp, Artificial Sequence
  Synthesized
cgggccaagagatctggaagcggcgccacaaatttctcactgctgaaacaggccggcgacgtggaagagaaccctggacct

SEQ ID NO: 40 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP